SEQUENCE LISTING (2 sequences)

SEQ ID NO: 1 - DNA, 6 bp, Artificial Sequence
  DNA Sequence 1
attgcc

SEQ ID NO: 2 - DNA, 6 bp, Artificial Sequence
  DNA Sequence 2
tatggc

(19) United States
(12) Patent Application Publication
(10) Pub. No.: US 2025/0346633 A1
(43) Pub. Date: Nov. 13, 2025

(54) MACOLACINS AND METHODS OF USE THEREOF

(71) Applicant: The Rockefeller University, New York, NY (US)

(72) Inventors: Sean Brady, New York, NY (US); Zongqiang Wang, New York, NY (US); Bimal Koirala, New York, NY (US)

(21) Appl. No.: 18/557,654

(22) PCT Filed: Apr. 27, 2022
(86) PCT No.: PCT/US22/26504

§ 371 Date: Oct. 27, 2023

Related U.S. Application Data
(60) Provisional application No. 63/180,348, filed on Apr. 27, 2021.
(60) Provisional application No. 63/270,777, filed on Oct. 22, 2021.

(51) Int. Cl. C07K 7/62; A61K 38/12

(52) U.S. Cl. CPC C07K 7/62; A61K 38/12

(57) ABSTRACT

The present invention provides methods, compositions, and articles of manufacture useful for the prophylactic and therapeutic amelioration and treatment of gram-positive bacteria, and related conditions. The present invention provides compositions and methods incorporating and utilizing macolacin antibiotics or derivatives or variants thereof.

### CROSS-REFERENCE TO RELATED APPLICATIONS

This application claims the priority under 35 U.S.C. § 119 (e) to U.S. Provisional Patent Application Ser. No. 63/180,348, filed Apr. 27, 2021, and U.S. Provisional Patent Application Ser. No. 63/270,777, filed Oct. 22, 2021, the disclosures of which are incorporated herein by reference in their entireties.

### STATEMENT OF GOVERNMENT FUNDING

This invention was made with government support under 1U19AI142731 and 5R35GM122559 awarded by the National Institutes of Health. The government has certain rights in the invention.

### BACKGROUND OF THE INVENTION

Despite the wide availability of antibiotics, infectious diseases remain a leading cause of death worldwide. In the absence of new therapies, mortality rates due to untreatable infections are predicted to rise more than tenfold by 2050. Natural products (NPs) made by cultured bacteria have been a major source of clinically useful antibiotics. In spite of decades of productivity, the use of bacteria in the search for new antibiotics was largely abandoned due to high rediscovery rates (Tringe, S. G. et al., 2005, Science 308, 554-557; Reddy, B. V. et al., 2012, Appl. Environ. Microbiol. 78, 3744-3752).

Furthermore, multidrug-resistant (MDR) gram-negative bacteria represent a serious and growing risk to public health (Tacconelli E et al., 2018, Lancet Infect Dis, 18:318-327; Ventola C L et al., 2015, 40:277-283). Many critical gram-negative active antibiotics in use today are either bacterial metabolites or inspired by bacterial metabolites (Payne D J et al., 2007, Nat Rev Drug Discov, 6:29-40; Imai Y et al., 2019, Nature, 576:459-464). In fact, the bacterial natural product colistin is used as the last line of defense against serious infections caused by a number of MDR Gram−negative pathogens, especially those with carbapenem resistance (Biswas S et al., 2012, Expert Review of Anti-Infective Therapy, 10:917-934; Liu Y Y et al., 2016, Lancet Infectious Diseases, 16:161-168).

Thus, there is a need in the art for new compositions and methods for treating infections. The present invention satisfies the need in the art.

### SUMMARY OF THE INVENTION

The present invention relates, in part, to a compound represented by Formula (I)

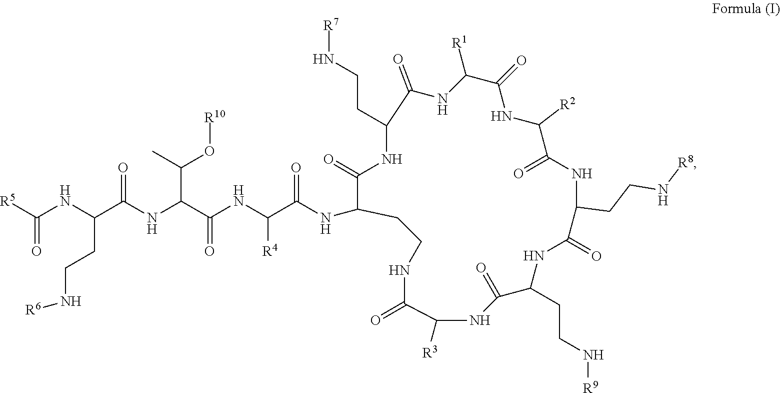

or a racemate, an enantiomer, a diastereomer, a pharmaceutically acceptable salt, or a derivative thereof.

In some embodiments, each R¹, R², R³, R⁴, R⁵, R⁶, R⁷, R⁸, R⁹, and R¹⁰ is independently selected from hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof. In some embodiments, each R¹, R², R³, R⁴, and R⁵ is independently selected from alkyl, alkenyl, aryl, aryl alkyl, aminoalkyl, hydroxyalkyl, or any combination thereof. In some embodiments, each R¹, R², R³, R⁴, and R⁵ is independently selected from linear C₁-C₁₀ alkyl, branched C₁-C₁₀ alkyl, linear aryl-C₁-C₁₀ alkyl, branched aryl-C₁-C₁₀ alkyl, linear amino-C₁-C₁₀ alkyl, amino-branched C₁-C₁₀ alkyl, linear hydroxy-C₁-C₁₀ alkyl, hydroxy-branched C₁-C₁₀ alkyl, linear C₁-C₁₀ alkenyl, branched C₁-C₁₀ alkenyl, linear aryl-C₁-C₁₀ alkenyl, branched aryl-C₁-C₁₀ alkenyl, linear amino-C₁-C₁₀ alkenyl, amino-branched C₁-C₁₀ alkenyl, linear hydroxy-C₁-C₁₀ alkenyl, hydroxy-branched C₁-C₁₀ alkenyl,

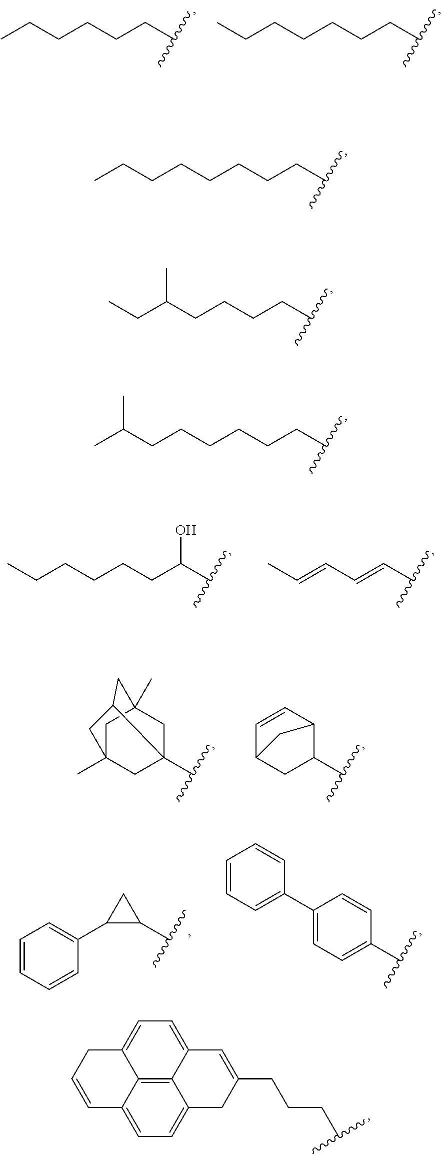

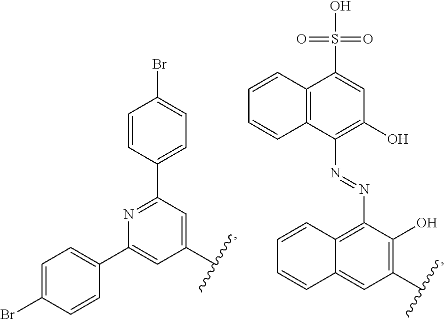 [SMILES: *c1cc(-c2ccc(Br)cc2)nc(-c2ccc(Br)cc2)c1 ; *c1cc2ccccc2c(/N=N/c2c(O)cc(S(=O)(=O)O)c3ccccc23)c1O]

or any combination thereof. In some embodiments, each R⁶, R⁷, R⁸, R⁹, and R¹⁰ is independently selected from hydrogen, sulfonyl, or alkyl sulfonyl. In one embodiment, the alkyl sulfonyl is methanesulfonyl.

In some embodiments, the compound represented by Formula (I) is a compound selected from:

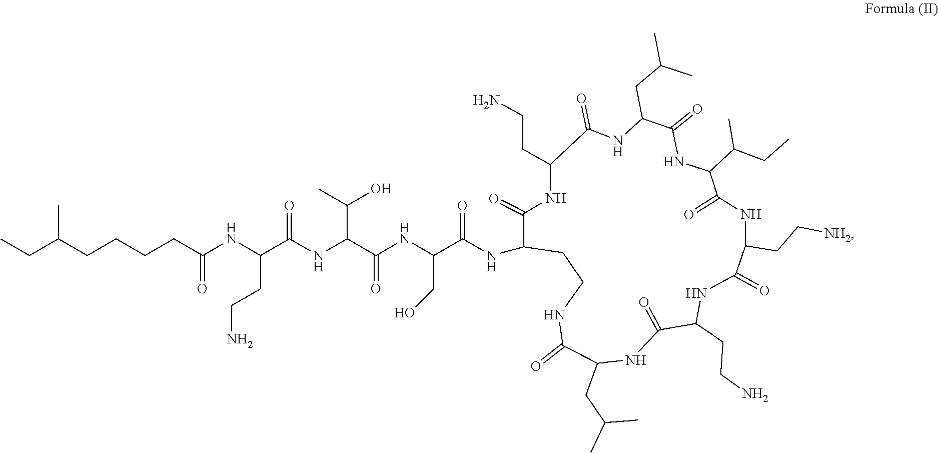 [SMILES: CCC(C)CCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]

or a racemate, an enantiomer, a diastereomer, a pharmaceutically acceptable salt, or a derivative

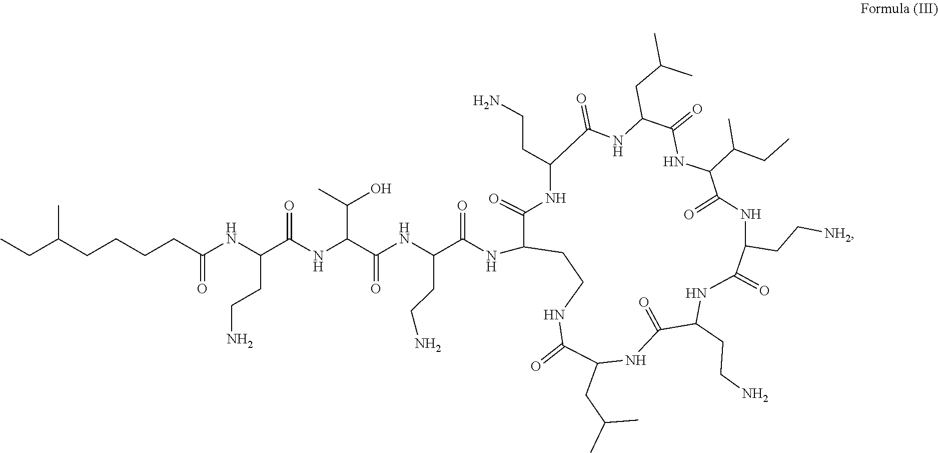 [SMILES: CCC(C)CCCCC(=O)NC(CCN)C(=O)NC(C(=O)NC(CCN)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]

or a racemate, an enantiomer, a diastereomer, a pharmaceutically acceptable salt, or a derivative thereof,

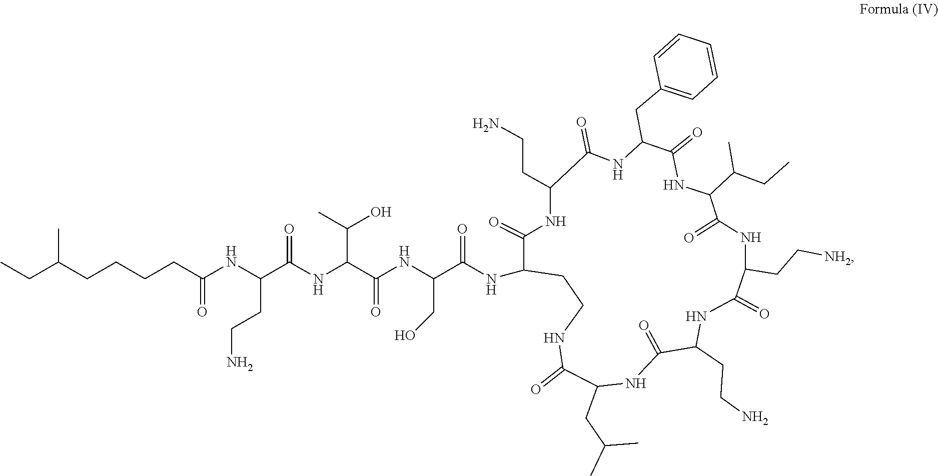 [SMILES: CCC(C)CCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(Cc2ccccc2)NC(=O)C(CCN)NC1=O)C(C)O]

or a racemate, an enantiomer, a diastereomer, a pharmaceutically acceptable salt, or a derivative

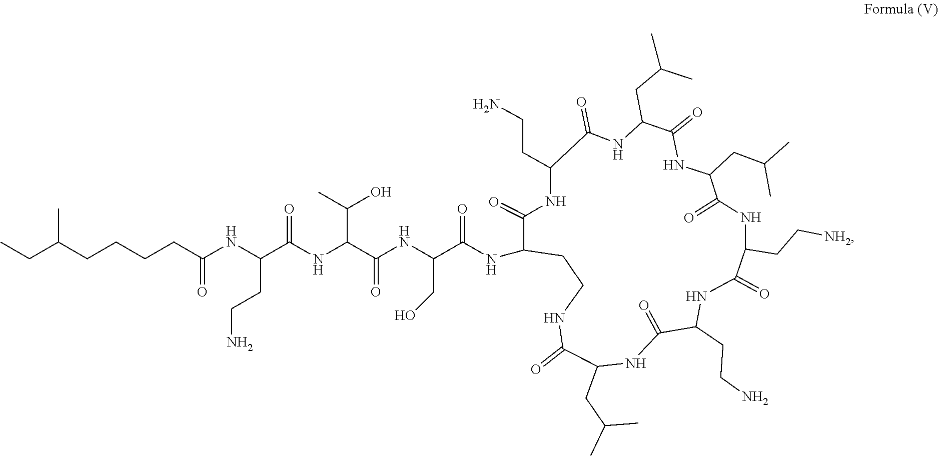 [SMILES: CCC(C)CCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(CC(C)C)NC(=O)C(CC(C)C)NC(=O)C(CCN)NC1=O)C(C)O]

or a racemate, an enantiomer, a diastereomer, a pharmaceutically acceptable salt, or a derivative thereof,

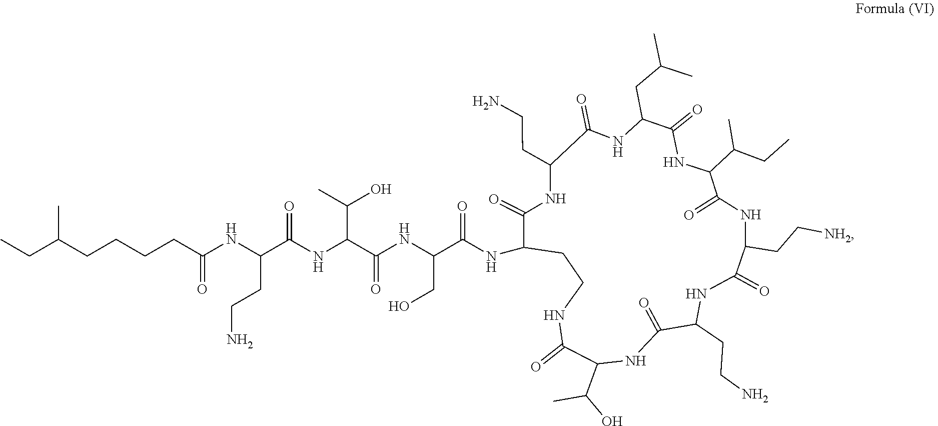 [SMILES: CCC(C)CCCCC(=O)NC(CCN)C(=O)NC(C(=O)NC(CO)C(=O)NC1CCNC(=O)C(C(C)O)NC(=O)C(CCN)NC(=O)C(CCN)NC(=O)C(C(C)CC)NC(=O)C(CC(C)C)NC(=O)C(CCN)NC1=O)C(C)O]

or a racemate, an enantiomer, a diastereomer, a pharmaceutically acceptable salt, or a derivative thereof,

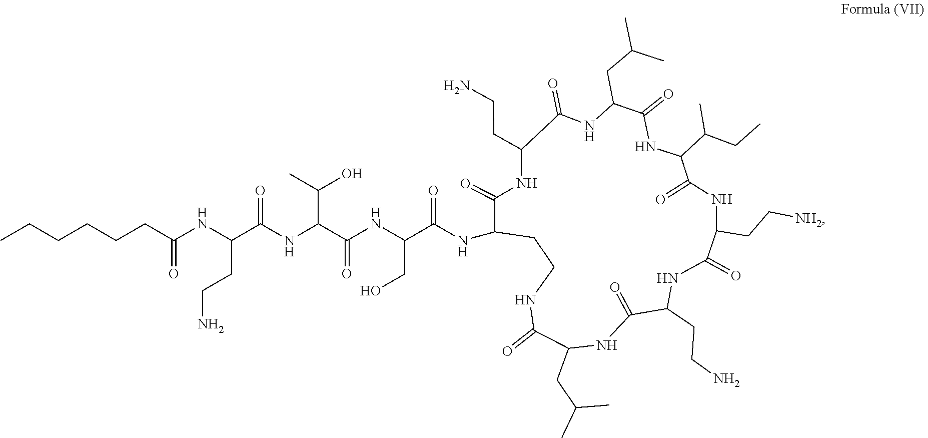 [SMILES: CCCCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]

or a racemate, an enantiomer, a diastereomer, a pharmaceutically acceptable salt, or a derivative thereof,

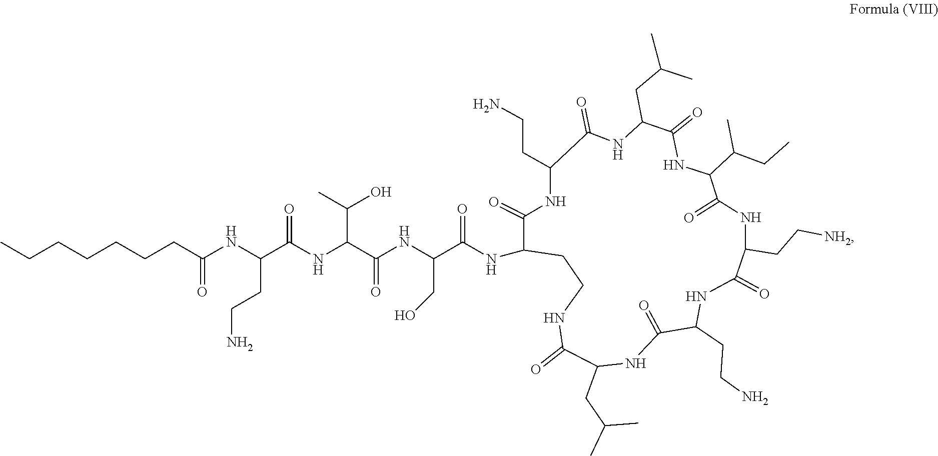 [SMILES: CCCCCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]

or a racemate, an enantiomer, a diastereomer, a pharmaceutically acceptable salt, or a derivative

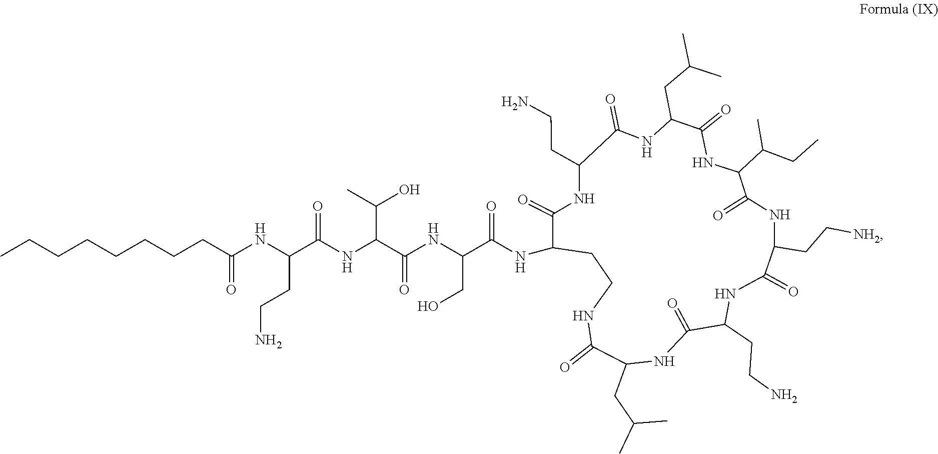 [SMILES: CCCCCCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]

or a racemate, an enantiomer, a diastereomer, a pharmaceutically acceptable salt, or a derivative thereof,

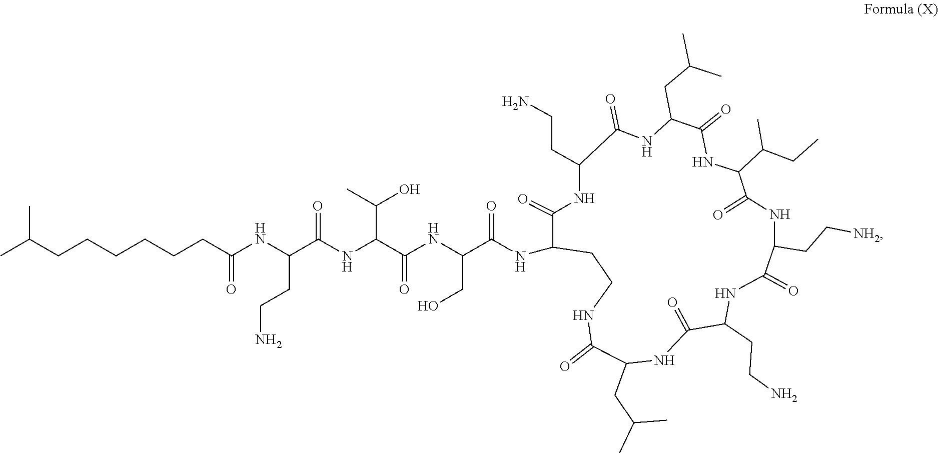 [SMILES: CCC(C)C1NC(=O)C(CC(C)C)NC(=O)C(CCN)NC(=O)C(NC(=O)C(CO)NC(=O)C(NC(=O)C(CCN)NC(=O)CCCCCCC(C)C)C(C)O)CCNC(=O)C(CC(C)C)NC(=O)C(CCN)NC(=O)C(CCN)NC1=O]

or a racemate, an enantiomer, a diastereomer, a pharmaceutically acceptable salt, or a derivative thereof,

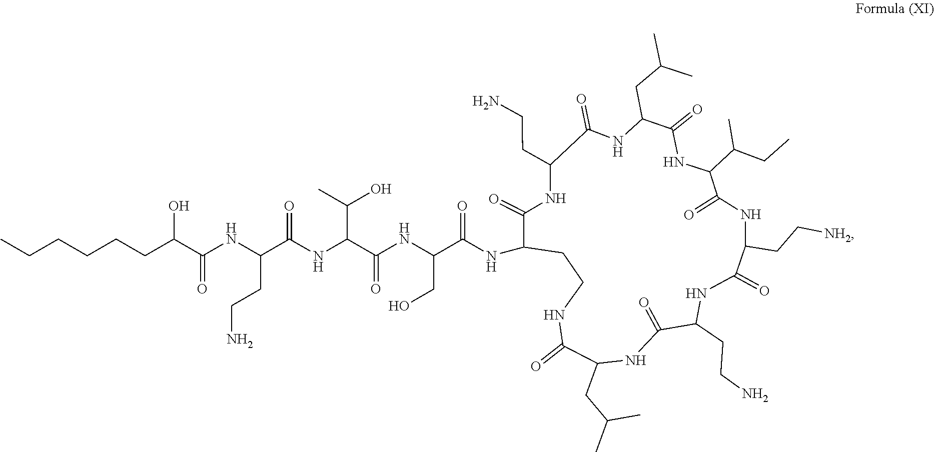 [SMILES: CCCCCCC(O)C(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]

or a racemate, an enantiomer, a diastereomer, a pharmaceutically acceptable salt, or a derivative

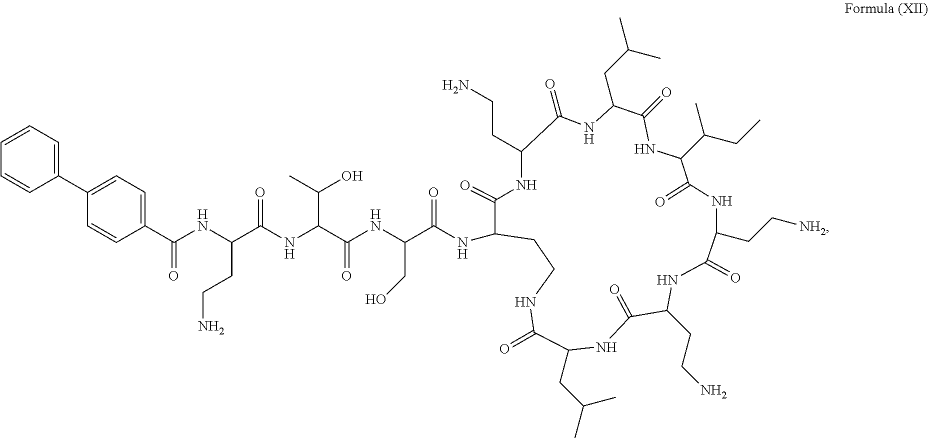 [SMILES: CCC(C)C1NC(=O)C(CC(C)C)NC(=O)C(CCN)NC(=O)C(NC(=O)C(CO)NC(=O)C(NC(=O)C(CCN)NC(=O)c2ccc(-c3ccccc3)cc2)C(C)O)CCNC(=O)C(CC(C)C)NC(=O)C(CCN)NC(=O)C(CCN)NC1=O]

or a racemate, an enantiomer, a diastereomer, a pharmaceutically acceptable salt, or a derivative thereof,

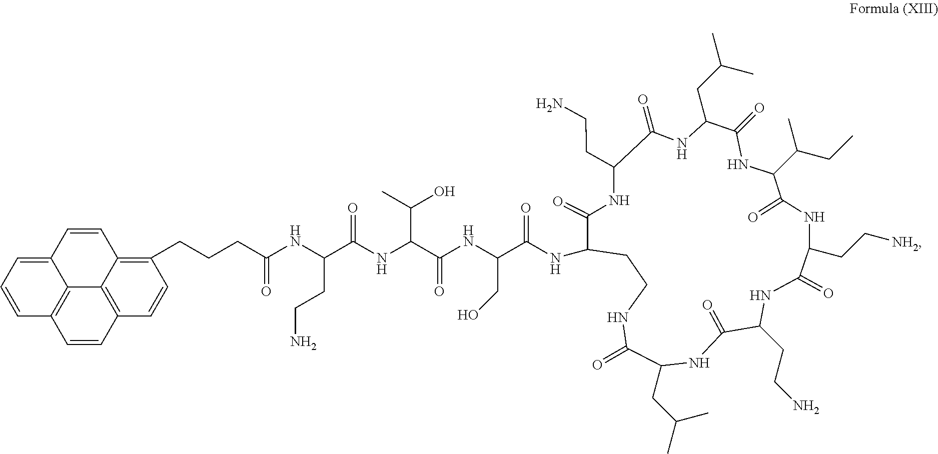 [SMILES: CCC(C)C1NC(=O)C(CC(C)C)NC(=O)C(CCN)NC(=O)C(NC(=O)C(CO)NC(=O)C(NC(=O)C(CCN)NC(=O)CCCc2ccc3ccc4cccc5ccc2c3c45)C(C)O)CCNC(=O)C(CC(C)C)NC(=O)C(CCN)NC(=O)C(CCN)NC1=O]

or a racemate, an enantiomer, a diastereomer, a pharmaceutically acceptable salt, or a derivative thereof,

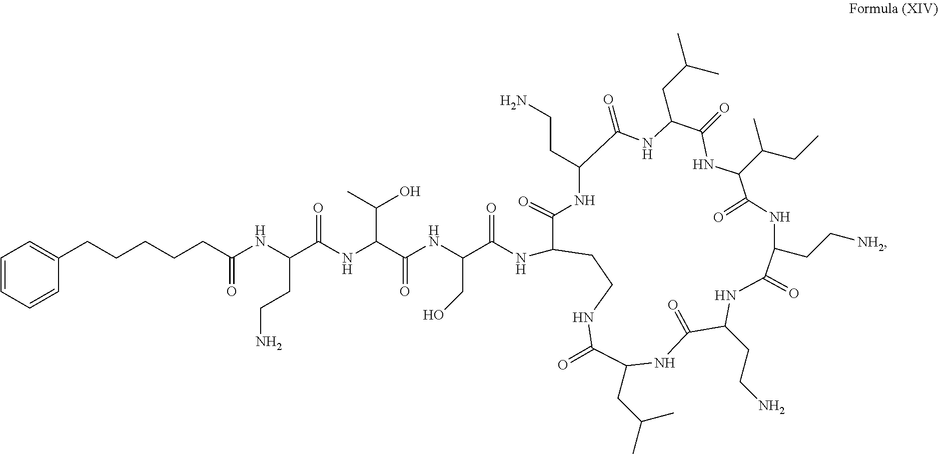 [SMILES: CCC(C)C1NC(=O)C(CC(C)C)NC(=O)C(CCN)NC(=O)C(NC(=O)C(CO)NC(=O)C(NC(=O)C(CCN)NC(=O)CCCCCc2ccccc2)C(C)O)CCNC(=O)C(CC(C)C)NC(=O)C(CCN)NC(=O)C(CCN)NC1=O]

or a racemate, an enantiomer, a diastereomer, a pharmaceutically acceptable salt, or a derivative

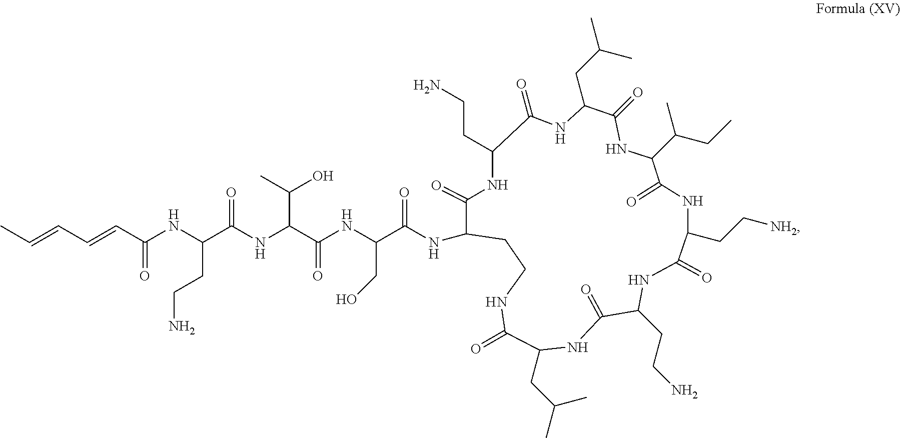 [SMILES: C/C=C/C=C/C(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]

or a racemate, an enantiomer, a diastereomer, a pharmaceutically acceptable salt, or a derivative thereof,

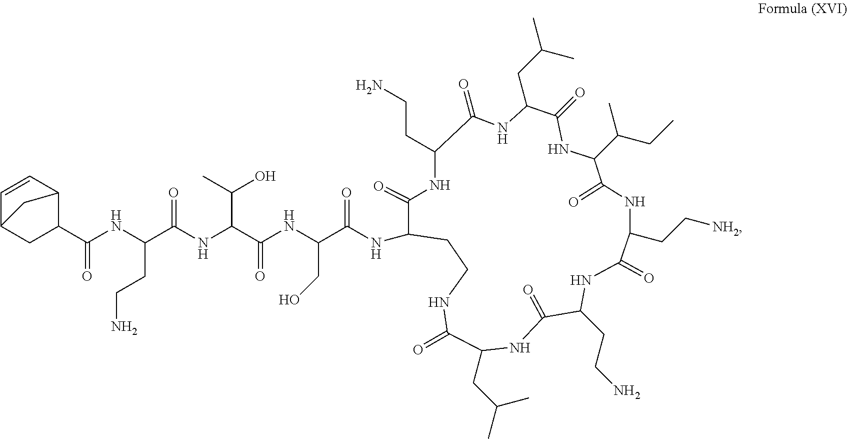 [SMILES: CCC(C)C1NC(=O)C(CC(C)C)NC(=O)C(CCN)NC(=O)C(NC(=O)C(CO)NC(=O)C(NC(=O)C(CCN)NC(=O)C2CC3C=CC2C3)C(C)O)CCNC(=O)C(CC(C)C)NC(=O)C(CCN)NC(=O)C(CCN)NC1=O]

or a racemate, an enantiomer, a diastereomer, a pharmaceutically acceptable salt, or a derivative thereof,

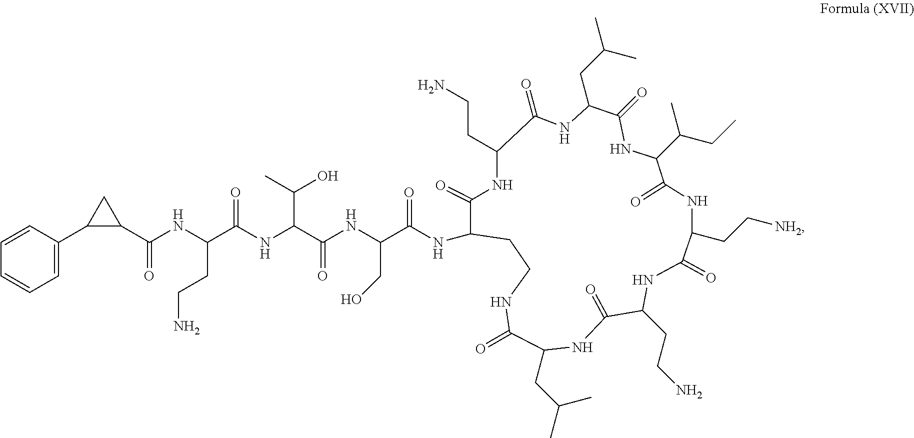 [SMILES: CCC(C)C1NC(=O)C(CC(C)C)NC(=O)C(CCN)NC(=O)C(NC(=O)C(CO)NC(=O)C(NC(=O)C(CCN)NC(=O)C2CC2c2ccccc2)C(C)O)CCNC(=O)C(CC(C)C)NC(=O)C(CCN)NC(=O)C(CCN)NC1=O]

or a racemate, an enantiomer, a diastereomer, a pharmaceutically acceptable salt, or a derivative

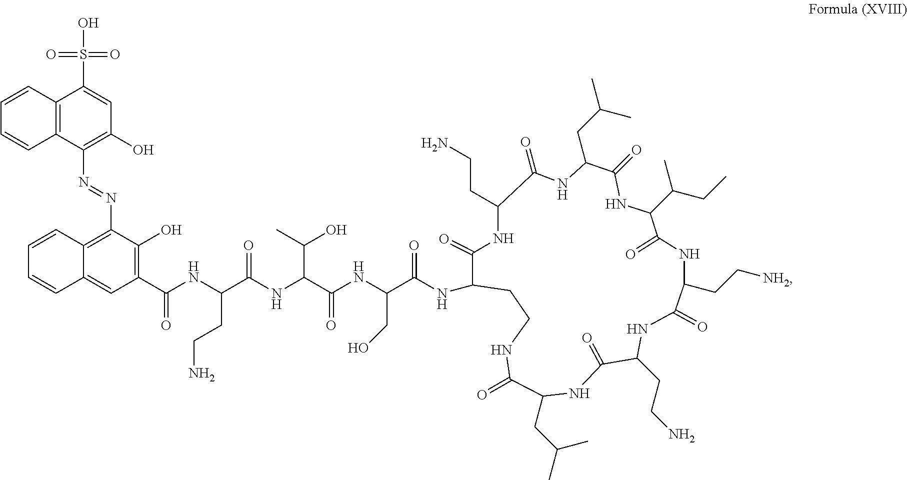 [SMILES: CCC(C)C1NC(=O)C(CC(C)C)NC(=O)C(CCN)NC(=O)C(NC(=O)C(CO)NC(=O)C(NC(=O)C(CCN)NC(=O)c2cc3ccccc3c(/N=N/c3c(O)cc(S(=O)(=O)O)c4ccccc34)c2O)C(C)O)CCNC(=O)C(CC(C)C)NC(=O)C(CCN)NC(=O)C(CCN)NC1=O]

or a racemate, an enantiomer, a diastereomer, a pharmaceutically acceptable salt, or a derivative thereof,

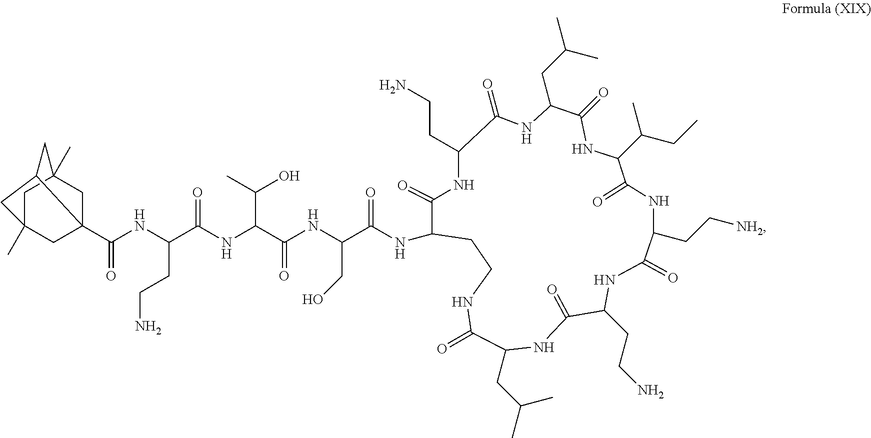 [SMILES: CCC(C)C1NC(=O)C(CC(C)C)NC(=O)C(CCN)NC(=O)C(NC(=O)C(CO)NC(=O)C(NC(=O)C(CCN)NC(=O)C23CC4(C)CC2CC(C)(C4)C3)C(C)O)CCNC(=O)C(CC(C)C)NC(=O)C(CCN)NC(=O)C(CCN)NC1=O]

or a racemate, an enantiomer, a diastereomer, a pharmaceutically acceptable salt, or a derivative thereof, or

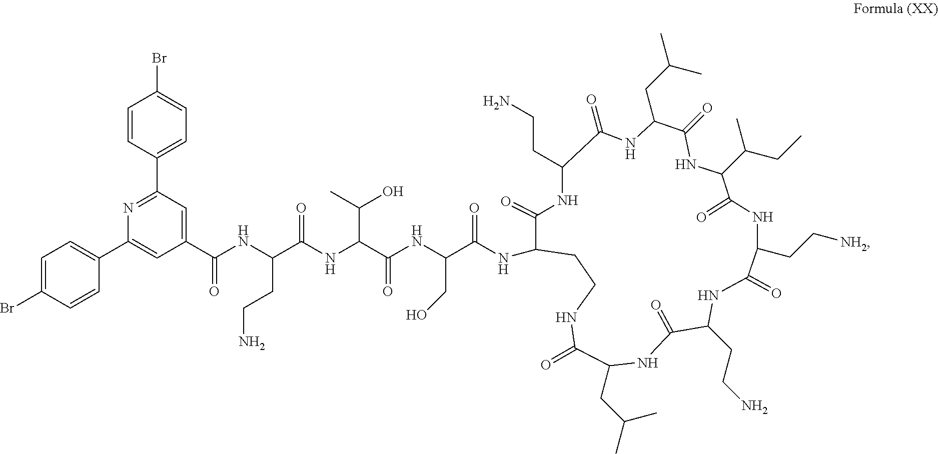 [SMILES: CCC(C)C1NC(=O)C(CC(C)C)NC(=O)C(CCN)NC(=O)C(NC(=O)C(CO)NC(=O)C(NC(=O)C(CCN)NC(=O)c2cc(-c3ccc(Br)cc3)nc(-c3ccc(Br)cc3)c2)C(C)O)CCNC(=O)C(CC(C)C)NC(=O)C(CCN)NC(=O)C(CCN)NC1=O]

or a racemate, an enantiomer, a diastereomer, a pharmaceutically acceptable salt, or a derivative thereof.

In one embodiment, the compound represented by Formula (I) is a compound represented by Formula (Ia)

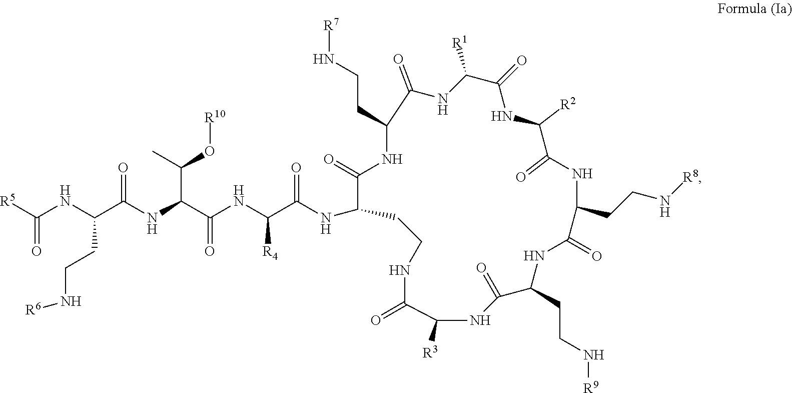

or a racemate, an enantiomer, a diastereomer, a pharmaceutically acceptable salt, or a derivative thereof.

In some embodiments, the compound represented by Formula (Ia) is a compound selected from:

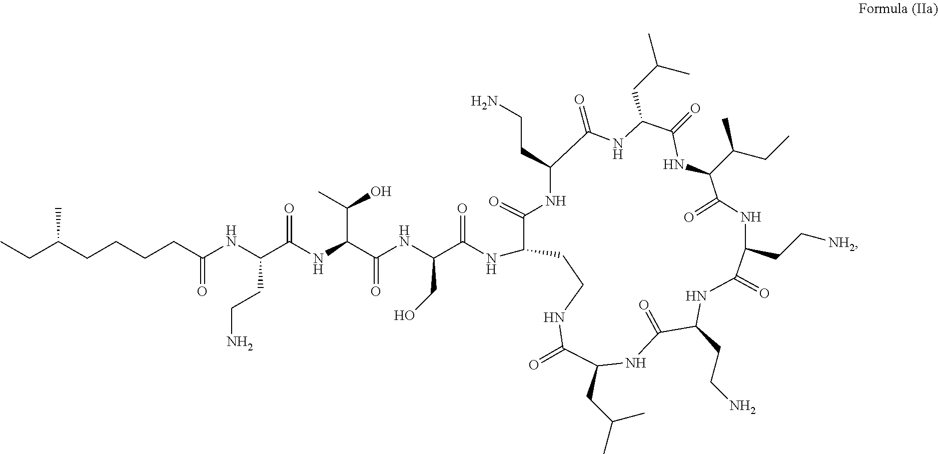 [SMILES: CC[C@H](C)CCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]

or a racemate, an enantiomer, a diastereomer, a pharmaceutically acceptable salt, or a derivative

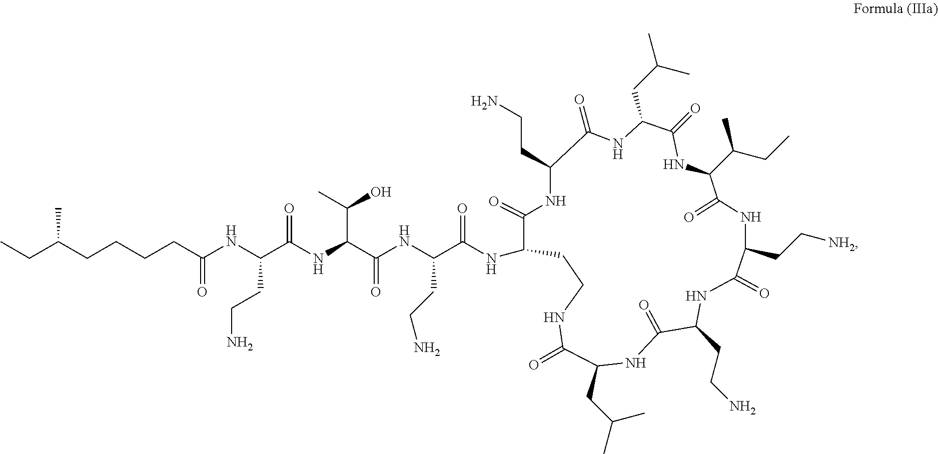 [SMILES: CC[C@H](C)CCCCC(=O)N[C@@H](CCN)C(=O)N[C@H](C(=O)N[C@@H](CCN)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]

or a racemate, an enantiomer, a diastereomer, a pharmaceutically acceptable salt, or a derivative thereof,

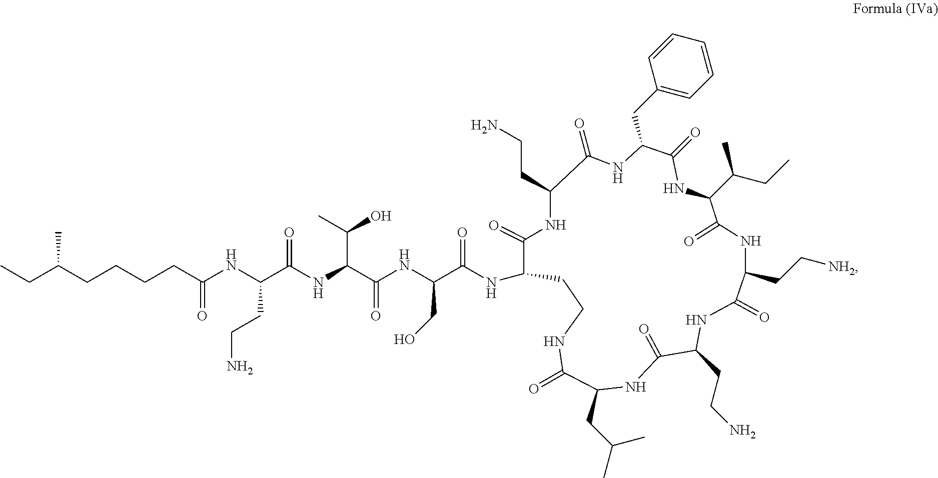 [SMILES: CC[C@H](C)CCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](Cc2ccccc2)NC(=O)[C@H](CCN)NC1=O)[C@@H](C)O]

or a racemate, an enantiomer, a diastereomer, a pharmaceutically acceptable salt, or a derivative thereof,

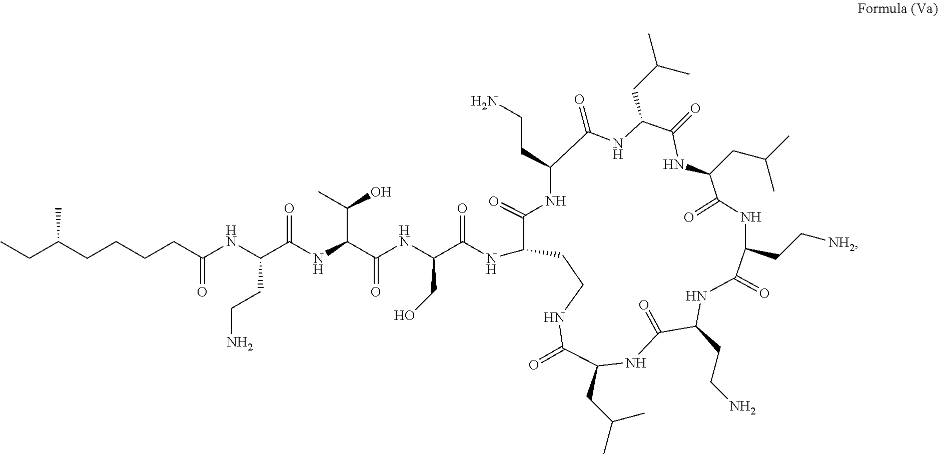 [SMILES: CC[C@H](C)CCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H](CC(C)C)NC(=O)[C@@H](CC(C)C)NC(=O)[C@H](CCN)NC1=O)[C@@H](C)O]

or a racemate, an enantiomer, a diastereomer, a pharmaceutically acceptable salt, or a derivative

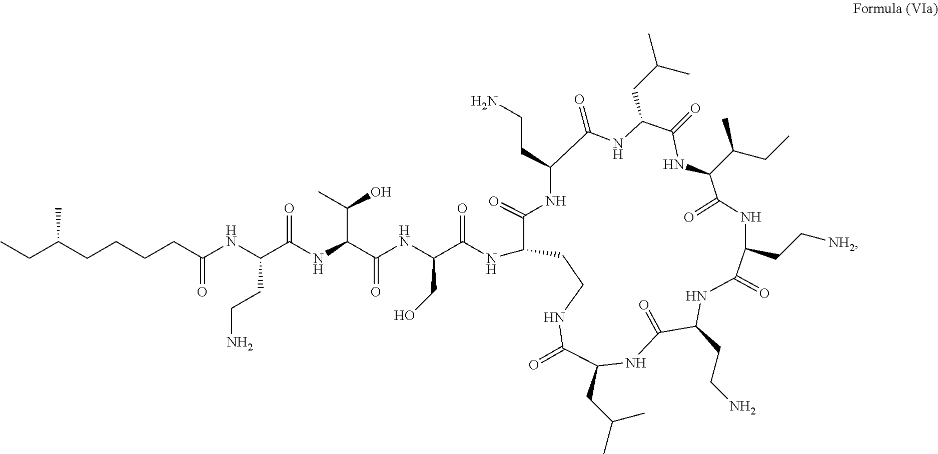 [SMILES: CC[C@H](C)CCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]

or a racemate, an enantiomer, a diastereomer, a pharmaceutically acceptable salt, or a derivative thereof,

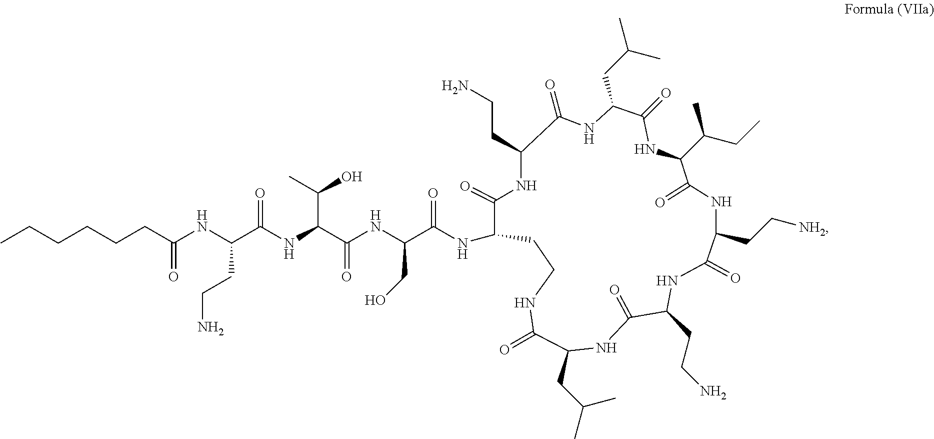 [SMILES: CCCCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]

or a racemate, an enantiomer, a diastereomer, a pharmaceutically acceptable salt, or a derivative thereof,

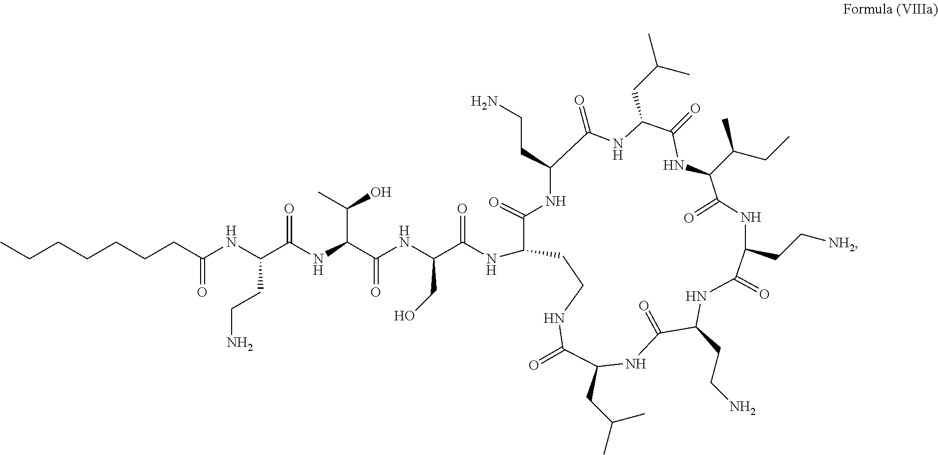 [SMILES: CCCCCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]

or a racemate, an enantiomer, a diastereomer, a pharmaceutically acceptable salt, or a derivative

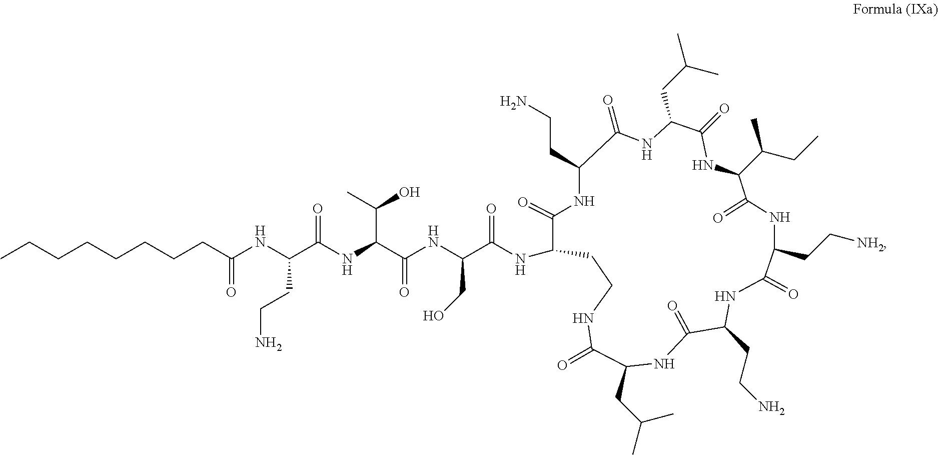 [SMILES: CCCCCCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]

or a racemate, an enantiomer, a diastereomer, a pharmaceutically acceptable salt, or a derivative thereof,

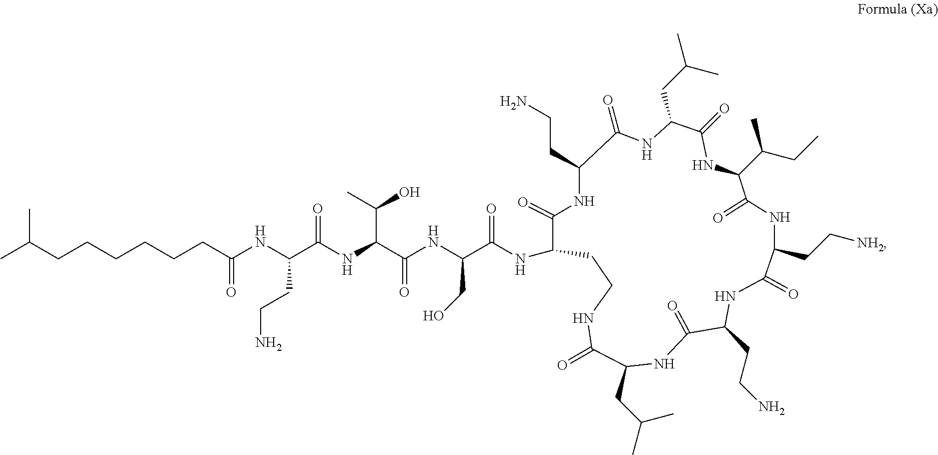 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)CCCCCCC(C)C)[C@@H](C)O)CCNC(=O)[C@H](CC(C)C)NC(=O)[C@H](CCN)NC(=O)[C@H](CCN)NC1=O]

or a racemate, an enantiomer, a diastereomer, a pharmaceutically acceptable salt, or a derivative thereof,

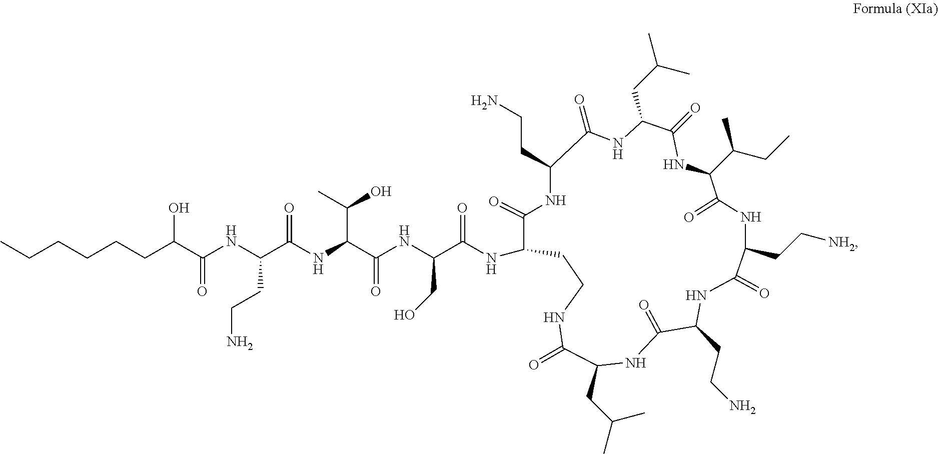 [SMILES: CCCCCCC(O)C(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]

or a racemate, an enantiomer, a diastereomer, a pharmaceutically acceptable salt, or a derivative

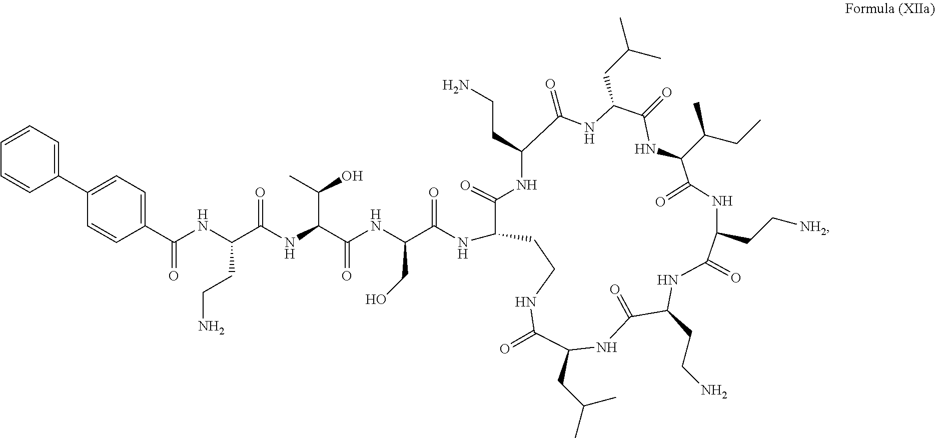 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)c2ccc(-c3ccccc3)cc2)[C@@H](C)O)CCNC(=O)[C@H](CC(C)C)NC(=O)[C@H](CCN)NC(=O)[C@H](CCN)NC1=O]

or a racemate, an enantiomer, a diastereomer, a pharmaceutically acceptable salt, or a derivative thereof,

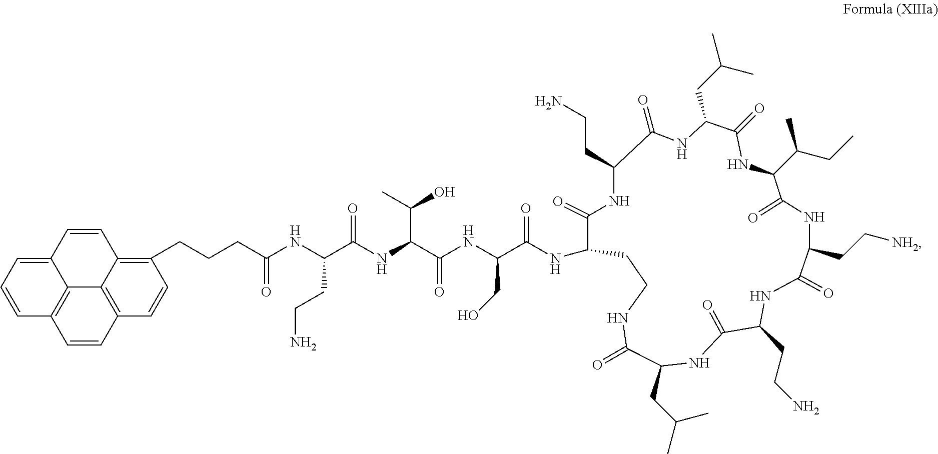 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)CCCc2ccc3ccc4cccc5ccc2c3c45)[C@@H](C)O)CCNC(=O)[C@H](CC(C)C)NC(=O)[C@H](CCN)NC(=O)[C@H](CCN)NC1=O]

or a racemate, an enantiomer, a diastereomer, a pharmaceutically acceptable salt, or a derivative thereof,

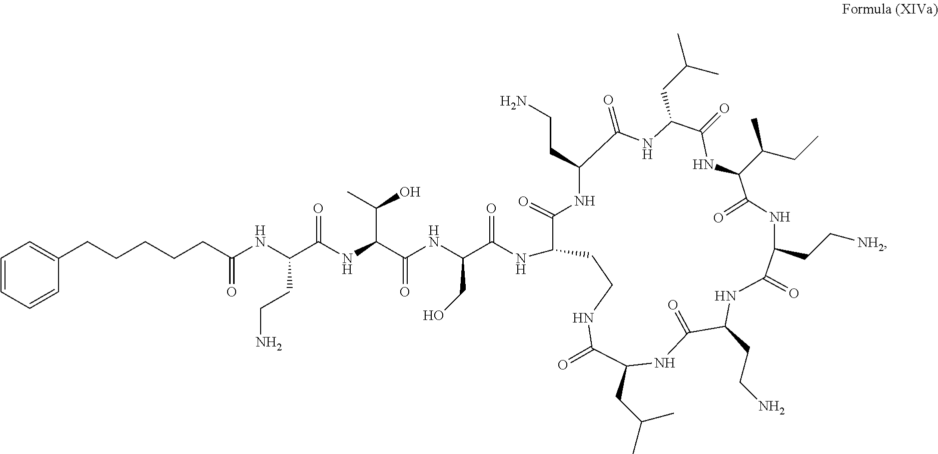 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)CCCCCc2ccccc2)[C@@H](C)O)CCNC(=O)[C@H](CC(C)C)NC(=O)[C@H](CCN)NC(=O)[C@H](CCN)NC1=O]

or a racemate, an enantiomer, a diastereomer, a pharmaceutically acceptable salt, or a derivative

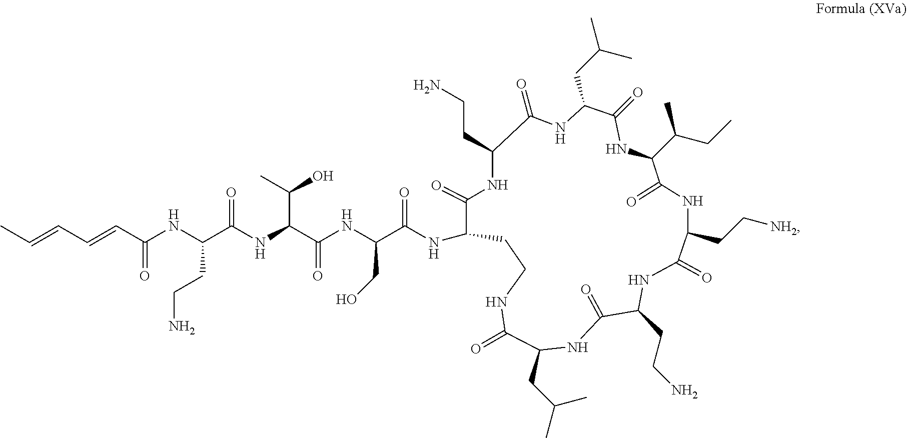 [SMILES: C/C=C/C=C/C(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]

or a racemate, an enantiomer, a diastereomer, a pharmaceutically acceptable salt, or a derivative thereof,

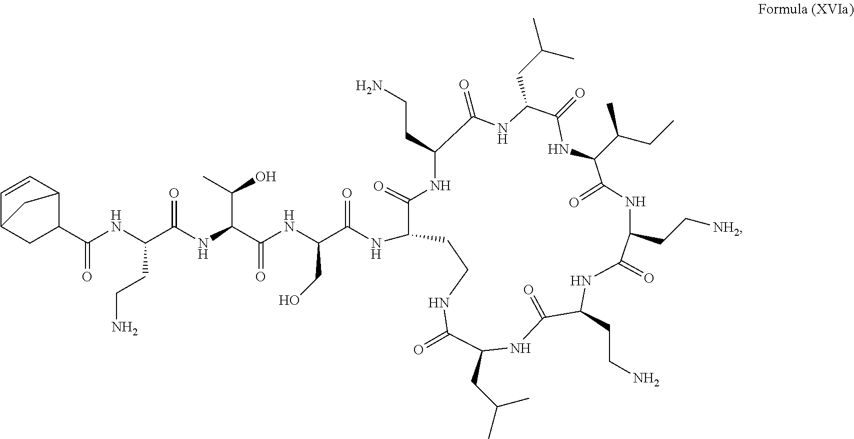 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)C2CC3C=CC2C3)[C@@H](C)O)CCNC(=O)[C@H](CC(C)C)NC(=O)[C@H](CCN)NC(=O)[C@H](CCN)NC1=O]

or a racemate, an enantiomer, a diastereomer, a pharmaceutically acceptable salt, or a derivative thereof,

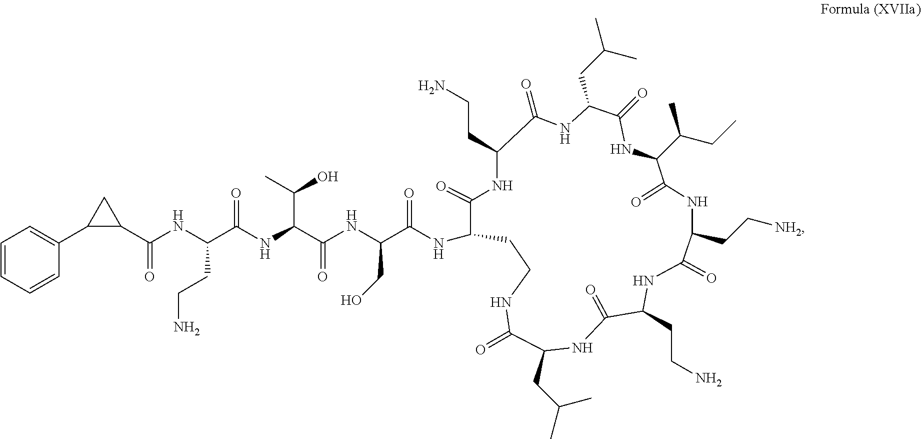 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)C2CC2c2ccccc2)[C@@H](C)O)CCNC(=O)[C@H](CC(C)C)NC(=O)[C@H](CCN)NC(=O)[C@H](CCN)NC1=O]

or a racemate, an enantiomer, a diastereomer, a pharmaceutically acceptable salt, or a derivative

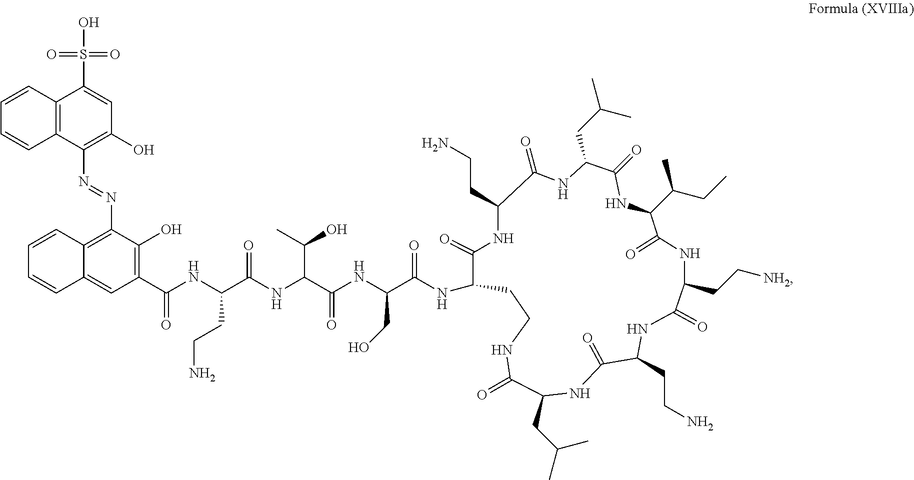 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)C(NC(=O)[C@H](CCN)NC(=O)c2cc3ccccc3c(/N=N/c3c(O)cc(S(=O)(=O)O)c4ccccc34)c2O)[C@@H](C)O)CCNC(=O)[C@H](CC(C)C)NC(=O)[C@H](CCN)NC(=O)[C@H](CCN)NC1=O]

or a racemate, an enantiomer, a diastereomer, a pharmaceutically acceptable salt, or a derivative thereof,

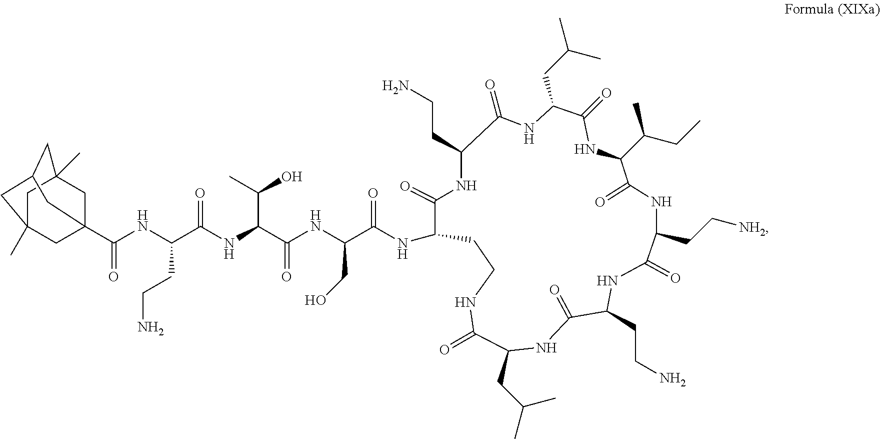 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)C23CC4CC(C)(CC(C)(C4)C2)C3)[C@@H](C)O)CCNC(=O)[C@H](CC(C)C)NC(=O)[C@H](CCN)NC(=O)[C@H](CCN)NC1=O]

or a racemate, an enantiomer, a diastereomer, a pharmaceutically acceptable salt, or a derivative thereof, or

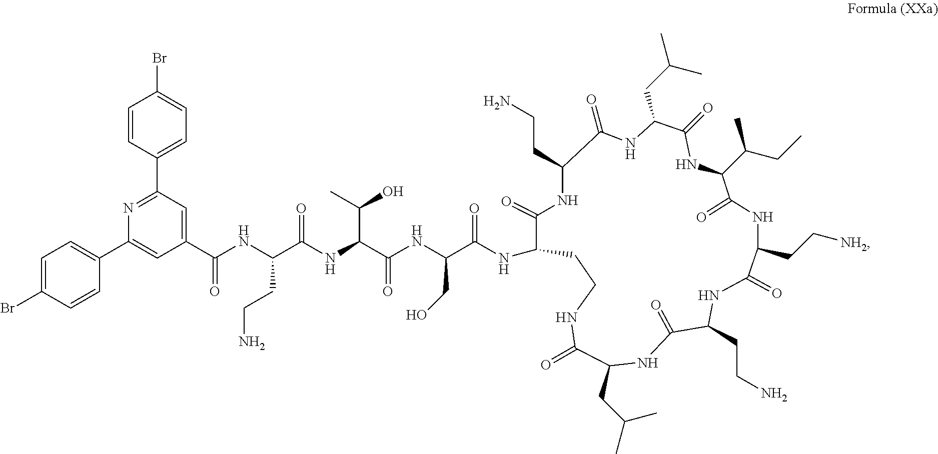 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)c2cc(-c3ccc(Br)cc3)nc(-c3ccc(Br)cc3)c2)[C@@H](C)O)CCNC(=O)[C@H](CC(C)C)NC(=O)[C@H](CCN)NC(=O)[C@H](CCN)NC1=O]

or a racemate, an enantiomer, a diastereomer, a pharmaceutically acceptable salt, or a derivative thereof.

In one aspect of the invention, the present invention relates, in part, to a pharmaceutical composition comprising at least one compound of the present invention.

In one aspect of the invention, the present invention relates, in part, to an isolated nucleic acid encoding a macolacin. In some embodiments, the macolacin comprises at least one compound of the present invention.

In one aspect of the invention, the present invention relates, in part, to a genetically engineered cell. In one embodiment, the cell expresses a macolacin.

In one aspect of the invention, the present invention relates, in part, to a method of treating or preventing a bacterial infection in a subject in need thereof. In some embodiments, the method comprises administering a composition comprising at least one compound of the present invention to the subject.

In some embodiments, the subject is exposed to or infected with a bacteria. In one embodiment, the bacteria is a gram positive bacteria. In one embodiment, the bacteria is a drug resistant bacteria.

In some embodiments, the method further comprises administering a second therapeutic. In one embodiment, the second therapeutic is an antibiotic.

In another aspect of the invention, the present invention relates, in part, to a method of inhibiting the growth of or killing a bacterial cell. In some embodiments, the method comprises contacting the bacterial cell with a composition comprising at least one compound of the present invention.

In another aspect of the invention, the present invention relates, in part, to a method of biosynthesizing a macolacin. In some embodiments, the method comprises providing a heterologous nucleic acid of the invention to a host, incubating the host in a growth medium, and isolating a macolacin from the host or the growth medium. In some embodiments, the macolacin comprises at least one compound of the present invention.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts a schematic representation of undiscovered congener BGCs may encode solutions to common antibiotic resistance mechanisms. Extensive use of antibiotics has resulted in the increased appearance of antibiotic resistant pathogens in the clinical setting. In nature a similar phenomenon is likely occurring in response to the natural production of antibiotics by bacteria. Nature however is not static, and the response to the development of resistance by some bacteria will be the selection of BGCs that encode variants of antibiotics that are capable circumventing common resistance mechanisms. Here, BGC guided chemical synthesis was used to identify a naturally occurring analog of colistin that is active against resistance encoded by the recently identified and now globally distributed mcr-1 gene.

FIG. 2, comprising FIG. 2A through FIG. 2E, depicts a schematic representation of discovery of macolacin. FIG. 2A depicts a schematic representation of the structure of colistin. FIG. 2B depicts a schematic representation of the mac gene cluster. The domain structure encoded by NRPS genes macA-macD. NRP synthesis is initiated from the Cs (condensation starter) domain. A C (condensation), A (adenylation), and T (thiolation) domain make a minimal NPRS module that extends the growing NRP by one amino acid. Inclusion of an epimerization (E) domain in the module alters the stereochemistry of the T domain bound amino acid. The TE (thioesterase) domain releases the mature NRP from the final T domain. FIG. 2C depicts representative results of analysis of the mac gene cluster A-domain substrate binding pockets. The ten residues used to determine substrate specificity are shown for each mac A-domain. FIG. 2D depicts representative results of comparison of the predicted macolacin decapeptide to decapeptides found in characterized polymyxin (poly) structures. The number of amino acids that each peptide differs from the consensus peptide derived from all known polymyxin structure is shown (Delta). FIG. 2E depicts a schematic representation of chemical synthesis of macolacin.

FIG. 3, comprising FIG. 3A through FIG. 3D, depicts representative results demonstrating antibacterial activity of macolacin. FIG. 3A depicts representative results demonstrating fold increase in minimum inhibitory concentration (MIC) for polymyxin, colistin, and macolacin upon introduction of the mer-1 resistance gene into K. pneumoniae or A. baumannii. FIG. 3B depicts representative results demonstrating disc diffusion assay (10 μg of antibiotic/disk) against K. pneumoniae and A. baumannii with or without the mcr-1 resistance gene. FIG. 3C depicts representative results demonstrating MIC of colistin or macolacin against K. pneumoniae and A. baumannii (n=2) upon addition of different cell wall components to the culture media. FIG. 3D depicts representative results demonstrating growth curves (n=3) for cultures of A. baumannii (blue) as well as A. baumannii in the presence of either the LpxC inhibitor CHIR-090 (green), one of three different antibiotics (colistin, macolacin, or kanamycin) (red) or both CHIR-090 and an additional antibiotic (purple).

FIG. 4 depicts representative structure activity relationship (SAR) of amino acid differences between macolacin and colistin. Macolacin differed from colistin by three amino acids: Ser3, Ile7, Leu10 (orange). Macolacin analogs synthesized with only two amino acid changes (orange) compared to colistin were tested for antibacterial activity (MIC μg/mL) against pathogens with or without either mcr-1 or phoP/Q (n=2). The fold increase in MIC upon introduction of either mer-1 or phoP/Q is shown in bold.

FIG. 5 depicts representative structural comparison of synthetic macolacin derivates. Structural differences compared to macolacin are depicted in blue.

FIG. 6 depicts a schematic representation of proposed macolacin biosynthetic pathway.

FIG. 7, comprising FIG. 7A through FIG. 7D, depicts representative phylogenetic trees constructed from A domain sequences associated with complete colistin and macolacin A BGC. Each A-domain sequence was extracted from the polymyxin-like BGCs was then aligned together with known characterized polymyxin BGCs (e.g., MIBIG IDs: BGC0000408, BGC0001192, BGC0001153) using the MUSCLE (Edgar R C et al., 2004, BMC Bioinformatics, 5:1-19113) alignment software. The resulting phylogenetic tree was visualized using iTOLv5 (Letunic I et al., 2005, Bioinformatics, 23:127-128) software. Red color represents hits in polymyxin clade. Blue color represents hits in macolacin clade. FIG. 7A depicts a representative phylogenetic tree of colistin A1 domain sequences. FIG. 7B depicts a representative phylogenetic tree of colistin A3 domain sequences. FIG. 7C depicts a representative phylogenetic tree of colistin A7 domain sequences. FIG. 7D depicts a representative phylogenetic tree of colistin A10 domain sequences.

FIG. 8, comprising FIG. 8A through FIG. 8D, depicts representative results demonstrating in vitro and in vivo activity of biphenyl-macolacin. FIG. 8A depicts representative MIC data for macolacin analogs with different lipid substituents (n=2). For pathogens that are not intrinsically resistant to colistin, the fold increase in MIC upon introduction of mcr-1 is shown. The fold increase in MIC upon introduction of either mcr-1 is shown in bold. S=colistin sensitive, R=colistin resistant. Col=colistin, Mac=macolacin. FIG. 8B depicts a schematic representation of structure of the most potent biphenyl-macolacin analog. FIG. 8C depicts results demonstrating CFU counts from a neutropenic thigh infection model using A. bauamnnii—SM1536-mcr-1. FIG. 8D depicts representative significant differences between groups that were analyzed by Mann-Whitney test (*** P<0.0005) (n=4 mice, n=8 thighs). Mean CFU counts and SD are shown.

FIG. 9 depicts representative results of pharmacokinetics assessment. Total plasma concentrations of macolacin, biphenyl-macolacin and colistin concentration versus time after administration of single subcutaneous dose of 10 mg/kg in neutropenic mice.

FIG. 10 depicts representative NGAL data for of biphenyl-macolacin and colistin in mice. The level of serum NGAL in colistin or biphenyl-macolacin treated mice. Significant differences between groups were analyzed by one-way analysis of variance (ANOVA) (*P<0.05) (n=6 mice). Date are presented as means±SD.

FIG. 11 depicts representative 1H NMR (600 MHZ, D₂O) spectrum of macolacin.

FIG. 12 depicts representative 13C NMR (600 MHZ, D₂O) spectrum of macolacin.

FIG. 13 depicts representative 1H NMR (600 MHZ, D₂O) spectrum of biphenyl-macolacin.

FIG. 14 depicts representative 13C NMR (600 MHz, D₂O) spectrum of Biphenyl-macolacin.

FIG. 15 depicts representative bacterial burdens in mouse thights. ªLimit of detection (LOD) for burden quantification was calculated as 100 CFU/g of thigh.

### DETAILED DESCRIPTION

The present invention is based, in part, on the unexpected discovery of macolacins as antibiotics which have activity against multidrug resistant pathogens. In one embodiment, the present invention provides compounds or a therapeutic compound comprising a desired activity. In one embodiment, the compound is an antibiotic. In one embodiment, the antibiotic compound of the invention can be used in the treatment of bacterial infections. In one embodiment, the antibiotic compound of the invention can be used in the treatment of gram positive bacterial infections. In certain embodiments, the use of the antibiotic compound of the invention in the treatment of bacterial infections optionally includes a pharmaceutically acceptable carrier, excipient or adjuvant.

In one embodiment, the compound can be biosynthesized via heterologous expression of a biosynthetic gene. Thus, in one aspect, the invention provides compounds and methods for synthesizing a macolacin compound. In one embodiment, the invention provides a nucleic acid encoding a macolacin. In one embodiment, the nucleic acid is an isolated nucleic acid. In one embodiment, the nucleic acid is transformed into a cell.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20%, +10%, +5%, +1%, or +0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

An “amino terminus modification group” refers to any molecule that can be attached to the amino terminus of a polypeptide. Similarly, a “carboxy terminus modification group” refers to any molecule that can be attached to the carboxy terminus of a polypeptide. Terminus modification groups include but are not limited to various water soluble polymers, peptides or proteins such as serum albumin, or other moieties that increase serum half-life of peptides.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

The term, “biologically active” or “bioactive” can mean, but is in no way limited to, the ability of an agent or compound to effectuate a physiological change or response. The response may be detected, for example, at the cellular level, for example, as a change in growth and/or viability, gene expression, protein quantity, protein modification, protein activity, or combination thereof; at the tissue level; at the systemic level; or at the organism level. For example, as used herein, biologically active molecules include but are not limited to any substance intended for diagnosis, cure, mitigation, treatment, or prevention of disease in humans or other animals, or to otherwise enhance physical or mental well-being of humans or animals. Examples of biologically active molecules include, but are not limited to, peptides, proteins, enzymes, small molecule drugs, dyes, lipids, nucleosides, oligonucleotides, cells, viruses, liposomes, microparticles and micelles. Classes of biologically active agents that are suitable for use with the invention include, but are not limited to, antibiotics, fungicides, anti-viral agents, anti-inflammatory agents, anti-tumor agents, cardiovascular agents, anti-anxiety agents, hormones, growth factors, steroidal agents, and the like.

The term “conservative mutations” refers to the substitution, deletion or addition of nucleic acids that alter, add or delete a single amino acid or a small number of amino acids in a coding sequence where the nucleic acid alterations result in the substitution of a chemically similar amino acid. Amino acids that may serve as conservative substitutions for each other include the following:
Basic: Arginine (R), Lysine (K), Histidine (H);
Acidic: Aspartic acid (D), Glutamic acid (E);
Neutral: Asparagine (N), Cysteine (C), Glutamine (Q), Methionine (M), Serine(S), Threonine (T);
Aliphatic: Alanine (A), Valine (V), Leucine (L), Isoleucine (I), Glycine (G);
Hydrophobic-Aromatic: Phenylalanine (F), Tyrosine (Y), Tryptophan (W);
Sulfur-containing: Methionine (M), Cysteine (C)·
Hydroxyl: Serine(S), Threonine (T);
Aminde: Asparagine (N), Glutamine (Q).

In addition, sequences that differ by conservative variations are generally homologous. In some instances, the following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M).

As used herein, “derivatives” are compositions formed from the native compounds either directly, by modification, or by partial substitution. As used herein, “analogs” are compositions that have a structure similar to, but not identical to, the native compound.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the terms “amino acid”, “amino acidic monomer”, or “amino acid residue” refer to any of the twenty naturally occurring amino acids including synthetic amino acids with unnatural side chains and including both D and L optical isomers.

In the context of the invention, term “natural amino acid” means any amino acid which is found naturally in vivo in a living being. Natural amino acids therefore include amino acids coded by mRNA incorporated into proteins during translation but also other amino acids found naturally in vivo which are a product or by-product of a metabolic process, such as for example ornithine which is generated by the urea production process by arginase from L-arginine. In the invention, the amino acids used can therefore be natural or not. Namely, natural amino acids generally have the L configuration but also, according to the invention, an amino acid can have the L or D configuration.

A “non-naturally encoded amino acid” refers to an amino acid that is not one of the 20 common amino acids or pyrolysine or selenocysteine. The term “non-naturally encoded amino acid” includes, but is not limited to, amino acids that occur naturally by modification of a naturally encoded amino acid (including but not limited to, the 20 common amino acids or pyrolysine and selenocysteine) but are not themselves incorporated into a growing polypeptide chain by the translation complex. Examples of naturally-occurring amino acids that are not naturally-encoded include, but are not limited to, N-acetylglucosaminyl-L-serine, N-acetylglucosaminyl-L-threonine, and O-phosphotyrosine.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

“Parenteral” administration of a composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof. Furthermore, peptides of the invention may include amino acid mimentics, and analogs. Recombinant forms of the peptides can be produced according to standard methods and protocols which are well known to those of skill in the art, including for example, expression of recombinant proteins in prokaryotic and/or eukaryotic cells followed by one or more isolation and purification steps, and/or chemically synthesizing peptides or portions thereof using a peptide sythesizer.

Conventional notation is used herein to portray polypeptide sequences: the left-hand end of a polypeptide sequence is the amino-terminus; the right-hand end of a polypeptide sequence is the carboxyl-terminus.

As used herein, a “peptidomimetic” is a compound containing non-peptidic structural elements that is capable of mimicking the biological action of a parent peptide. A peptidomimetic may or may not comprise peptide bonds.

The term “recombinant polypeptide” as used herein is defined as a polypeptide produced by using recombinant DNA methods. A host cell that comprises a recombinant polynucleotide is referred to as a “recombinant host cell.” A gene which is expressed in a recombinant host cell wherein the gene comprises a recombinant polynucleotide, produces a “recombinant polypeptide.”

The term “pharmacological composition,” “therapeutic composition,” “therapeutic formulation” or “pharmaceutically acceptable formulation” can mean, but is in no way limited to, a composition or formulation that allows for the effective distribution of an agent provided by the invention, which is in a form suitable for administration to the physical location most suitable for their desired activity, e.g., systemic administration.

Non-limiting examples of agents suitable for formulation with the, e.g., compounds provided by the instant invention include: cinnamoyl, PEG, phospholipids or lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999).

The term “pharmaceutically acceptable” or “pharmacologically acceptable” can mean, but is in no way limited to, entities and compositions that do not produce an adverse, allergic or other untoward reaction when administered to an animal, or a human, as appropriate.

The term “pharmaceutically acceptable carrier” or “pharmacologically acceptable carrier” can mean, but is in no way limited to, any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient. Disease and disorder are used interchangeably herein.

The phrase “therapeutically effective amount,” as used herein, refers to an amount that is sufficient or effective to prevent or treat (delay or prevent the onset of, prevent the progression of, inhibit, decrease or reverse) a disease or condition, including alleviating symptoms of such diseases.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “compound,” as used herein, unless otherwise indicated, refers to any specific chemical compound disclosed herein. In one embodiment, the term also refers to stereoisomers and/or optical isomers (including racemic mixtures) or enantiomerically enriched mixtures of disclosed compounds.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and includes straight, branched chain, or cyclic substituent groups. Examples include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. The term “alkyl,” unless otherwise noted, is also meant to include those derivatives of alkyl defined in more detail below, such as “heteroalkyl”, “haloalkyl” and “homoalkyl”.

As used herein, the term “substituted alkyl” means alkyl, as defined above, substituted by one, two or three substituents selected from the group consisting of halogen, —OH, alkoxy, —NH₂, —N(CH₃)₂, —C(═O)OH, trifluoromethyl, —C═N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkylene” by itself or as part of another molecule means a divalent radical derived from an alkane, as exemplified by (—CH₂-)ₙ. By way of example only, such groups include, but are not limited to, groups having 24 or fewer carbon atoms such as the structures-CH₂CH₂- and —CH₂CH₂CH₂CH₂—. The term “alkylene,” unless otherwise noted, is also meant to include those groups described below as “heteroalkylene.”

As used herein, the terms “alkoxy,” “alkylamino” and “alkylthio” are used in their conventional sense, and refer to alkyl groups linked to molecules via an oxygen atom, an amino group, a sulfur atom, respectively.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to ring atoms. Illustrative examples of cycloalkyl groups include, but are not limited to, the following moieties:

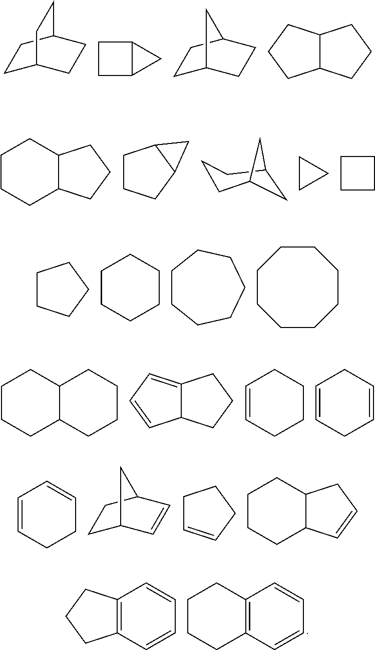

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon carbon double bond or one carbon carbon triple bond.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, Si, P, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O-CH₂—CH₂—CH₃, —CH₂—CH₂—CH₂—OH, —CH₂—CH₂—NH-CH₃, —CH₂—S-CH₂—CH₃, and —CH₂CH₂—S(═O)—CH₃. Up to two heteroatoms may be consecutive, such as, for example, —CH₂—NH-OCH₃, or —CH₂—CH₂—S—S-CH₃.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

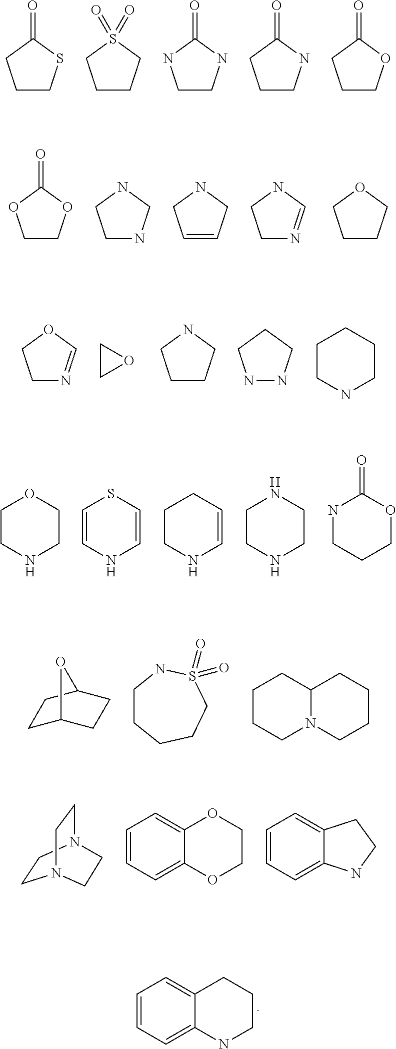

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl.

Preferred are phenyl and naphthyl, most preferred is phenyl.

As used herein, the term “aryl-(C₁-C₄)alkyl” means a functional group wherein a one to three carbon alkylene chain is attached to an aryl group, e.g., —CH₂CH₂-phenyl. Preferred is aryl-CH₂- and aryl-CH(CH₃)—. The term “substituted aryl-(C₁-C₄)alkyl” means an aryl-(C₁-C₄)alkyl functional group in which the aryl group is substituted. Preferred is substituted aryl(CH₂)—. Similarly, the term “heteroaryl-(C₁-C₄)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₄)alkyl” means a heteroaryl-(C₁-C₄)alkyl functional group in which the heteroaryl group is substituted. Preferred is substituted heteroaryl-(CH₂)—.

Examples of heteroaryl groups include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “amino aryl” refers to an aryl moiety which contains an amino moiety. Such amino moieties may include, but are not limited to primary amines, secondary amines, tertiary amines, masked amines, or protected amines. Such tertiary amines, masked amines, or protected amines may be converted to primary amine or secondary amine moieties. Additionally, the amine moiety may include an amine-like moiety which has similar chemical characteristics as amine moieties, including but not limited to chemical reactivity.

As used herein, the term “substituted” means that an atom or group of atoms has replaced hydrogen as the substituent attached to another group. For aryl, aryl-(C₁-C₄)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. In yet another embodiment, the substituents are independently selected from the group consisting of C₁₋₆ alkyl, —OH, C₁₋₆ alkoxy, halo, amino, acetamido and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic, with straight being preferred.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl, or substituted or unsubstituted phenyl], —C(═O)N[H or alkyl]₂, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O) CH₃, —ON(O)₂, and —C(═O)OH. In yet one embodiment, the substituents are independently selected from the group consisting of C₁₋₆ alkyl, —OH, C₁₋₆ alkoxy, halo, amino, acetamido, oxo and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative can also be a small molecule that differs in structure from the reference molecule, but retains the essential properties of the reference molecule. An analog or derivative may change its interaction with certain other molecules relative to the reference molecule. An analog or derivative molecule may also include a salt, an adduct, tautomer, isomer, or other variant of the reference molecule.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based, in part, on the unexpected discovery of macolacins as antibiotics which have activity against multidrug resistant pathogens. In one embodiment, the present invention provides compounds (e.g., compounds represented by Formula (I)-(XX)) or a therapeutic compound comprising a desired activity. In one embodiment, the compound is an antibiotic. In one embodiment, the antibiotic compound of the invention can be used in the treatment of bacterial infections. In one embodiment, the antibiotic compound of the invention can be used in the treatment of gram positive bacterial infections. In certain embodiments, the use of the antibiotic compound of the invention in the treatment of bacterial infections optionally includes a pharmaceutically acceptable carrier, excipient or adjuvant.

In one embodiment, the compound can be biosynthesized via heterologous expression of a biosynthetic gene. Thus, in one aspect, the invention provides compounds and methods for synthesizing a macolacin compound. In one embodiment, the invention provides a nucleic acid encoding a macolacin. In one embodiment, the nucleic acid is an isolated nucleic acid. In one embodiment, the nucleic acid is transformed into a cell.

### Compounds

In one aspect, the present invention provides novel macolacin compounds. In one embodiment, the invention provides a macolacin compound or a racemate, an enantiomer, a diastereomer thereof, a pharmaceutically acceptable salt, or a derivative thereof.

In one embodiment, the macolacin compound is a compound of general Formula (I)

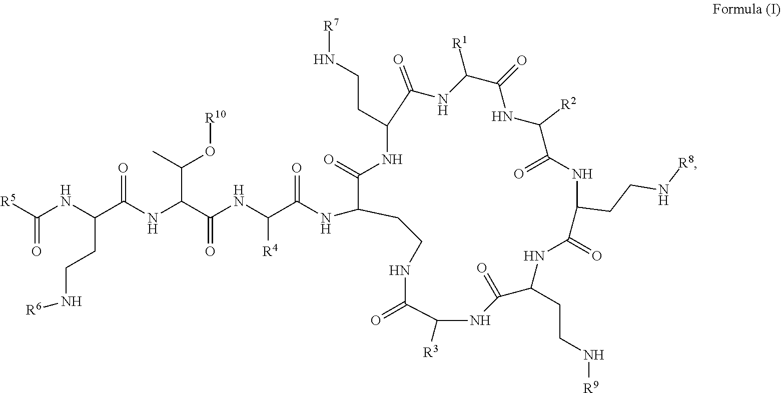

or a racemate, an enantiomer, a diastereomer, a pharmaceutically acceptable salt, or a derivative thereof.

In various embodiments, R¹ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof. For example, in some embodiments, R¹ is alkyl, alkenyl, aryl, aryl alkyl, aminoalkyl, hydroxyalkyl, or any combination thereof. In other embodiments, R¹ is linear C₁-C₁₀ alkyl, branched C₁-C₁₀ alkyl, linear aryl-C₁-C₁₀ alkyl, branched aryl-C₁-C₁₀ alkyl, linear amino-C₁-C₁₀ alkyl, amino-branched C₁-C₁₀ alkyl, linear hydroxy-C₁-C₁₀ alkyl, hydroxy-branched C₁-C₁₀ alkyl, linear C₁-C₁₀ alkenyl, branched C₁-C₁₀ alkenyl, linear aryl-C₁-C₁₀ alkenyl, branched aryl-C₁-C₁₀ alkenyl, linear amino-C₁-C₁₀ alkenyl, amino-branched C₁-C₁₀ alkenyl, linear hydroxy-C₁-C₁₀ alkenyl, hydroxy-branched C₁-C₁₀ alkenyl,

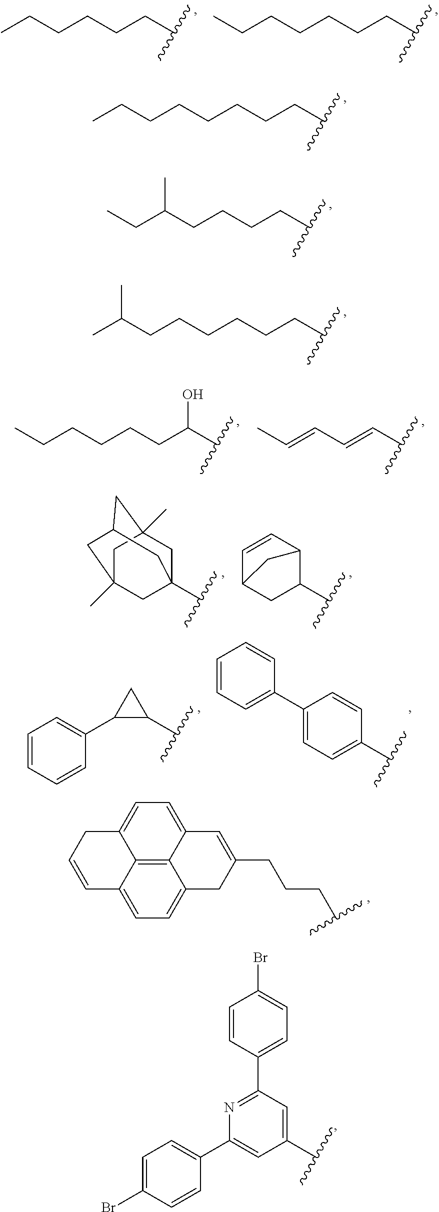

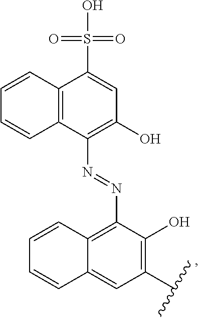 [SMILES: *c1cc2ccccc2c(/N=N/c2c(O)cc(S(=O)(=O)O)c3ccccc23)c1O]

or any combination thereof.

In various embodiments, R² is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, —O, —NO₂, —CN, sulfoxy, sulfonyl, alkyl sulfonyl, secondary amide, tertiary amide, an amino acid, or any combinations thereof. For example, in some embodiments, R² is alkyl, alkenyl, aryl, aryl alkyl, aminoalkyl, hydroxyalkyl, or any combination thereof. In other embodiments, R² is linear C₁-C₁₀ alkyl, branched C₁-C₁₀ alkyl, linear aryl-C₁-C₁₀ alkyl, branched aryl-C₁-C₁₀ alkyl, linear amino-C₁-C₁₀ alkyl, amino-branched C₁-C₁₀ alkyl, linear hydroxy-C₁-C₁₀ alkyl, hydroxy-branched C₁-C₁₀ alkyl, linear C₁-C₁₀ alkenyl, branched C₁-C₁₀ alkenyl, linear aryl-C₁-C₁₀ alkenyl, branched aryl-C₁-C₁₀ alkenyl, linear amino-C₁-C₁₀ alkenyl, amino-branched C₁-C₁₀ alkenyl, linear hydroxy-C₁-C₁₀ alkenyl, hydroxy-branched C₁-C₁₀ alkenyl,

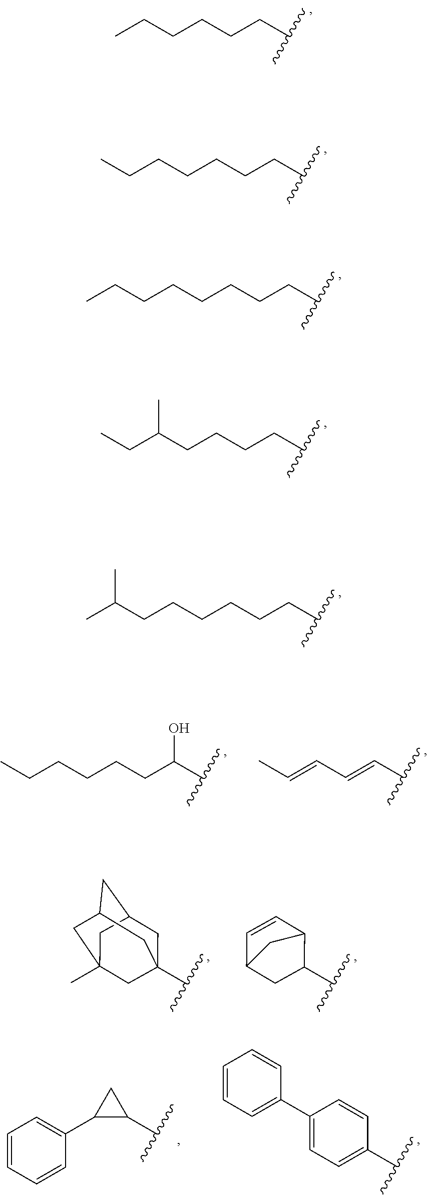

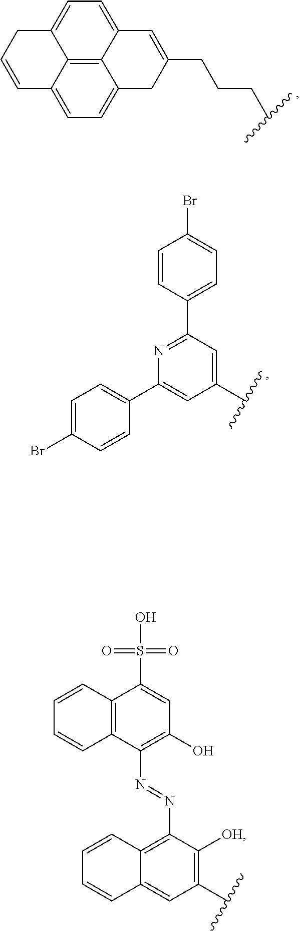 [SMILES: *CCCC1=Cc2ccc3c4c(ccc(c24)C1)C=CC3 ; *c1cc(-c2ccc(Br)cc2)nc(-c2ccc(Br)cc2)c1 ; *c1cc2ccccc2c(/N=N/c2c(O)cc(S(=O)(=O)O)c3ccccc23)c1O]

or any combination thereof.

In various embodiments, R³ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, —O, —NO₂, —CN, sulfoxy, sulfonyl, alkyl sulfonyl, secondary amide, tertiary amide, an amino acid, or any combinations thereof. For example, in some embodiments, R³ is alkyl, alkenyl, aryl, aryl alkyl, aminoalkyl, hydroxyalkyl, or any combination thereof. In other embodiments, R³ is linear C₁-C₁₀ alkyl, branched C₁-C₁₀ alkyl, linear aryl-C₁-C₁₀ alkyl, branched aryl-C₁-C₁₀ alkyl, linear amino-C₁-C₁₀ alkyl, amino-branched C₁-C₁₀ alkyl, linear hydroxy-C₁-C₁₀ alkyl, hydroxy-branched C₁-C₁₀ alkyl, linear C₁-C₁₀ alkenyl, branched C₁-C₁₀ alkenyl, linear aryl-C₁-C₁₀ alkenyl, branched aryl-C₁-C₁₀ alkenyl, linear amino-C₁-C₁₀ alkenyl, amino-branched C₁-C₁₀ alkenyl, linear hydroxy-C₁-C₁₀ alkenyl, hydroxy-branched C₁-C₁₀ alkenyl,

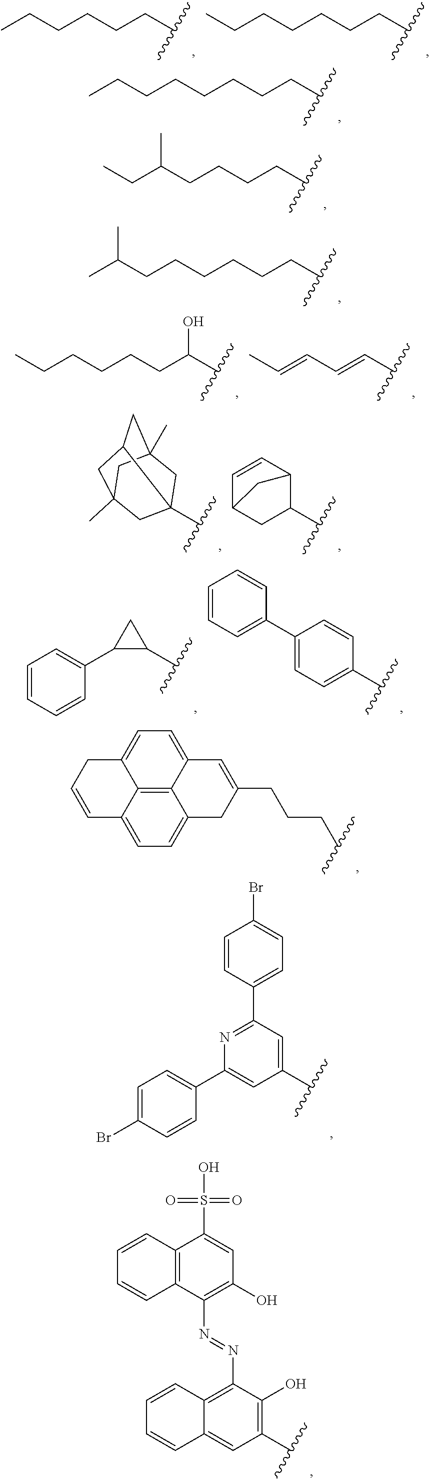

or any combination thereof.

In various embodiments, R⁴ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof. For example, in some embodiments, R⁴ is alkyl, alkenyl, aryl, aryl alkyl, aminoalkyl, hydroxyalkyl, or any combination thereof. In other embodiments, R⁴ is linear C₁-C₁₀ alkyl, branched C₁-C₁₀ alkyl, linear aryl-C₁-C₁₀ alkyl, branched aryl-C₁-C₁₀ alkyl, linear amino-C₁-C₁₀ alkyl, amino-branched C₁-C₁₀ alkyl, linear hydroxy-C₁-C₁₀ alkyl, hydroxy-branched C₁-C₁₀ alkyl, linear C₁-C₁₀ alkenyl, branched C₁-C₁₀ alkenyl, linear aryl-C₁-C₁₀ alkenyl, branched aryl-C₁-C₁₀ alkenyl, linear amino-C₁-C₁₀ alkenyl, amino-branched C₁-C₁₀ alkenyl, linear hydroxy-C₁-C₁₀ alkenyl, hydroxy-branched C₁-C₁₀ alkenyl,

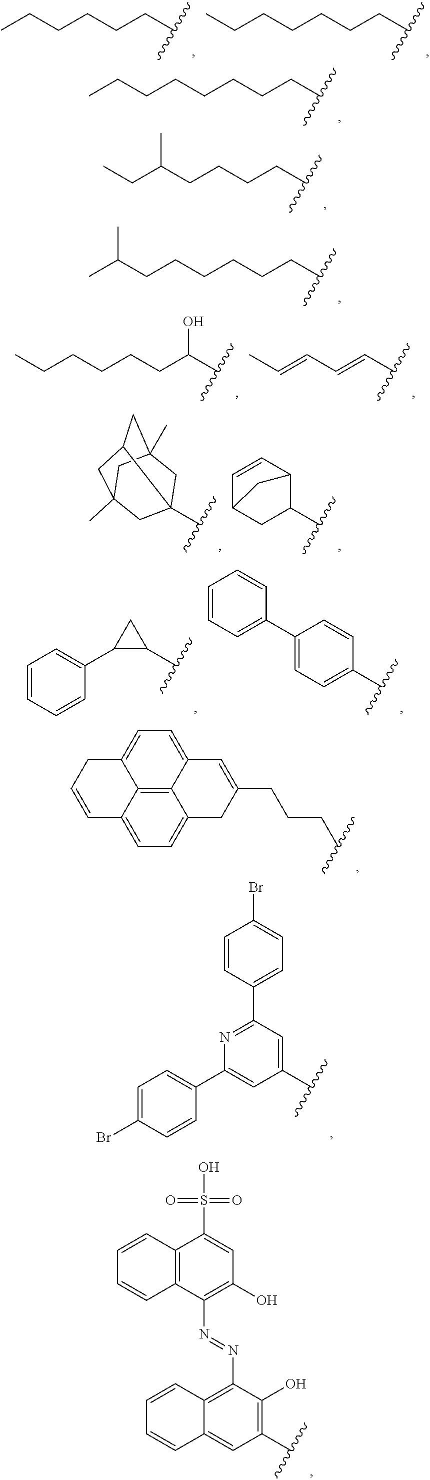

or any combination thereof.

In various embodiments, R⁵ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, —O, —NO₂, —CN, sulfoxy, sulfonyl, alkyl sulfonyl, secondary amide, tertiary amide, an amino acid, or any combinations thereof. For example, in some embodiments, R⁵ is alkyl, alkenyl, aryl, aryl alkyl, aminoalkyl, hydroxyalkyl, or any combination thereof. In other embodiments, R⁵ is linear C₁-C₁₀ alkyl, branched C₁-C₁₀ alkyl, linear aryl-C₁-C₁₀ alkyl, branched aryl-C₁-C₁₀ alkyl, linear amino-C₁-C₁₀ alkyl, amino-branched C₁-C₁₀ alkyl, linear hydroxy-C₁-C₁₀ alkyl, hydroxy-branched C₁-C₁₀ alkyl, linear C₁-C₁₀ alkenyl, branched C₁-C₁₀ alkenyl, linear aryl-C₁-C₁₀ alkenyl, branched aryl-C₁-C₁₀ alkenyl, linear amino-C₁-C₁₀ alkenyl, amino-branched C₁-C₁₀ alkenyl, linear hydroxy-C₁-C₁₀ alkenyl, hydroxy-branched C₁-C₁₀ alkenyl,

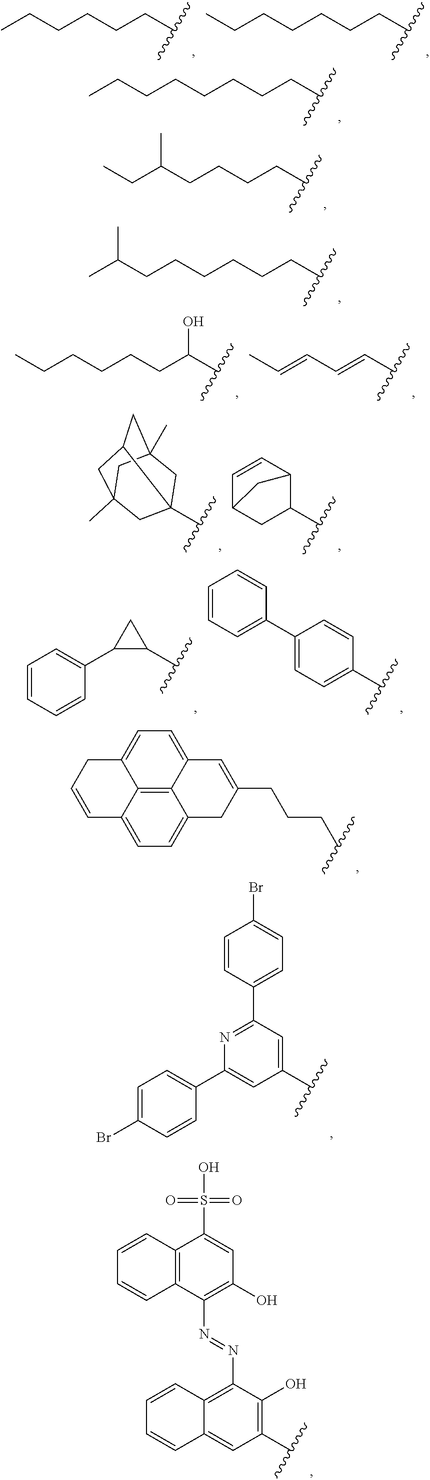

or any combination thereof.

In various embodiments, R⁶ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof. For example, in one embodiment, R⁶ is hydrogen. In one embodiment, R⁶ is alkyl sufonyl. In one embodiment, alkyl sulfonyl is methanesulfonyl.

In various embodiments, R⁷ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof. For example, in one embodiment, R⁷ is hydrogen. In one embodiment, R⁷ is alkyl sufonyl. In one embodiment, alkyl sulfonyl is methanesulfonyl.

In various embodiments, R⁸ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, —O, —NO₂, —CN, sulfoxy, sulfonyl, alkyl sulfonyl, secondary amide, tertiary amide, an amino acid, or any combinations thereof. For example, in one embodiment, R⁸ is hydrogen. In one embodiment, R⁸ is alkyl sufonyl. In one embodiment, alkyl sulfonyl is methanesulfonyl.

In various embodiments, R⁹ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, —O, —NO₂, —CN, sulfoxy, sulfonyl, alkyl sulfonyl, secondary amide, tertiary amide, an amino acid, or any combinations thereof. For example, in one embodiment, R⁹ is hydrogen. In one embodiment, R⁹ is alkyl sufonyl. In one embodiment, alkyl sulfonyl is methanesulfonyl.

In various embodiments, R¹⁰ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof. For example, in one embodiment, R¹⁰ is hydrogen. In one embodiment, R¹⁰ is alkyl sufonyl. In one embodiment, alkyl sulfonyl is methanesulfonyl.

For example, in some embodiments, each R¹, R², R³, R⁴, and R⁵ is independently aryl alkyl, aminoalkyl, hydroxyalkyl, or any combination thereof. In other embodiments, each R¹, R², R³, R⁴, and R⁵ is independently linear C₁-C₁₀ alkyl, branched C₁-C₁₀ alkyl, linear aryl-C₁-C₁₀ alkyl, branched aryl-C₁-C₁₀ alkyl, linear amino-C₁-C₁₀ alkyl, amino-branched C₁-C₁₀ alkyl, linear hydroxy-C₁-C₁₀ alkyl, hydroxy-branched C₁-C₁₀ alkyl, or any combination thereof.

For example, in one embodiment, the compound represented by Formula (I) is a compound represented by Formula (II)

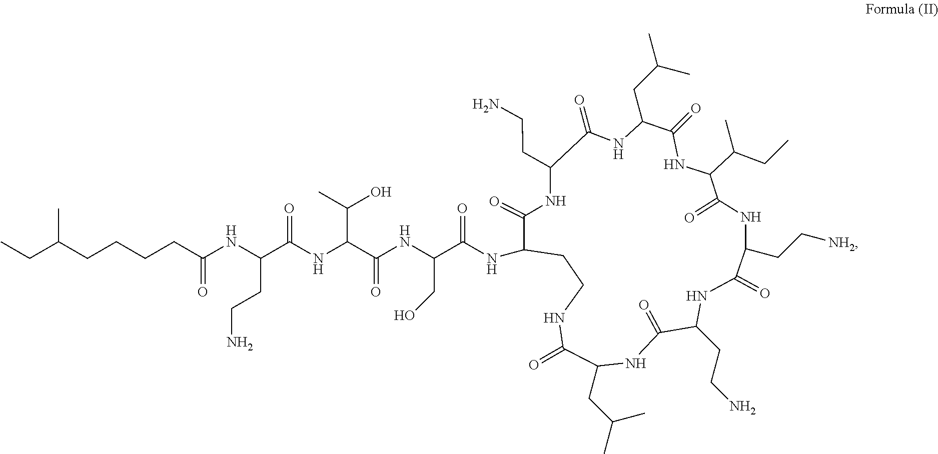 [SMILES: CCC(C)CCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]

or a racemate, an enantiomer, a diastereomer, a pharmaceutically acceptable salt, or a derivative thereof.

In one embodiment, the compound represented by Formula (I) is a compound represented by Formula (III)

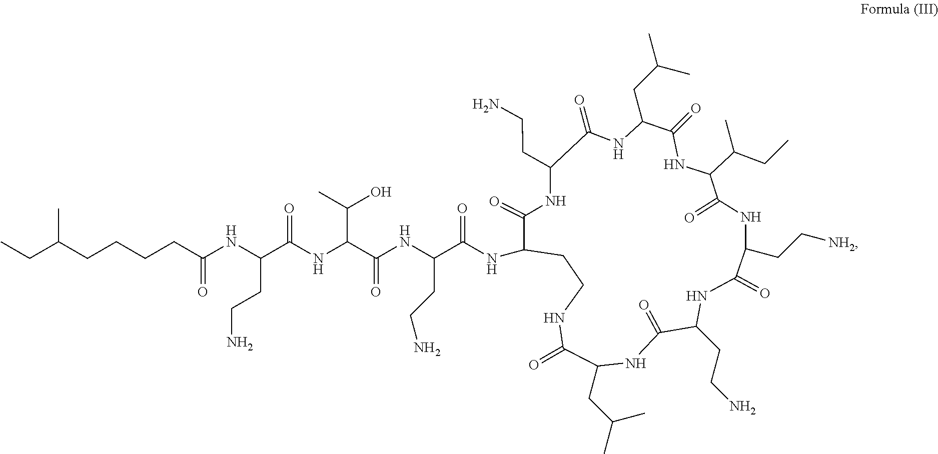 [SMILES: CCC(C)CCCCC(=O)NC(CCN)C(=O)NC(C(=O)NC(CCN)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]

or a racemate, an enantiomer, a diastereomer, a pharmaceutically acceptable salt, or a derivative thereof.

In one embodiment, the compound represented by Formula (I) is a compound represented by Formula (IV)

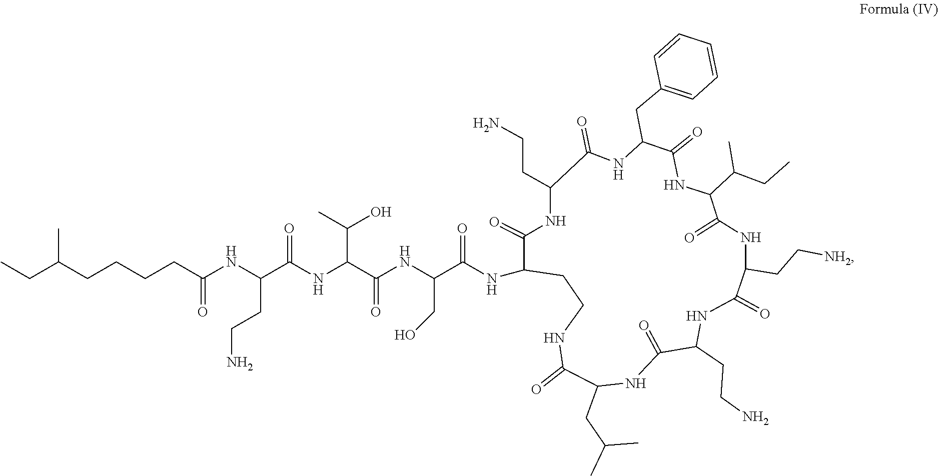 [SMILES: CCC(C)CCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(Cc2ccccc2)NC(=O)C(CCN)NC1=O)C(C)O]

or a racemate, an enantiomer, a diastereomer, a pharmaceutically acceptable salt, or a derivative thereof.

In one embodiment, the compound represented by Formula (I) is a compound represented by Formula (V)

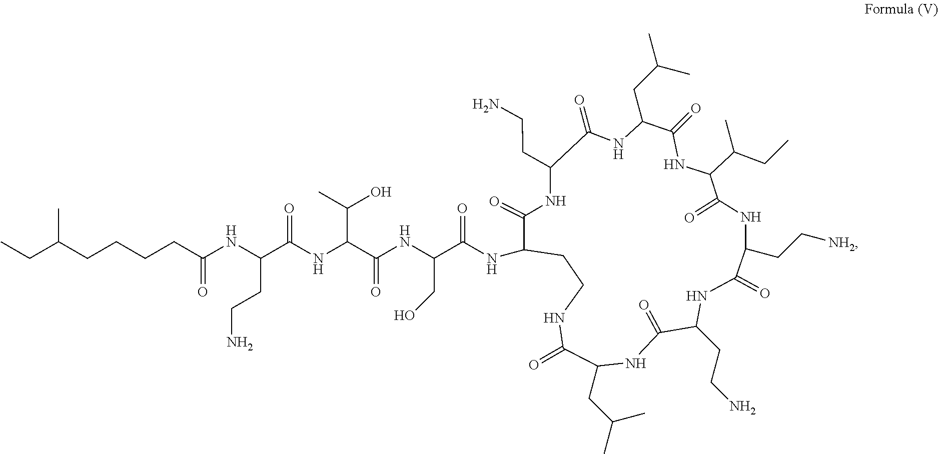 [SMILES: CCC(C)CCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]

or a racemate, an enantiomer, a diastereomer, a pharmaceutically acceptable salt, or a derivative thereof.

In one embodiment, the compound represented by Formula (I) is a compound represented by Formula (VI)

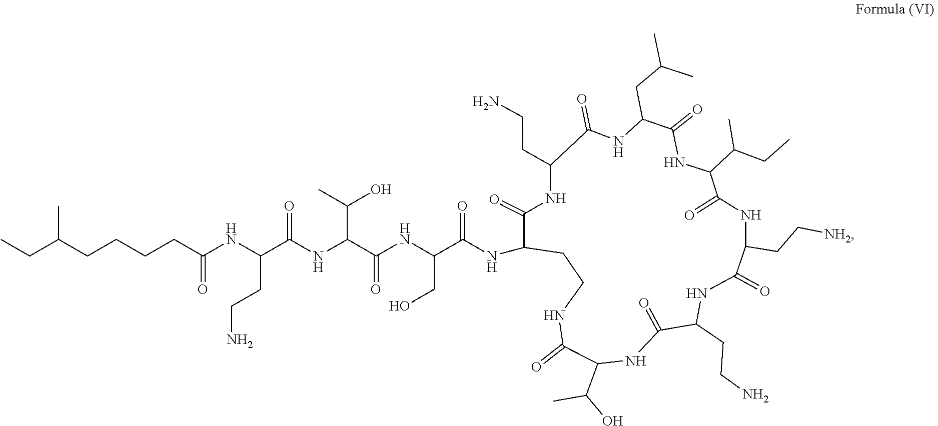 [SMILES: CCC(C)CCCCC(=O)NC(CCN)C(=O)NC(C(=O)NC(CO)C(=O)NC1CCNC(=O)C(C(C)O)NC(=O)C(CCN)NC(=O)C(CCN)NC(=O)C(C(C)CC)NC(=O)C(CC(C)C)NC(=O)C(CCN)NC1=O)C(C)O]

or a racemate, an enantiomer, a diastereomer, a pharmaceutically acceptable salt, or a derivative thereof.

In one embodiment, the compound represented by Formula (I) is a compound represented by Formula (VII)

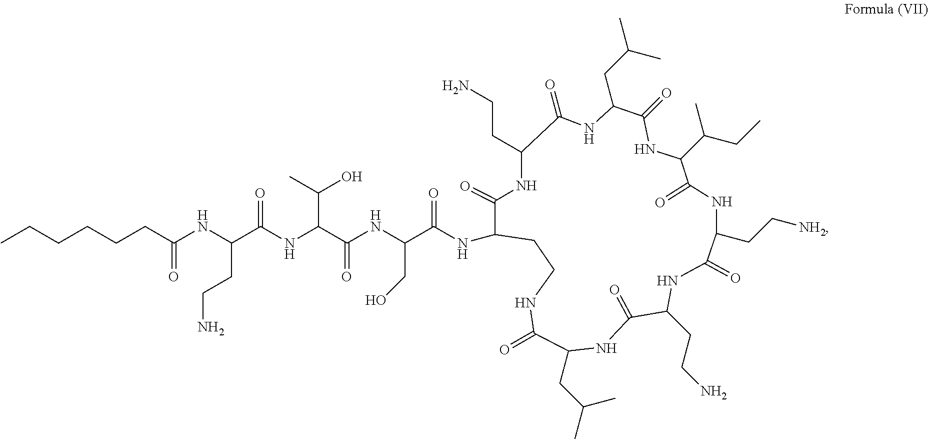 [SMILES: CCCCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]

or a racemate, an enantiomer, a diastereomer, a pharmaceutically acceptable salt, or a derivative thereof.

In one embodiment, the compound represented by Formula (I) is a compound represented by Formula (VIII)

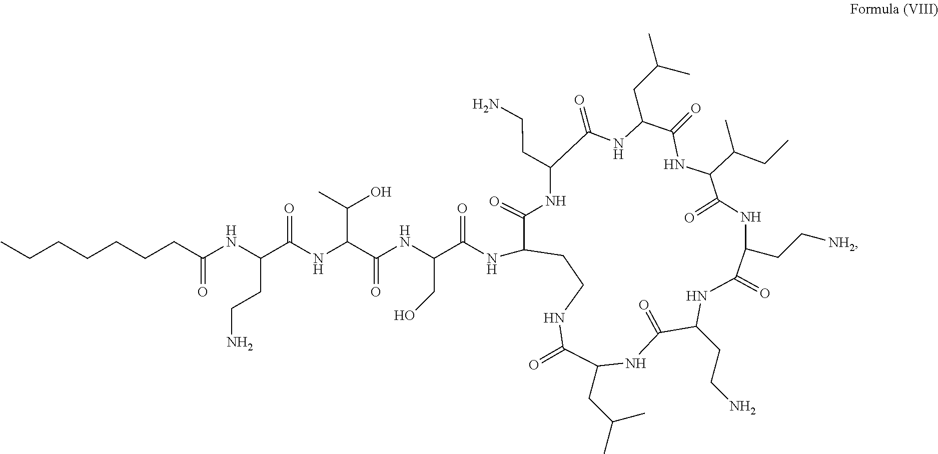 [SMILES: CCCCCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]

or a racemate, an enantiomer, a diastereomer, a pharmaceutically acceptable salt, or a derivative thereof.

In one embodiment, the compound represented by Formula (I) is a compound represented by Formula (IX)

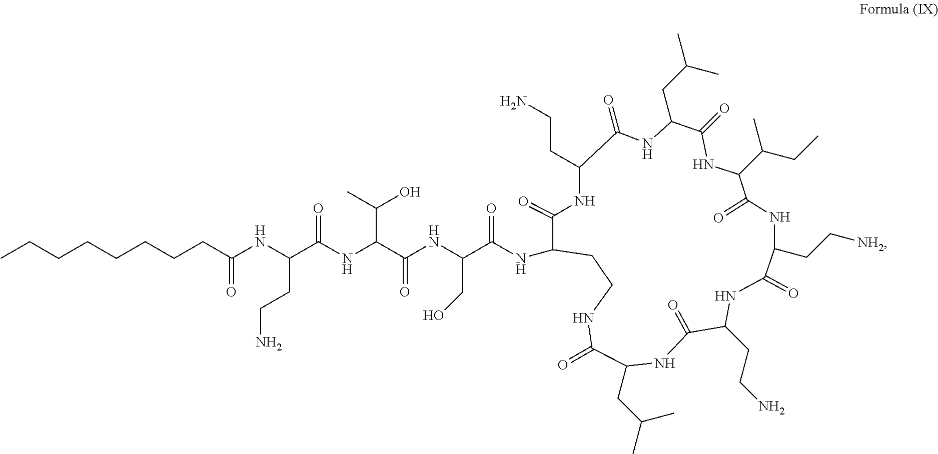 [SMILES: CCCCCCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]

or a racemate, an enantiomer, a diastereomer, a pharmaceutically acceptable salt, or a derivative thereof.

In one embodiment, the compound represented by Formula (I) is a compound represented by Formula (X)

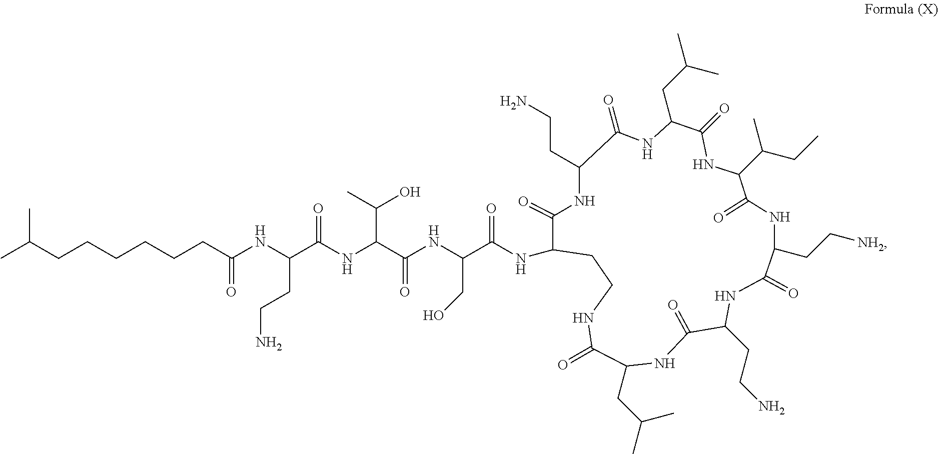 [SMILES: CCC(C)C1NC(=O)C(CC(C)C)NC(=O)C(CCN)NC(=O)C(NC(=O)C(CO)NC(=O)C(NC(=O)C(CCN)NC(=O)CCCCCCC(C)C)C(C)O)CCNC(=O)C(CC(C)C)NC(=O)C(CCN)NC(=O)C(CCN)NC1=O]

or a racemate, an enantiomer, a diastereomer, a pharmaceutically acceptable salt, or a derivative thereof.

In one embodiment, the compound represented by Formula (I) is a compound represented by Formula (XI)

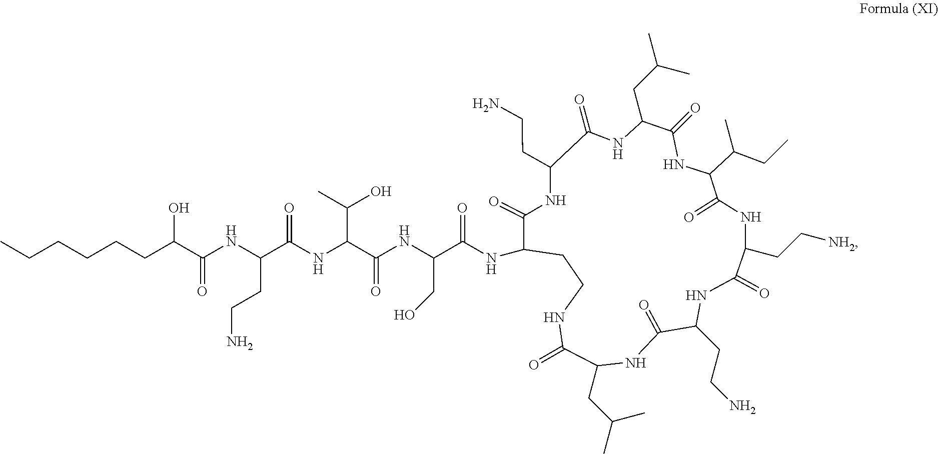 [SMILES: CCCCCCC(O)C(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]

or a racemate, an enantiomer, a diastereomer, a pharmaceutically acceptable salt, or a derivative thereof.

In one embodiment, the compound represented by Formula (I) is a compound represented by Formula (XII)

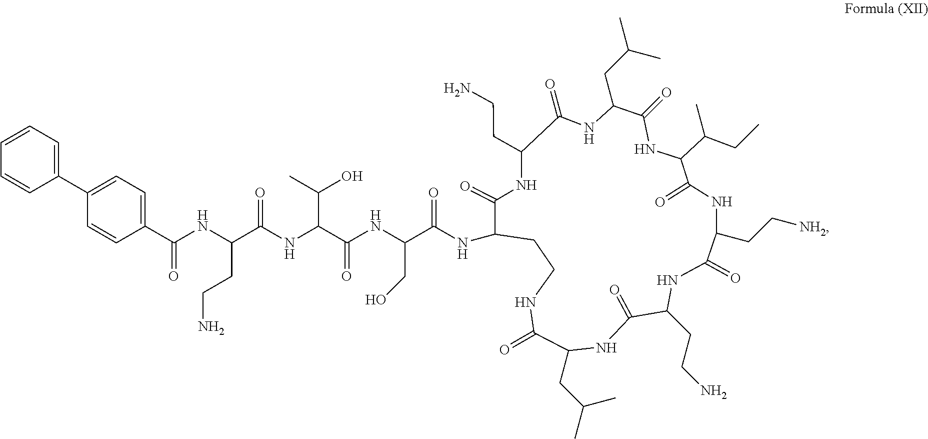 [SMILES: CCC(C)C1NC(=O)C(CC(C)C)NC(=O)C(CCN)NC(=O)C(NC(=O)C(CO)NC(=O)C(NC(=O)C(CCN)NC(=O)c2ccc(-c3ccccc3)cc2)C(C)O)CCNC(=O)C(CC(C)C)NC(=O)C(CCN)NC(=O)C(CCN)NC1=O]

or a racemate, an enantiomer, a diastereomer, a pharmaceutically acceptable salt, or a derivative thereof.

In one embodiment, the compound represented by Formula (I) is a compound represented by Formula (XIII)

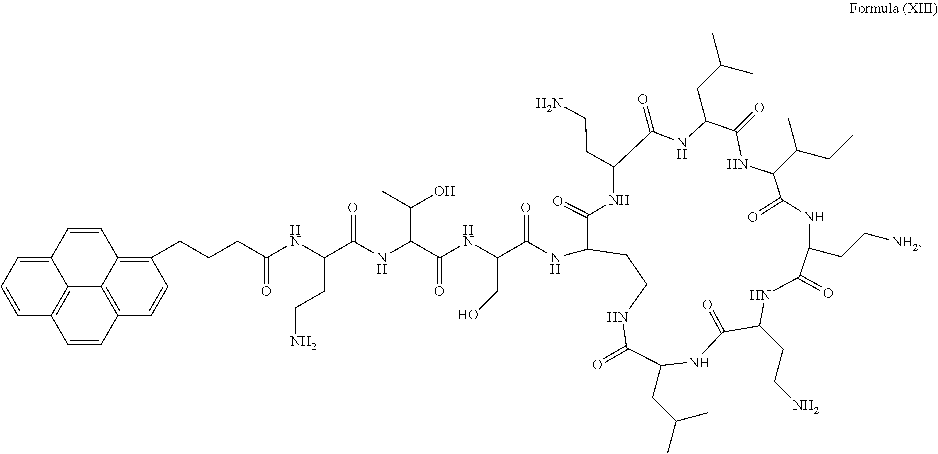 [SMILES: CCC(C)C1NC(=O)C(CC(C)C)NC(=O)C(CCN)NC(=O)C(NC(=O)C(CO)NC(=O)C(NC(=O)C(CCN)NC(=O)CCCc2ccc3ccc4cccc5ccc2c3c45)C(C)O)CCNC(=O)C(CC(C)C)NC(=O)C(CCN)NC(=O)C(CCN)NC1=O]

or a racemate, an enantiomer, a diastereomer, a pharmaceutically acceptable salt, or a derivative thereof.

In one embodiment, the compound represented by Formula (I) is a compound represented by Formula (XIV)

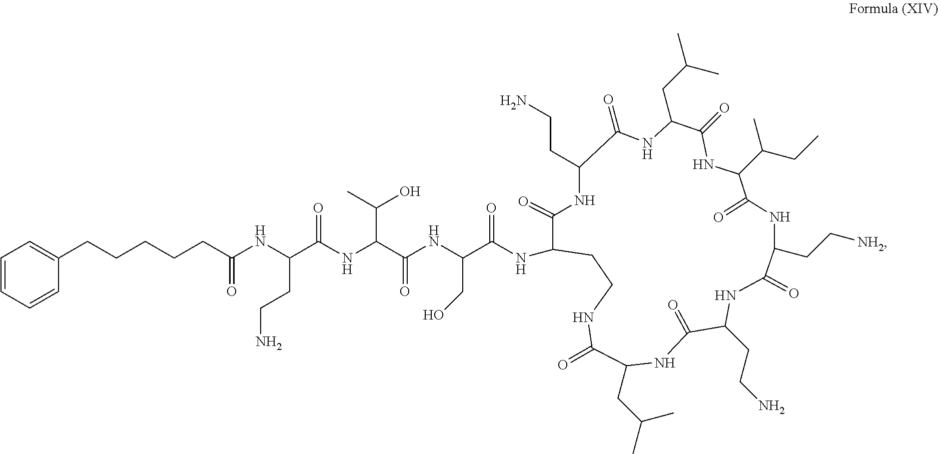 [SMILES: CCC(C)C1NC(=O)C(CC(C)C)NC(=O)C(CCN)NC(=O)C(NC(=O)C(CO)NC(=O)C(NC(=O)C(CCN)NC(=O)CCCCCc2ccccc2)C(C)O)CCNC(=O)C(CC(C)C)NC(=O)C(CCN)NC(=O)C(CCN)NC1=O]

or a racemate, an enantiomer, a diastereomer, a pharmaceutically acceptable salt, or a derivative thereof.

In one embodiment, the compound represented by Formula (I) is a compound represented by Formula (XV)

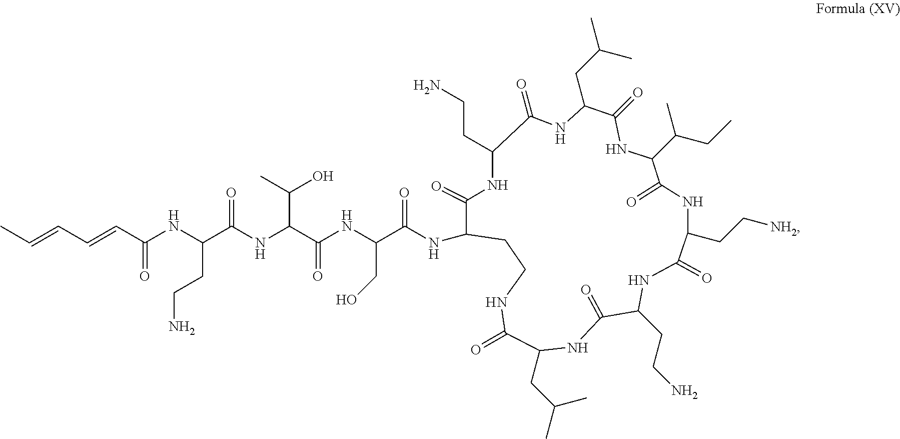 [SMILES: C/C=C/C=C/C(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]

or a racemate, an enantiomer, a diastereomer, a pharmaceutically acceptable salt, or a derivative thereof.

In one embodiment, the compound represented by Formula (I) is a compound represented by Formula (XVI)

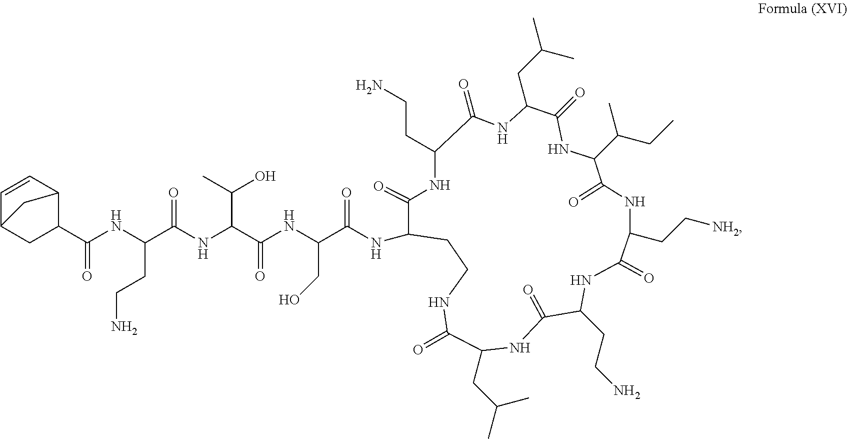 [SMILES: CCC(C)C1NC(=O)C(CC(C)C)NC(=O)C(CCN)NC(=O)C(NC(=O)C(CO)NC(=O)C(NC(=O)C(CCN)NC(=O)C2CC3C=CC2C3)C(C)O)CCNC(=O)C(CC(C)C)NC(=O)C(CCN)NC(=O)C(CCN)NC1=O]

or a racemate, an enantiomer, a diastereomer, a pharmaceutically acceptable salt, or a derivative thereof.

In one embodiment, the compound represented by Formula (I) is a compound represented by Formula (XVII)

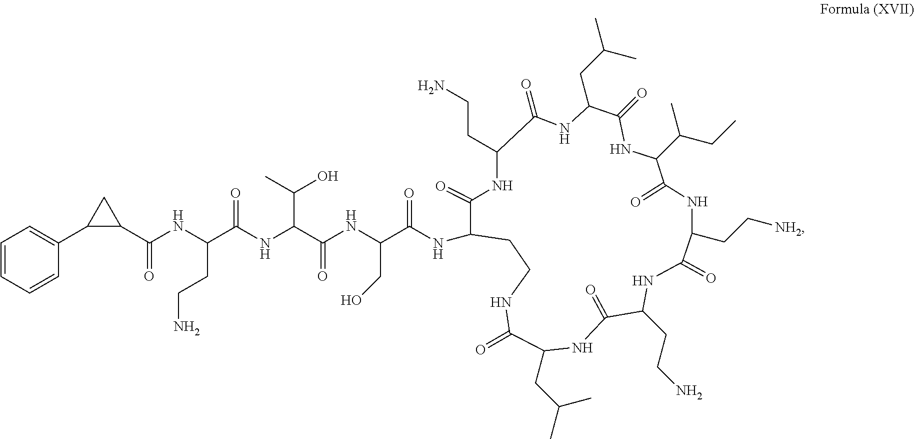 [SMILES: CCC(C)C1NC(=O)C(CC(C)C)NC(=O)C(CCN)NC(=O)C(NC(=O)C(CO)NC(=O)C(NC(=O)C(CCN)NC(=O)C2CC2c2ccccc2)C(C)O)CCNC(=O)C(CC(C)C)NC(=O)C(CCN)NC(=O)C(CCN)NC1=O]

or a racemate, an enantiomer, a diastereomer, a pharmaceutically acceptable salt, or a derivative thereof.

In one embodiment, the compound represented by Formula (I) is a compound represented by Formula (XVIII)

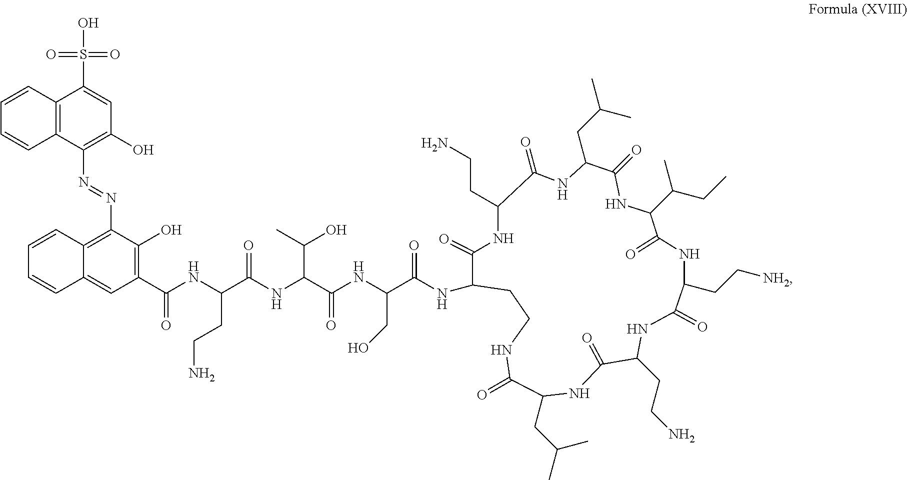 [SMILES: CCC(C)C1NC(=O)C(CC(C)C)NC(=O)C(CCN)NC(=O)C(NC(=O)C(CO)NC(=O)C(NC(=O)C(CCN)NC(=O)c2cc3ccccc3c(/N=N/c3c(O)cc(S(=O)(=O)O)c4ccccc34)c2O)C(C)O)CCNC(=O)C(CC(C)C)NC(=O)C(CCN)NC(=O)C(CCN)NC1=O]

or a racemate, an enantiomer, a diastereomer, a pharmaceutically acceptable salt, or a derivative thereof.

In one embodiment, the compound represented by Formula (I) is a compound represented by Formula (XIX)

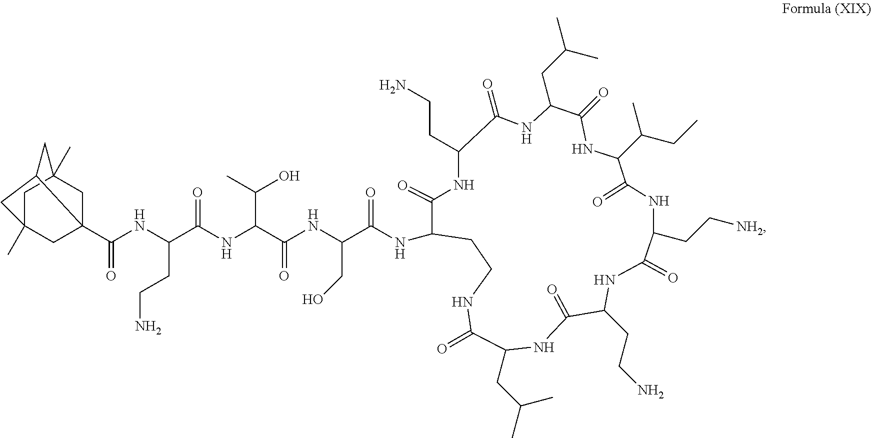 [SMILES: CCC(C)C1NC(=O)C(CC(C)C)NC(=O)C(CCN)NC(=O)C(NC(=O)C(CO)NC(=O)C(NC(=O)C(CCN)NC(=O)C23CC4(C)CC2CC(C)(C4)C3)C(C)O)CCNC(=O)C(CC(C)C)NC(=O)C(CCN)NC(=O)C(CCN)NC1=O]

or a racemate, an enantiomer, a diastereomer, a pharmaceutically acceptable salt, or a derivative thereof.

In one embodiment, the compound represented by Formula (I) is a compound represented by Formula (XX)

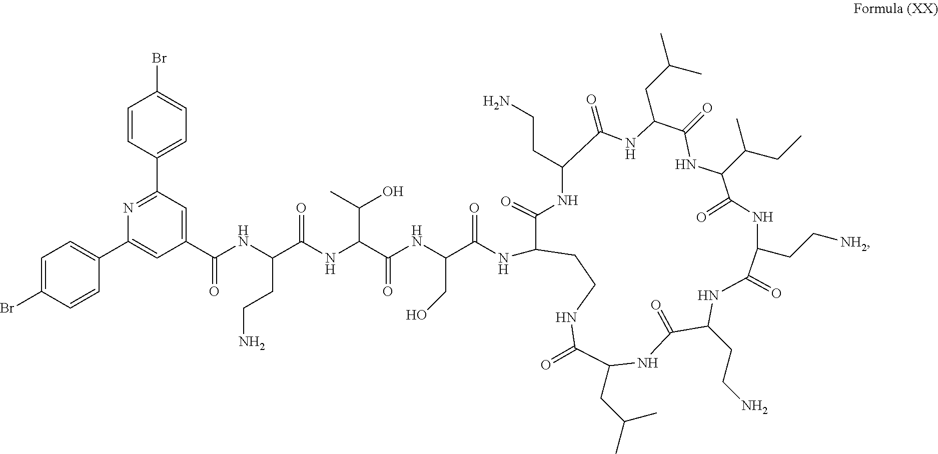 [SMILES: CCC(C)C1NC(=O)C(CC(C)C)NC(=O)C(CCN)NC(=O)C(NC(=O)C(CO)NC(=O)C(NC(=O)C(CCN)NC(=O)c2cc(-c3ccc(Br)cc3)nc(-c3ccc(Br)cc3)c2)C(C)O)CCNC(=O)C(CC(C)C)NC(=O)C(CCN)NC(=O)C(CCN)NC1=O]

or a racemate, an enantiomer, a diastereomer, a pharmaceutically acceptable salt, or a derivative thereof.

In some embodiments, the compound represented by Formula (I) is a compound represented by Formula (Ia)

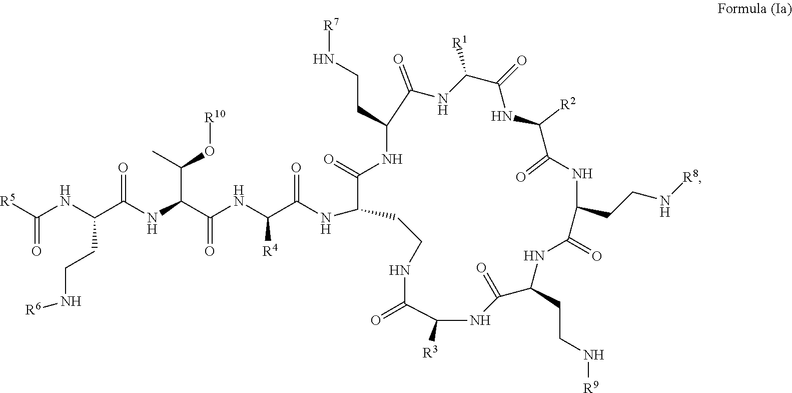

or a racemate, an enantiomer, a diastereomer, a pharmaceutically acceptable salt, or a derivative thereof.

For example, in one embodiment, the compound represented by Formula (Ia) is a compound represented by Formula (IIa)

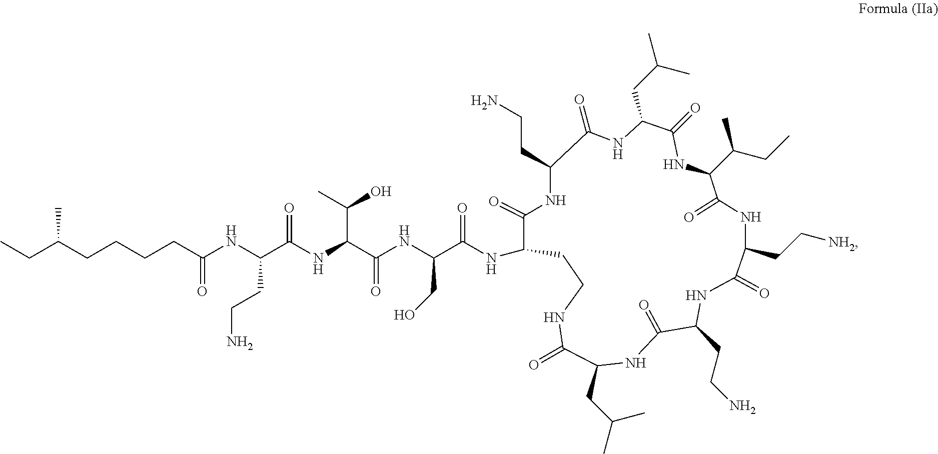 [SMILES: CC[C@H](C)CCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]

or a racemate, an enantiomer, a diastereomer, a pharmaceutically acceptable salt, or a derivative thereof.

In one embodiment, the compound represented by Formula (Ia) is a compound represented by Formula (IIIa)

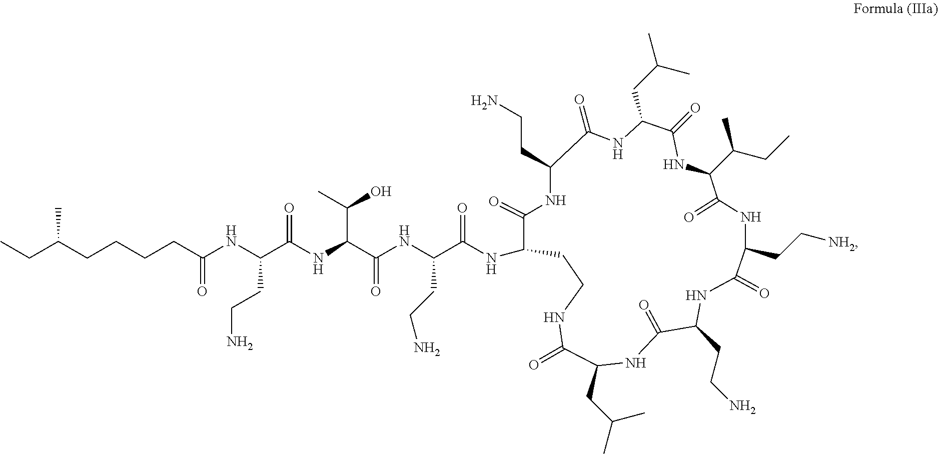 [SMILES: CC[C@H](C)CCCCC(=O)N[C@@H](CCN)C(=O)N[C@H](C(=O)N[C@@H](CCN)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]

or a racemate, an enantiomer, a diastereomer, a pharmaceutically acceptable salt, or a derivative thereof.

In one embodiment, the compound represented by Formula (Ia) is a compound represented by Formula (IVa)

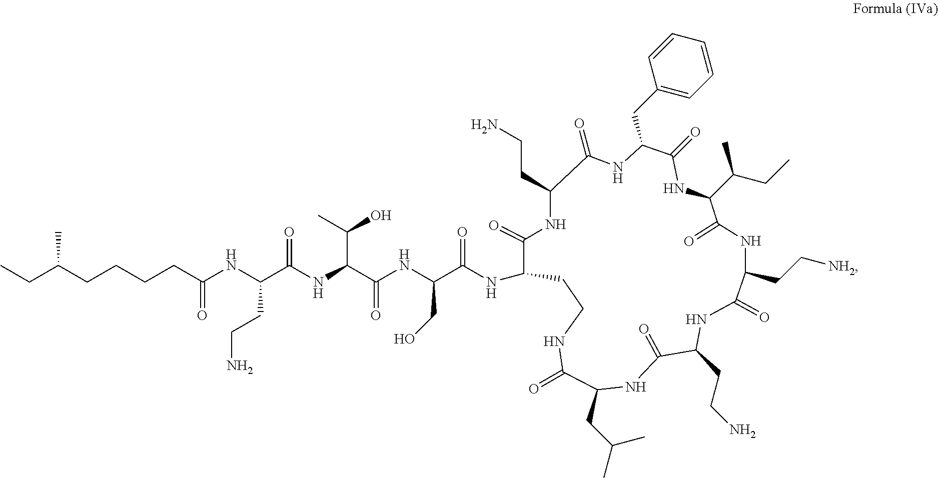 [SMILES: CC[C@H](C)CCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](Cc2ccccc2)NC(=O)[C@H](CCN)NC1=O)[C@@H](C)O]

or a racemate, an enantiomer, a diastereomer, a pharmaceutically acceptable salt, or a derivative thereof.

In one embodiment, the compound represented by Formula (Ia) is a compound represented

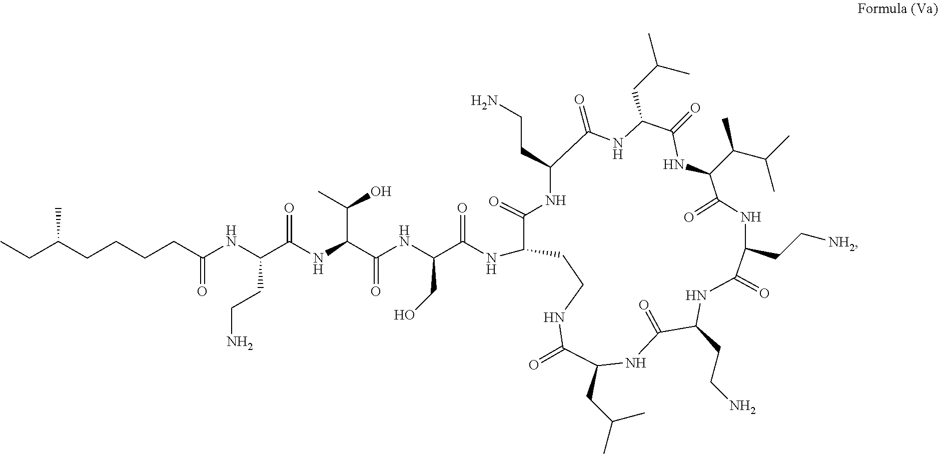 [SMILES: CC[C@H](C)CCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)C(C)C)NC(=O)[C@@H](CC(C)C)NC(=O)[C@H](CCN)NC1=O)[C@@H](C)O]

by Formula (Va) or a racemate, an enantiomer, a diastereomer, a pharmaceutically acceptable salt, or a derivative thereof.

In one embodiment, the compound represented by Formula (Ia) is a compound represented by Formula (VIa)

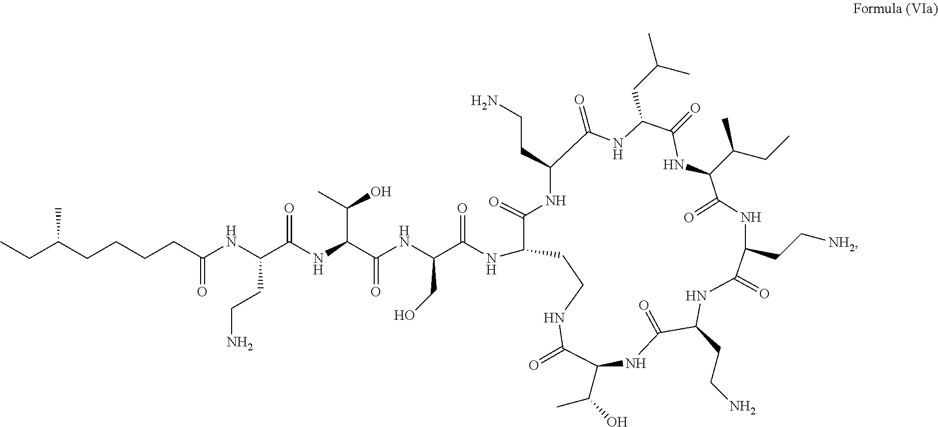 [SMILES: CC[C@H](C)CCCCC(=O)N[C@@H](CCN)C(=O)N[C@H](C(=O)N[C@H](CO)C(=O)N[C@H]1CCNC(=O)[C@H]([C@@H](C)O)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]

or a racemate, an enantiomer, a diastereomer, a pharmaceutically acceptable salt, or a derivative thereof.

In one embodiment, the compound represented by Formula (Ia) is a compound represented by Formula (VIIa)

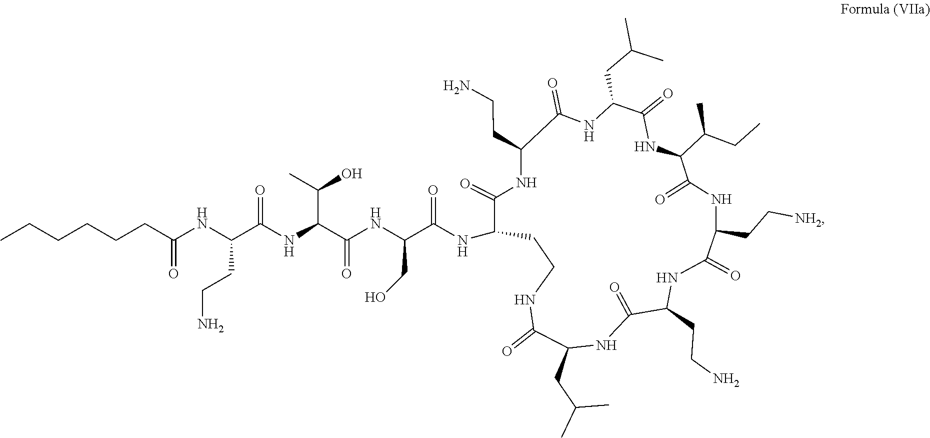 [SMILES: CCCCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]

or a racemate, an enantiomer, a diastereomer, a pharmaceutically acceptable salt, or a derivative thereof.

In one embodiment, the compound represented by Formula (Ia) is a compound represented by Formula (VIIIa)

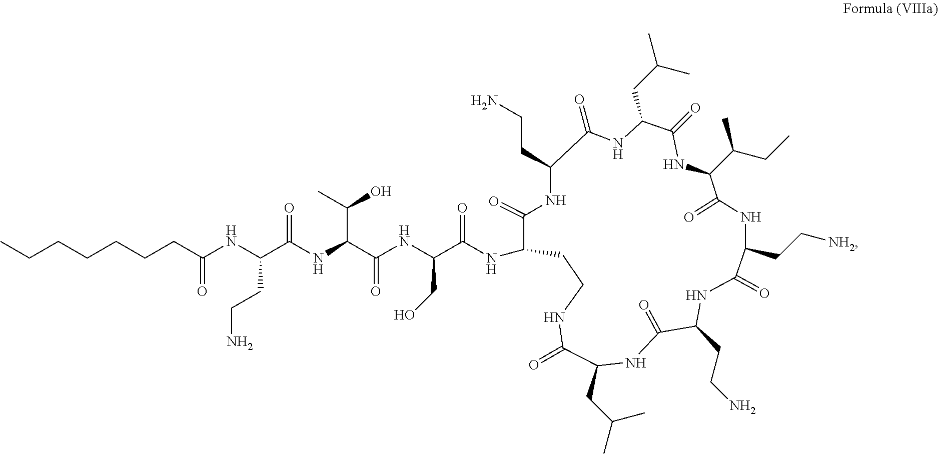 [SMILES: CCCCCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]

or a racemate, an enantiomer, a diastereomer, a pharmaceutically acceptable salt, or a derivative thereof.

In one embodiment, the compound represented by Formula (Ia) is a compound represented by Formula (IXa)

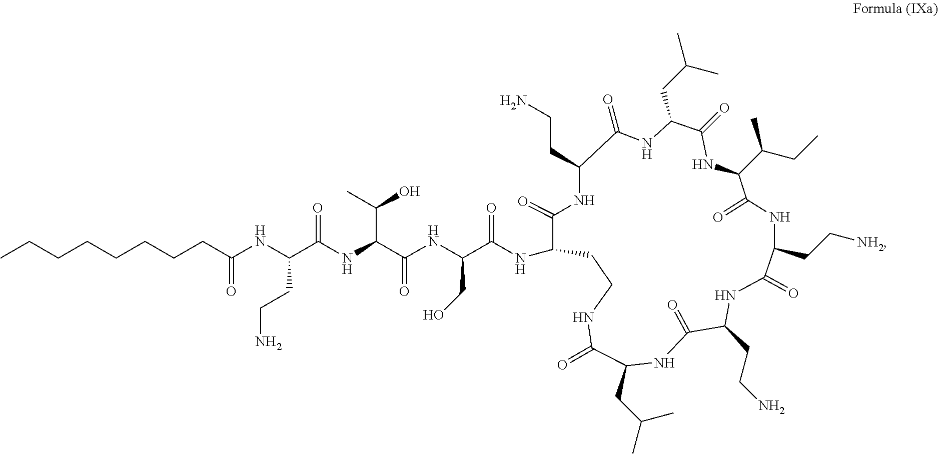 [SMILES: CCCCCCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]

or a racemate, an enantiomer, a diastereomer, a pharmaceutically acceptable salt, or a derivative thereof.

In one embodiment, the compound represented by Formula (Ia) is a compound represented by Formula (Xa)

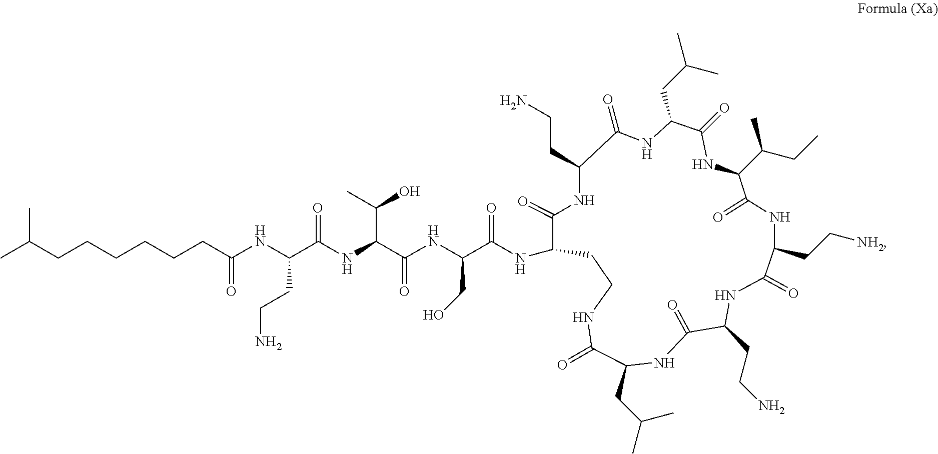 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)CCCCCCC(C)C)[C@@H](C)O)CCNC(=O)[C@H](CC(C)C)NC(=O)[C@H](CCN)NC(=O)[C@H](CCN)NC1=O]

or a racemate, an enantiomer, a diastereomer, a pharmaceutically acceptable salt, or a derivative thereof.

In one embodiment, the compound represented by Formula (Ia) is a compound represented by Formula (XIa)

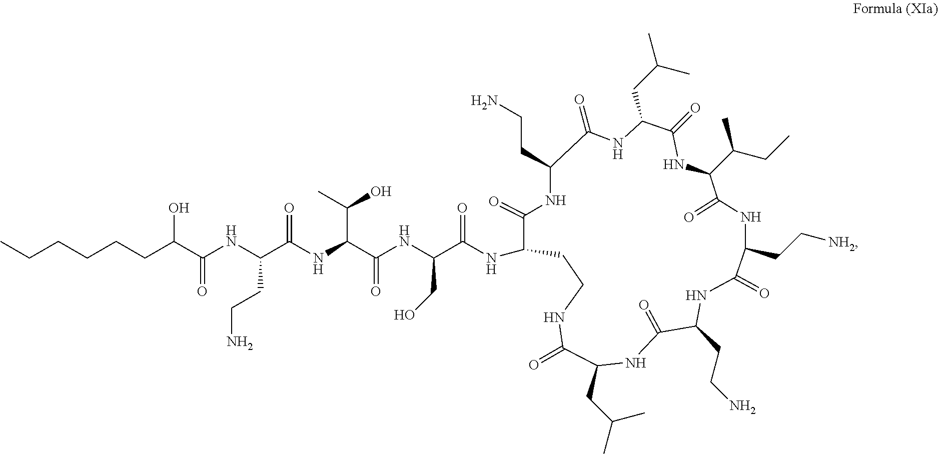 [SMILES: CCCCCCC(O)C(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]

or a racemate, an enantiomer, a diastereomer, a pharmaceutically acceptable salt, or a derivative thereof.

In one embodiment, the compound represented by Formula (Ia) is a compound represented by Formula (XIIa)

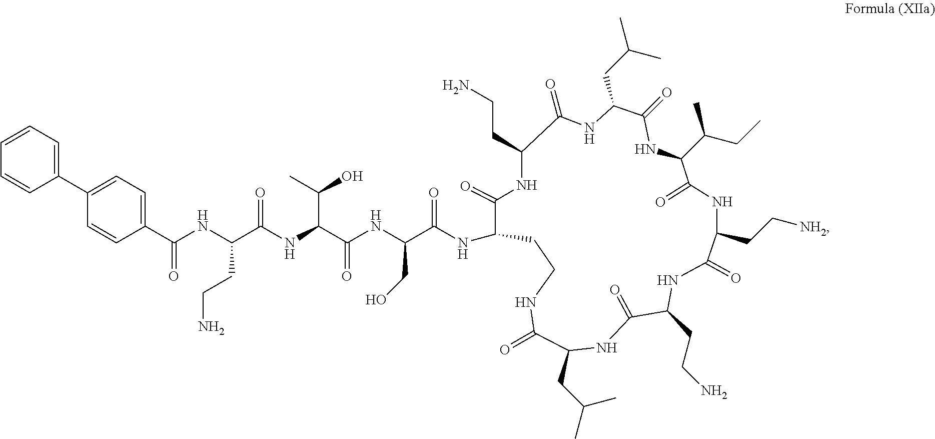 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)c2ccc(-c3ccccc3)cc2)[C@@H](C)O)CCNC(=O)[C@H](CC(C)C)NC(=O)[C@H](CCN)NC(=O)[C@H](CCN)NC1=O]

or a racemate, an enantiomer, a diastereomer, a pharmaceutically acceptable salt, or a derivative thereof.

In one embodiment, the compound represented by Formula (Ia) is a compound represented by Formula (XIIIa)

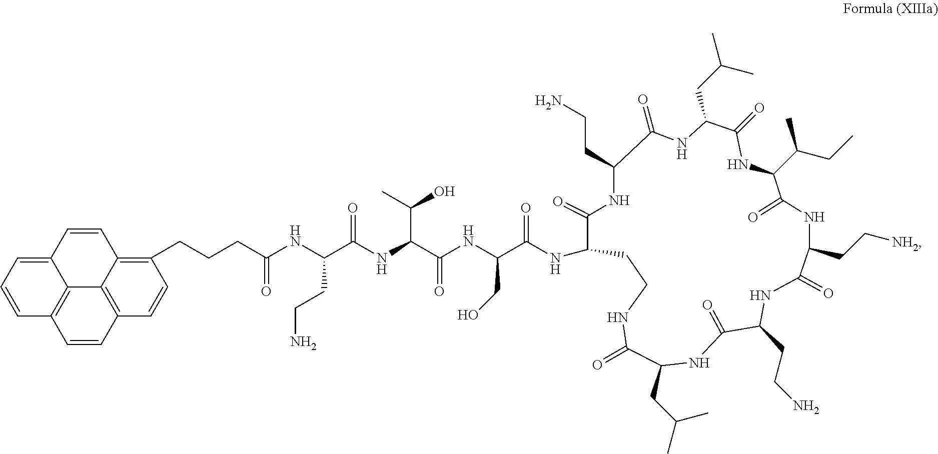 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)CCCc2ccc3ccc4cccc5ccc2c3c45)[C@@H](C)O)CCNC(=O)[C@H](CC(C)C)NC(=O)[C@H](CCN)NC(=O)[C@H](CCN)NC1=O]

or a racemate, an enantiomer, a diastereomer, a pharmaceutically acceptable salt, or a derivative thereof.

In one embodiment, the compound represented by Formula (Ia) is a compound represented by Formula (XIVa)

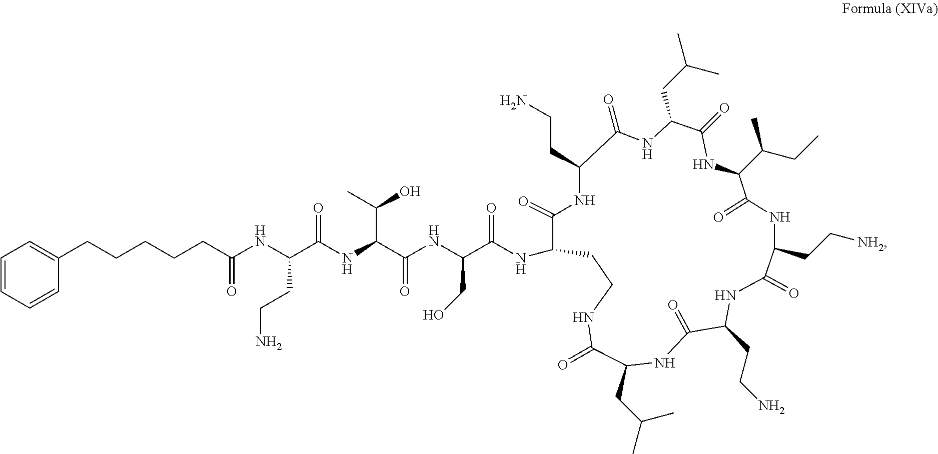 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)CCCCCc2ccccc2)[C@@H](C)O)CCNC(=O)[C@H](CC(C)C)NC(=O)[C@H](CCN)NC(=O)[C@H](CCN)NC1=O]

or a racemate, an enantiomer, a diastereomer, a pharmaceutically acceptable salt, or a derivative thereof.

In one embodiment, the compound represented by Formula (Ia) is a compound represented by Formula (XVa)

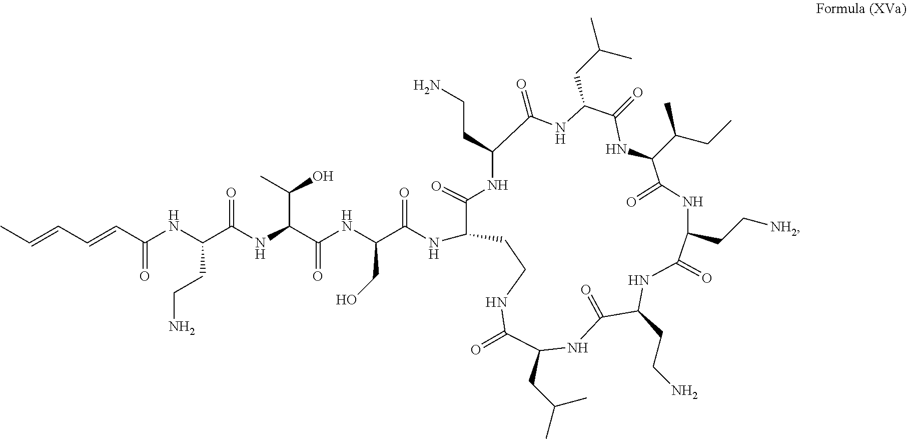 [SMILES: C/C=C/C=C/C(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]

or a racemate, an enantiomer, a diastereomer, a pharmaceutically acceptable salt, or a derivative thereof.

In one embodiment, the compound represented by Formula (Ia) is a compound represented by Formula (XVIa)

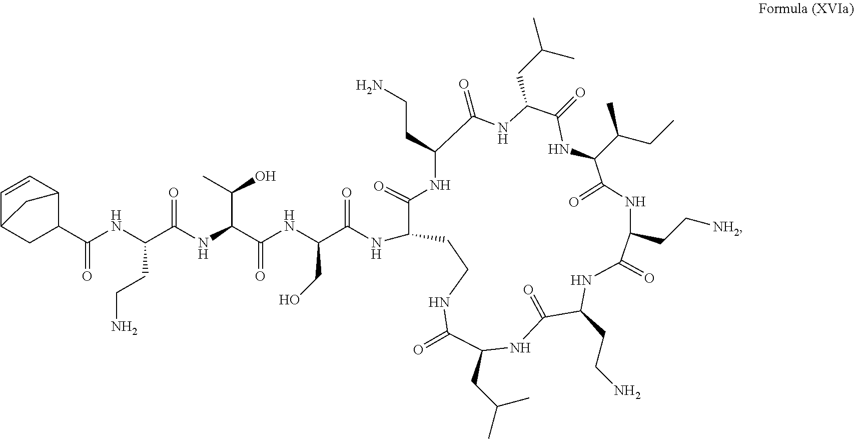 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)C2CC3C=CC2C3)[C@@H](C)O)CCNC(=O)[C@H](CC(C)C)NC(=O)[C@H](CCN)NC(=O)[C@H](CCN)NC1=O]

or a racemate, an enantiomer, a diastereomer, a pharmaceutically acceptable salt, or a derivative thereof.

In one embodiment, the compound represented by Formula (Ia) is a compound represented by Formula (XVIIa)

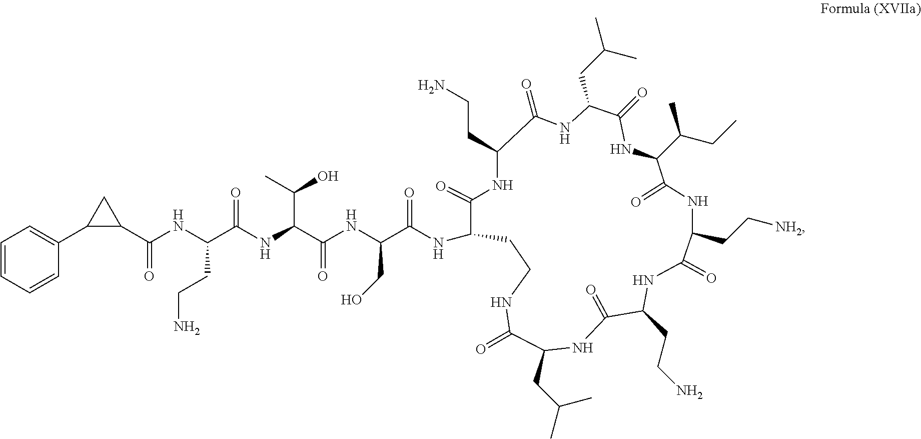 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)C2CC2c2ccccc2)[C@@H](C)O)CCNC(=O)[C@H](CC(C)C)NC(=O)[C@H](CCN)NC(=O)[C@H](CCN)NC1=O]

or a racemate, an enantiomer, a diastereomer, a pharmaceutically acceptable salt, or a derivative thereof.

In one embodiment, the compound represented by Formula (Ia) is a compound represented by Formula (XVIIIa)

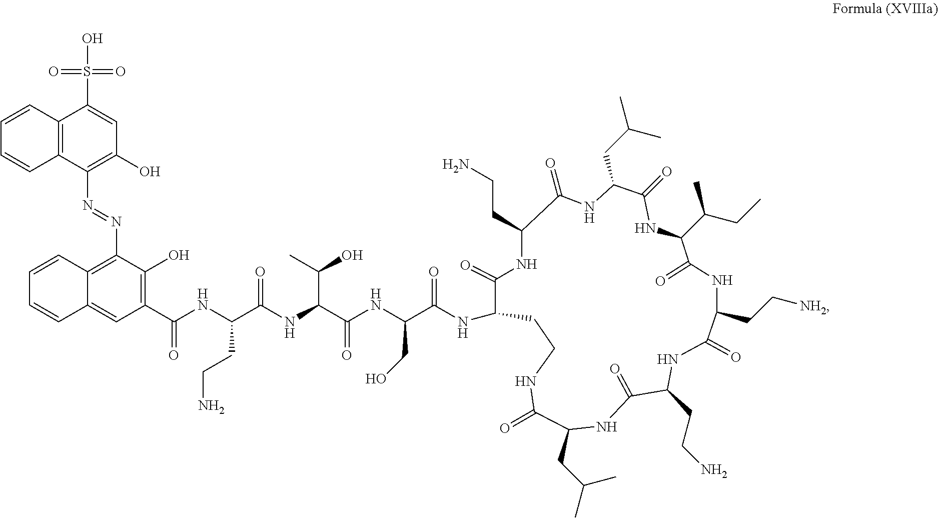 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)c2cc3ccccc3c(/N=N/c3c(O)cc(S(=O)(=O)O)c4ccccc34)c2O)[C@@H](C)O)CCNC(=O)[C@H](CC(C)C)NC(=O)[C@H](CCN)NC(=O)[C@H](CCN)NC1=O]

or a racemate, an enantiomer, a diastereomer, a pharmaceutically acceptable salt, or a derivative thereof.

In one embodiment, the compound represented by Formula (Ia) is a compound represented by Formula (XIXa)

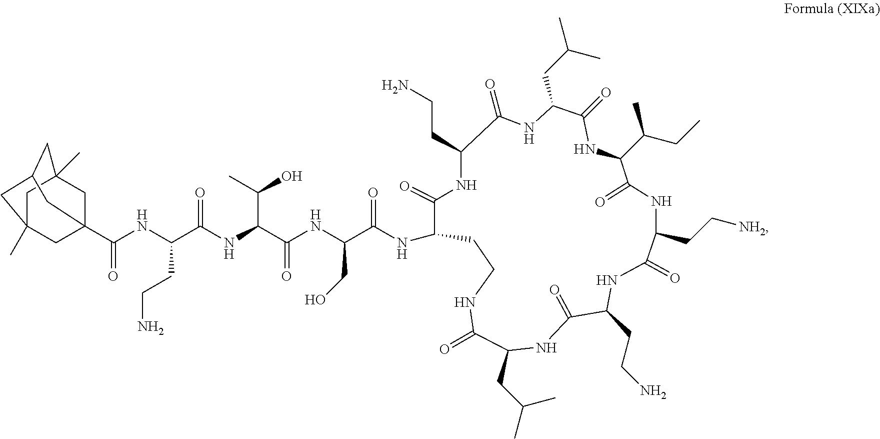 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)C23CC4CC(C)(CC(C)(C4)C2)C3)[C@@H](C)O)CCNC(=O)[C@H](CC(C)C)NC(=O)[C@H](CCN)NC(=O)[C@H](CCN)NC1=O]

or a racemate, an enantiomer, a diastereomer, a pharmaceutically acceptable salt, or a derivative thereof.

In one embodiment, the compound represented by Formula (Ia) is a compound represented by Formula (XXa)

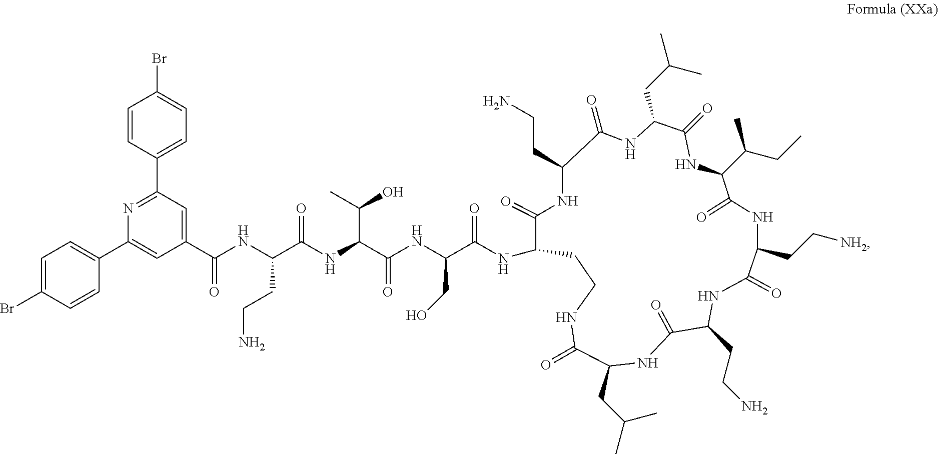 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)c2cc(-c3ccc(Br)cc3)nc(-c3ccc(Br)cc3)c2)[C@@H](C)O)CCNC(=O)[C@H](CC(C)C)NC(=O)[C@H](CCN)NC(=O)[C@H](CCN)NC1=O]

or a racemate, an enantiomer, a diastereomer, a pharmaceutically acceptable salt, or a derivative thereof.

The compounds described herein may form salts with acids or bases, and such salts are included in the present invention. The term “salts” embraces addition salts of free acids or free bases that are compounds of the invention.

In one aspect, the present invention relates, in part, to compositions comprising one or more compounds of the present invention. In some embodiments, the composition comprises one or more compounds having the structure of Formulae (I)-(XX), or a racemate, an enantiomer, a diastereomer, a pharmaceutically acceptable salt, or a derivative thereof. In some embodiments, the composition is the pharmaceutical composition.

### Methods of Generating Compounds

In one aspect, the present invention relates, in part, to a method of generating one or more compounds of the present invention. In various embodiments, the compounds of the present invention can be generated using any method known to those of skill in the art. For example, in one embodiment, the compounds can be synthesized using any method known to those of skill in the art.

For example, the compounds of the present invention may be synthesized using techniques well-known in the art of organic synthesis. The starting materials and intermediates required for the synthesis may be obtained from commercial sources or synthesized according to methods known to those skilled in the art.

Alternatively, the compounds can be biosynthesized via heterologous expression of a biosynthetic gene. For example, in one embodiment, biosynthesizing macolacins comprises providing a heterologous nucleic acid of the invention to a host, incubating the host in a growth medium, and isolating a macolacin from the host or the growth medium. In one embodiment, the macolacin is isolated from the growth medium. In one embodiment, providing a heterologous nucleic acid to the host comprises transforming the host with the heterologous nucleic acid.

The term “heterologous nucleic acid” as used herein refers to a nucleic acid sequence, which has been introduced into the host organism, wherein said host does not endogenously comprise said nucleic acid. For example, said heterologous nucleic acid may be introduced into the host organism by recombinant methods. Thus, the genome of the host organism has been augmented by at least one incorporated heterologous nucleic acid sequence. It will be appreciated that typically the genome of a recombinant host described herein is augmented through the stable introduction of one or more heterologous nucleic acids encoding one or more macolacins.

In another embodiment, the present invention provides methods of generating macolacins via isolated nucleic acids and vectors encoding a macolacin. In one embodiment, when the nucleic acids and vectors are administered to a subject, they produce a macolacin. In one embodiment, when the nucleic acids and vectors are administered to a subject, they produce an antibacterial effect.

The nucleic acid sequences include both the DNA sequence that is transcribed into RNA and the RNA sequence that is translated into a polypeptide. According to other embodiments, the polynucleotides of the invention are inferred from the amino acid sequence of the polypeptides of the invention. As is known in the art several alternative polynucleotides are possible due to redundant codons, while retaining the biological activity of the translated polypeptides.

It is to be understood explicitly that the scope of the present invention encompasses homologs, analogs, variants, fragments, derivatives and salts, including shorter and longer polynucleotides as well as polynucleotide analogs with one or more nucleic acid substitution, as well as nucleic acid derivatives, non-natural nucleic acids and synthetic nucleic acids as are known in the art, with the stipulation that these modifications must preserve the activity of the original molecule. The invention should be construed to include any and all isolated nucleic acids which are homologous to the nucleic acids described and referenced herein.

The skilled artisan would understand that the nucleic acids of the invention encompass a RNA or a DNA sequence comprising a sequence of the invention, and any modified forms thereof, including chemical modifications of the DNA or RNA which render the nucleotide sequence more stable when it is cell free or when it is associated with a cell. Chemical modifications of nucleotides may also be used to enhance the efficiency with which a nucleotide sequence is taken up by a cell or the efficiency with which it is expressed in a cell. Any and all combinations of modifications of the nucleotide sequences are contemplated in the present invention.

Vectors include, but are not limited to, plasmids, expression vectors, recombinant viruses, any form of recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

In one embodiment, the vector is a plasmid. The plasmid may comprise one or more sequences encoding macolacins described herein. The plasmid may further comprise an initiation codon, which may be upstream of the coding sequence, and a stop codon, which may be downstream of the coding sequence. The initiation and termination codon may be in frame with the coding sequence.

The plasmid may also comprise a promoter that is operably linked to the coding sequence The promoter operably linked to the coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, or human metalothionein.

The promoter may also be a tissue specific promoter, such as a muscle or skin specific promoter, natural or synthetic. Examples of such promoters are described in US patent application publication no. US20040175727, the contents of which are incorporated herein in its entirety.

The plasmid may also comprise a polyadenylation signal, which may be downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, CA).

The plasmid may also comprise an enhancer upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference.

The plasmid may also comprise a mammalian origin of replication in order to maintain the plasmid extrachromosomally and produce multiple copies of the plasmid in a cell. The plasmid may be pVAX1, pCEP4 or pREP4 from Invitrogen (San Diego, CA), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may also comprise a regulatory sequence, which may be well suited for gene expression in a cell into which the plasmid is administered. The coding sequence may comprise a codon that may allow more efficient transcription of the coding sequence in the host cell. In one embodiment, the plasmid may be pTARa (Invitrogen, San Diego, Calif.) plasmid.

Also provided herein is a linear nucleic acid vaccine, or linear expression cassette (“LEC”). The LEC may be any linear DNA devoid of any phosphate backbone. The DNA may encode one or more macolacins. The LEC may contain a promoter, an intron, a stop codon, a polyadenylation signal. The expression of the antigen may be controlled by the promoter. The LEC may not contain any antibiotic resistance genes and/or a phosphate backbone. The LEC may not contain other nucleic acid sequences unrelated to the desired macolacin expression.

The LEC may be derived from any plasmid capable of being linearized. The plasmid may be capable of expressing the macolacin. The plasmid may be any expression vector capable of expressing the DNA.

In one embodiment, viral vectors are provided herein which are capable of delivering a nucleic acid of the invention to a cell. The expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Suitable host organisms include microorganisms, plant cells, and plants. The microorganism can be any microorganism suitable for expression of heterologous nucleic acids. In one embodiment the host organism of the invention is a eukaryotic cell. In another embodiment the host organism is a prokaryotic cell. In one embodiment, the host organism is a fungal cell such as a yeast or filamentous fungus. In one embodiment the host organism may be a yeast cell.

The host organism may also be a plant. plant or plant cell can be transformed by having a heterologous nucleic acid integrated into its genome, i.e., it can be stably transformed. Stably transformed cells typically retain the introduced nucleic acid with each cell division. A plant or plant cell can also be transiently transformed such that the recombinant gene is not integrated into its genome. Transiently transformed cells typically lose all or some portion of the introduced nucleic acid with each cell division such that the introduced nucleic acid cannot be detected in daughter cells after a certain number of cell divisions.

In one embodiment, the host is an engineered cell that expresses a macolacin. The genetically modified cell according to the invention may be constructed from any suitable host cell. The host cell may be an unmodified cell or may already be genetically modified. The cell may be a prokaryote cell, a eukaryote cell, a plant cell or an animal cell.

In one embodiment, the engineered cell is modified by way of introducing genetic material into the cell in order for the cell to produce a macolacin. In one embodiment, the engineered cell is modified by way of transforming a nucleic acid of the invention into the cell.

In one embodiment, the engineered cell produces a compound of Formula (I). In some embodiments, the engineered cell produces at least one compound of Formula (I)-(XX). For example, in one embodiment, the engineered cell produces a compound of Formula (II). In one embodiment, the engineered cell produces a compound of Formula (XII).

In one embodiment, the engineered cell produces a compound of Formula (Ia). In some embodiments, the engineered cell produces at least one compound of Formula (Ia)-(XXa). For example, in one embodiment, the engineered cell produces a compound of Formula (IIa). In one embodiment, the engineered cell produces a compound of Formula (XIIa).

In one embodiment, the cell is a eukaryotic cell. In one embodiment, the cell may be a human cell, a non-human mammalian cell, a non-mammalian vertebrate cell, an invertebrate cell, an insect cell, a plant cell, a yeast cell, or a single cell eukaryotic organism. In one embodiment, the cell may be an adult cell or an embryonic cell (e.g., an embryo). In one embodiment, the cell may be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, unipotent stem cells and others.

In one embodiment, the cell is a cell line cell. Non-limiting examples of suitable mammalian cells include Chinese hamster ovary (CHO) cells, baby hamster kidney (BHK) cells; mouse myeloma NSO cells, mouse embryonic fibroblast 3T3 cells (NIH3T3), mouse B lymphoma A20 cells; mouse melanoma B16 cells; mouse myoblast C2C12 cells; mouse myeloma SP2/0 cells; mouse embryonic mesenchymal C₃H-10T1/2 cells; mouse carcinoma CT26 cells, mouse prostate DuCuP cells; mouse breast EMT6 cells; mouse hepatoma Hepalclc7 cells; mouse myeloma J5582 cells; mouse epithelial MTD-1A cells; mouse myocardial MyEnd cells; mouse renal RenCa cells; mouse pancreatic RIN-5F cells; mouse melanoma X64 cells; mouse lymphoma YAC-1 cells; rat glioblastoma 9L cells; rat B lymphoma RBL cells; rat neuroblastoma B35 cells; rat hepatoma cells (HTC); buffalo rat liver BRL 3A cells; canine kidney cells (MDCK); canine mammary (CMT) cells; rat osteosarcoma D17 cells; rat monocyte/macrophage DH82 cells; monkey kidney SV-40 transformed fibroblast (COS7) cells; monkey kidney CVI-76 cells; African green monkey kidney (VERO-76) cells; human embryonic kidney cells (HEK293, HEK293T); human cervical carcinoma cells (HELA); human lung cells (W138); human liver cells (Hep G2); human U2—OS osteosarcoma cells, human A549 cells, human A-431 cells, human SW48 cells, human HCT116 cells, and human K562 cells. An extensive list of mammalian cell lines may be found in the American Type Culture Collection catalog (ATCC, Manassas, Va.).

In one embodiment, the cell can be a prokaryotic cell or a eukaryotic cell. In one embodiment, the cell is a prokaryotic cell. In one embodiment, the cell is a genetically engineered bacteria cell.

In one embodiment, the genetically engineered bacteria cell is a non-pathogenic bacteria cell. In some embodiments, the genetically engineered bacteria cell is a commensal bacteria cell. In some embodiments, the genetically engineered bacteria cell is a probiotic bacteria cell. In some embodiments, the genetically engineered bacteria cell is a naturally pathogenic bacteria cell that is modified or mutated to reduce or eliminate pathogenicity. Exemplary bacteria include, but are not limited to Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii.

In one embodiment, the host is a Streptomyces albus cell.

In some embodiments, the genetically engineered bacteria are Escherichia coli strain Nissle 1917 (E. coli Nissle), a Gram−negative bacterium of the Enterobacteriaceae family that “has evolved into one of the best characterized probiotics” (Ukena et al., 2007). The strain is characterized by its complete harmlessness (Schultz, 2008), and has GRAS (generally recognized as safe) status (Reister et al., 2014, emphasis added). Genomic sequencing confirmed that E. coli Nissle lacks prominent virulence factors (e.g., E. coli a-hemolysin, P-fimbrial adhesins) (Schultz, 2008). In addition, it has been shown that E. coli Nissle does not carry pathogenic adhesion factors, does not produce any enterotoxins or cytotoxins, is not invasive, and not uropathogenic (Sonnenborn et al., 2009). As early as in 1917, E. coli Nissle was packaged into medicinal capsules, called Mutaflor, for therapeutic use. E. coli Nissle has since been used to treat ulcerative colitis in humans in vivo (Rembacken et al., 1999), to treat inflammatory bowel disease, Crohn's disease, and pouchitis in humans in vivo (Schultz, 2008), and to inhibit enteroinvasive Salmonella, Legionella, Yersinia, and Shigella in vitro (Altenhoefer et al., 2004). It is commonly accepted that E. coli Nissle's therapeutic efficacy and safety have convincingly been proven (Ukena et al., 2007).

One of ordinary skill in the art would appreciate that the genetic modifications disclosed herein may be modified and adapted for other species, strains, and subtypes of bacteria.

### Treatment Methods

In one aspect, the invention provides methods of treating or preventing an infection in a subject in need thereof. In some embodiments, the method comprises administering to the subject an effective amount of a composition comprising at least one compound of the invention (e.g., at least one compound of Formula (I)-(XX)). In some embodiments, the method comprises administering to the subject an effective amount of a composition comprising at least one nucleic acid of the invention.

In some embodiments, the method treats or prevents a bacterial infection. In one embodiment, the method treats or prevents a gram-positive bacterial infection. In one embodiment, the bacterial infection is resistant to antibiotics. For example, in one embodiment, the bacterial infection is resistant to one or more of, beta-lactams, including methicillin, oxacillin, or penicillin, tetracyclines, gentamicin, kanamycin, erythromycin, spectinomycin, and vancomycin.

Exemplary bacterial infections that may be treated by way of the present invention includes, but is not limited to, infections caused by bacteria from the taxonomic genus of Bacillus, Bartonella, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio, and Yersinia. In some embodiments, the bacterial infection is an infection of Acinetobacter baumannii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterobacter cloacae, Enterococcus faecalis, Enterococcus faecium, Escherichia coli, Francisella tularensis, Haemophilus influenzae, Helicobacter pylori, Klebsiella species, Klebsiella pneumoniae, Legionella pneumophila, Leptospira interrogans, Leptospira santarosai, Leptospira weilii, Leptospira noguchii, Listeria monocytogenes, Morexella species, Moraxella osloensis, Mycobacterium leprae, Mycobacterium tuberculosis, Mycobacterium ulcerans, Mycoplasma pneumoniae, Neisseria gonorrhoeae, Neisseria meningitidis, Proteus species, Proteus vulgaris, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, or Yersinia pseudotuberculosis. In one embodiment, the bacterial infection is a Listeria monocytogenes infection.

In one embodiment, the bacterial infection is an infection of S. aureus USA300, S. aureus COL, S. aureus BAA-42, S. aureus NRS100, S. aureus NRS108, S. aureus NRS140, S. aureus NRS146, E. faecium VRE, E. faecium Com15, S. pneumoniae, S. mutans, B. subtilis, L. rhamnosus, E. coli, C. albicans, or C. neoformans.

Exemplary diseases caused by bacterial infections which may be treated using compositions of the present invention, include but are not limited to, bacterially mediated meningitis, sinus tract infections, pneumonia, endocarditis, pancreatitis, appendicitis, gastroenteritis, biliary tract infections, soft tissue infections, urinary tract infections, cystitis, pyelonephritis, osteomyelitis, bacteremia, Actinomycosis, Whooping cough, Secondary bacterial pneumonia, Lyme disease (B. burgdorferi), Relapsing fever, Brucellosis, Enteritis, bloody diarrhea, Guillain-Barré syndrome, Atypical pneumonia, Trachoma, Neonatal conjunctivitis, Neonatal pneumonia, Nongonococcal urethritis (NGU), Urethritis, Pelvic inflammatory disease, Epididymitis, Prostatitis, Lymphogranuloma venereum (LGV), Psittacosis, Botulism: Mainly muscle weakness and paralysis, Pseudomembranous colitis, Anaerobic cellulitis, Gas gangrene Acutefood poisoning, Tetanus, and Diphtheria.

However, the invention should not be limited to only treating bacterial infection. The invention encompasses compounds having an antimicrobial activity including but not limited to antibacterial, antimycobacterial, antifungal, antiviral and the likes.

In one aspect, the invention provides methods of killing a bacterial cell or inhibiting the grown of a bacterial cell. In some embodiments, the method comprises administering to the cell an effective amount of a composition comprising at least one compound of the invention. In some embodiments, the method comprises administering to the cell an effective amount of a composition comprising at least one nucleic acid of the invention. In one embodiment the bacterial cell is a gram positive bacterial cell. In one embodiment, the bacterial cell is resistant to antibiotics. For example, in one embodiment, the bacterial cell is resistant to one or more of, beta-lactams, including methicillin, oxacillin, or penicillin, tetracyclines, gentamicin, kanamycin, erythromycin, spectinomycin, and vancomycin.

In another aspect, the invention provides compositions and methods for treating and/or preventing a disease or disorder related to the detrimental growth and/or proliferation of a bacterial cell in vivo, ex vivo or in vitro. In certain embodiments, the method comprises administering a composition comprising an effective amount of a composition provided by the invention to a subject, wherein the composition is effective in inhibiting or preventing the growth and/or proliferation of a bacterial cell. In certain embodiments, the bacterial cell is a Gram−positive bacterial cell, e.g., a bacteria of a genera such as Staphylococcus, Streptococcus, Enterococcus, (which are cocci) and Bacillus, Corynebacterium, Nocardia, Clostridium, Actinobacteria, and Listeria (which are rods and can be remembered by the mnemonic obconical), Mollicutes, bacteria-like Mycoplasma, Actinobacteria.

In certain embodiments, the bacterial cell is a Gram−bacteria cell, e.g., a bacteria of a genera such as Acinetobacter, Citrobacter, Enterobacter, Enterococcus, Escherichia, Helicobacter, Hemophilus, Klebsiella, Legionella, Moraxella, Neisseria, Proteus, Pseudomonas, Salmonella, Staphylococcus, and Yersinia. The compounds as described herein and compositions comprising them may thus be for use in the treatment of bacterial infections by the above-mentioned Gram+or Gram−bacteria.

In one embodiment, the method further comprises administering a second therapeutic agent. In one embodiment, the second therapeutic agent is an antibiotic agent. In one embodiment, the compound of the invention and the at least one additional antibiotic agent act synergistically in preventing, reducing or disrupting microbial growth.

Non-limiting examples of the at least one additional antibiotic agents include levofloxacin, doxycycline, neomycin, clindamycin, minocycline, gentamycin, rifampin, chlorhexidine, chloroxylenol, methylisothizolone, thymol, a-terpineol, cetylpyridinium chloride, hexachlorophene, triclosan, nitrofurantoin, erythromycin, nafcillin, cefazolin, imipenem, astreonam, gentamicin, sulfamethoxazole, vancomycin, ciprofloxacin, trimethoprim, rifampin, metronidazole, clindamycin, teicoplanin, mupirocin, azithromycin, clarithromycin, ofoxacin, lomefloxacin, norfloxacin, nalidixic acid, sparfloxacin, pefloxacin, amifloxacin, gatifloxacin, moxifloxacin, gemifloxacin, enoxacin, fleroxacin, minocycline, linexolid, temafloxacin, tosufloxacin, clinafloxacin, sulbactam, clavulanic acid, amphotericin B, fluconazole, itraconazole, ketoconazole, nystatin, penicillins, cephalosporins, carbepenems, beta-lactams antibiotics, aminoglycosides, macrolides, lincosamides, glycopeptides, tetracylines, chloramphenicol, quinolones, fucidines, sulfonamides, trimethoprims, rifamycins, oxalines, streptogramins, lipopeptides, ketolides, polyenes, azoles, echinocandines, and any combination thereof.

In one embodiment, the compositions of the invention find use in removing at least a portion of or reducing the number of microorganisms and/or biofilm-embedded microorganisms attached to the surface of a medical device or the surface of a subject's body (such as the skin of the subject, or a mucous membrane of the subject, such as the vagina, anus, throat, eyes or ears). In one embodiment, the compositions of the invention find further use in coating the surface of a medical device, thus inhibiting or disrupting microbial growth and/or inhibiting or disrupting the formation of biofilm on the surface of the medical device. The compositions of the invention find further use in preventing or reducing the growth or proliferation of microorganisms and/or biofilm-embedded microorganisms on the surface of a medical device or on the surface of a subject's body. However, the invention is not limited to applications in the medical field. Rather, the invention includes using a macolacin compound or an analog thereof as an antimicrobial and/or antibiofilm agent in any setting.

The composition of the invention may be administered to a patient or subject in need in a wide variety of ways, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the composition is administered systemically to the subject. In one embodiment, the compositions of the present invention are administered to a patient by i.v. injection. In one embodiment, the composition is administered locally to the subject. In one embodiment, the compositions of the present invention are administered to a patient topically. Any administration may be a single application of a composition of invention or multiple applications. Administrations may be to single site or to more than one site in the individual to be treated. Multiple administrations may occur essentially at the same time or separated in time.

In one aspect, the compositions of the invention may be in the form of a coating that is applied to the surface of a medical device or the surface of a subject's body. In one embodiment, the coating prevents or hinders microorganisms and/or biofilm-embedded microorganisms from growing and proliferating on at least one surface of the medical device or at least one surface of the subject's body. In another embodiment, the coating facilitates access of antimicrobial agents to the microorganisms and/or biofilm-embedded microorganisms, thus helping prevent or hinder the microorganisms and/or biofilm-embedded microorganisms from growing or proliferating on at least one surface of the medical device or at least one surface of the subject's body. The compositions of the invention may also be in the form of a liquid or solution, used to clean the surface of medical device or the surface of a subject's body, on which microorganisms and/or biofilm-embedded microorganisms live and proliferate. Such cleaning of the medical device or body surface may occur by flushing, rinsing, soaking, or any additional cleaning method known to those skilled in the art, thus removing at least a portion of or reducing the number of microorganisms and/or biofilm-embedded microorganisms attached to at least one surface of the medical device or at least one surface of the subject's body.

Subjects to which administration of the pharmaceutical compositions of the invention is contemplated include, but are not limited to, humans and other primates, mammals including but not limited to non-human mammals such as non-human primates, cattle, pigs, horses, sheep, cats, and dogs.

Pharmaceutical compositions of the present invention may be administered in a manner appropriate to the disease to be treated (or prevented). The quantity and frequency of administration will be determined by such factors as the condition of the subject, and the type and severity of the subject's disease, although appropriate dosages may be determined by clinical trials.

When “therapeutic amount” is indicated, the precise amount of the compositions of the present invention to be administered can be determined by a physician with consideration of individual differences in age, weight, disease type, extent of disease, and condition of the patient (subject).

Dosage and Formulation (Pharmaceutical compositions)

The invention also encompasses the use of pharmaceutical compositions comprising a compound of the invention, a nucleic acid of the invention, or salts thereof. Such a pharmaceutical composition may comprise of at least one a compound of the invention, a nucleic acid of the invention, or salts thereof in a form suitable for administration to a subject, or the pharmaceutical composition may comprise at least one a compound of the invention, a nucleic acid of the invention, or salts thereof, and one or more pharmaceutically acceptable carriers, one or more additional ingredients, or some combination of these. The compound or nucleic acid of the invention may be present in the pharmaceutical composition in the form of a physiologically acceptable salt, such as in combination with a physiologically acceptable cation or anion, as is well known in the art.

Administration of the therapeutic agent in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the subject, and whether prevention or treatment is to be achieved. Such factors can be readily determined by the clinician employing animal models or other test systems which are well known to the art

The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

In one embodiment, the pharmaceutical compositions useful for practicing the methods of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

Typically, dosages which may be administered in a method of the invention to a mammal, preferably a human, range in amount from 0.5 μg to about 50 mg per kilogram of body weight of the mammal, while the precise dosage administered will vary depending upon any number of factors, including but not limited to, the type of mammal and type of disease state being treated, the age of the mammal and the route of administration. Preferably, the dosage of the compound will vary from about 1 μg to about 10 mg per kilogram of body weight of the mammal. More preferably, the dosage will vary from about 3 μg to about 5 mg per kilogram of body weight of the mammal.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

The compounds and polypeptides (active ingredients) of this invention can be formulated and administered to treat a variety of disease states by any means that produces contact of the active ingredient with the agent's site of action in the body of the organism. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Accordingly, the pharmaceutical composition of the present invention may be delivered via various routes and to various sites in a mammal body to achieve a particular effect (see, e.g., Rosenfeld et al., 1991; Rosenfeld et al., 1991a; Jaffe et al., supra; Berkner, supra). One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal, as well as topical administration.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the pharmaceutical composition in the particular host.

In one embodiment, the compositions of the invention are formulated using one or more pharmaceutically acceptable excipients or carriers. In one embodiment, the pharmaceutical compositions of the invention comprise a therapeutically effective amount of a compound or conjugate of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

The present invention also provides pharmaceutical compositions comprising one or more of the compositions described herein. Formulations may be employed in admixtures with conventional excipients, i.e., pharmaceutically acceptable organic or inorganic carrier substances suitable for administration to subject. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, PA), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

In an embodiment, the composition includes an anti-oxidant and a chelating agent that inhibits the degradation of one or more components of the composition. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the HMW-HA or other composition of the invention in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

The compound may be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments there between.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating the same or another disease as that treated by the compositions of the invention) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound or conjugate of the invention, alone or in combination with a second pharmaceutical agent; and instructions for using the compound or conjugate to treat, prevent, or reduce one or more symptoms of a disease in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject, or delivering an imaging or diagnostic agent to a subject.

Routes of administration of any of the compositions of the invention include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

These methods described herein are by no means all-inclusive, and further methods to suit the specific application will be apparent to the ordinary skilled artisan. Moreover, the effective amount of the compositions can be further approximated through analogy to compounds known to exert the desired effect.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: Macolacin, Natural Solution to mcr-1 Encoded Colistin Resistance in Gram−Negative

### Pathogens

Colistin binds the lipid A moiety of lipopolysaccharides (LPS), disrupting membrane integrity, and ultimately causing cell death. Unfortunately, the extensive use of colistin in animal production, and its increasing use in human pharmacotherapy, has led to a troubling rise in the appearance of resistance in clinical isolates (Jeannot K et al., 2017, Int J Antimicrob Agents, 49:526-535). Of particular concern is the recent appearance and rapid global spread of the plasmid-borne mobilized colistin resistance (mcr-1) gene and its relatives. Mcr-1 encodes a phosphoethanolamine (PEtN) transferase that appends PEtN to a phosphate on lipid A thereby reducing the electrostatic interaction between colistin and LPS and rendering bacteria resistant to colistin antibiosis. Since first being observed in 2015, mor-1 has been detected around the world in clinical isolates of numerous Gram−negative pathogens including MDR Klebsiella pneumoniae, Acinetobacter baumannii, Pseudomonas aeruginosa and Enterobacter species (Liu Y Y et al., 2017, Antimicrobial Agents and Chemotherapy, 61:6; Schwarz S et al., 2016, J Antimicrob Chemother, 71:2066-2070; Hameed F et al., 2019, Rev Soc Bras Med Trop, 52; Tian G B et al., 2017, Lancet Infect Dis, 17:577)

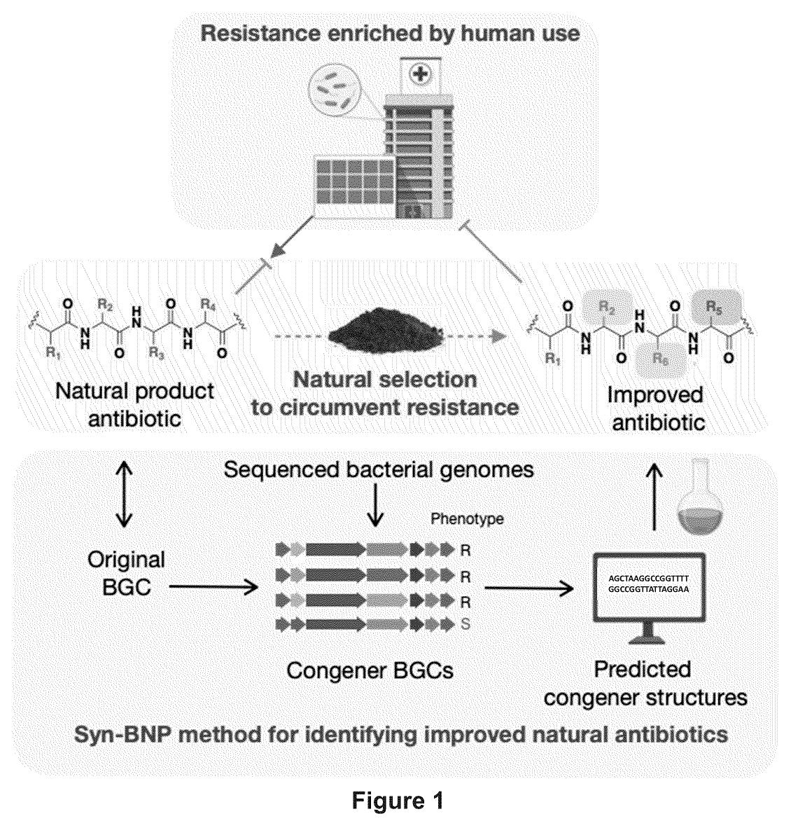

As is seen for most natural product antibiotics, colistin is part of a collection of structurally related metabolites that are encoded by evolutionarily related but distinct BGCs. Colistin belongs to the polymyxin family of antibiotics, which are cationic cyclic lipo-decapeptides that arise from nonribosomal peptide synthetase (NRPS) BGCs found in the genomes of Paenibacillus spp. Across this family of antibiotics, structures differ slightly by both peptide sequence and the specific lipid that is attached to the N-terminus of the decapeptide. The ecological significance of the evolution of collections of structural analogs in place of a single winning natural antibiotic structure may differ from one natural product family to the next; however, the evolution of antibiotic resistance is undoubtedly one of the key drivers of this structural diversification. While occurring on dramatically different time scales, clinical and environmental associated antibiotic resistance arise from the same pool of potential resistance genes, indicating that naturally occurring congeners, which have evolved to circumvent environmental resistance mechanisms, are likely useful for addressing antibiotic resistance that has evolved in the health care setting (FIG. 1). In the case of mcr-1 mediated colistin resistance, this possibility was especially intriguing as bacteria that are intrinsically resistant to colistin often contain chromosomally encoded PEtN transferases that were likely to drive, long before the recent global mobilization of mcr-1, the natural selection for colistin like antibiotics that circumvent this lipid A modification. Such an antibiotic is particularly appealing as it is not only useful for addressing mcr-1 encoded resistance, but also potentially useful against a number of difficult to treat pathogens, e.g., Neisseria gonorrohoeae, that are intrinsically resistant to colistin due to chromosomally encoded PEtN transferases.

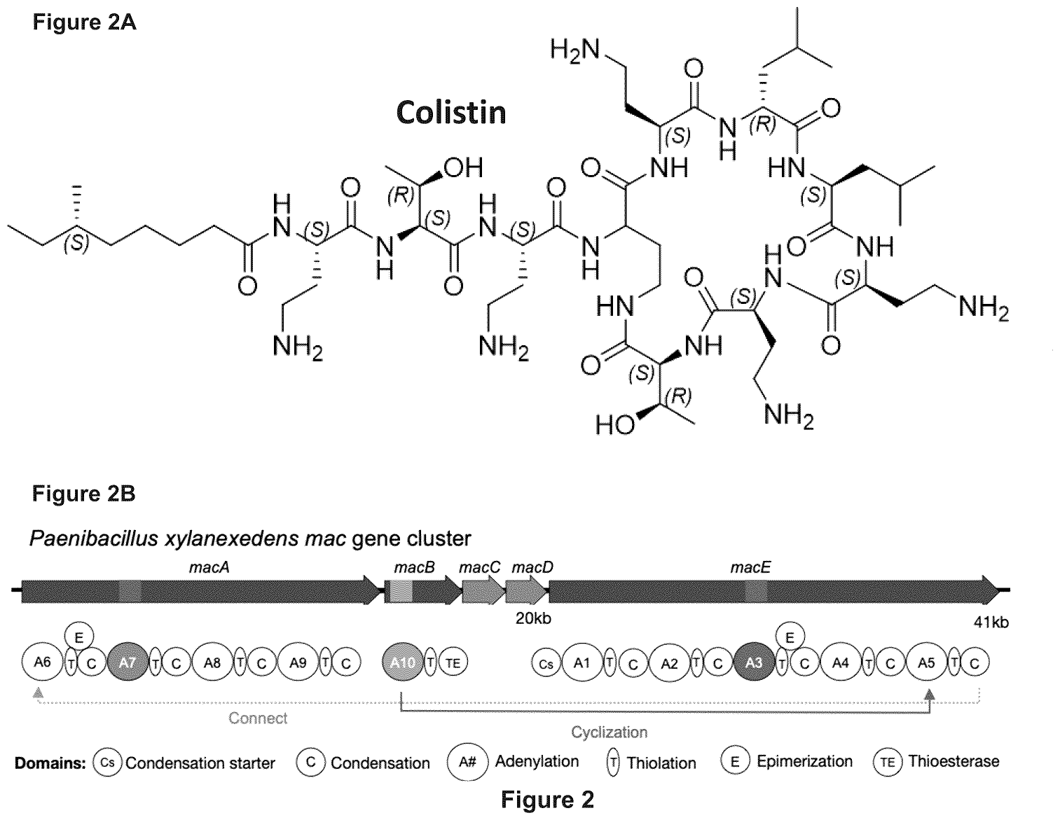

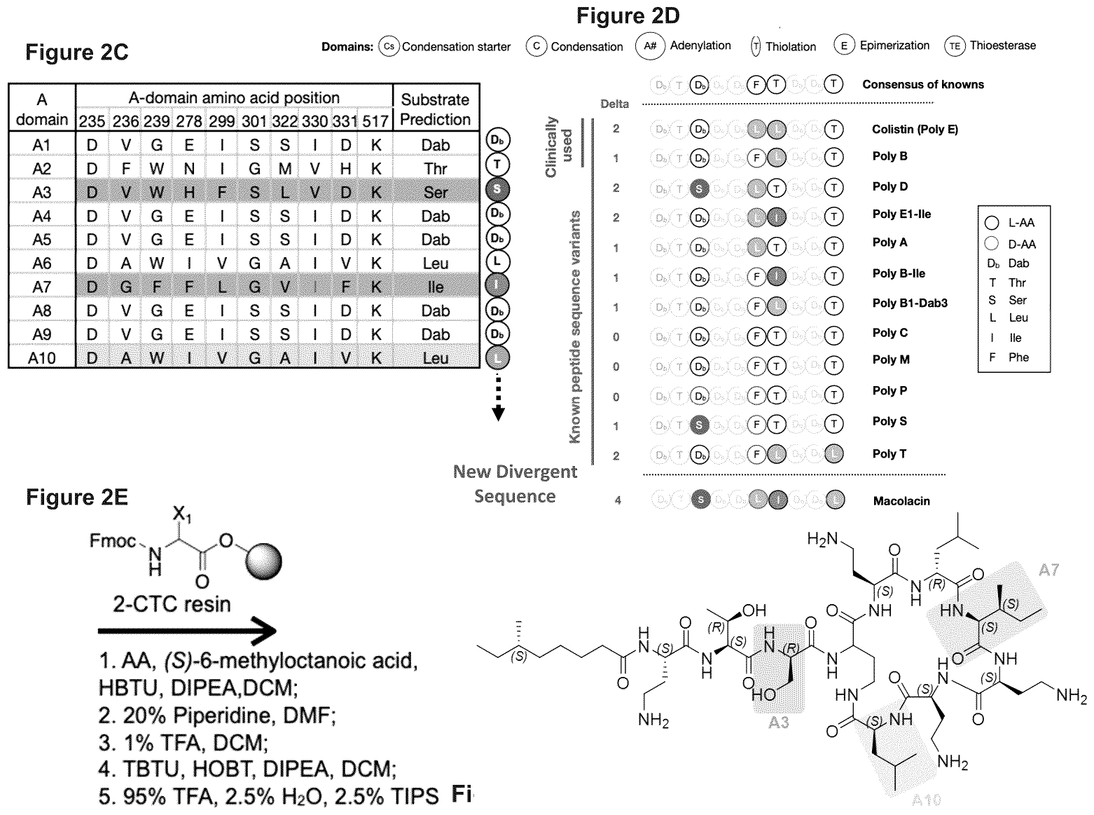

Due to historical difficulties with culturing bacteria as well as difficulties with activating BGCs in laboratory fermentation studies, only a subset of the naturally occurring congeners within a family of antibiotics is likely represented among characterized natural products (Rutledge P J et al., 2015, Nat Rev Microbiol, 13:509-523). The recent exponential growth in genomic and metagenomic sequence data provides a window into bacterial BGCs that have until now remained functionally inaccessible in the search for new antibiotics. In an effort to systematically identify naturally occurring polymyxin family members, the present studies focused on searching 10,858 sequenced bacterial genomes for polymyxin/colistin-like BGCs (FIG. 2A and FIG. 2B). This led to the identification of thirty-five BGCs that were likely to encode polymyxin family antibiotics. Each BGC contained the same gene content and gene organization as was seen in previously characterized polymyxin-family BGCs and each was likely to encode an N-acylated decapeptide.

Nonribosomal peptides (NRPs) are produced by sets of multi domain modules that extend the growing peptide one amino acid per module. A typical minimal NRPS module contains an adenylation (A), a condensation (C), and a thiolation (T) domain, which activate an amino acid substrate, catalyze the new peptide bond formation, and carry the growing peptide, respectively (FIG. 2B; Sussmuth R D et al., 2017, Angew Chem Int Ed Engl, 56:3770-3821). The specific amino acid incorporated into the growing nonribosomal peptide by an NRPS module can be empirically determined based on the ten amino acids that line the A-domain substrate binding pocket (Stachelhaus T et L., 1999, Chem Biol, 6:493-505). To determine the specific linear decapeptide that is produced by each predicted polymyxin family BGC, each A-domain substrate binding pocket was compared to the ten amino acid signatures seen in a collection of characterized A-domains (FIG. 2C and Table 1).

TABLE 1
Macolacin A domain analysis. Bold, italic text (I) indicates a difference
between the signature code of macolacin and the closest known signature
from the BGC of a characterized natural product.

| | Macolacin A domains | | | | | | | | | | |
|---|---|---|---|---|---|---|---|---|---|---|---|
| Domain | 235 | 236 | 239 | 278 | 299 | 301 | 322 | 330 | 331 | 517 | Prediction |
| A1 | D | V | G | E | I | S | S | I | D | K | Dab |
| A2 | D | F | W | N | I | G | M | V | H | K | Thr |
| A3 | D | V | W | H | F | S | L | V | D | K | Ser |
| A4 | D | V | G | E | I | S | S | I | D | K | Dab |
| A5 | D | V | G | E | I | S | S | I | D | K | Dab |
| A6 | D | A | W | I | V | G | A | I | V | K | Leu |
| A7 | D | G | F | F | L | G | V | I | F | K | Ile |
| A8 | D | V | G | E | I | S | S | I | D | K | Dab |
| A9 | D | V | G | E | I | S | S | I | D | K | Dab |
| A10 | D | A | W | I | V | G | A | I | V | K | Leu |
| | Closest characterized A domain | | | | | | | | | | |
| Domain | 235 | 236 | 239 | 278 | 299 | 301 | 322 | 330 | 331 | 517 | Source |
| A1 | D | V | G | E | I | S | S | I | D | K | Poly- |
| | | | | | | | | | | | myxin B |
| A2 | D | F | W | N | I | G | M | V | H | K | Poly- |
| | | | | | | | | | | | myxin B |
| A3 | D | V | W | H | F | S | L | V | D | K | Poly- |
| | | | | | | | | | | | myxin B |
| A4 | D | V | G | E | I | S | S | I | D | K | Poly- |
| | | | | | | | | | | | myxin B |
| A5 | D | V | G | E | I | S | S | I | D | K | Poly- |
| | | | | | | | | | | | myxin B |
| A6 | D | A | W | I | V | G | A | I | V | K | Poly- |
| | | | | | | | | | | | myxin B |
| A7 | D | G | F | F | L | G | V | V | F | K | Bacitracin |
| A8 | D | V | G | E | I | S | S | I | D | K | Poly- |
| | | | | | | | | | | | myxin B |
| A9 | D | V | G | E | I | S | S | I | D | K | Poly- |
| | | | | | | | | | | | myxin B |
| A10 | D | A | W | I | V | G | A | I | V | K | Poly |
| | | | | | | | | | | | myxin B |

Known polymyxin congeners did not differ significantly from the consensus peptide that arises from comparing all characterized antibiotics in the family (Rabanal F et al., 2017, Nat Prod Rep, 34:886-908; Li J et al., 2019, Polymyxin Antibiotics: From Laboratory Bench To Bedside, 1145: V-VI). Based on the A-domain specificity analyses, most sequenced polymyxin family BGCs were similarly indicated to produce NRP decapeptides that are either identical or nearly identical to those seen in previously characterized natural products (i.e., two or fewer amino acid changes) (FIG. 2D).

However, in one case, referred to as the mac BGC, the bioinformatically predicted decapeptide differed from the consensus polymyxin family antibiotic sequence by four amino acids, which was a larger difference than was seen in any previously reported congeners. Like colistin it contained a leucine instead of the more commonly seen phenylalanine at position 6. In addition, at positions 3, 7, and 10, it contained a serine, an isoleucine, and a leucine instead of the 2,4-diaminobutyric acid (Dab), leucine, and threonine that were found in the consensus structure. As one of the strongest selective pressures for the creation of new congener structures was likely the development of resistance to previous antibiotics in a family, this divergent structure was of particular interest because of the chance it is likely to represent the most evolved natural response to antibiotic resistance seen to date.

Although natural product isolation has traditionally relied on the analysis of bacterial fermentation broths, this process remains resource intensive and is limited by the fact that the majority of bacterial BGCs remain silent in laboratory-based fermentation studies (Tomm H A et al., 2019, J Ind Microbiol Biotechnol, 46:1381-1400). With the increasing accuracy of bioinformatic algorithms for predicting natural product structures, total chemical synthesis of the bioinformatically predicted BGC product (i.e., a synthetic bioinformatic natural product, syn-BNP) provides an alternate and potentially more straightforward method for accessing some small molecules encoded by sequenced BGCs (Chu J et al., 2016, Nat Chem Biol, 12:1004-1006; Chu J et al., 2019, J Am Chem Soc, 141:15737-15741; Chu J et al., 2020, J Am Chem Soc, 142:14158-14168). The absence of post NRPS tailoring enzymes in polymyxin family BGCs means that the final linear peptide encoded by a BGC in this family can be predicted solely based on A-domain substrate specificity analysis. To access the predicted product of the mac BGC, solid phase peptide synthesis (SPPS) was first used to generate its bioinformatically predicted linear decapeptide product (FIG. 2E). This was then N-terminally acylated with(S)-6-methyloctanoic acid, which is the lipid most commonly seen in this family of antibiotics. Cyclization through the 2,4-diaminobutyric acid at position 4 and deprotection gave a syn-BNP that was named macolacin (mcr-1 active colistin-like antibiotic).

### Minimum Inhibitory Concentration (MIC) and Mode of Action

Initially, macolacin was tested for activity against colistin sensitive ESKAPE pathogens, which represent a taxonomically diverse collection of bacteria that is commonly associated with antibiotic-resistant nosocomial infections (e.g., E. faecium, S. aureus, K. pneumoniae, A. baumannii, P. aeruginosa, and Enterobacter species). Consistent with colistin and other polymyxin family antibiotics, macolacin showed potent, narrow spectrum Gram−negative activity. MICs against Gram−negative pathogens ranged from 1-4 μg/mL. Macolacin and colistin are essentially equipotent against K. pneumoniae, A. baumannii, and macolacin is slightly less active than colistin against P. aeruginosa and E. cloacae. At the highest concentration tested (128 μg/mL), no activity against Gram−positive pathogens was observed (Table 2).

TABLE 2
MIC values of macolacin, polymyxin B, and colistin against
a panel of sensitive and resistant ESKAPE pathogens (n =
2). S = colistin sensitive (MIC < 2 μg/mL), R
= colistin resistant (MIC > 2 μg/mL).

| | | | Polymyxin | Colistin | Macolacin |
|---|---|---|---|---|---|
| Strain | S/R | | MIC μg/mL | | |
| ESKAPE pathogens | | | | | |
| E. faecium Com15 | R | | >128 | >128 | >128 |
| S. aureus SH1000 | R | | >128 | >128 | >128 |
| K. pneumoniae 10031 | S | | 1 | 0.5 | 1 |
| A. baumannii 17978 | S | | 0.5 | 1 | 1 |
| P. aeruginosa PA01 | S | | 2 | 1 | 4 |
| E. cloacae 0150 | S | | 1 | 1 | 4 |
| Clinical Isolates | | | | | |
| K. pneumoniae 0497 | R | mcr-1 | 32 | 32 | 2 |
| S. typhimurium 0635 | R | mcr-1 | 16 | 16 | 4 |
| mcr-1 engineered pairs | | | | | |
| K. pneumoniae | S | | 1 | 1 | 1 |
| ATCC13883 | | | | | |
| K. pneumoniae | R | mcr-1 | 128 | 64 | 4 |
| ATCC13883 | | | | | |
| (pMQ124-mcr-1) | | | | | |
| A. baumannii | S | | 0.5 | 1 | 1 |
| ATCC17978 | | | | | |
| A. baumannii | R | mcr-1 | 16 | 32 | 2 |
| ATCC17978 | | | | | |
| (pMQ124xlab1-mcr-1) | | | | | |
| A. baumannii SM1536 | S | | 1 | 0.5 | 2 |
| A. baumannii SM1536 | R | mcr-1 | 128 | 128 | 8 |
| (pMQ124xlab1-mcr-1) | | | | | |
| phoPQ engineered | | | | | |
| pairs | | | | | |
| E. cloacae | R | | 1 | 1 | 1 |
| ATCC13047 | | | | | |
| (ΔphoP/Q) | | | | | |
| E. cloacae | R | phoP/Q | 32 | 32 | 2 |
| ATCC13047 | | | | | |
| (ΔphoP/Q + phoP/Q) | | | | | |

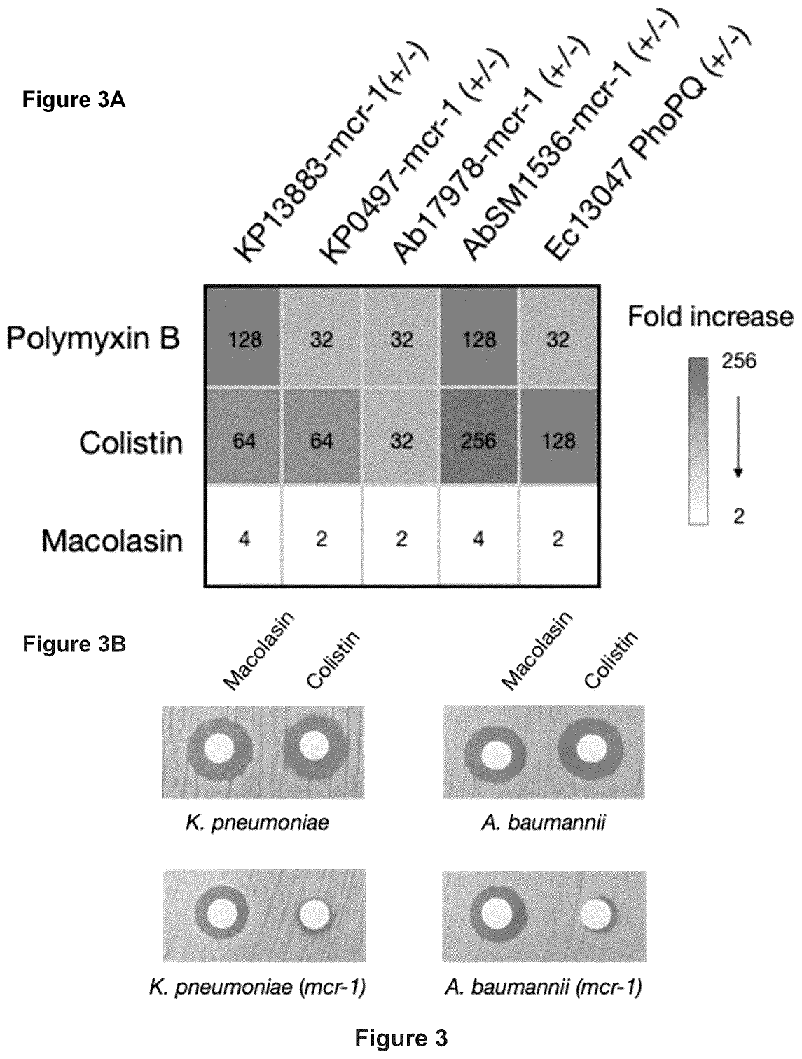

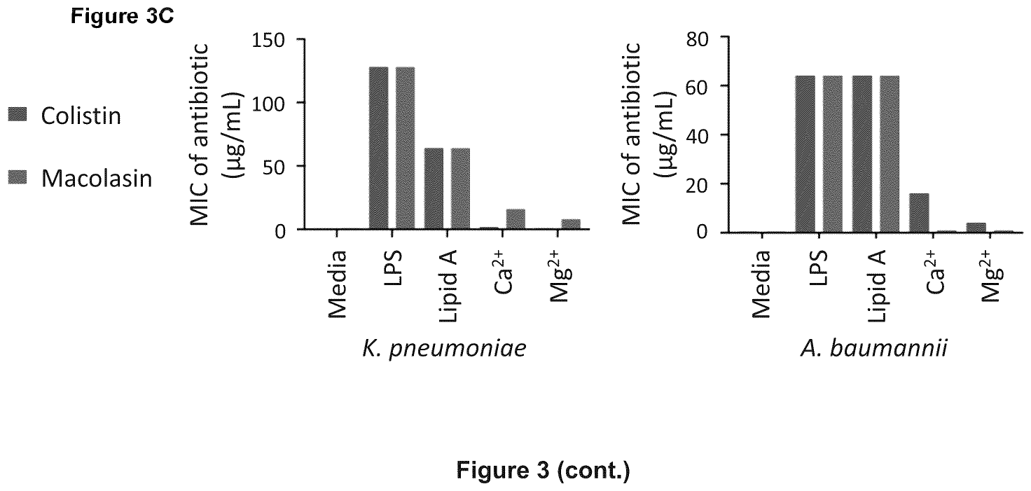

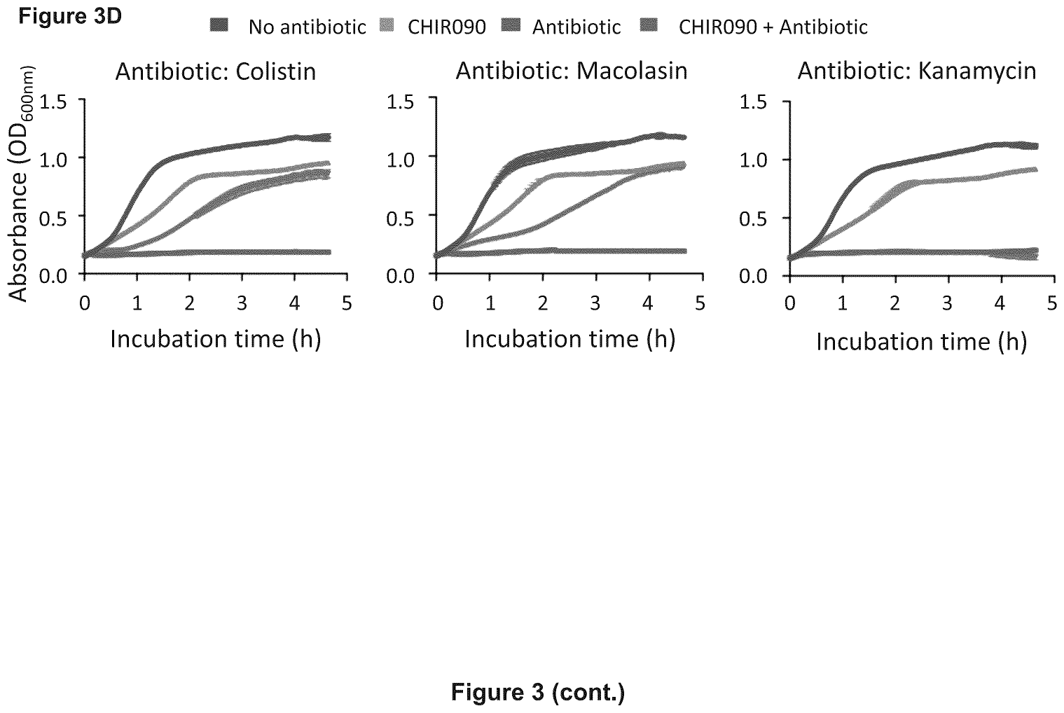

To specifically examine the activity of macolacin against PEtN transferase conferred colistin resistance, pairs of colistin sensitive and resistant (>32 μg/mL) strains of K. pneumoniae and A. baumannii, that were generated by transformation with a plasmid expressing mcr-1 (pMQ124-mcr-1 or pMQ124xlab1-mcr-1, respectively), were used. In the case of colistin and polymyxin B, mcr-1 expression led to a 32-fold or greater (up to 256-fold) increase in MIC. Against the same colistin sensitive and resistant strain pairs, macolacin showed only a 2- to 4-fold increase in MIC, even at the highest levels of colistin resistance (Table 2, FIG. 3A, and FIG. 3B). Although macolacin's activity largely mimicked that of colistin and polymyxin B against colistin sensitive pathogens, it provided significantly improved activity against colistin resistant pathogens.

Another common lipid A modification that confers colistin resistance is the addition of 4-amino-4-deoxy-L-arabinose (L-Ara4N) to a phosphate on the lipid A (Kang K N et al., 2019, Mol Microbiol, 111:1604-1616). This intrinsic resistance mechanism is controlled by the activation of phoP/Q or pmrA/B, two-component regulators that control the expression of L-Ara4N transferase genes (e.g., arnT). Chromosomal mutations in this regulatory system often confer colistin resistance in clinical isolates. When MICs were compared against E. cloacae in which phoP/Q was deleted to a strain where this deletion was rescued by transformation with a plasmid that expresses phoP/Q, a similar phenomenon was observed to the one that was observed with mcr-1 resistance. The knockout strain was sensitive to polymyxin family antibiotics (MIC 1 μg/mL) while the engineered strain was sensitive to macolacin (MIC 2 μg/mL) but resistant to colistin as well as polymyxin B.

Although not bound by any particular theory, it was hypothesized that macolacin's ability to overcome colistin resistance results from either having a distinct mode of action from colistin or from its unique structure retaining the ability to interact with modified lipid A. As seen with colistin, the addition of either lipid A or lipopolysaccharide (LPS) to the assay media caused a significant increase in macolacin's MIC (FIG. 3C).

Although antibiosis suppression in this assay was indicative of macolacin retaining the ability to interact with lipid A, it alone did not rule out the possibility of macolacin's antibiosis arising from a different molecular target. In A. baumannii, LPS was not essential and therefore lipid A biosynthesis inhibitors, like the LpxC inhibitor CHIR-09022, did not prevent A. baumannii growth in the laboratory (Nagy E et al., 2019, J Bacteriol, 201:22; Moffatt J H et al., 2010, Antimicrob Agents Chemother, 54:4971-4977; Wei J R et al., 2017, MSphere, 2:e00199-00117; Richie D L et al., 2016, PLOS One, 11:e0160918). Although CHIR-090 did not inhibit A. baumannii growth, its inhibition of lipid A biosynthesis prevented the production of LPS thereby rescuing A. baumannii from colistin toxicity.

If macolacin's antibiosis remained due to its interaction with lipid A, its activity was similarly suppressed in the presence of CHIR-090. Although A. baumannii cultures treated with CHIR-090 alone grew more slowly than untreated cultures, they reached saturation after 48 hours (FIG. 3D). At concentrations above their MICs both macolacin and colistin completely abrogated A. baumannii growth over this same time period. The inclusion of CHIR-090 to cultures exposed to either macolacin or colistin rescued A. baumannii growth, indicating that macolacin not only retained the ability to bind lipid A but that its antibiosis likely remained the result of this interaction (FIG. 3D).

### Structure Activity Relationship (SAR) of the Peptide Macrocycle

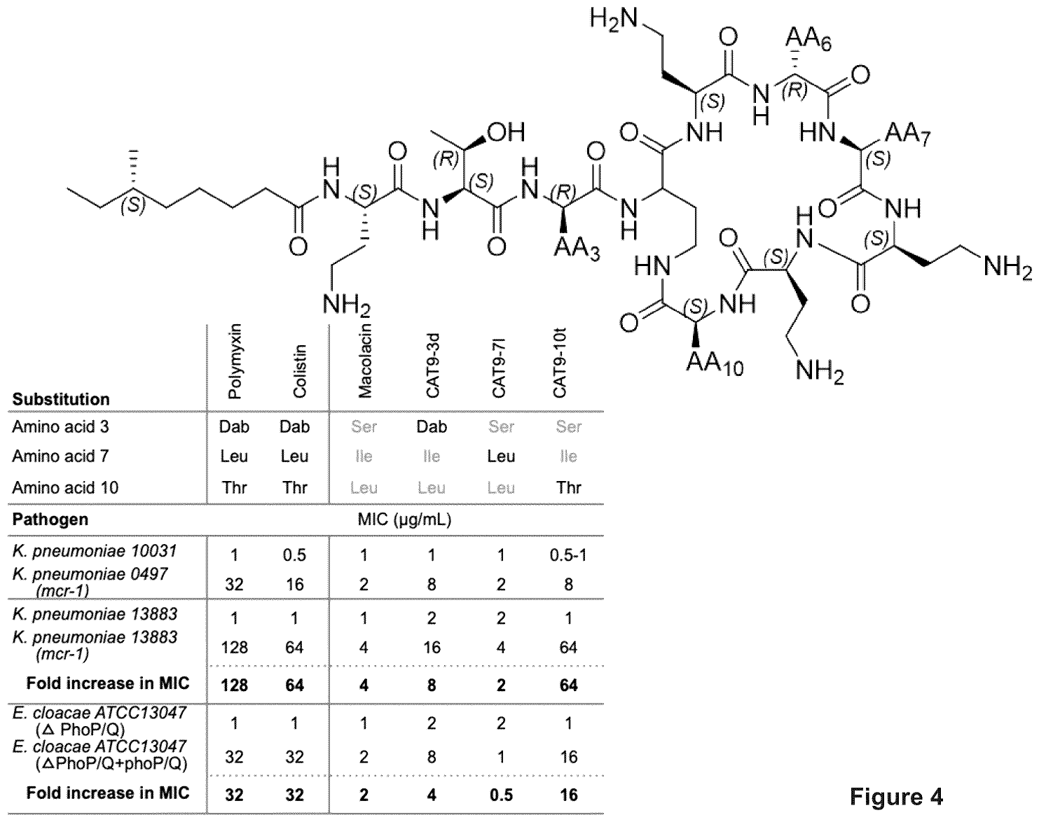

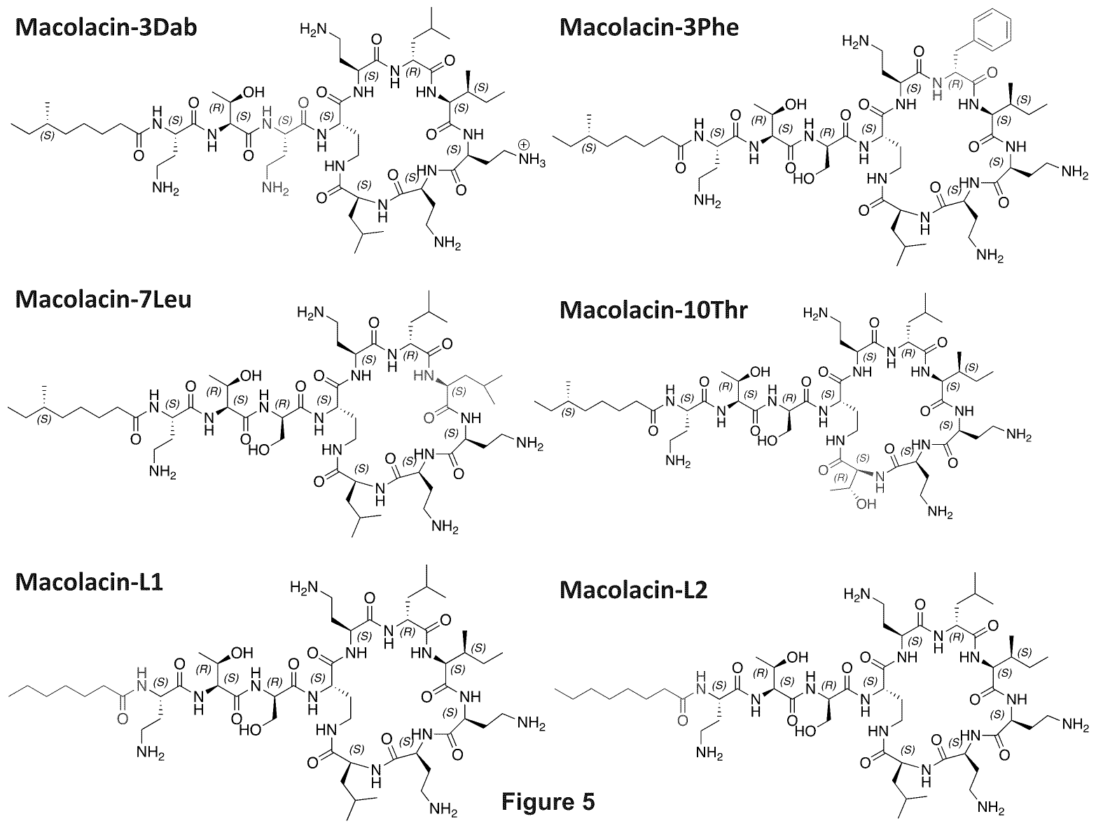

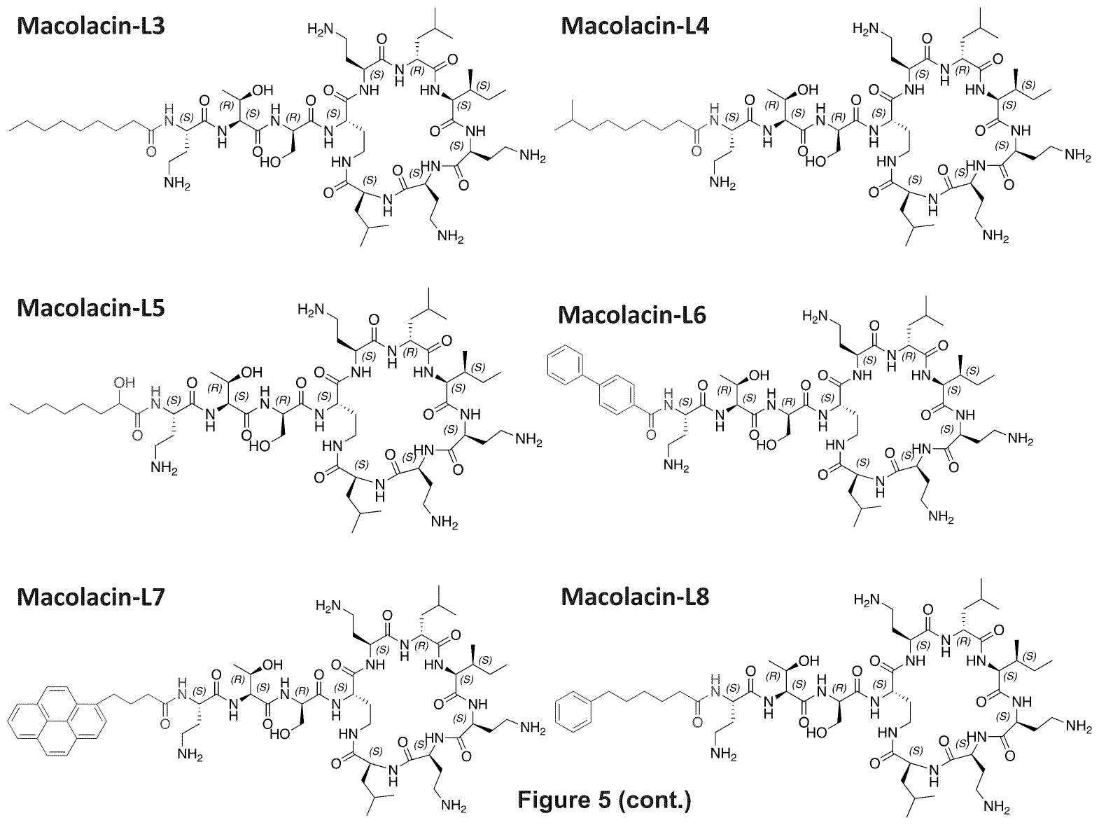

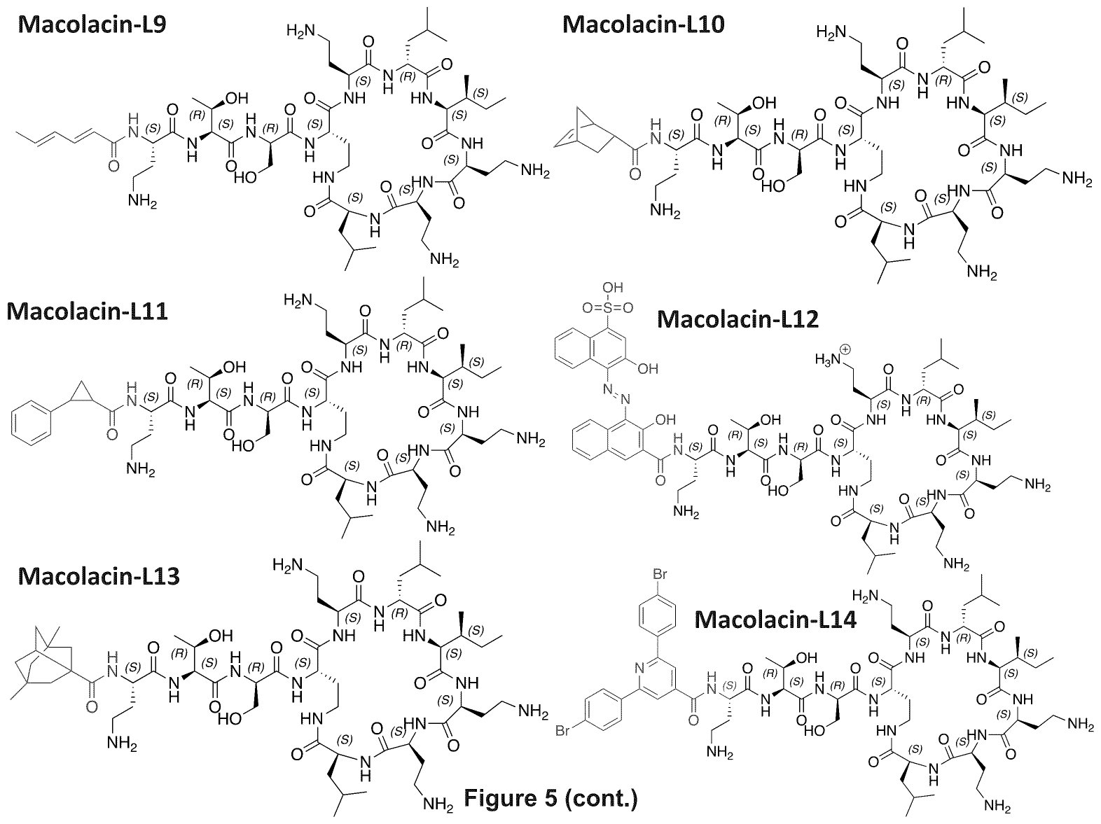

Macolacin differs from colistin by three amino acids. To determine which of these changes is critical for its ability to overcome mcr-1 encoded resistance, a set of structures with unique two amino acid changes was synthesized (FIG. 4 and FIG. 5). The change of leucine to isoleucine at position 7 (macolacin-71) had little effect on mcr-1 encoded resistance. Individually, the changes at position 3 (Dab to Ser) or 10 (Thr to Leu) each appeared to provide some protection against colistin resistance (MICs≥8 μg/mL); however, potent antibiosis was only seen when both position 3 was serine and position 10 was leucine (MIC 1-2 μg/mL). The same pattern of activity was seen for phoP/Q regulated colistin resistance in E. cloacae (FIG. 4).

Although serine and leucine have individually been seen at these positions in characterized polymyxin congeners, these substitutions are rare compared to other observed amino acid changes (i.e., two cases for Ser and one case for Leu) and they have never appeared together in the same congener. In polymyxin family structures, the side chain of a D-configured amino acid at position 3 and an L-configured amino acid at position 10 were very close in three-dimensional space and likely interacted together with lipid A to counter common amine containing modifications (e.g., PEtN or Ara4N; Velkov T et al., 2013, Future Microbiology, 8:711-724). As the binding of colistin to lipid A is largely driven by electrostatic interactions, compensating for appending a primary amine onto a lipid A phosphate involved replacing a positively charged Dab with a neutral residue (Ser).

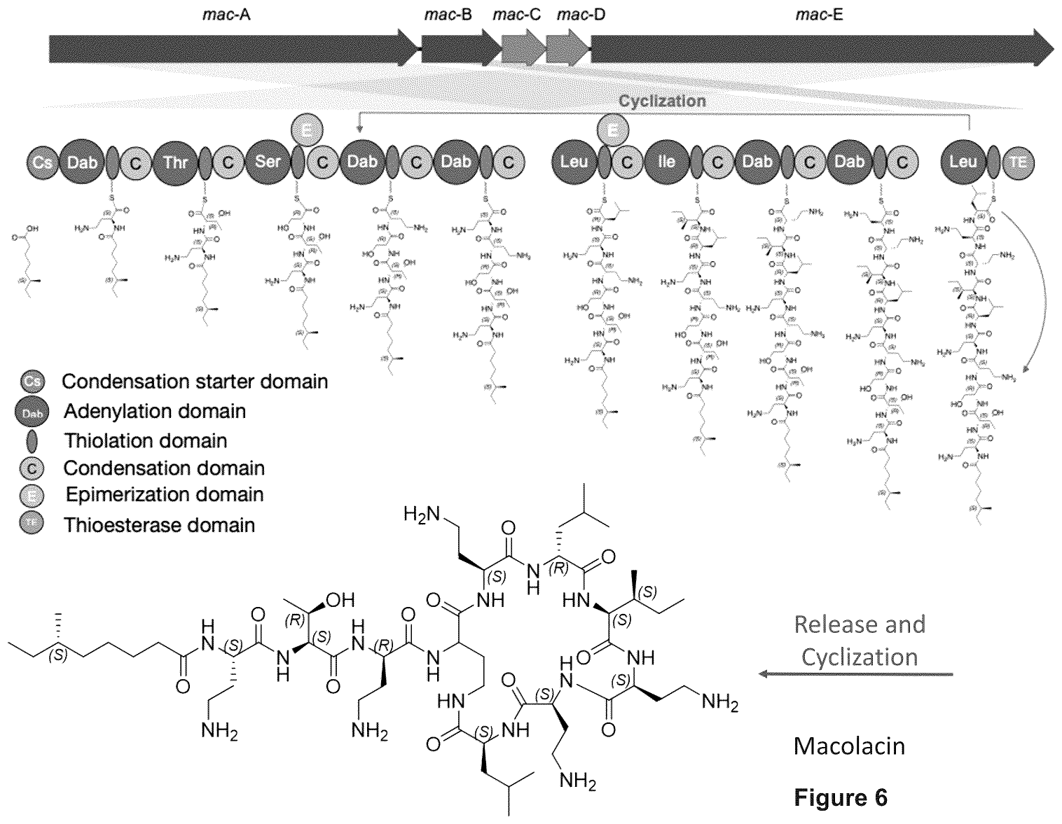

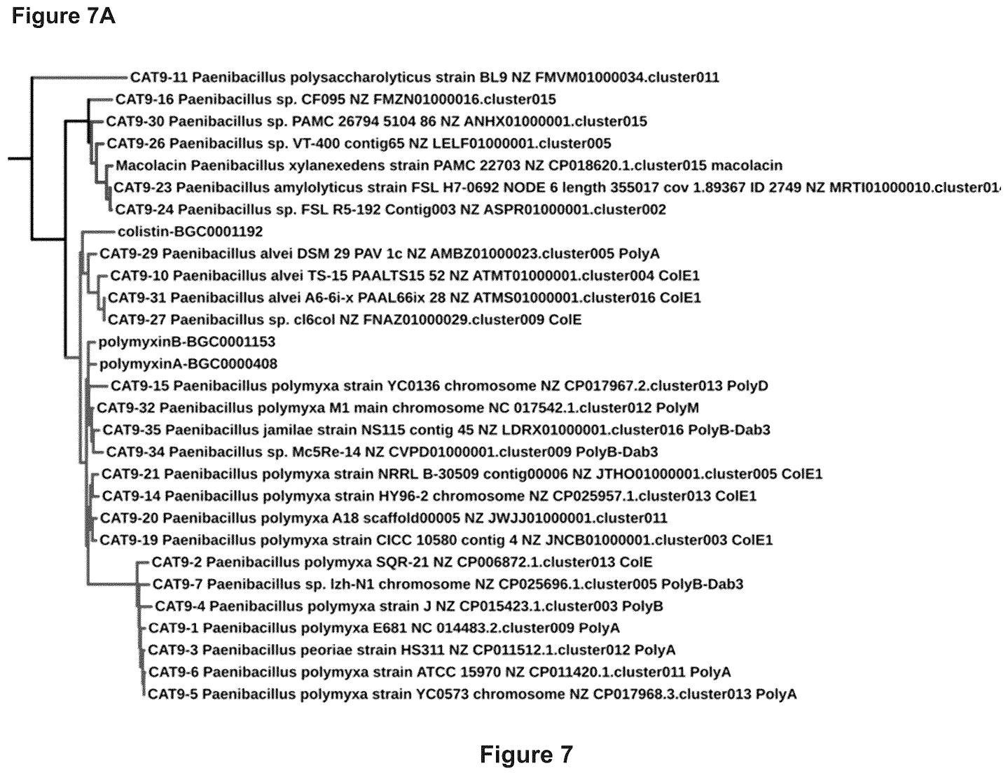

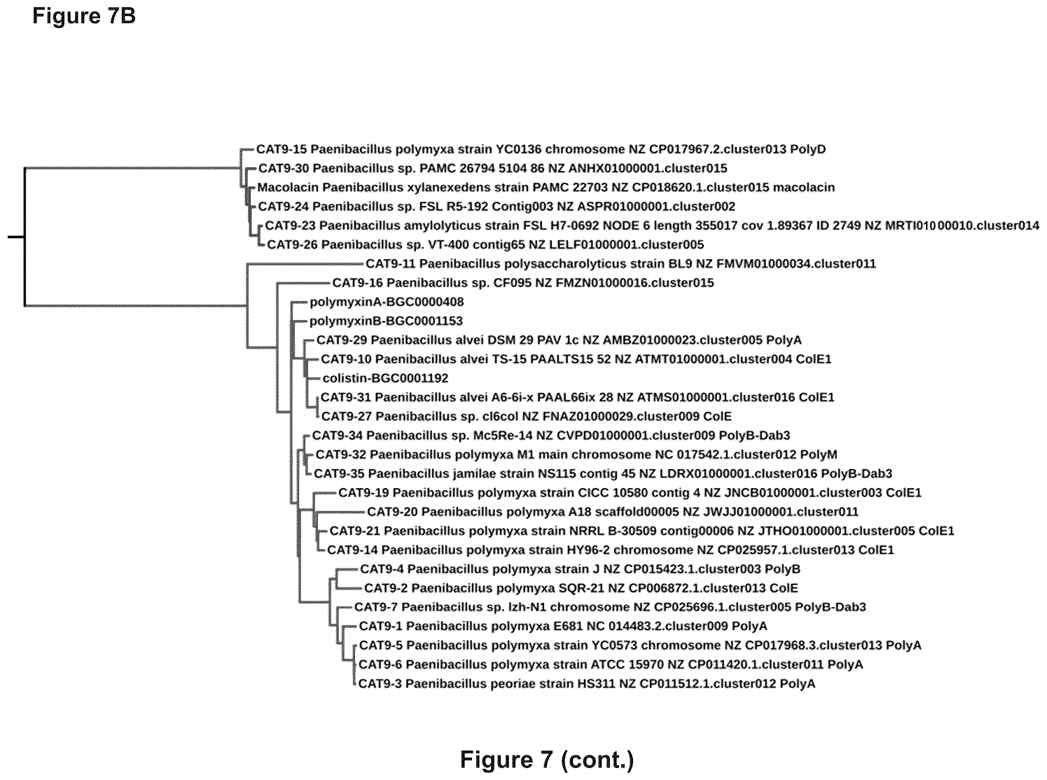

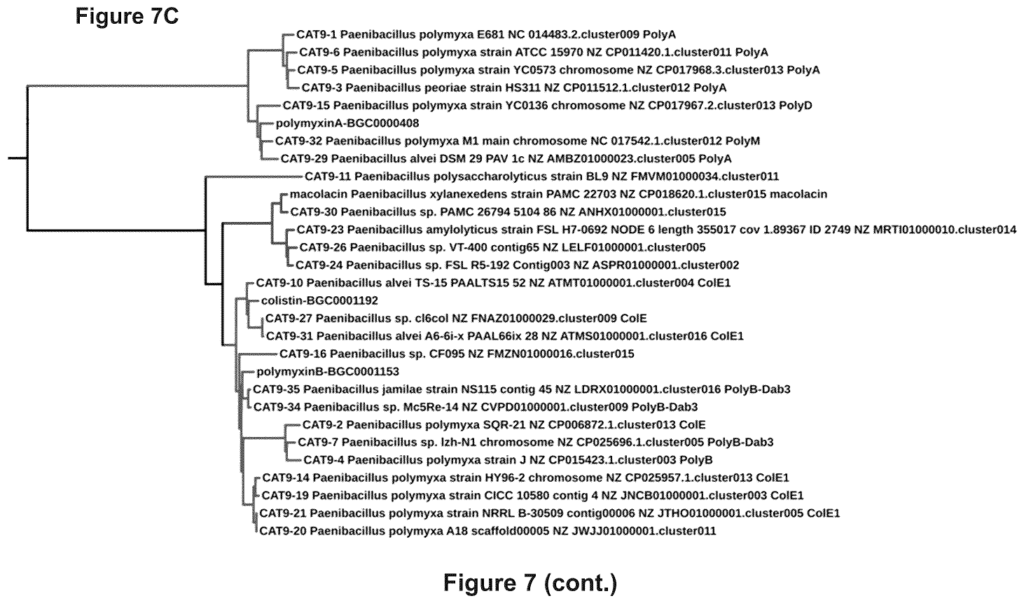

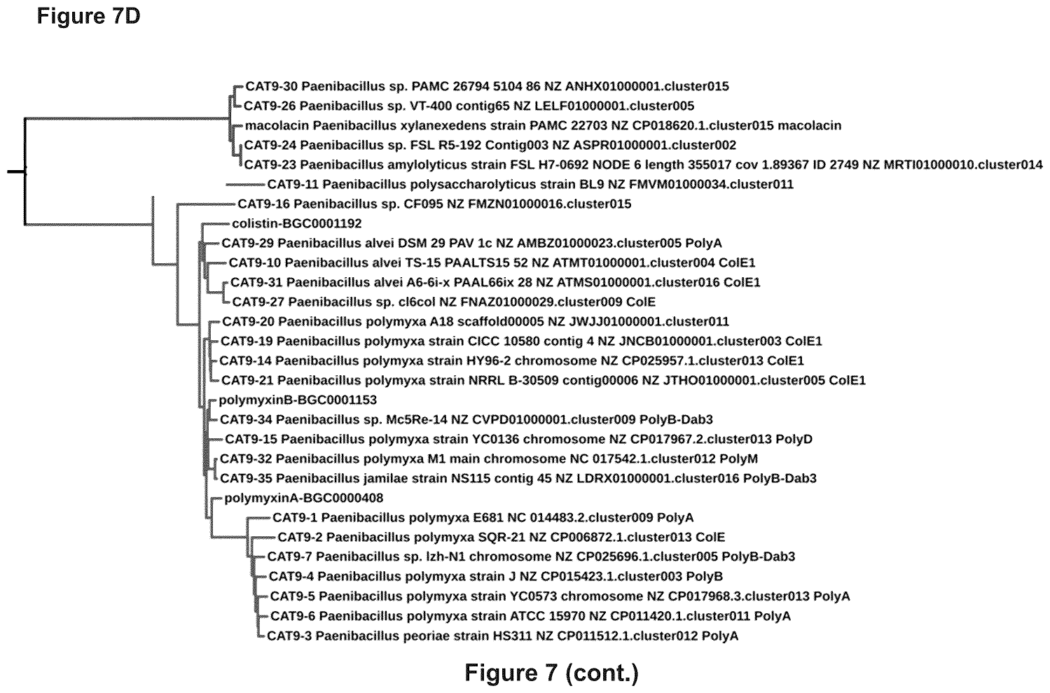

The organization and gene content of the mac gene cluster mimics that of other polymyxin family BGCs (FIG. 6) indicating a common evolutionary ancestor. Phylogenetic analyses of gene sequences from polymyxin BGCs showed a more significant divergence of mac gene sequences than was seen for genes from most other predicted polymyxin-family BGCs (FIG. 7). This divergence undoubtedly long predated the recent global spread of mcr-1. Although the selection of the mac BGCs was likely in response to genome integrated PEtN transferase or intrinsic phoP/Q resistance genes; the fact that clinical and natural resistance arose from the same limited pool of potential resistance genes meant this natural solution is useful for combating antibiotic resistant pathogens in the clinical setting.

Improved Antibiosis against Colistin Resistant Pathogens

Among antibiotic resistant Gram−negative pathogens, CRAB are classified as the highest level threat by the Center for Disease Control (CDC) in the United States (Centers for Disease Control and Prevention, 2019, Antibiotic resistance threats in the United States). Colistin has emerged as a critical therapeutic option for the treatment of these pathogens. Unfortunately, strains containing mcr-1, or a related mer gene, are increasingly found among clinical isolates around the world (Ling Z et al., 2020, J Antimicrob Chemother, 75:3087-3095). For this reason, translational efforts focused on developing a macolacin analog that is effective at treating highly colistin resistant CRAB infections. Against A. baumannii that is resistant to ≤32 g/mL of colistin macolacin retained potent antibacterial activity (MIC 2 μg/mL). For extremely highly colistin resistant A. baumannii (MIC>128 μg/mL), macolacin's MIC increased slightly to 8 μg/mL. Although macolacin was significantly more potent than colistin, its MIC exceeded the threshold set by many global health agencies for clinical use (Satlin M et al., 2020, Clinical Infectious Diseases, 71:523-529). A very similar activity profile was seen for the intrinsically resistant Neisseria gonorrhoeae, which, like A. baumannii, is considered a highest level threat pathogen by the CDC. Colistin was completely inactive against N. gonorrhoeae (MIC>128 μg/mL), while macolacin had an MIC of 8 μg/mL.

Before beginning the animal studies with macolacin, the studies sought to improve the activity of macolacin against colistin resistant pathogens. Although not bound by any particular theory, it was hypothesized that the macolacin peptide macrocycle has already been naturally optimized to interact with both modified and nonmodified lipid A head groups, and that it would prove challenging to further improve the activity of macolacin through modifications of its peptide structure. The lipid, on the other hand, makes nonspecific hydrophobic interactions with the long acyl substituents of lipid A.

Naturally occurring polymyxins are commonly N-terminally acylated with(S)-6-methyloctanoic acid and, therefore, macolacin was originally generated using this lipid. Previous structure activity studies with polymyxins have found that the length of the lipid is important and that hydrophilic substituents on the lipid decrease antimicrobial activity (Sakura N et al., 2004, Bulletin of the Chemical Society of Japan, 77:1915-1924; Tsubery H et al., 2001, Peptides, 22:1675-1681). Within the relatively narrow parameters defined by these previous studies, a collection of differentially N-acylated macolacin analogs was generated to test for improved activity against highly resistant CRAB. As a result, this study identified a biphenyl lipid analog (biphenyl-macolacin) was more active than macolacin against most of the pathogens tested. Colistin and biphenyl-macolacin showed similarly low cytotoxicity to human cells (IC₅₀>512 μg/mL Table 3).

TABLE 3
Cytotoxicity (IC50) values of macolacin
and biphenyl-macolacin against HEK293.

| Compounds | IC50 (μg/mL) |
|---|---|
| Colistin | >512 |
| Biphenyl-macolacin | >512 |

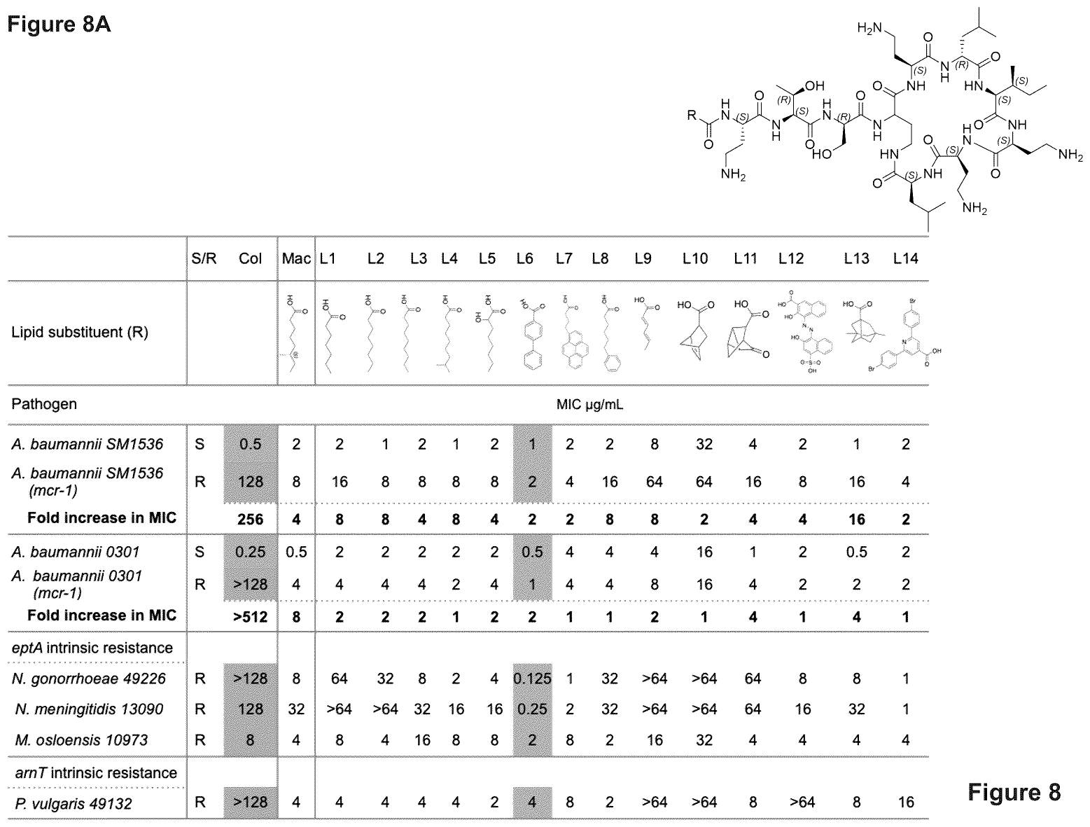

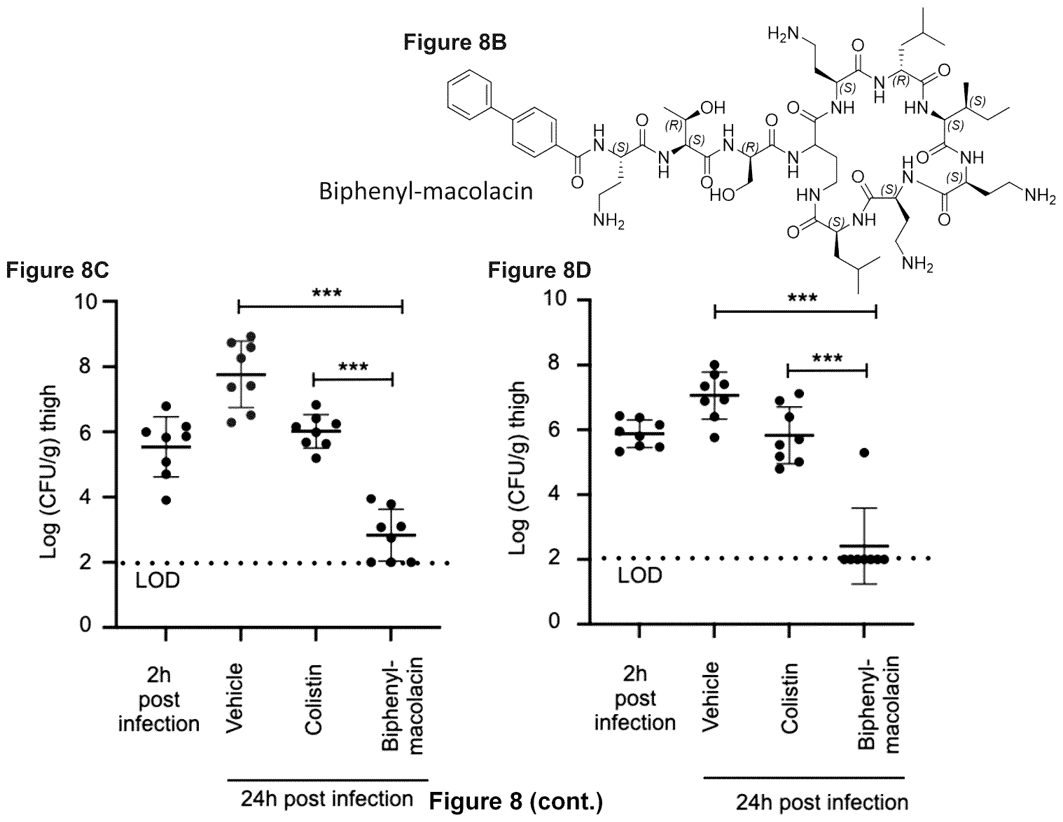

Against CRAB, as well as a panel of XDR (extremely drug resistant) A. baumannii clinical isolates, biphenyl-macolacin had MICs of <2 μg/mL even when these bacteria were transformed with mcr-1 (FIG. 8A, Table 4).

TABLE 4
MIC data for XDR A. baumannii with and without mcr-1. MIC data below the
empty line was obtained from CDC & FDA Antibiotic Resistant Isolate Bank.

| A. baumannii strain | 0287 | 0287 | 0295 | 0295 | 0301 | 0301 | 0286 | 0282 | 0296 |
|---|---|---|---|---|---|---|---|---|---|
| mcr-1 genotype | − | + | − | + | − | + | − | − | − |
| Colistin phenotype | S | R | S | R | S | R | S | S | S |
| Antibiotic MIC (μg/ml) | | | | | | | | | |
| Macolacin | 1 | 1 | 0.5 | 4 | 0.5 | 4 | 2 | 1 | 1 |
| Biphenyl-macolacin | 1 | 1 | 0.5 | 2 | 0.5 | 1 | 2 | 1 | 1 |
| Colistin | 0.5 | 64 | 0.25 | >128 | 0.25 | >128 | 1 | 0.5 | 0.25 |
| Amikacin | 32 | | 4 | | 64 | | >64 | >64 | >64 |
| Ampicillin | >32 | | >32 | | >32 | | 32 | >32 | >32 |
| Cefepime | 32 | | >32 | | >32 | | 32 | >32 | >32 |
| Cefotaxime | >64 | | >64 | | >64 | | >64 | >64 | >64 |
| Ceftazidime | 128 | | >128 | | 128 | | 128 | >128 | >128 |
| Ceftriaxone | >32 | | >32 | | >32 | | >32 | >32 | >32 |
| Ciprofloxacin | >8 | | >8 | | >8 | | >8 | >8 | >8 |
| Doripenem | >8 | | >8 | | >8 | | >8 | >8 | >8 |
| Gentamicin | 4 | | 4 | | 8 | | >16 | >16 | >16 |
| Imipenem | 64 | | >64 | | >64 | | >64 | 64 | 64 |
| Imipenem/relebactam | >16 | | >16 | | >16 | | >16 | >16 | >16 |
| Imipenem + chelators | >32 | | >32 | | >32 | | >32 | >32 | >32 |
| Levofloxacin | 8 | | >8 | | >8 | | 8 | 8 | >8 |
| Meropenem | >8 | | >8 | | >8 | | >8 | >8 | >8 |
| Minocycline | <=4 | | 16 | | 16 | | 8 | 8 | <=4 |
| Piperacillin | >128 | | >128 | | >128 | | >128 | >128 | >128 |
| Tetracycline | >32 | | >32 | | >32 | | >32 | >32 | 32 |
| Tigecycline | <=0.5 | | 4 | | 4 | | 1 | 1 | 2 |
| Tobramycin | >16 | | 1 | | >16 | | >16 | >16 | >16 |
| Trimethoprim | >8 | | >8 | | >8 | | >8 | >8 | >8 |

Biphenyl-macolacin was also active against a number of intrinsically colistin resistant pathogens. In the case of N. gonorrhoeae, which is colistin resistant due to a chromosomally encoded PEtN transferase, biphenyl-macolacin was particularly potent with an MIC of 0.125 μg/mL. In the case of Proteus vulgaris, a common cause of urinary tract infections that is intrinsically colistin resistant due the modification of its LPS with L-Ara4N, biphenyl-macolacin had an MIC of 4 μg/mL, while colistin and polymyxin had MICs>128 μg/mL.

### Animal Studies

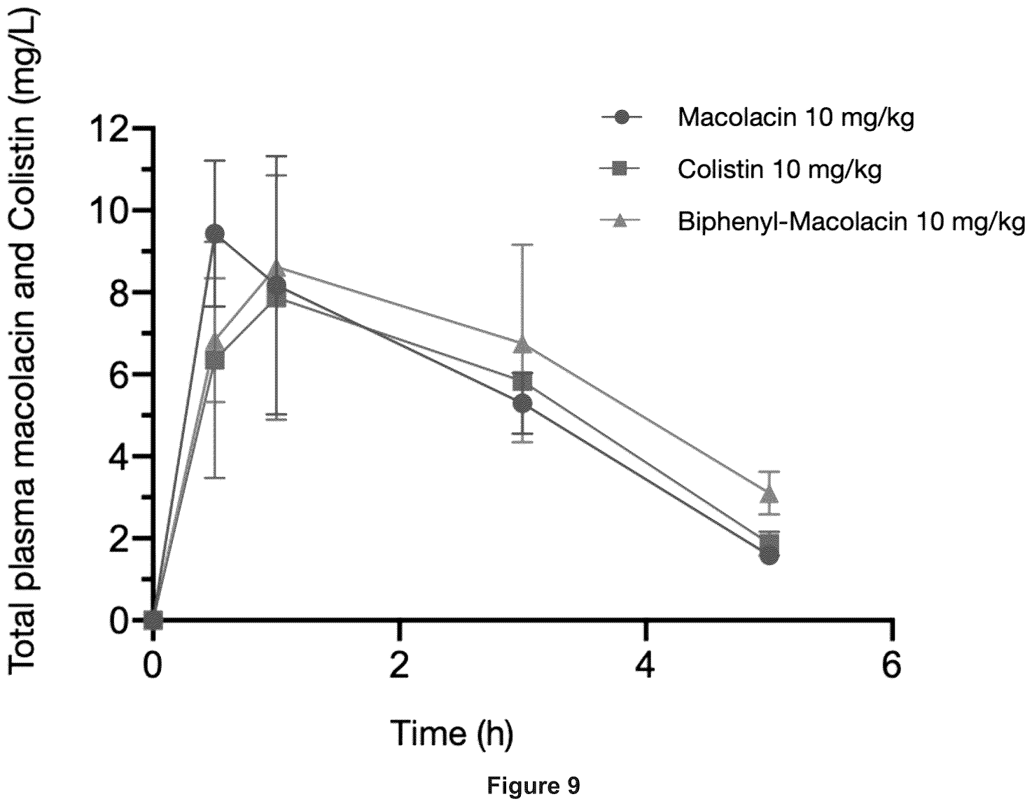

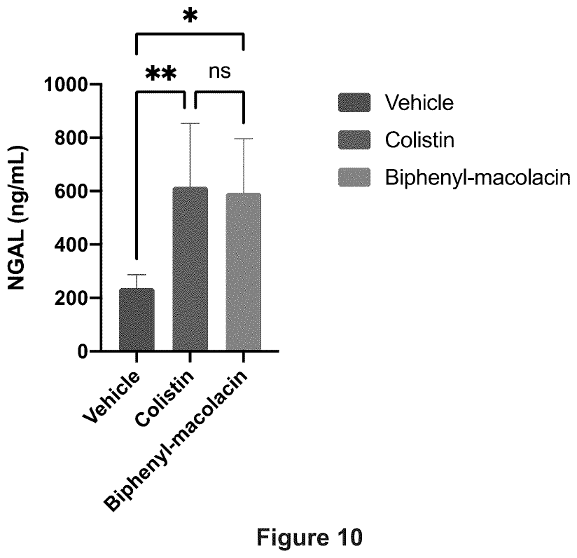

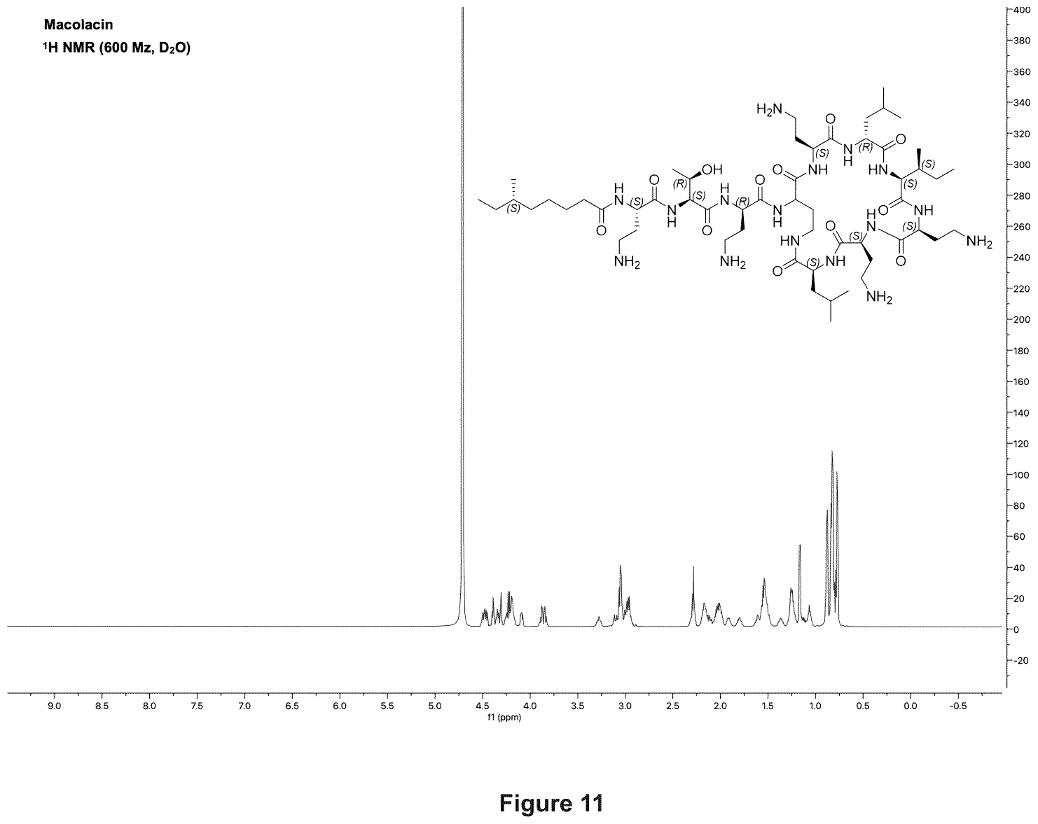

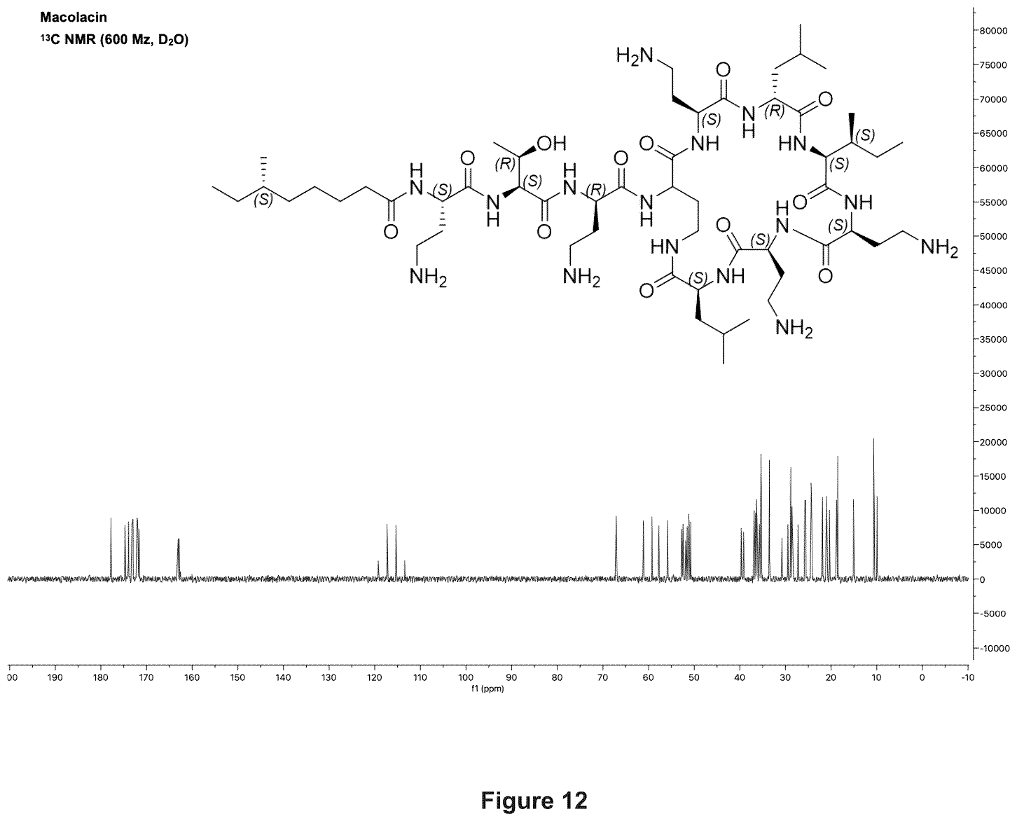

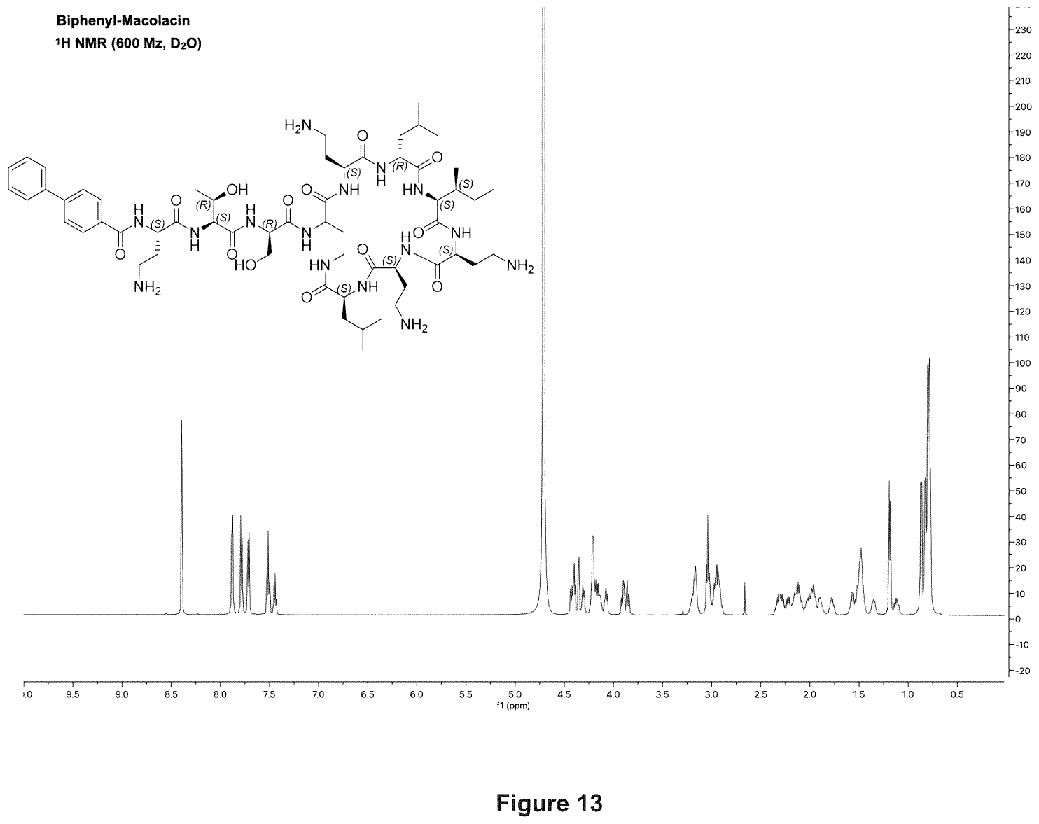

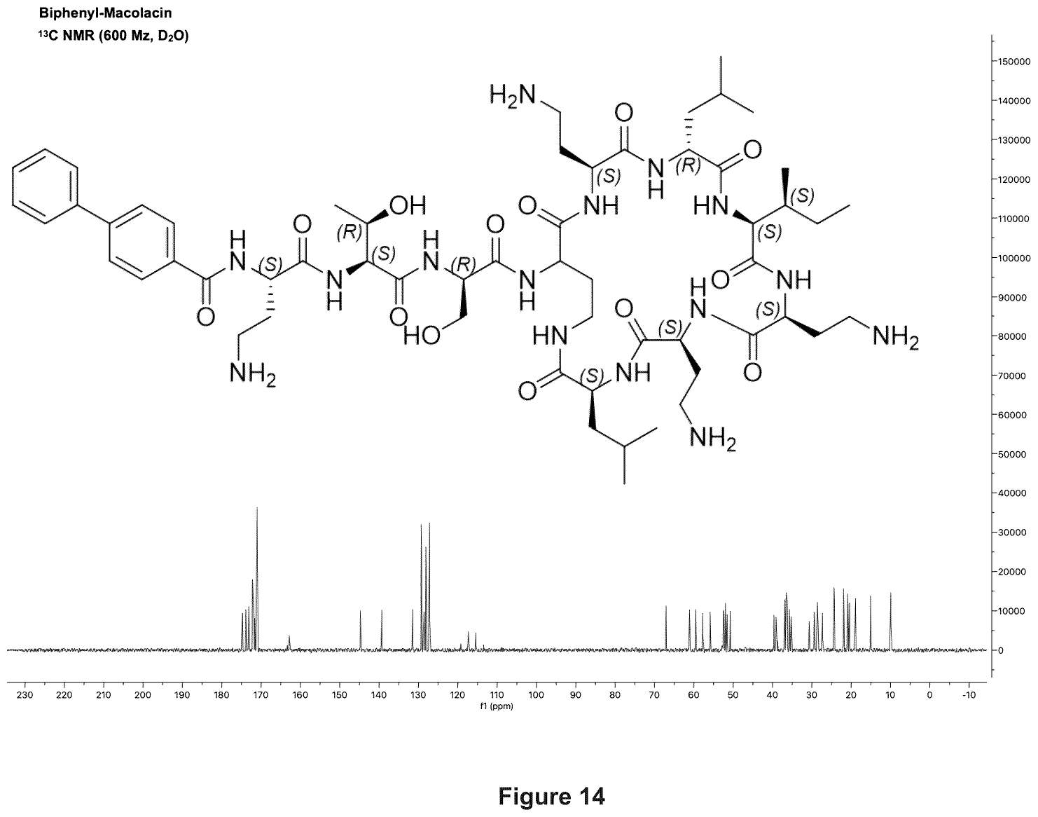

A neutropenic thigh infection model was used to evaluate the in-vivo efficacy of biphenyl-macolacin in vivo. In these studies, two different highly colistin resistant strains of A. baumannii were used. One was a CRAB (A. baumannii—SM1536) that was transformed with mcr-1 to give a colistin resistant strain (A. baumannii—SM1536-mcr-1). An XDR A. baumannii clinical isolate (A. baumannii-0301), that was resistant to all antibiotics that were tested, was also used (FIG. 8A, Table 4; Lutgring J D et al., 2018, J Clin Microbiol, 56). When it was transformed with mcr-1 to give a PDR (pan drug resistant, XDR with colistin resistance) strain, colistin's MIC increased to >64 μg/ml, but the activity of biphenyl-macolacin remained <2 μg/ml. For both studies, two hours after mice were exposed to the pathogen, they were subcutaneously given either biphenyl-macolacin (20 mg/kg), colistin (20 mg/kg), or vehicle alone (0.9% saline) at 6 hour intervals. The pharmacokinetic profiles of subcutaneous dosed colistin and biphenyl-macolacin indicated parity in plasma drug exposure for both compounds (FIG. 9). Colistin and biphenyl-macolacin also induced similar levels of neutrophil gelatinase associated lipocalin (NGAL), which is a biomarker for nephrotoxicity (FIG. 10; Devarajan P et al., 2008, Scand J Clin Lab Invest Suppl, 241:89-94; Wang J et al., 2020, Front Pharmacol 11:1146; Bolignano D et al., 2008, Am J Kidney Dis, 52:595-605). After 24 hours the bacterial burden in each infected thigh was determined based on colony forming units (CFUs) that arose from homogenized tissue samples.

In these studies, colistin did not reduce the bacterial burden below the level of the initial infection. However, against both A. baumannii strains, biphenyl-macolacin showed significant antibiosis activity, resulting in an almost 5 log₁₀ reduction in CFUs compared to the vehicle control group and a 3 log₁₀ reduction in CFUs compare to the colistin treatment group (p<0.0005 for both). In majority of biphenyl-macolacin treated thighs, no pathogen colonies were observed upon plating infected tissue, indicating biphenyl-macolacin completely cleared the infection by this pathogen for which few therapeutic options currently exist.

In conclusion, colistin's extensive use in livestock and human healthcare has resulted in the transfer of mcr-1 from the environment into the clinical setting thus threatening its use as an antibiotic of last resort against a number of MDR Gram−negative pathogens. As mcr-1-like PEtN transferase genes are common in the soil microbiome, it was reasoned that natural selection led to colistin congeners that are capable of circumventing this troubling resistance mechanism. By coupling genome mining methods to identify polymyxin family-like BGCs in sequenced bacterial genomes with syn-BNP methods, macolacin was identified as a colistin-like antibiotic that was active against colistin resistant Gram−negative pathogens that was mediated by either mcr-1 or intrinsic transferase genes (eptA and arnT).

Although the ecological history that led to the selection of the mac BGC is not known, the present studies indicated that PEtN transferase resistance is a significant driving force behind the selection of this BGC. Optimization of the lipid substituent in macolacin produced biphenyl-macolacin, which showed potent activity against colistin resistant Gram−negative bacteria that are resistant to colistin due to the acquisition of mcr-1 as well as intrinsically colistin resistant pathogens (e.g., N. gonorrhoeae). Biphenyl-macolacin was also active in vivo activity against both CRAB and XDR A. baumannii containing mcr-1, providing a new easily scaled therapeutic lead for this extremely troubling antibiotic resistant pathogen. The systematic exploration of naturally occurring congeners of other antibiotics, whose utility is threatened by the rise of resistance in clinical settings, is similarly useful to revitalizing activity against MDR pathogens.

In summary, gram-negative bacteria are responsible for an increasing number of deaths from antibiotic resistant infections. The bacterial natural product, colistin, is considered the last line of defense against a number of Gram−negative pathogens. The recent global spread of the plasmid-borne mobilized colistin resistance gene (mcr-1, phosphoethanolamine (PEtN) transferase gene) threatens colistin's utility. Bacterial-derived antibiotics often appear in nature as collections of similar structures that are encoded by evolutionarily related biosynthetic gene clusters (BGCs). This structural diversity is at least in part likely a response to the development of natural resistance, which often mechanistically mimics clinical resistance. The present studies described search of sequenced bacterial genomes identified a BGC that was likely to encode the most structurally divergent colistin congener described to-date. Total chemical synthesis of this structure produced macolacin, which was active against Gram−negative pathogens containing either mcr-1 as well as those intrinsically resistant pathogens with chromosomally encoded PEtN transferase genes. These included extremely drug resistant (XDR) Acinetobacter baumannii and intrinsically colistin resistant Neisseria gonorrhoeae which, due to a lack of effective treatment options, were considered among the highest level Gram−negative threats. In a mouse neutropenic infection model, a biphenyl analog of macolacin proved to be very effective against colistin resistant XDR Acinetobacter baumannii, thus providing a new naturally inspired and easily produced therapeutic lead for this problematic antibiotic resistant pathogen. As such, the present example described new colistin-analog, macolacin, and the development and use thereof.

The materials and methods employed in the present experiments are now described herein.

Identification and Bioinformatic Analysis of the Macolacin (mac) Biosynthetic Gene Cluster

A total of 36,957 nonribosomal peptide synthetase (NRPS) biosynthetic gene clusters (BGCs) from 10,858 bacterial genomes were downloaded from antiSMASH-db (version 2.0) (Blin K et al., 2019, Nucleic Acids Res, 47:625-630). The offline software package antiSMASH (v5.1.2, bacterial version) (Blin K et al., 2019, Nucleic Acids Res, 47:81-87) and BLAST were used to identify NRPS BGCs that resemble (by gene content, gene organization and sequence identity) BGCs known to encode polymyxin-family antibiotics (e.g., MIBIG IDs: BGC0000408, BGC0001192, BGC0001153). The linear NRP product of each predicted polymyxin/colistin-like BGC was determined using an A-domain substrate binding pocket analysis (Stachelhaus T et al., 1999, Chem Biol, 6:493-505). In this analysis each NRPS A-domain in a predicted polymyxin/colistin-like BGC was analyzed using the online antiSMASH 5.0 (bacterial version) web tool to identify the 10-amino acids that make up its A-domain substrate binding pocket (i.e., amino acids 235, 236, 239, 278, 299, 301, 322, 330, 331, and 517). Each unknown A-domain substrate signature sequence was compared to a database of A-domain signatures from characterized BGCs to predict its amino acid substrate.

### Peptide Synthesis

Linear peptides were synthesized using standard solid phase Fmoc chemistry. Each peptide was synthesized using 2-Chlorotyityl resin preloaded with the first amino acid (0.2 g, 0.455 mmol/g). Resin was swollen in DCM for 30 min at room temperature and then washed with DMF (3×10.0 mL). The coupling proceeded by addition of the subsequent Fmoc protected amino acid (3.0 eq), 3.0 eq of DIPEA and 2.85 eq of HBTU in DMF (2.0 mL) to the resin. The resin was agitated under N₂ for 60 min at room temperature and then washed with DMF (3×20.0 mL). The Fmoc protecting group was removed by addition of 20% piperidine in DMF (10.0 mL) to the resin with agitation under N₂ for another 30 min. The resin was then washed with DMF (5×20.0 mL). The procedure of coupling and deprotection was repeated for each remaining amino acid. Fatty acids were activated and coupled to the N-termini of the resin bound linear peptide using the same procedure. The final linear lipo-peptide was cleaved from the resin by treatment with 10 mL of 1% TFA in DCM and stirring for 5 min. This was repeated twice and then air dried overnight. Cleaved linear lipo-peptide (0.2 mmol, 1 eq) was cyclized by dissolving in DCM (250.0 mL) containing TBTU (2.0 eq), HOBT (2.0 eq) and DIPEA (10 eq). After 1 h of stirring at room temperature, the mixture was washed twice with 1M HCl (100 mL) and then dried under vacuum. The dried crude cyclic lipo-peptide was treated with a cleavage cocktail (TFA: TIPS: H2O=95%: 2.5%: 2.5%, 20.0 mL) for 1.5 h. Cold isopropyl ether was added to precipitate the TFA treated peptide and the precipitate was collected by centrifugation (3,000×g, 5 min). Crude peptide pellets were dissolved in 5 mL methanol and then dried under vacuum overnight. Pure cyclic lipo-peptides were obtained by semipreparative HPLC.

### Microbial Susceptibility Assay

Syn-BNPs were tested against the panel of Gram−positive and Gram−negative pathogens detailed in Table 5.

TABLE 5
Bacterial strain information.

| | | | Selective |
|---|---|---|---|
| Name of Bacteria | Strain | Resistant gene | Antibiotic |
| Enterococcus faecium | Com15 | N/A | N/A |
| Staphylococcus aureus | SH1000 | N/A | N/A |
| Klebsiella pneumoniae | 10031 | N/A | N/A |
| Klebsiella pneumoniae | 0497 | N/A | N/A |
| Klebsiella pneumoniae | ATCC13883 | N/A | N/A |
| Acinetobacter baumannii | ATCC17978 | N/A | N/A |
| Acinetobacter baumannii | SM1536 | N/A | N/A |
| Pseudomonas aeruginosa | PAO1 | N/A | N/A |
| Enterobacter cloacae | 0150 | N/A | N/A |
| Salmonella typhimurium | 0635 | N/A | N/A |
| Acinetobacter baumannii | CDC 0282-0301 | N/A | N/A |
| Neisseria gonorrhoeae | ATCC 49226 | EptA | N/A |
| Neisseria meningitidis | ATCC 13090 | EptA | N/A |
| Moraxella osloensis | ATCC10973 | EptA | N/A |
| Proteus vulgaris | ATCC 49132 | ArnT | N/A |
| Enterobacter cloacae | ATCC13047 | ΔphoP/Q | N/A |
| Klebsiella pneumoniae | ATCC13883 | pBCSK-mcr-1 | 30 μg/mL |
| | | | Chloramphenicol |
| Acinetobacter baumannii | ATCC17978 | pMQ124xlab1-mcr-1 | 50 μg/mL |
| | | | Gentamicin |
| Acinetobacter baumannii | SM1536 | pMQ124xlab1-mcr-1 | 50 μg/mL |
| | | | Gentamicin |
| Acinetobacter baumannii | CDC 0282-0301 | pMQ124xlab1-mcr-1 | 50 μg/mL |
| | | | Gentamicin |
| Enterobacter cloacae | ATCC13047 | ΔphoP/Q + pMMBKn- | 50 μg/mL |
| | | phoP/Q − 6xhis | Kanamycin |

N/A: not applicable.

MIC assays were conducted following the protocol recommended by the Clinical and Laboratory Standards Institute (Testing E C et al., 2016, EUCAST: Växjö; Wikler M A et al., 2006, CLSI (NCCLS), 26: M7-A7). Briefly, all assays were performed in duplicate (n=2) in non-treated 96-well microliter plates (THERMO SCIENTIFIC™ Nunc MicroWell 96-Well Microplates, Non-treated polystyrene plates). Syn-BNP peptides were dissolved in sterile DMSO (ATCC, USA) to give 12.8 mg/mL stock solutions. Polymyxin B sulfate (Sigma, USA) and colistin sulfate (Sigma, USA) were used as positive controls. Stock solutions were diluted across 96-well plates using a 2-fold serial dilution to give a concentration range of 256 to 0.25 μg/mL in 50 μL of LB broth per well. The top and bottom rows of each plate were filled with 100 μL of LB broth without compound to avoid edge effects. The last well in each row was treated as a negative control. It contained bacteria but no test compounds. A single colony of each assayed bacterial strain was inoculated into 5 mL of LB broth medium and grown overnight at 37° C. (200 rpm). For assays using bacteria containing mcr-1 expression plasmids, the appropriate selection antibiotic was added to keep the plasmid stable (concentrations are listed in Table 5). Saturated overnight cultures were diluted 5,000-fold in fresh LB, and then 50 μL was transferred into individual wells of a 96-well plate. Finally, each well contained a total volume of 100 μL to give a final assay concentration range of 128 to 0.125 μg/mL for each compound. MIC values were determined by visual inspection to identify the minimum concentration that completely prevented bacterial growth after 16 h at 37° C.

### CHIR-090 Inhibition Assay

To test microbial susceptibility of LPS-deficient bacteria to macolacin and colistin, the LPS inhibitor CHIR-090 was used to treat A. baumannii together with either macolacin, colistin or kanamycin (Bojkovic J et al., 2015, 2015, J Bacteriol, 198:731-741). Colistin (Sigma, USA) and kanamycin (Sigma, USA) were used as positive and negative controls, respectively. A single colony of A. baumnanii ATCC17978 was inoculated into 5 mL of LB broth and grown overnight at 37° C. Stationary-phase cultures were then diluted with fresh LB to an optical density (OD) at 600 nm of 0.2 and used as starter cultures for susceptibility assays. Assays were performed in triplicate in a 96-well plate. Assay wells contained 180 μL of starter culture bacteria, antibiotic at a final concentration of 10× its MIC and 10 μL of CHIR-090 (8 μg/mL, Sigma, USA). The final volume of each well was adjusted to 200 μL with LB. The Plate was covered with a clear lid and statically incubated at 37° C. in a Tecan plate reader (Infinite M Nano). The absorbance of each well was continuously measured at an absorbance wavelength of OD 600 nm at every 15 min for 48 h. For each condition growth curves were run on three unique colonies (n=3). The growth curve at each concentration was plotted in Prism 9.0.

### Pharmacokinetics Assessment

All pharmacokinetic animal studies were ethically reviewed and carried out in accordance with the Institutional Animal Care and Use Committee of Hackensack Meridian Health under protocol number 269.01. Six-week old CD-1 female mice (20-25 g) were used in pharmacokinetic studies. Macolacin and biphenyl-macolacin were administered as a single dose by subcutaneous injection at 10 mg/kg using 0.9% saline vehicle. Aliquots of 20 μL of blood were taken by puncture of the lateral tail vein from each mouse (n=2 per route and dose) at 30 minutes, 1, 3, and 5 hours post-dose and captured in CB300 blood collection tubes containing K2EDTA and stored on ice. Plasma was recovered after centrifugation and stored at −80° C. until analyzed by high pressure liquid chromatography coupled to tandem mass spectrometry. 1 mg/mL stocks of macolacin and biphenyl-macolacin in water were serially diluted in 50% acetonitrile/water to generate standard curves and quality control spiking solutions. Standards and QCs were created by adding 10 μL of spiking solutions to 90 μL of drug free plasma (CD-1 K2EDTA Mouse, Bioreclamation IVT). 10 μL of control, standard, QC, or study samples were added to 10 μL of internal standard. Verapamil (Sigma Aldrich) was used as an internal standard for macolacin, and macolacin was used as an internal standard for biphenyl-macolacin. The compounds were extracted by adding 100 μL of 50% Methanol/50% (10% Trichloroacetic acid in water) precipitation solvent. Extracts were vortexed for 5 minutes and centrifuged at 4,000 rpm for 5 minutes. 75 μL of supernatant was transferred for HPLC-MS/MS analysis and diluted with 75 μL of Milli-Q deionized water. LC-MS/MS analysis was performed on a Sciex Applied Biosystems Qtrap 6500+triple-quadrupole mass spectrometer coupled to a Shimadzu Nexera×2 UHPLC system to quantify each drug in plasma. Chromatography was performed on a Phenomenex Luna Omega Polar C18 (2.1×100 mm; particle size, 3 μm) using a reversed phase gradient. Milli-Q deionized water with 0.1% formic acid was used for the aqueous mobile phase and 0.1% formic acid in acetonitrile for the organic mobile phase. Multiple-reaction monitoring of parent/daughter transitions in electrospray positive-ionization mode was used to quantify the analytes. The double charged ions were used for macolacin and biphenyl-macolacin. The following MRM transitions were used for maclamycin (585.15/240.90), maclamycin-L10 (604.91/152.00), and Verapamil (455.40/165.00). Sample analysis was accepted if the concentrations of the quality control samples were within 20% of the nominal concentration. Data processing was performed using Analyst software (version 1.6.2; Applied Biosystems Sciex).

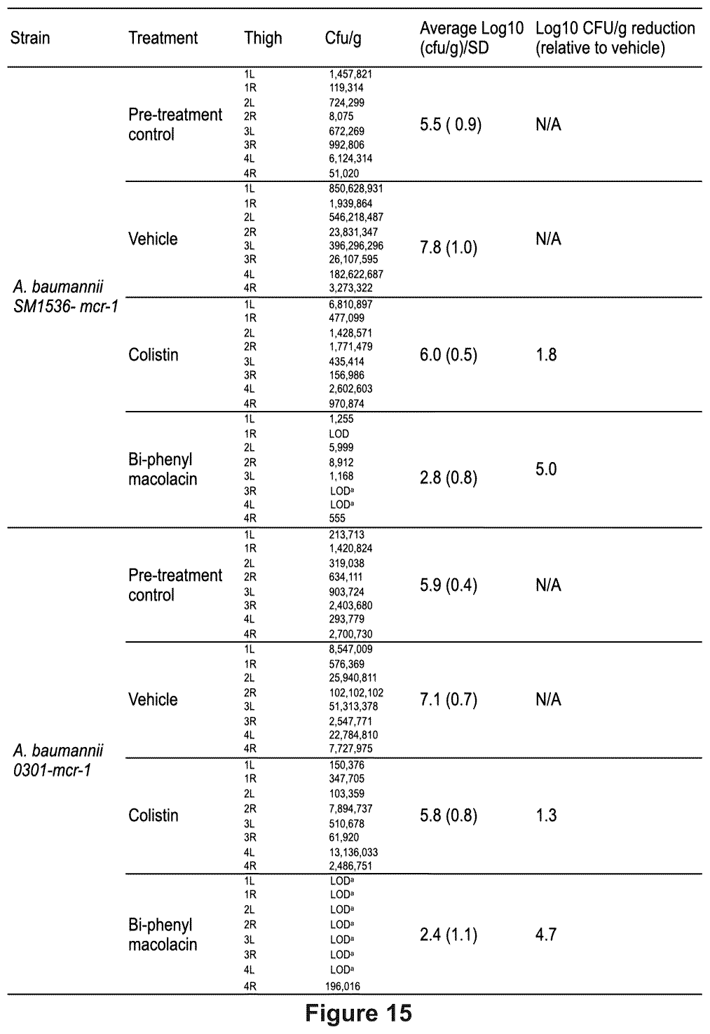

### Neutropenic Thigh Infection Model (FIG. 15)

Six-week old female outbred Swiss Webster mice (20-25 g) were used for this experiment. Mice were housed in individually ventilated cages (IVC) and maintained in accordance with American Association for Accreditation of Laboratory Care criteria. Acinetobacter baumannii SM1536-(mcr-1) or A. baumannii-0301-(mcr-1) was grown in cation adjusted Mueller Hinton (MH) broth containing 50 μg/mL of gentamycin at 37° C. with shaking overnight. The cultures were centrifuged, supernatant aspirated and the bacteria were gently washed twice in sterile saline. The optical density was checked at 600 nm and diluted so that the bacterial suspension provided a challenge inoculum of approximately 1.0×106 CFU per mouse thigh in a volume of 0.05 mL. Inoculum counts were verified by viable counts on Mueller Hinton Agar plates spread with proper dilutions of the inoculum and incubated at 37° C. for 24 h. Mice were rendered neutropenic by receiving 150 mg/kg and 100 mg/kg of cyclophosphamide via IP injection on day-4 and day-1 prior to infection, respectively. Mice were given 100 μL of vehicle (0.9% saline), colistin (20 mg/k) or biphenyl-macolacin (20 mg/k) at 2, 8, 14, & 20 hours post infection via subcutaneous injection. At 2 h post infection, mice in the untreated control infection group (n=4 mice/n=8 thighs) were humanely euthanized by CO₂ narcosis to determine the starting thigh bacterial burden. All mice were closely monitored post-infection for morbidity. Any abnormal clinical signs were recorded if observed. Mice were humanely euthanized by CO₂ narcosis at the experimental endpoint of 24 h post infection (n=4 mice/n=8 thighs for each condition). Thigh muscles were aseptically removed, weighed, homogenized and enumerated for bacterial burden by CFU counts after plating on MH agar containing 50 μg/mL of gentamicin. The treatment efficacy was determined both as the bacterial burden reduction in the thighs relative to the vehicle and colistin treated controls. All graphic data are expressed as columnar average data points by group and were statistically analyzed using computer Prism software (Prism 9). Burden differences between testing and control groups was assessed by post-hoc analysis, using the Mann-Whitney comparison test. A P value of <0.05 was considered statistically significant. These animal studies were carried out in accordance with the Institutional Animal Care and Use Committees at the Hackensack Meridian Health's under protocol 260 and the Rockefeller University under protocol 19032-H.

MTT (3-(4,5-Dimethyl-2-thiazolyl)-2,5-diphenyl-2H-tetrazolium bromide) Cytotoxicity Assay

An MTT (3-(4,5-Dimethyl-2-thiazolyl)-2,5-diphenyl-2H-tetrazolium bromide) assay was used to determine cytotoxicity as previously described (Carmichael J et al., 1987, Cancer Res, 47:936-942). In brief, HT29 cells cultured in DMEM (with 10% fetal bovine serum) were passaged in a 96-well plate with a density of 2,500 cells/well and cultured for 24 h at 37° C. with 5% CO₂. Compounds with different concentrations were then added into each well. After 48 h of incubation, the media was removed and MTT solution (0.45 mg/mL) was added into each well. After 3 h of incubation, the solution was aspirated. Precipitated formazan crystals were dissolved by additional of 100 μL solubilization solution (40% DMF, 16% SDS and 2% acetic acid in H₂O). The absorbance of each well was measured at OD₅₇₀ₙₘ using a microplate reader (Epoch Microplate Spectrophotometer, BioTek). Taxol was used as the positive control. IC50 values were calculated by Prism 9.0 as the concentration of each compound required for 50% inhibition of cell growth related to the no compound controls. All the experiments were performed triplicates (n=3).

### Nephrotoxicity Assay

Six-week old female outbred Swiss Webster mice (20-25 g) were used in this experiment. After 3 days of acclimation, mice were randomly divided into 3 groups (n=6 in each group): vehicle group, colistin sulfate group and biphenyl-macolacin group. Mice were subcutaneous injected (SC) with 100 μL of 0.9% saline (vehicle group), 20 mg/kg colistin sulfate or 20 mg/kg biphenyl-macolacin for 7 consecutive days. Serum was collected from blood samples 12 h after the last dose. The concentration of serum Neutrophil Gelatinase Associated Lipocalin (NGAL) was measured using a commercially available mouse NGAL ELISA Kit (R&D system, USA). The ELISA assay followed the manufacture's instruction. The animal study was carried out in accordance with the Institutional Animal Care and Use Committees at the Rockefeller University under protocol 19032-H.

TABLE 6
High-resolution mass spectrometry data of all syn-BNP peptides. All HRMS data were
collected in positive ionization model with a mass range from m/z 200-2000.

| | Molecular | Theoretical | Observed | | |
|---|---|---|---|---|---|
| Syn-BNPs | formular [M] | [M + H]⁺ | [M + H]⁺ | Mass error | |
| macolacin | C₅₄H₁₀₁N₁₅O₁₃ | 1168.7776 | 1168.7787 | 0.9 | ppm |
| macolacin-3Dab | C₅₅H₁₀₄N₁₆O₁₂ | 1181.8092 | 1181.8074 | 1.5 | ppm |
| macolacin-7Leu | C₅₄H₁₀₁N₁₅O₁₃ | 1168.7776 | 1168.7759 | 1.4 | ppm |
| macolacin-10Thr | C₅₂H₉₈N₁₅O₁₃ | 1156.7412 | 1156.7379 | 2.8 | ppm |
| macolacin-L1 | C₅₂H₉₇N₁₅O₁₃ | 1140.7463 | 1140.7424 | 3.4 | ppm |
| macolacin-L2 | C₅₃H₉₉N₁₅O₁₃ | 1154.7620 | 1154.7566 | 4.6 | ppm |
| macolacin-L3 | C₅₄H₁₀₁N₁₅O₁₃ | 1168.7776 | 1168.7751 | 2.1 | ppm |
| macolacin-L4 | C₅₅H₁₀₃N₁₅O₁₃ | 1182.7933 | 1182.7882 | 4.3 | ppm |
| macolacin-L5 | C₅₃H₉₉N₁₅O₁₄ | 1170.7569 | 1170.7531 | 3.2 | ppm |
| macolacin-L6 | C₅₈H₉₃N₁₅O₁₃ | 1208.7150 | 1208.7091 | 4.8 | ppm |
| macolacin-L7 | C₆₅H₉₉N₁₅O₁₃ | 1298.7620 | 1298.7574 | 3.5 | ppm |
| macolacin-L8 | C₅₄H₁₀₁N₁₅O₁₃ | 1202.7620 | 1202.7547 | 6.0 | ppm |
| macolacin-L9 | C₅1H₉₁N₁₅O₁₃ | 1122.6994 | 1122.6926 | −6.0 | ppm |
| macolacin-L10 | C₅₃H₉₃N₁₅O₁₃ | 1148.7150 | 1148.7120 | −2.6 | ppm |
| macolacin-L11 | C₅₅H₉₃N₁₅O₁₃ | 1172.7150 | 1172.7184 | 2.9 | ppm |
| macolacin-L12 | C₆₆H₉₇N₁₇O₁₈S | 1448.6991 | 1448.7029 | 2.6 | ppm |
| macolacin-L13 | C₅₈H₁₀₃N₁₅O₁₃ | 1218.7933 | 1218.7906 | −2.2 | ppm |
| macolacin-L14 | C₆₃H₉₄Br₂N₁₆O₁₃ | 1441.5626 | 1441.5622 | −0.3 | ppm |

TABLE 7
Accession number of polymyxin-like BGCs.

| Polymyxin- | | |
|---|---|---|
| like BGCs | Organism Name | Accession code |
| Colistin | Paenibacillus alvei | KP262070.1 |
| Polymyxin A | Paenibacillus polymyxa | EU371992.1 |
| Polymyxin B | Paenibacillus polymyxa | JN660148.1 |
| Macolacin | Paenibacillus_{—}xylanexedens_strain_PAMC_22703 | NZ_CP018620.1.cluster015 |
| CAT9-1 | Paenibacillus_{—}polymyxa_E681 | NC_014483.2.cluster009 |
| CAT9-2 | Paenibacillus_{—}polymyxa_SQR-21 | NZ_CP006872.1.cluster013 |
| CAT9-3 | Paenibacillus_{—}peoriae_strain_HS311 | NZ_CP011512.1.cluster012 |
| CAT9-4 | Paenibacillus_{—}polymyxa_strain_J | NZ_CP015423.1.cluster003 |
| CAT9-5 | Paenibacillus_{—}polymyxa_strain_YC0573_chromosome | NZ_CP017968.3.cluster013 |
| CAT9-6 | Paenibacillus_{—}polymyxa_strain_ATCC_15970 | NZ_CP011420.1.cluster011 |
| CAT9-7 | Paenibacillus_sp._lzh-N1_chromosome | NZ_CP025696.1.cluster005 |
| CAT9-8 | Paenibacillus_sp._FSL_R5- | NZ_MRTS01000010.cluster016 |
| | 0765_NODE_42_length_26692_cov_1.71286_ID_5187 | |
| CAT9-9 | Paenibacillus_{—}assamensis_DSM_18201_H580DRAFT_scaffold00005.5 | NZ_KE383837.1.cluster005 |
| CAT9-10 | Paenibacillus_{—}alvei_TS-15_PAALTS15_52 | NZ_ATMT01000001.cluster004 |
| CAT9-11 | Paenibacillus_{—}polysaccharolyticus_strain_BL9 | NZ_FMVM01000034.cluster011 |
| CAT9-12 | Paenibacillus_{—}amylolyticus_strain_FSL_F4- | NZ_MRTG01000010.cluster006 |
| | 0260_NODE_28_length_22572_cov_1.32332_ID_3095 | |
| CAT9-13 | Paenibacillus_{—}amylolyticus_strain_FSL_J3- | NZ_MRTO01000010.cluster009 |
| | 0122_NODE_28_length_25344_cov_1.50965_ID_3188 | |
| CAT9-14 | Paenibacillus_{—}polymyxa_strain_HY96-2_chromosome | NZ_CP025957.1.cluster013 |
| CAT9-15 | Paenibacillus_{—}polymyxa_strain_YC0136_chromosome | NZ_CP017967.2.cluster013 |
| CAT9-16 | Paenibacillus_sp._CF095 | NZ_FMZN01000016.cluster015 |
| CAT9-18 | Paenibacillus_{—}polymyxa_ATCC_842_PPt02_scaffold1 | NZ_GL905390.1.cluster013 |
| CAT9-19 | Paenibacillus_{—}polymyxa_strain_CICC_10580_contig_4 | NZ_JNCB01000001.cluster003 |
| CAT9-20 | Paenibacillus_{—}polymyxa_A18_scaffold00005 | NZ_JWJJ01000001cluster011 |
| CAT9-21 | Paenibacillus_{—}polymyxa_strain_NRRL_B-30509_contig00006 | NZ_JTHO01000001.cluster005 |
| CAT9-22 | Paenibacillus_{—}polymyxa_strain_EBL06_Scaffold18 | NZ_KQ004561.1.cluster015 |
| CAT9-23 | Paenibacillus_{—}amylolyticus_strain_FSL_H7- | NZ_MRTI01000010.cluster014 |
| | 0692_NODE_6_length_355017_cov_1.89367_ID_2749 | |
| CAT9-24 | Paenibacillus_sp._FSL_R5-192_Contig003 | NZ_ASPR01000001.cluster002 |
| CAT9-25 | Paenibacillus_sp._FSL_H7-689_Contig007 | NZ_ASPU01000002.cluster003 |
| CAT9-26 | Paenibacillus_sp._VT-400_contig65 | NZ_LELF01000001. cluster005 |
| CAT9-27 | Paenibacillus_sp._cl6col | NZ_FNAZ01000029.cluster009 |
| CAT9-28 | Paenibacillus_sp._UNC217MF_BP95DRAFT_scaffold00014.14_C | NZ_KK366027.1.cluster011 |
| CAT9-29 | Paenibacillus_{—}alvei_DSM_29_PAV_1c | NZ_AMBZ01000023.cluster005 |
| CAT9-30 | Paenibacillus_sp._PAMC_26794_5104_86 | NZ_ANHX01000001.cluster015 |
| CAT9-31 | Paenibacillus_{—}alvei_A6-6i-x_PAAL66ix_28 | NZ_ATMS01000001.cluster016 |
| CAT9-32 | Paenibacillus_{—}polymyxa_M1_main_chromosome | NC_017542.1.cluster012 |
| CAT9-33 | Paenibacillus_{—}polymyxa_CR1 | NC_023037.2.cluster011 |
| CAT9-34 | Paenibacillus_sp._Mc5Re-14 | NZ_CVPD01000001.cluster009 |
| CAT9-35 | Paenibacillus_{—}jamilae_strain_NS115_contig_45 | NZ_LDRX01000001.cluster016 |

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A compound represented by Formula (I) 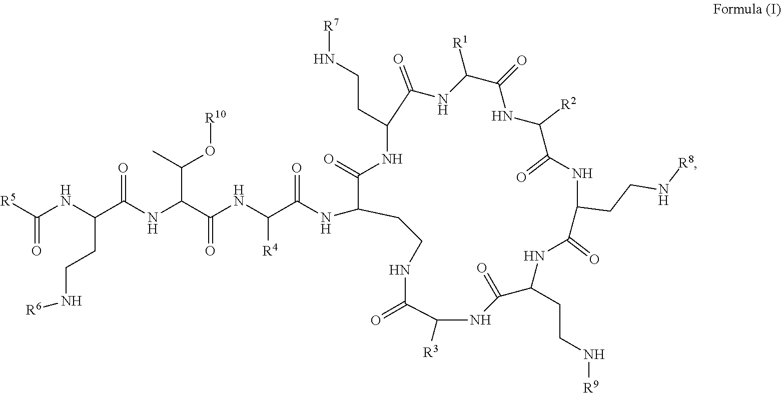
or a racemate, an enantiomer, a diastereomer, a pharmaceutically acceptable salt, or a derivative thereof,
wherein each R¹, R², R³, R⁴, R⁵, R⁶, R⁷, R⁸, R⁹, and R¹⁰ is independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, and any combinations thereof.

2. The compound of claim 1, wherein each R¹, R², R³, R⁴, and R⁵ is independently selected from the group consisting of alkyl, alkenyl, aryl, aryl alkyl, aminoalkyl, hydroxyalkyl, and any combination thereof.

3. The compound of claim 2, wherein each R¹, R², R³, R⁴, and R⁵ is independently selected from the group consisting of linear C₁-C₁₀ alkyl, branched C₁-C₁₀ alkyl, linear aryl-C₁-C₁₀ alkyl, branched aryl-C₁-C₁₀ alkyl, linear amino-C₁-C₁₀ alkyl, amino-branched C₁-C₁₀ alkyl, linear hydroxy-C₁-C₁₀ alkyl, hydroxy-branched C₁-C₁₀ alkyl, linear C₁-C₁₀ alkenyl, branched C₁-C₁₀ alkenyl, linear aryl-C₁-C₁₀ alkenyl, branched aryl-C₁-C₁₀ alkenyl, linear amino-C₁-C₁₀ alkenyl, amino-branched C₁-C₁₀ alkenyl, linear hydroxy-C₁-C₁₀ alkenyl, hydroxy-branched C₁-C₁₀ alkenyl, 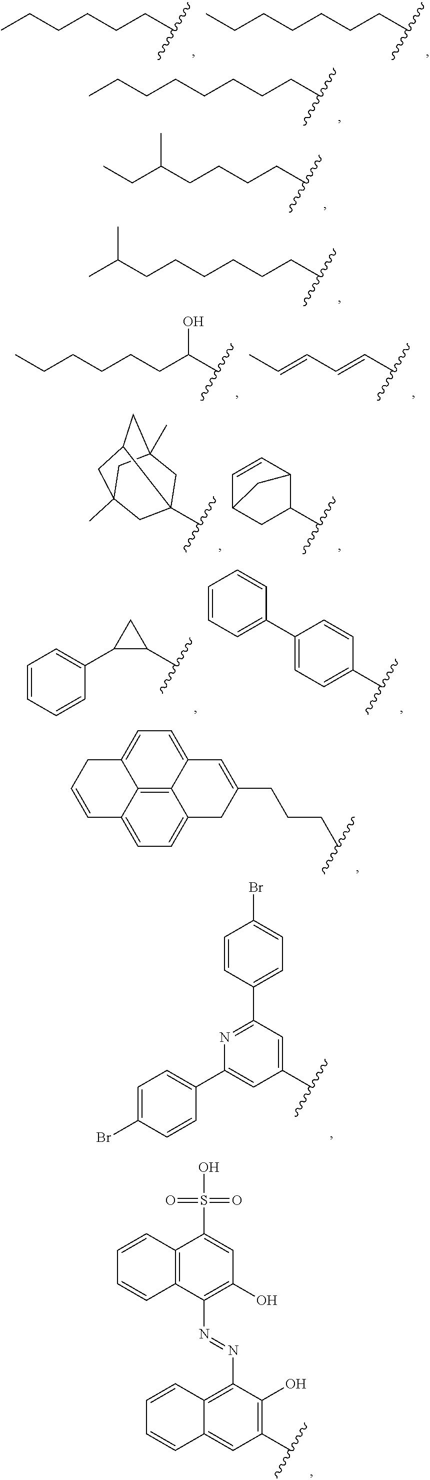
and any combination thereof.

4. The compound of claim 1, wherein each R⁶, R⁷, R⁸, R⁹, and R¹⁰ is independently selected from the group consisting of hydrogen, sulfonyl, and alkyl sulfonyl.

5. The compound of claim 4, wherein the alkyl sulfonyl is methanesulfonyl.

6. The compound of claim 1, wherein the compound represented by Formula (I) is a compound selected from the group consisting of: 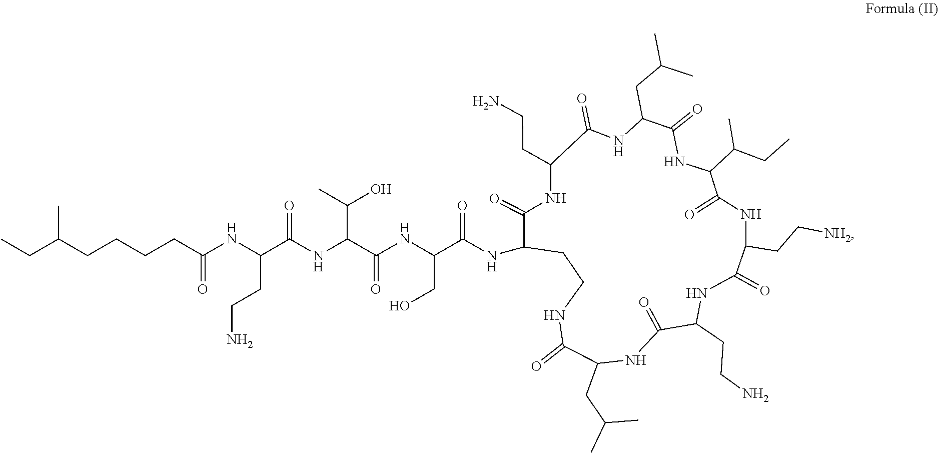 [SMILES: CCC(C)CCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]
or a racemate, an enantiomer, a diastereomer, a pharmaceutically acceptable salt, or a derivative thereof, 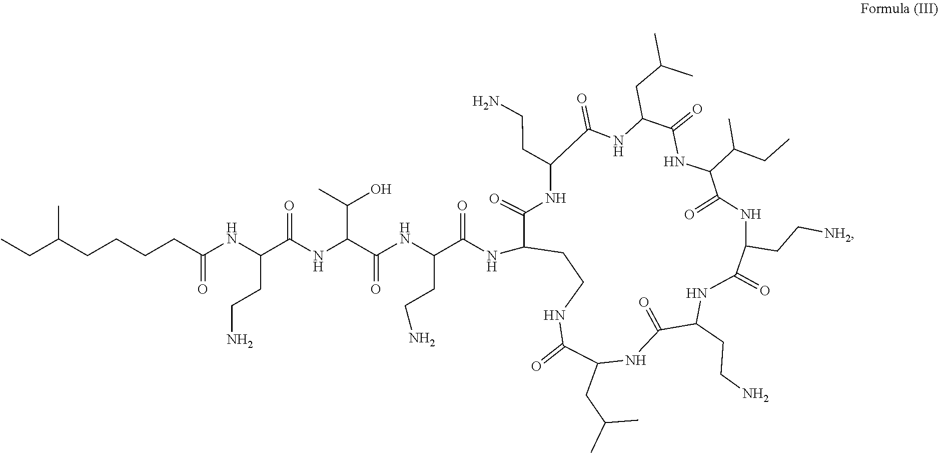 [SMILES: CCC(C)CCCCC(=O)NC(CCN)C(=O)NC(C(=O)NC(CCN)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]
or a racemate, an enantiomer, a diastereomer, a pharmaceutically acceptable salt, or a derivative 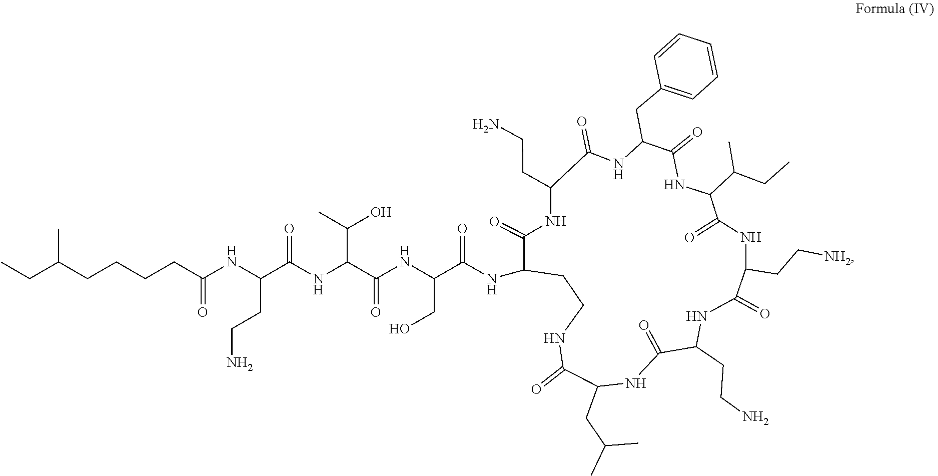 [SMILES: CCC(C)CCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(Cc2ccccc2)NC(=O)C(CCN)NC1=O)C(C)O]
or a racemate, an enantiomer, a diastereomer, a pharmaceutically acceptable salt, or a derivative therof, 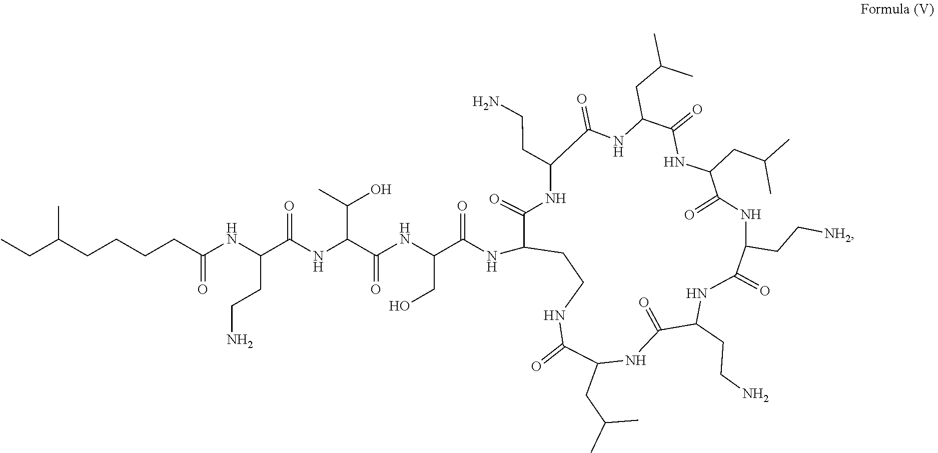 [SMILES: CCC(C)CCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(CC(C)C)NC(=O)C(CC(C)C)NC(=O)C(CCN)NC1=O)C(C)O]
or a racemate, an enantiomer, a diastereomer, a pharmaceutically acceptable salt, or a derivative thereof, 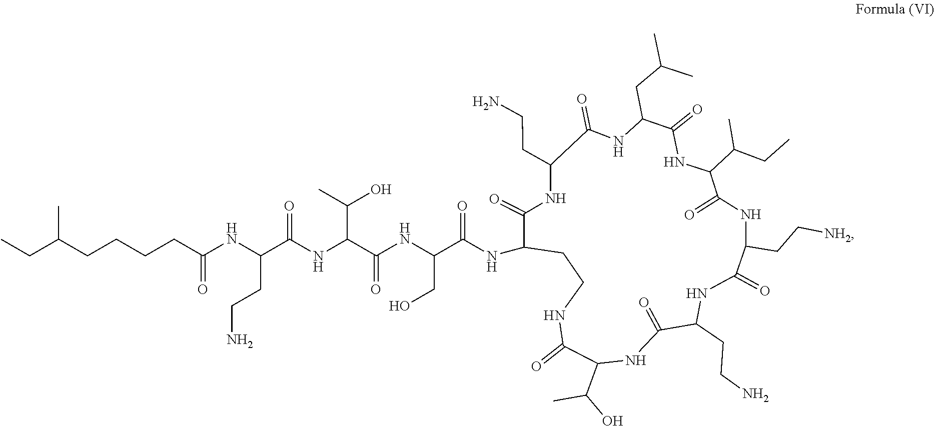 [SMILES: CCC(C)CCCCC(=O)NC(CCN)C(=O)NC(C(=O)NC(CO)C(=O)NC1CCNC(=O)C(C(C)O)NC(=O)C(CCN)NC(=O)C(CCN)NC(=O)C(C(C)CC)NC(=O)C(CC(C)C)NC(=O)C(CCN)NC1=O)C(C)O]
or a racemate, an enantiomer, a diastereomer, a pharmaceutically acceptable salt, or a derivative 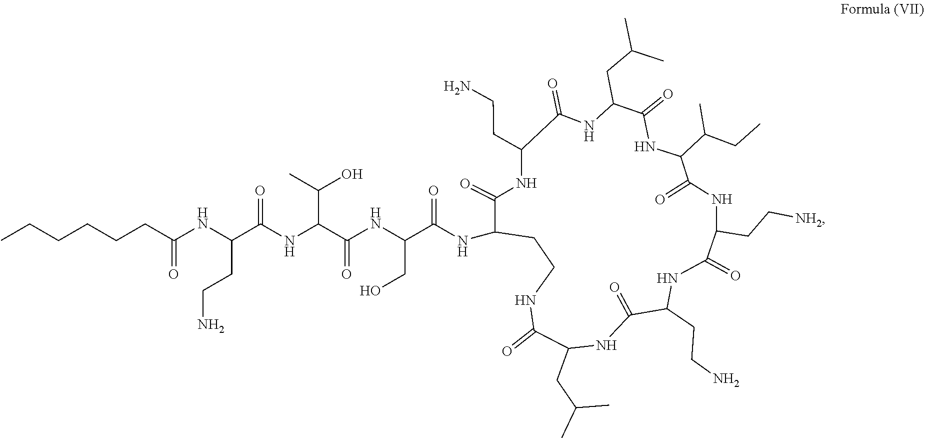 [SMILES: CCCCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]
or a racemate, an enantiomer, a diastereomer, a pharmaceutically acceptable salt, or a derivative 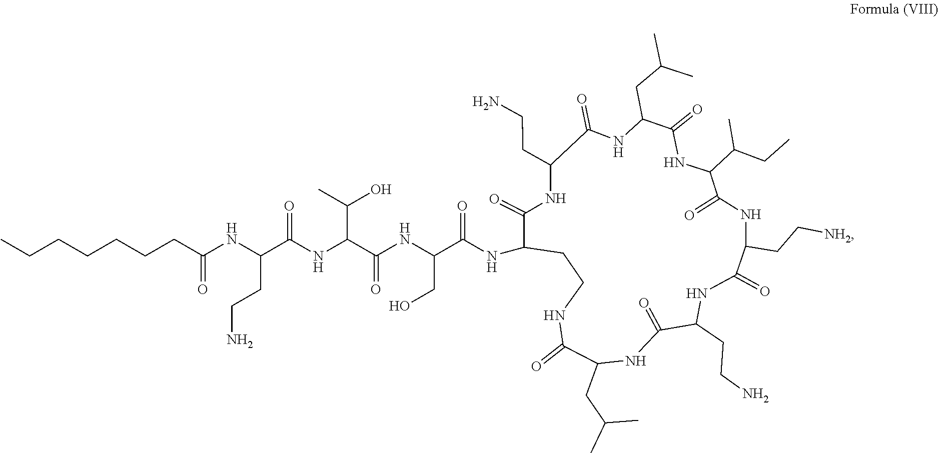 [SMILES: CCCCCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]
or a racemate, an enantiomer, a diastereomer, a pharmaceutically acceptable salt, or a derivative thereof, 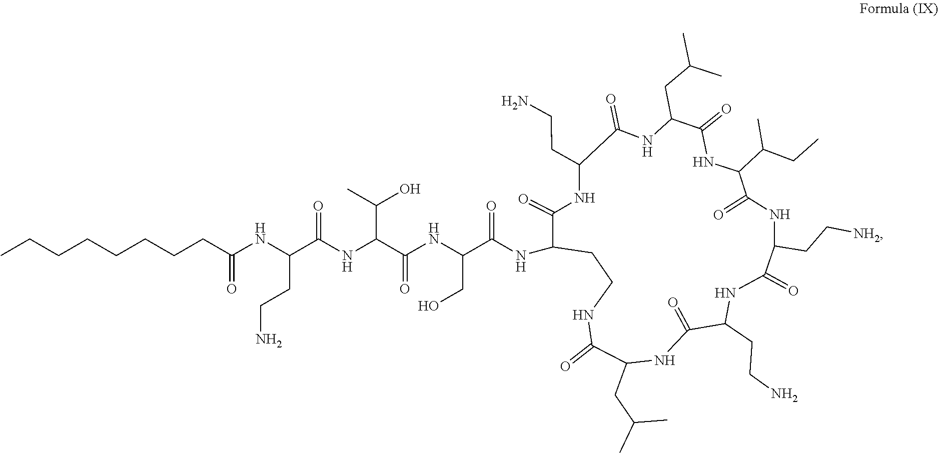 [SMILES: CCCCCCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]
or a racemate, an enantiomer, a diastereomer, a pharmaceutically acceptable salt, or a derivative 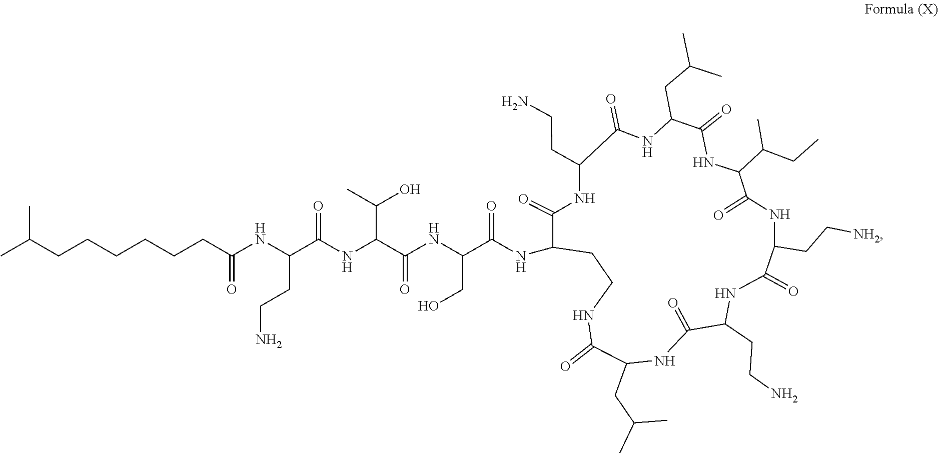 [SMILES: CCC(C)C1NC(=O)C(CC(C)C)NC(=O)C(CCN)NC(=O)C(NC(=O)C(CO)NC(=O)C(NC(=O)C(CCN)NC(=O)CCCCCCC(C)C)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative thereof, 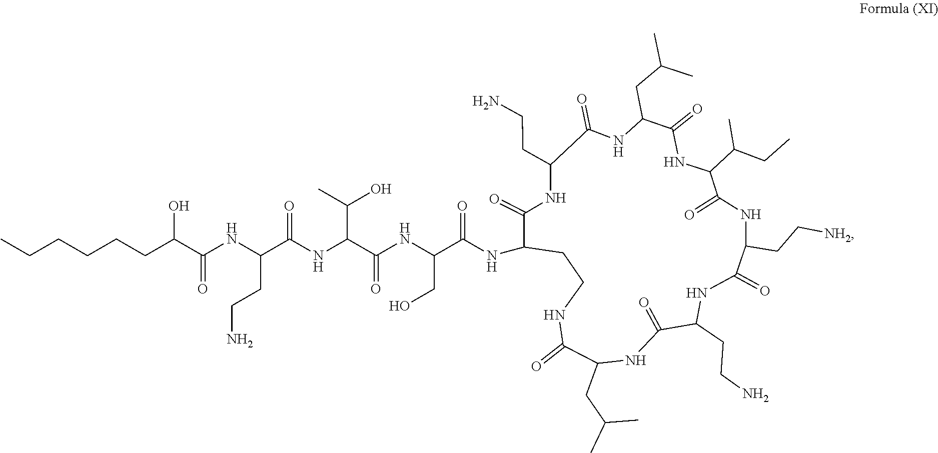 [SMILES: CCCCCCC(O)C(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]
or a racemate, an enantiomer, a diastereomer, a pharmaceutically acceptable salt, or a derivative 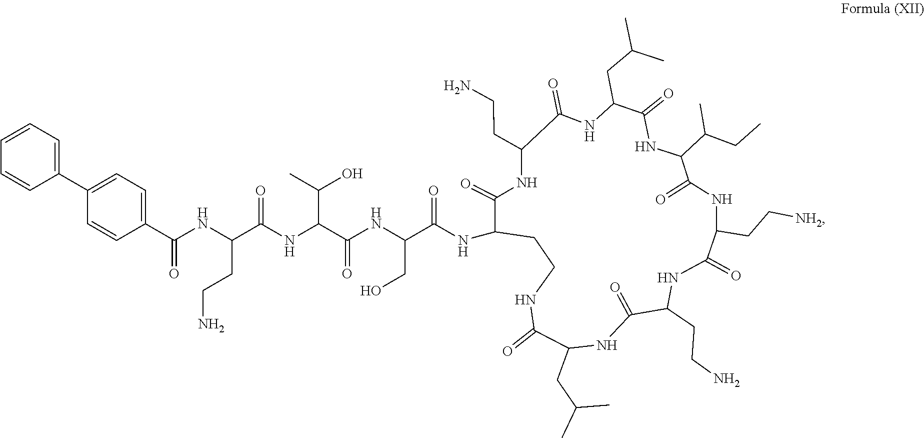 [SMILES: CCC(C)C1NC(=O)C(CC(C)C)NC(=O)C(CCN)NC(=O)C(NC(=O)C(CO)NC(=O)C(NC(=O)C(CCN)NC(=O)c2ccc(-c3ccccc3)cc2)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative thereof, 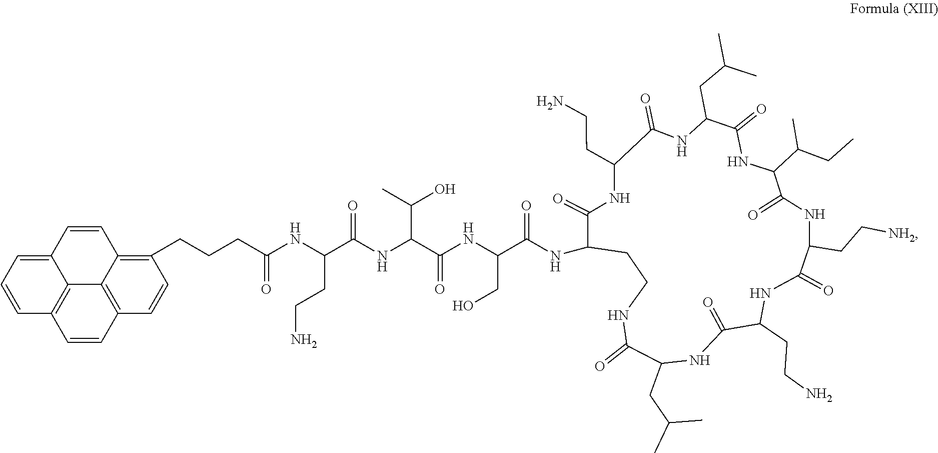 [SMILES: CCC(C)C1NC(=O)C(CC(C)C)NC(=O)C(CCN)NC(=O)C(NC(=O)C(CO)NC(=O)C(NC(=O)C(CCN)NC(=O)CCCc2ccc3ccc4cccc5ccc2c3c45)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative 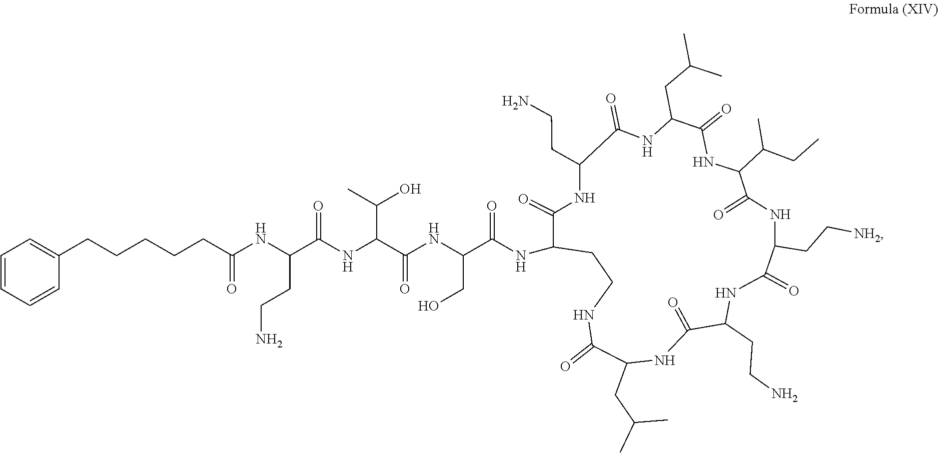 [SMILES: CCC(C)C1NC(=O)C(CC(C)C)NC(=O)C(CCN)NC(=O)C(NC(=O)C(CO)NC(=O)C(NC(=O)C(CCN)NC(=O)CCCCCc2ccccc2)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative thereof, 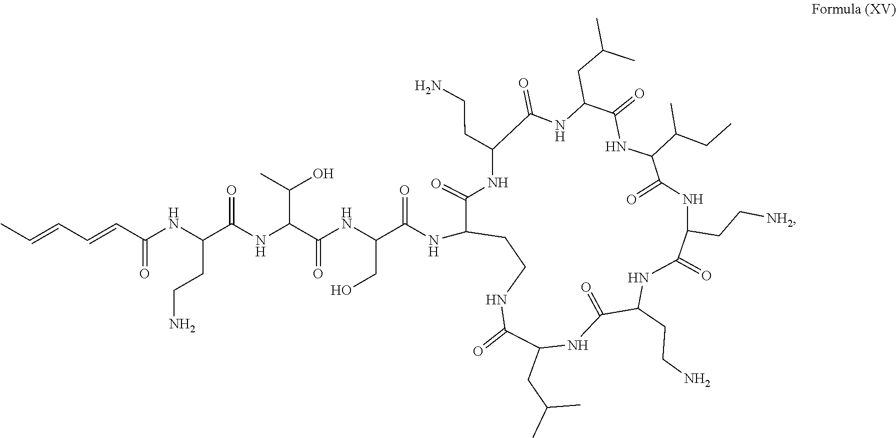 [SMILES: C/C=C/C=C/C(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]
or a racemate, an enantiomer, a diastereomer, a pharmaceutically acceptable salt, or a derivative 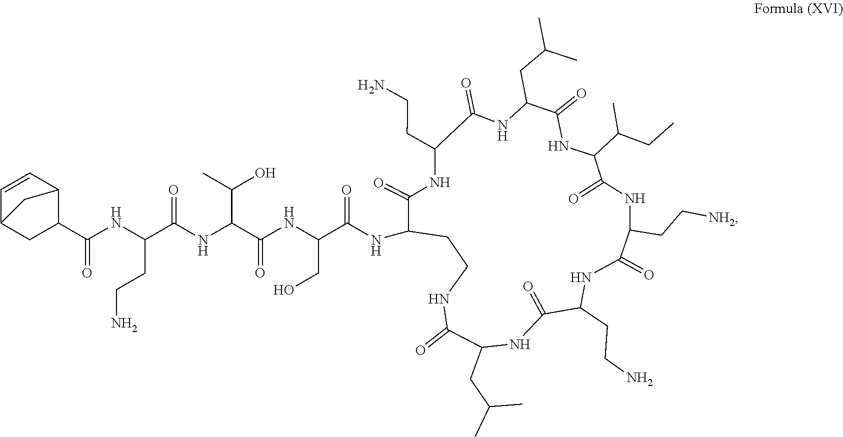 [SMILES: CCC(C)C1NC(=O)C(CC(C)C)NC(=O)C(CCN)NC(=O)C(NC(=O)C(CO)NC(=O)C(NC(=O)C(CCN)NC(=O)C2CC3C=CC2C3)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative thereof, 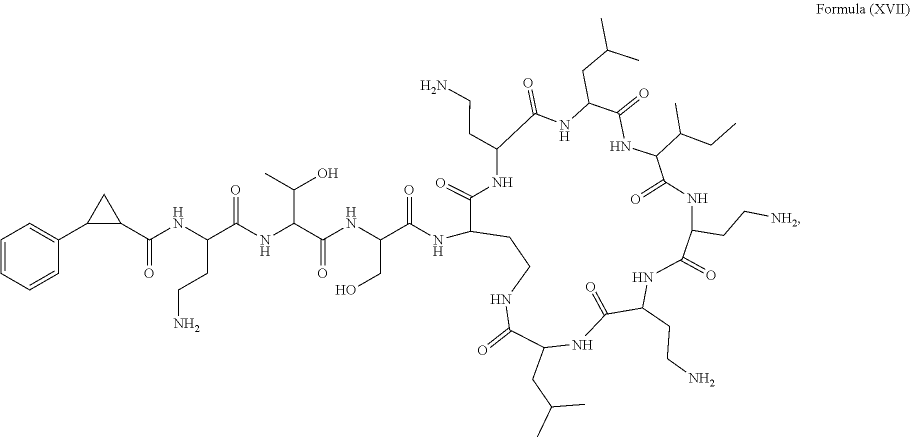 [SMILES: CCC(C)C1NC(=O)C(CC(C)C)NC(=O)C(CCN)NC(=O)C(NC(=O)C(CO)NC(=O)C(NC(=O)C(CCN)NC(=O)C2CC2c2ccccc2)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative 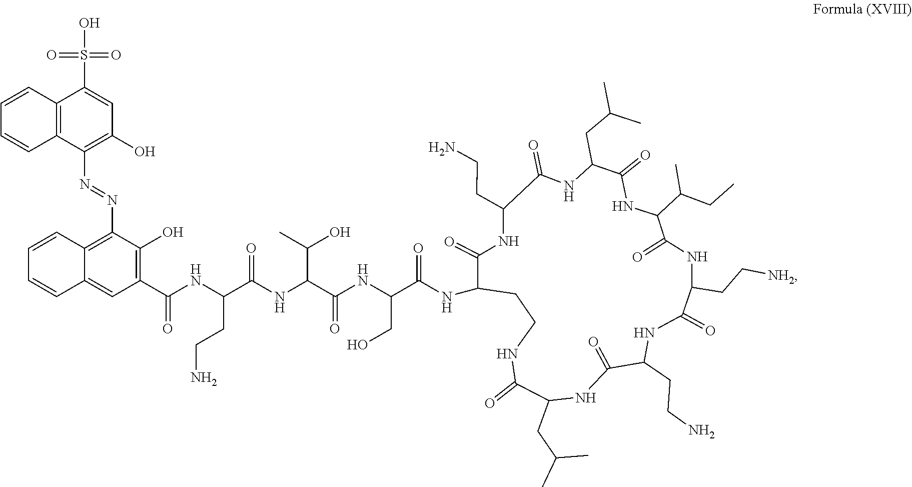 [SMILES: CCC(C)C1NC(=O)C(CC(C)C)NC(=O)C(CCN)NC(=O)C(NC(=O)C(CO)NC(=O)C(NC(=O)C(CCN)NC(=O)c2cc3ccccc3c(/N=N/c3c(O)cc(S(=O)(=O)O)c4ccccc34)c2O)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative thereof, 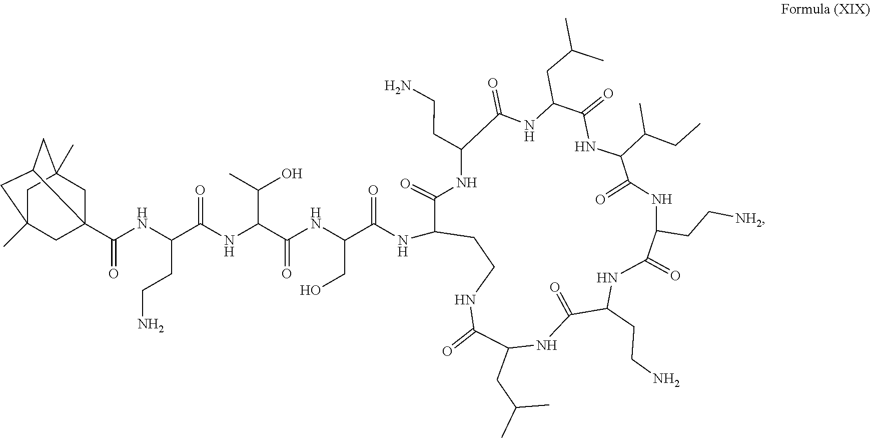 [SMILES: CCC(C)C1NC(=O)C(CC(C)C)NC(=O)C(CCN)NC(=O)C(NC(=O)C(CO)NC(=O)C(NC(=O)C(CCN)NC(=O)C23CC4(C)CC2CC(C)(C4)C3)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative thereof, and 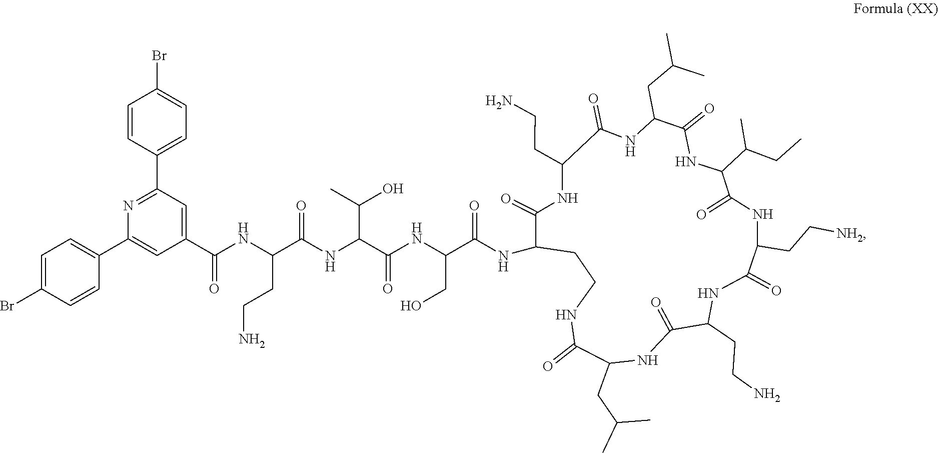 [SMILES: CCC(C)C1NC(=O)C(CC(C)C)NC(=O)C(CCN)NC(=O)C(NC(=O)C(CO)NC(=O)C(NC(=O)C(CCN)NC(=O)c2cc(-c3ccc(Br)cc3)nc(-c3ccc(Br)cc3)c2)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative thereof.

7. The compound of claim 1, wherein the compound represented by Formula (I) is a compound represented by Formula (Ia) 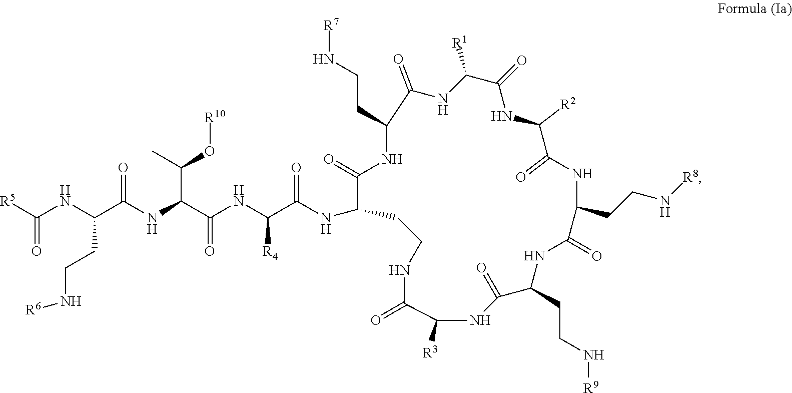
or a racemate, an enantiomer, a diastereomer, a pharmaceutically acceptable salt, or a derivative thereof,
wherein each R¹, R², R³, R⁴, R⁵, R⁶, R⁷, R⁸, R⁹, and R¹⁰ is independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, and any combinations thereof.

8. The compound of claim 7, wherein each R¹, R², R³, R⁴, and R⁵ is independently selected from the group consisting of alkyl, alkenyl, aryl, aryl alkyl, aminoalkyl, hydroxyalkyl, and any combination thereof.

9. The compound of claim 8, wherein each R¹, R², R³, R⁴, and R⁵ is independently selected from the group consisting of linear C₁-C₁₀ alkyl, branched C₁-C₁₀ alkyl, linear aryl-C₁-C₁₀ alkyl, branched aryl-C₁-C₁₀ alkyl, linear amino-C₁-C₁₀ alkyl, amino-branched C₁-C₁₀ alkyl, linear hydroxy-C₁-C₁₀ alkyl, hydroxy-branched C₁-C₁₀ alkyl, linear C₁-C₁₀ alkenyl, branched C₁-C₁₀ alkenyl, linear aryl-C₁-C₁₀ alkenyl, branched aryl-C₁-C₁₀ alkenyl, linear amino-C₁-C₁₀ alkenyl, amino-branched C₁-C₁₀ alkenyl, linear hydroxy-C₁-C₁₀ alkenyl, hydroxy-branched C₁-C₁₀ alkenyl, 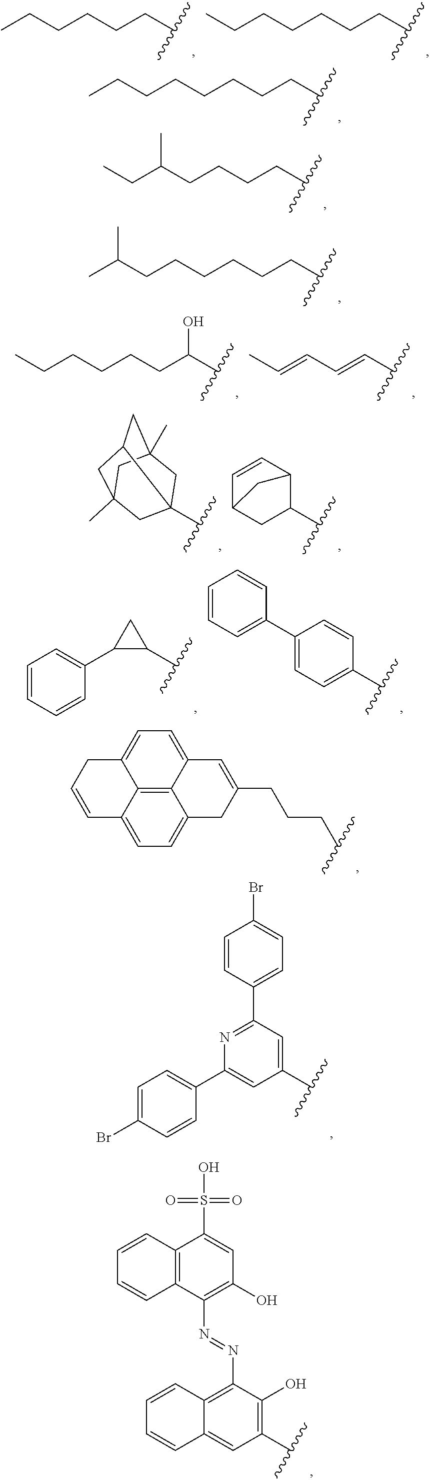
and any combination thereof.

10. The compound of claim 7, wherein the compound represented by Formula (Ia) is a compound selected from the group consisting of: 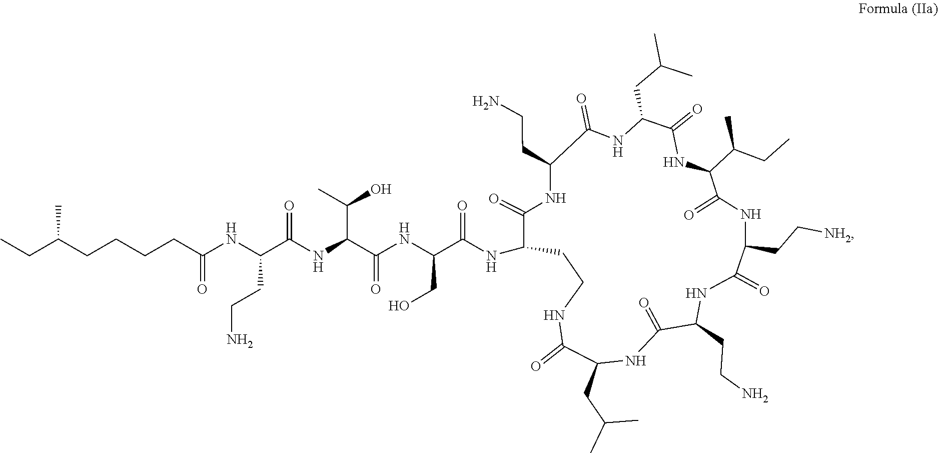 [SMILES: CC[C@H](C)CCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative thereof, 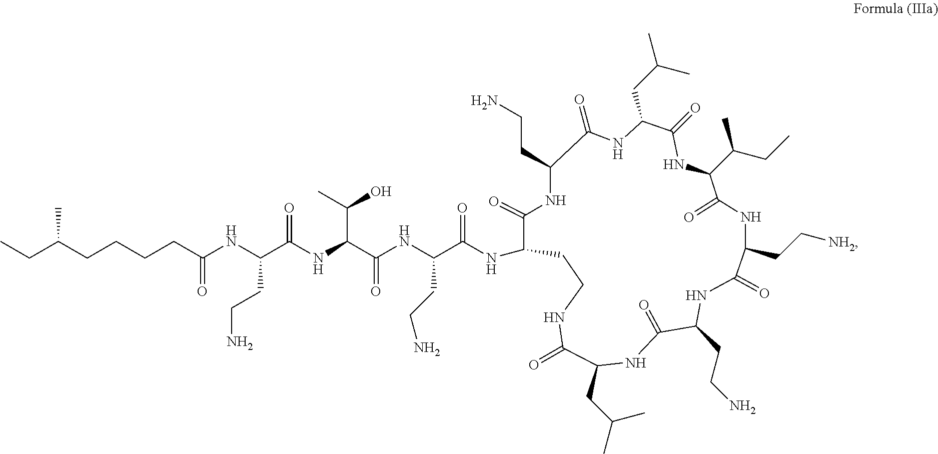 [SMILES: CC[C@H](C)CCCCC(=O)N[C@@H](CCN)C(=O)N[C@H](C(=O)N[C@@H](CCN)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative 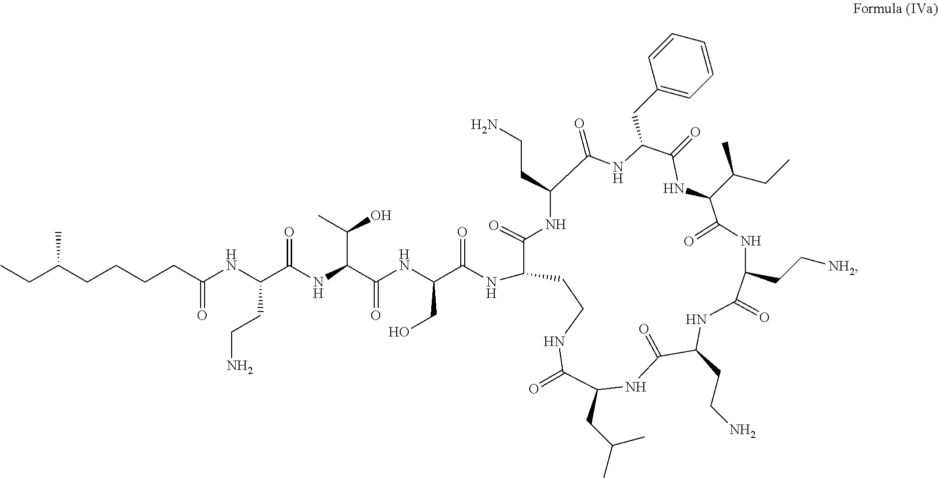 [SMILES: CC[C@H](C)CCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](Cc2ccccc2)NC(=O)[C@H](CCN)NC1=O)[C@@H](C)O]
or a racemate, an enantiomer, a diastereomer, a pharmaceutically acceptable salt, or a derivative thereof, 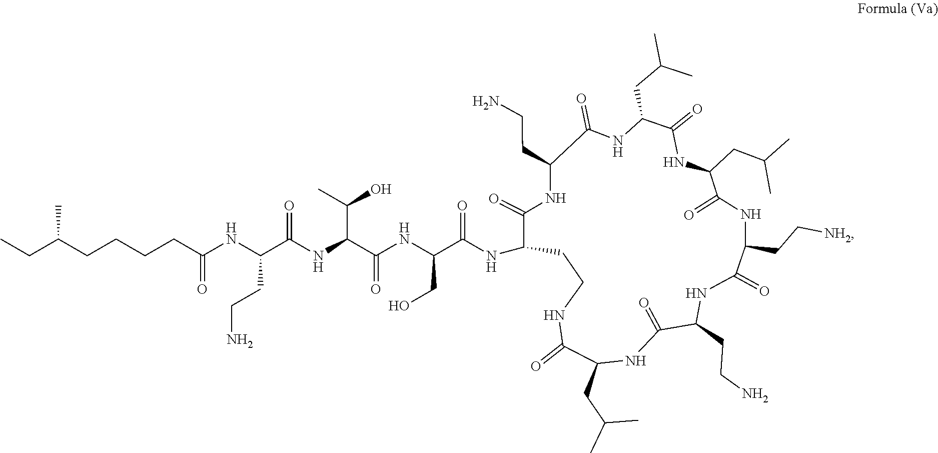 [SMILES: CC[C@H](C)CCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H](CC(C)C)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative 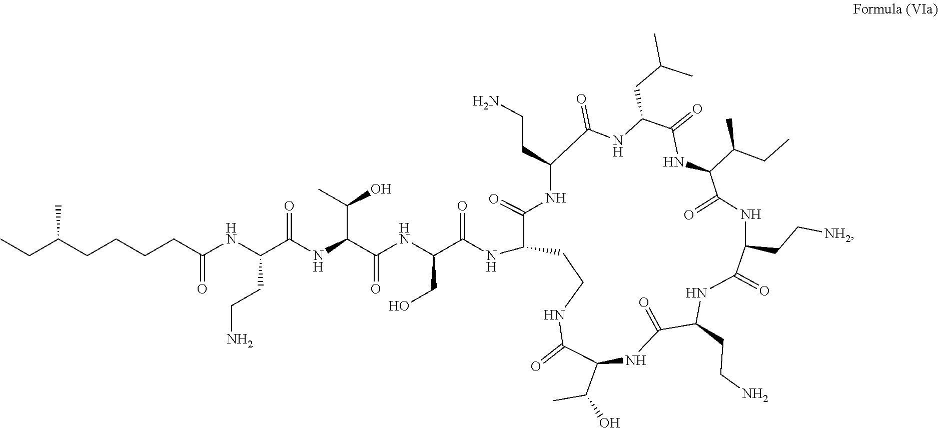 [SMILES: CC[C@H](C)CCCCC(=O)N[C@@H](CCN)C(=O)N[C@H](C(=O)N[C@H](CO)C(=O)N[C@H]1CCNC(=O)[C@H]([C@@H](C)O)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative thereof, 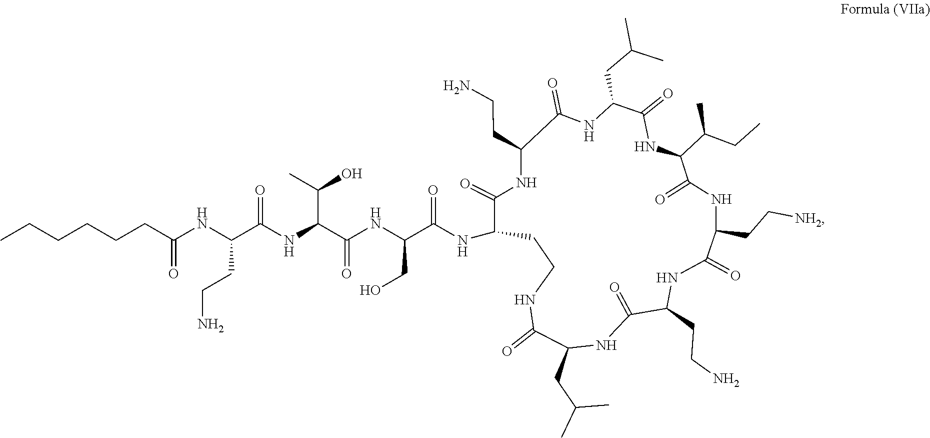 [SMILES: CCCCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative 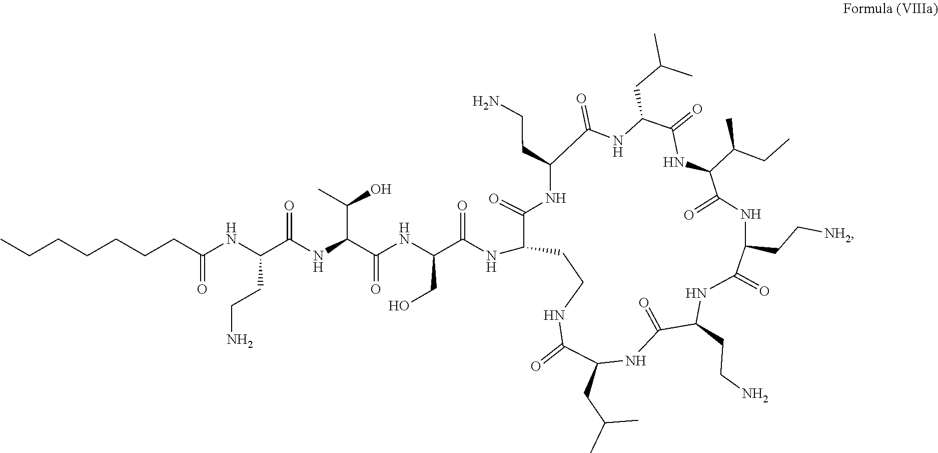 [SMILES: CCCCCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative 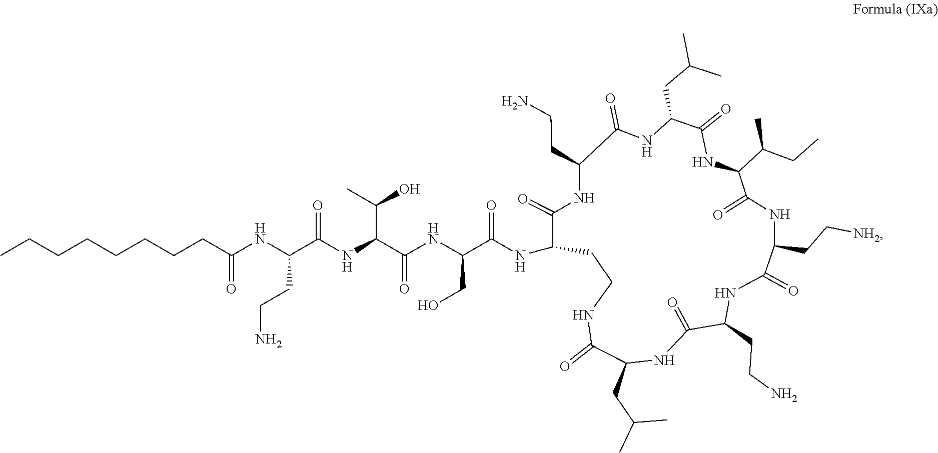 [SMILES: CCCCCCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative thereof, 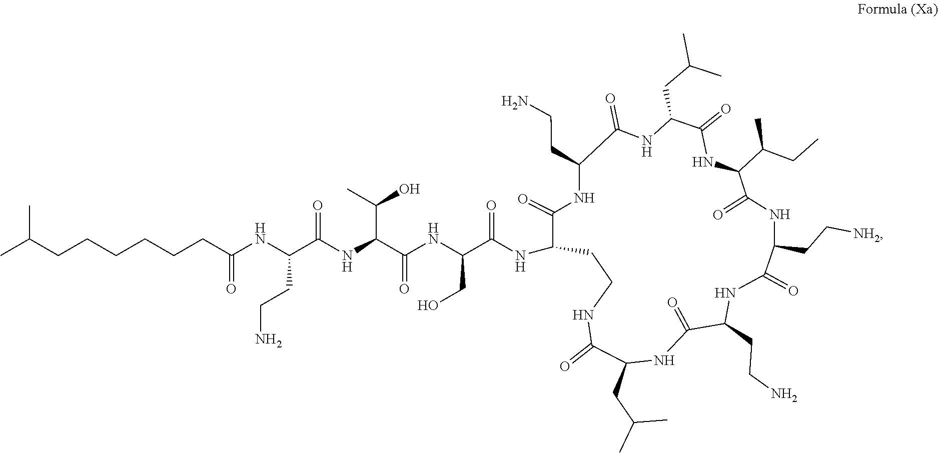 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)CCCCCCC(C)C)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative 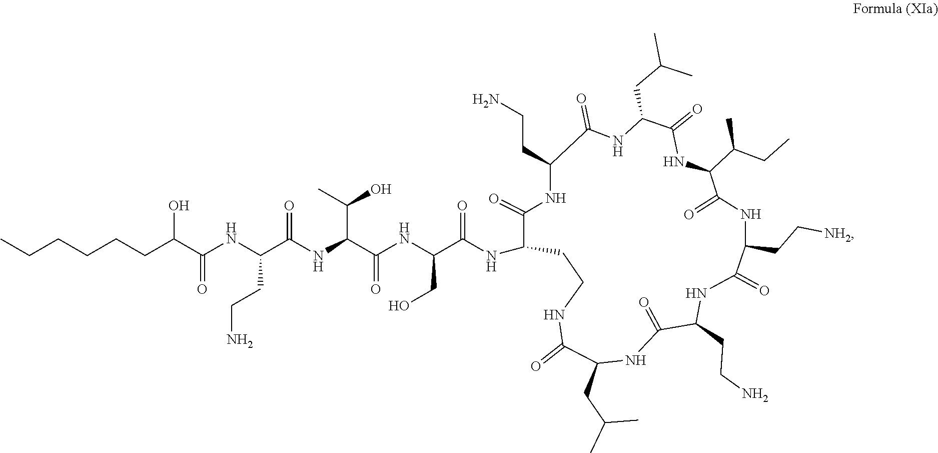 [SMILES: CCCCCCC(O)C(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative thereof, 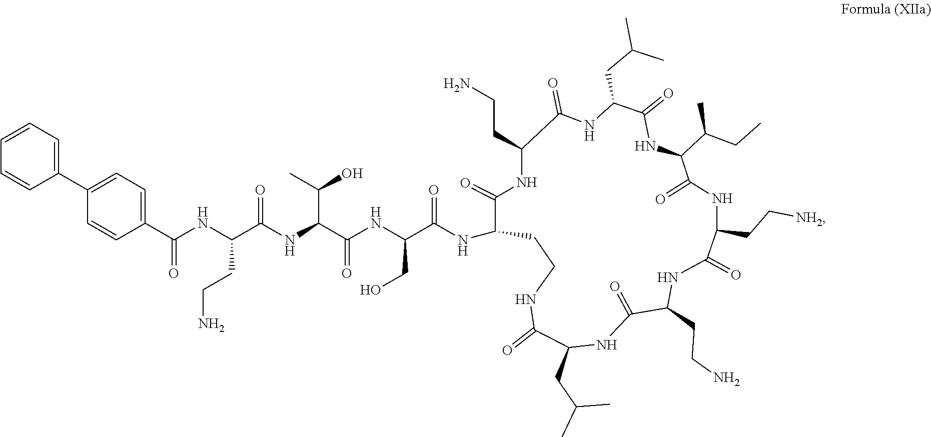 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)c2ccc(-c3ccccc3)cc2)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative 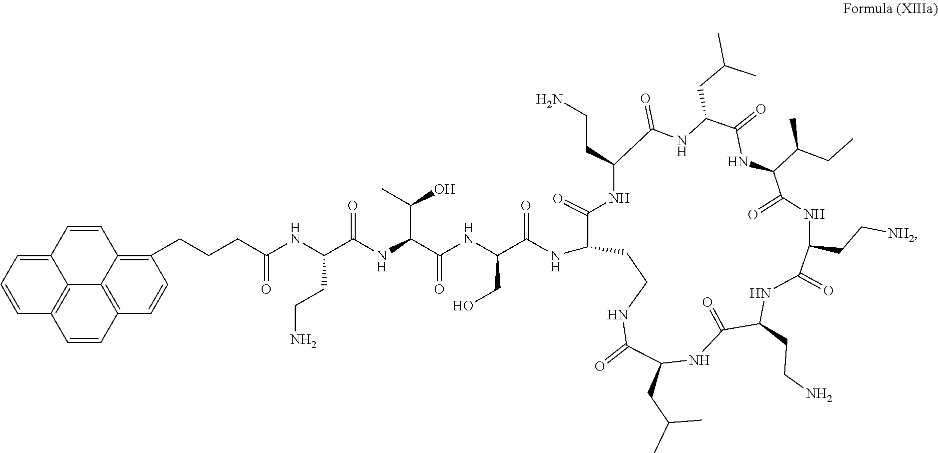 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)CCCc2ccc3ccc4cccc5ccc2c3c45)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative thereof, 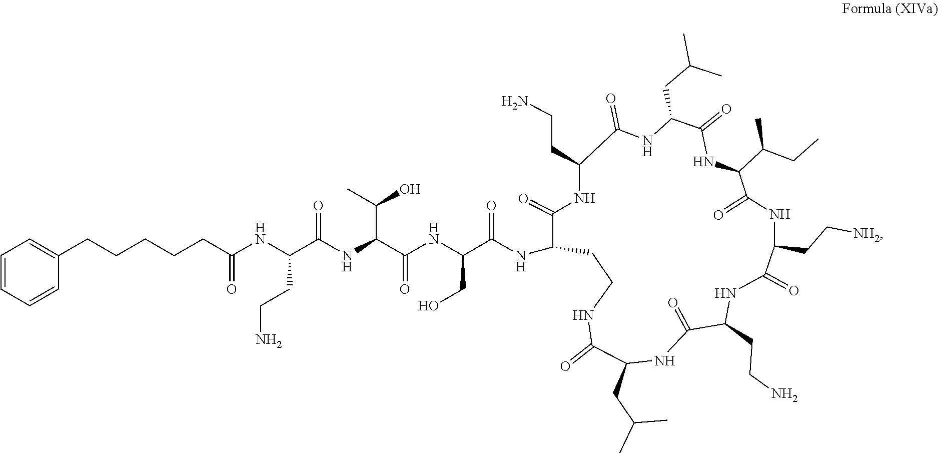 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)CCCCCc2ccccc2)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative 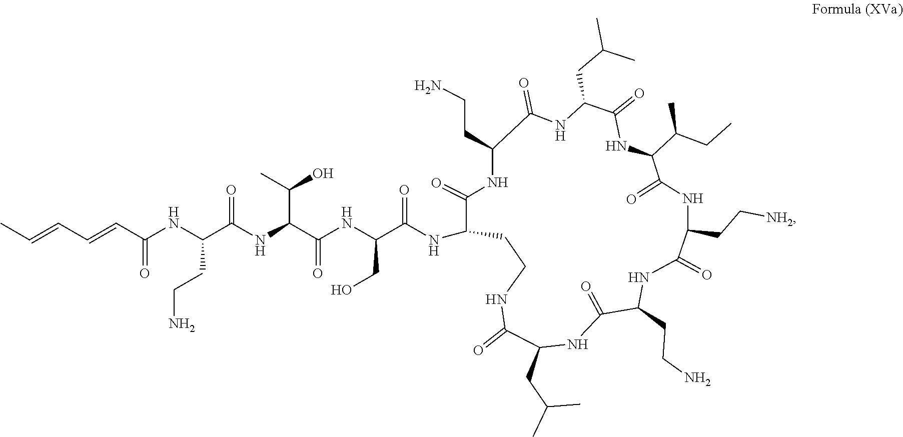 [SMILES: C/C=C/C=C/C(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative thereof, 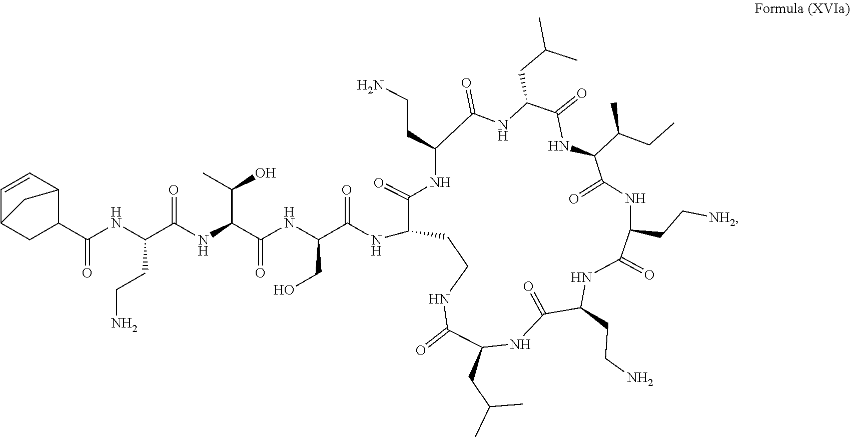 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)C2CC3C=CC2C3)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative 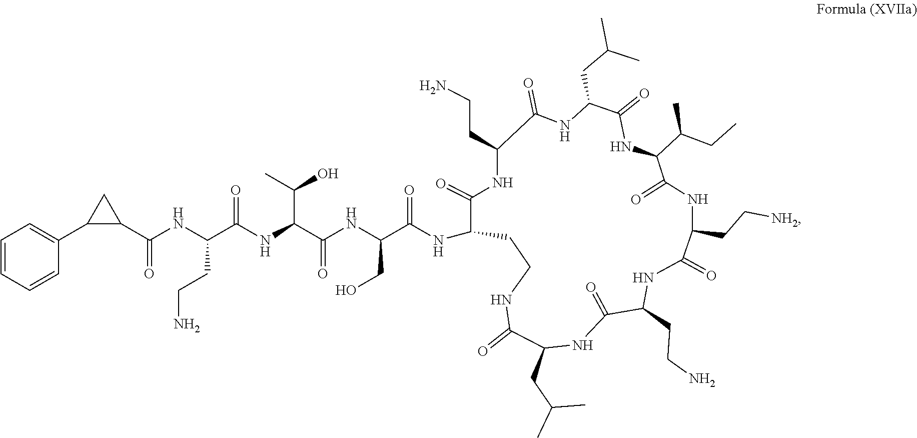 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)C2CC2c2ccccc2)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative thereof, 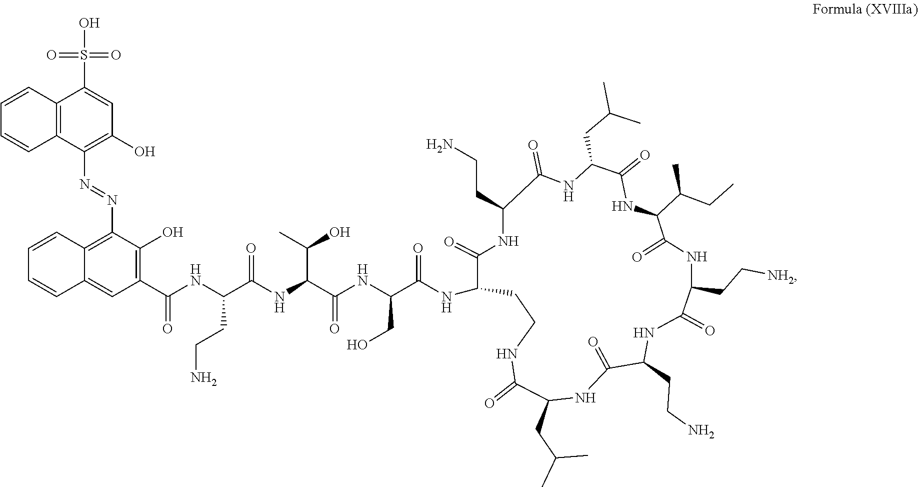 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)c2cc3ccccc3c(/N=N/c3c(O)cc(S(=O)(=O)O)c4ccccc34)c2O)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative 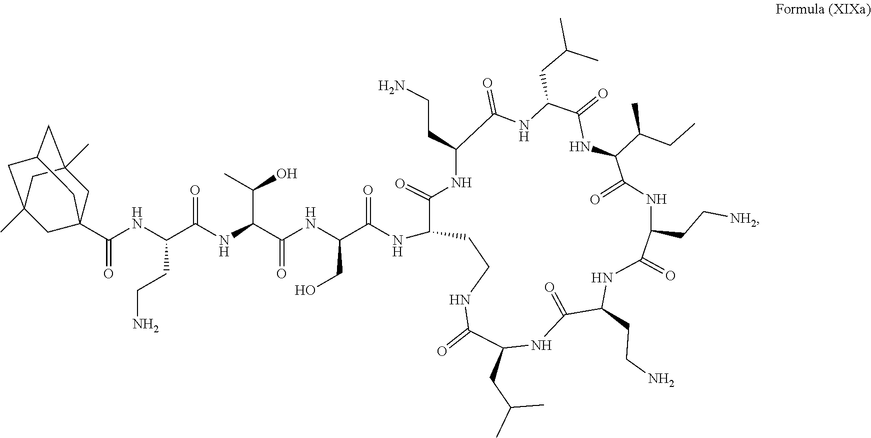 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)C23CC4CC(C)(CC(C)(C4)C2)C3)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative thereof, and 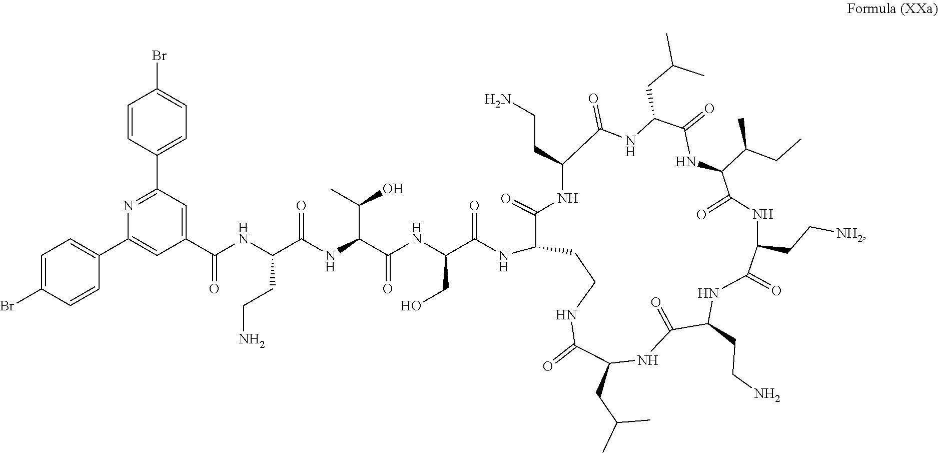 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)c2cc(-c3ccc(Br)cc3)nc(-c3ccc(Br)cc3)c2)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative thereof.

11. A pharmaceutical composition comprising a compound of claim 1.

12. An isolated nucleic acid encoding a macolacin.

13. The isolated nucleic acid of claim 12, wherein the macolacin comprises a compound represented by Formula (I) 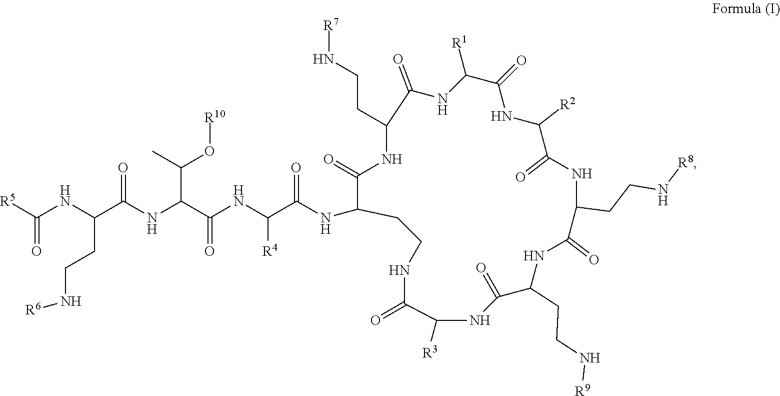
or a racemate, an enantiomer, a diastereomer, a pharmaceutically acceptable salt, or a derivative thereof,
wherein each R¹, R², R³, R⁴, R⁵, R⁶, R⁷, R⁸, R⁹, and R¹⁰ is independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, and any combinations thereof.

14. The isolated nucleic acid of claim 13, wherein each R¹, R², R³, R⁴, and R⁵ is independently selected from the group consisting of alkyl, alkenyl, aryl, aryl alkyl, aminoalkyl, hydroxyalkyl, and any combination thereof.

15. The isolated nucleic acid of claim 14, wherein each R¹, R², R³, R⁴, and R⁵ is independently selected from the group consisting of linear C₁-C₁₀ alkyl, branched C₁-C₁₀ alkyl, linear aryl-C₁-C₁₀ alkyl, branched aryl-C₁-C₁₀ alkyl, linear amino-C₁-C₁₀ alkyl, amino-branched C₁-C₁₀ alkyl, linear hydroxy-C₁-C₁₀ alkyl, hydroxy-branched C₁-C₁₀ alkyl, linear C₁-C₁₀ alkenyl, branched C₁-C₁₀ alkenyl, linear aryl-C₁-C₁₀ alkenyl, branched aryl-C₁-C₁₀ alkenyl, linear amino-C₁-C₁₀ alkenyl, amino-branched C₁-C₁₀ alkenyl, linear hydroxy-C₁-C₁₀ alkenyl, hydroxy-branched C₁-C₁₀ alkenyl, 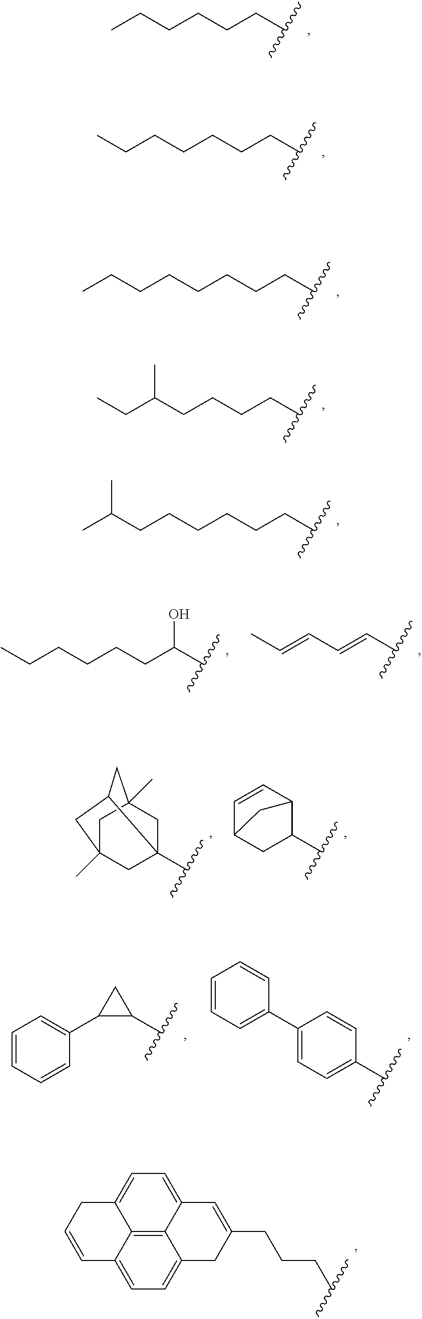 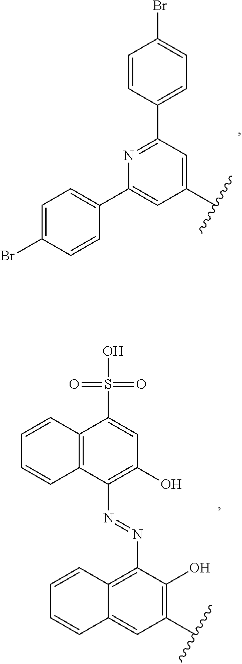 [SMILES: *c1cc(-c2ccc(Br)cc2)nc(-c2ccc(Br)cc2)c1 ; *c1cc2ccccc2c(/N=N/c2c(O)cc(S(=O)(=O)O)c3ccccc23)c1O]
and any combination thereof.

16. The isolated nucleic acid of claim 13, wherein the compound represented by Formula (I) is a compound selected from the group consisting of: 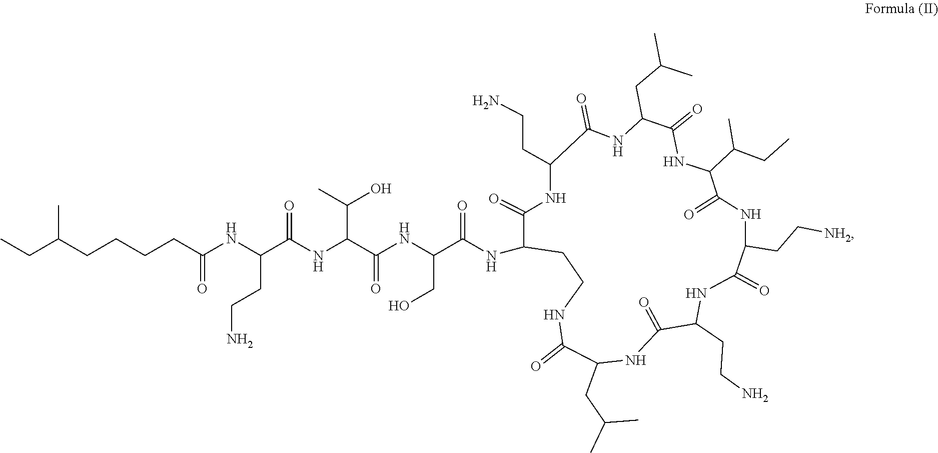 [SMILES: CCC(C)CCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]
or a racemate, an enantiomer, a diastereomer, a pharmaceutically acceptable salt, or a derivative 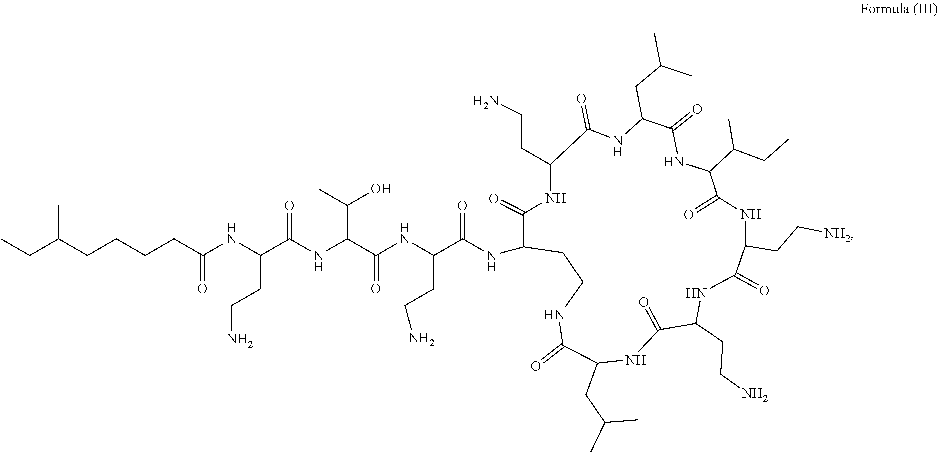 [SMILES: CCC(C)CCCCC(=O)NC(CCN)C(=O)NC(C(=O)NC(CCN)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]
or a racemate, an enantiomer, a diastereomer, a pharmaceutically acceptable salt, or a derivative thereof, 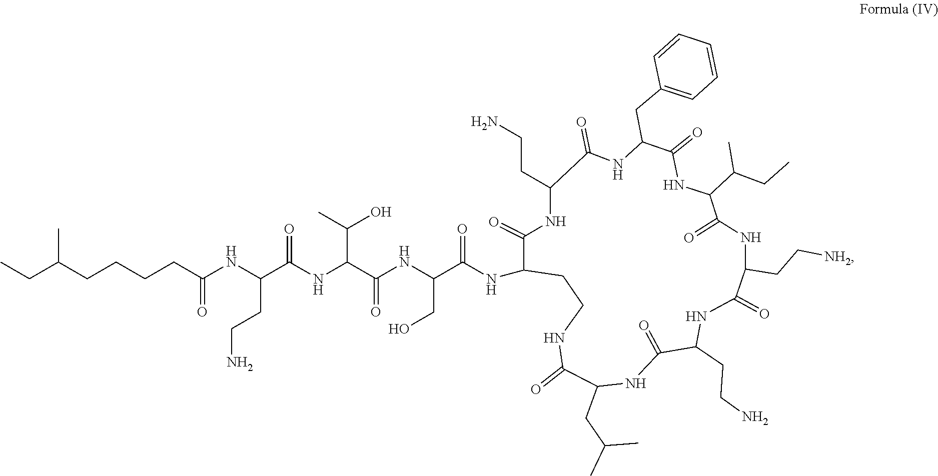 [SMILES: CCC(C)CCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(Cc2ccccc2)NC(=O)C(CCN)NC1=O)C(C)O]
or a racemate, an enantiomer, a diastereomer, a pharmaceutically acceptable salt, or a derivative 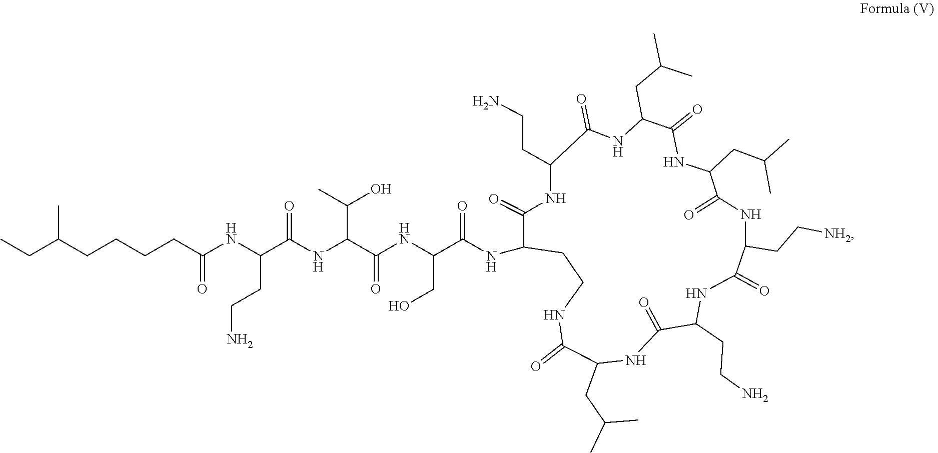 [SMILES: CCC(C)CCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(CC(C)C)NC(=O)C(CC(C)C)NC(=O)C(CCN)NC1=O)C(C)O]
or a racemate, an enantiomer, a diastereomer, a pharmaceutically acceptable salt, or a derivative thereof, 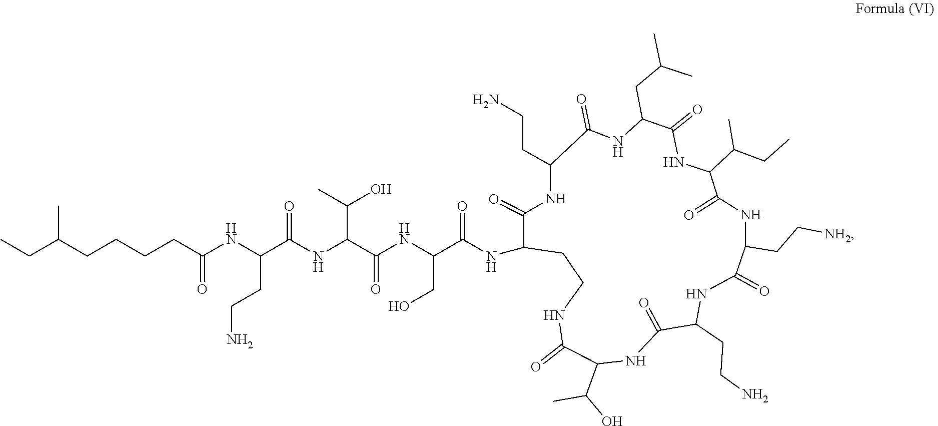 [SMILES: CCC(C)CCCCC(=O)NC(CCN)C(=O)NC(C(=O)NC(CO)C(=O)NC1CCNC(=O)C(C(C)O)NC(=O)C(CCN)NC(=O)C(CCN)NC(=O)C(C(C)CC)NC(=O)C(CC(C)C)NC(=O)C(CCN)NC1=O)C(C)O]
or a racemate, an enantiomer, a diastereomer, a pharmaceutically acceptable salt, or a derivative 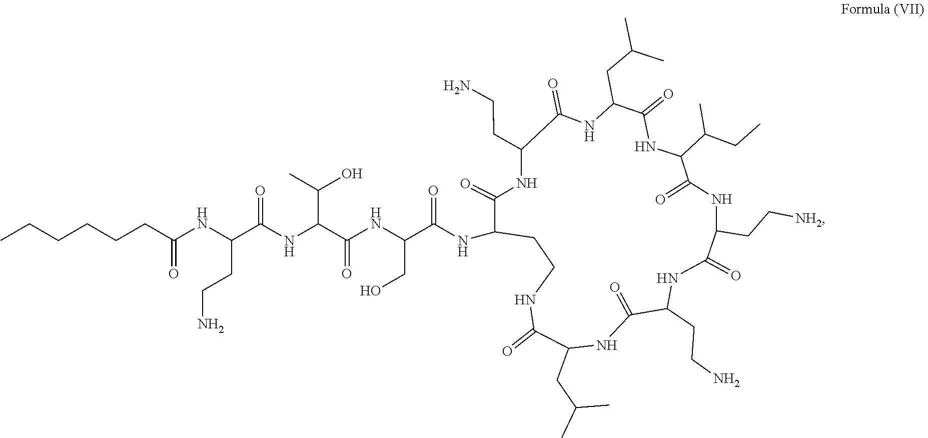 [SMILES: CCCCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]
or a racemate, an enantiomer, a diastereomer, a pharmaceutically acceptable salt, or a derivative 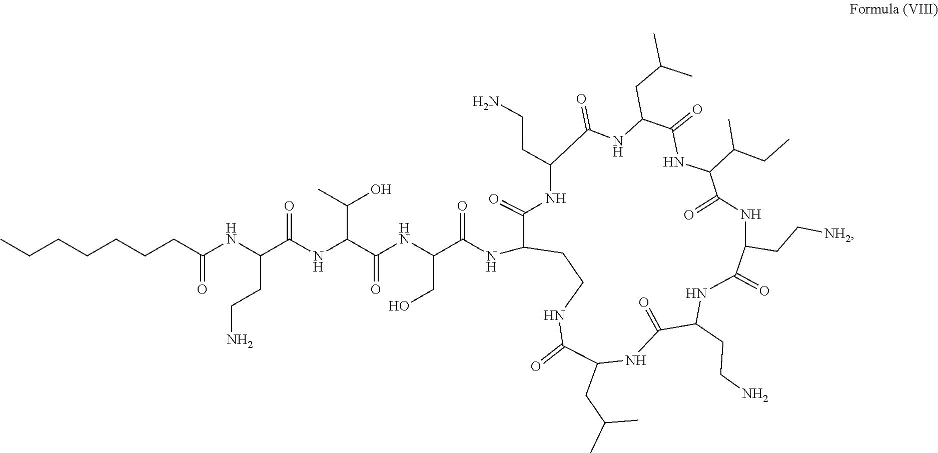 [SMILES: CCCCCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]
or a racemate, an enantiomer, a diastereomer, a pharmaceutically acceptable salt, or a derivative thereof, 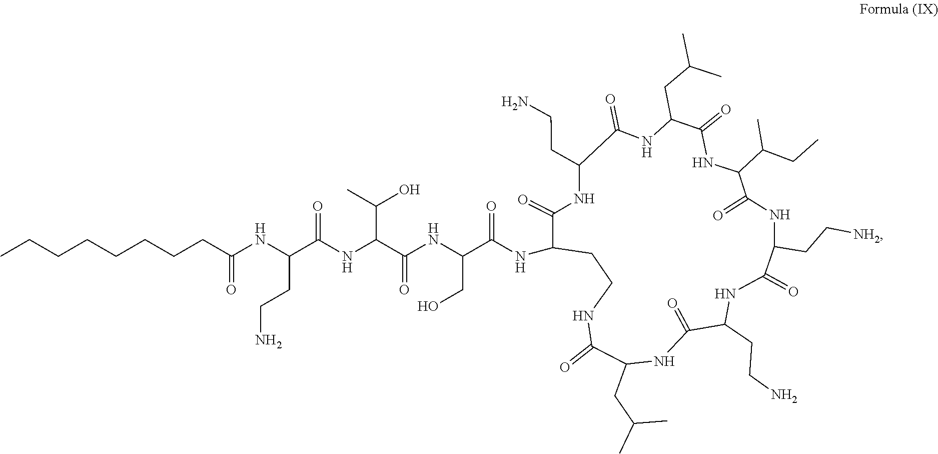 [SMILES: CCCCCCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]
or a racemate, an enantiomer, a diastereomer, a pharmaceutically acceptable salt, or a derivative 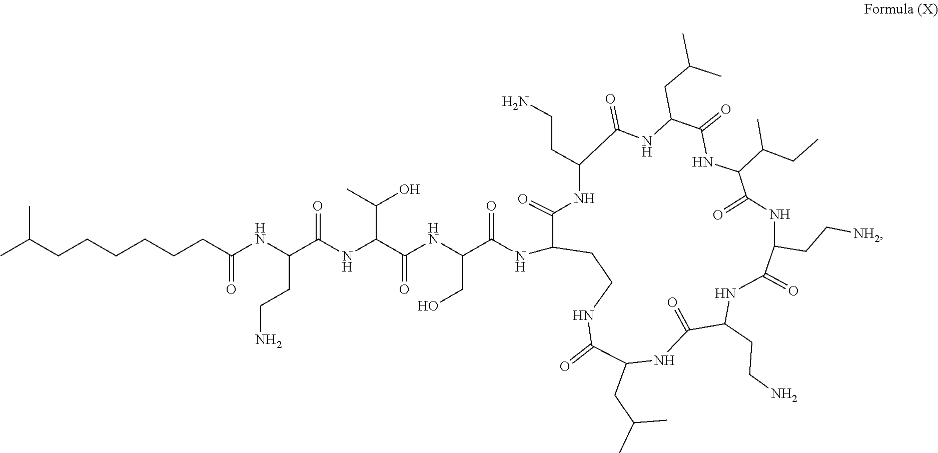 [SMILES: CCC(C)C1NC(=O)C(CC(C)C)NC(=O)C(CCN)NC(=O)C(NC(=O)C(CO)NC(=O)C(NC(=O)C(CCN)NC(=O)CCCCCCC(C)C)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative thereof, 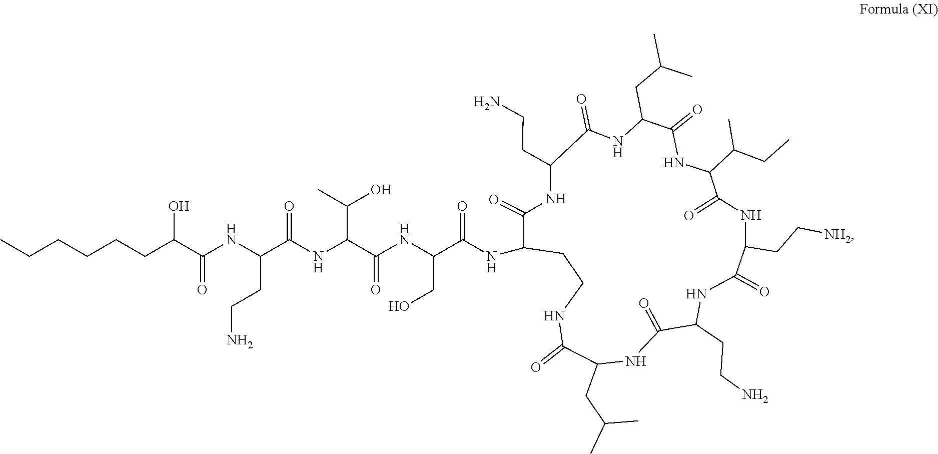 [SMILES: CCCCCCC(O)C(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]
or a racemate, an enantiomer, a diastereomer, a pharmaceutically acceptable salt, or a derivative 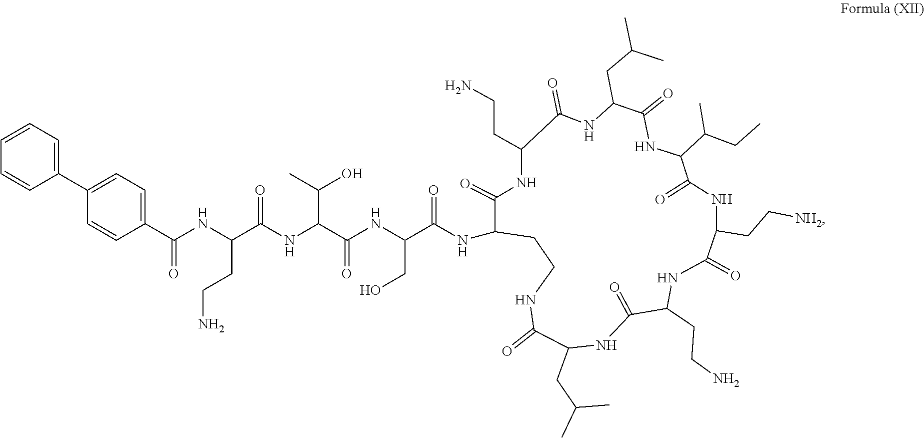 [SMILES: CCC(C)C1NC(=O)C(CC(C)C)NC(=O)C(CCN)NC(=O)C(NC(=O)C(CO)NC(=O)C(NC(=O)C(CCN)NC(=O)c2ccc(-c3ccccc3)cc2)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative thereof, 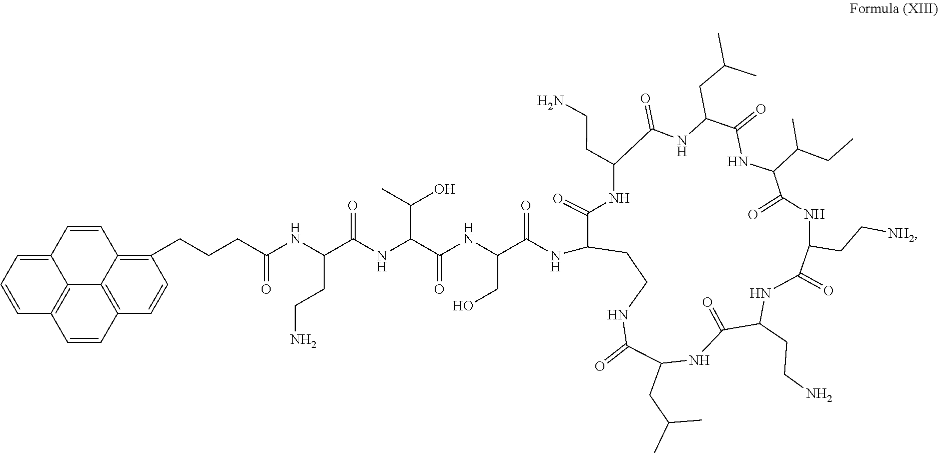 [SMILES: CCC(C)C1NC(=O)C(CC(C)C)NC(=O)C(CCN)NC(=O)C(NC(=O)C(CO)NC(=O)C(NC(=O)C(CCN)NC(=O)CCCc2ccc3ccc4cccc5ccc2c3c45)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative 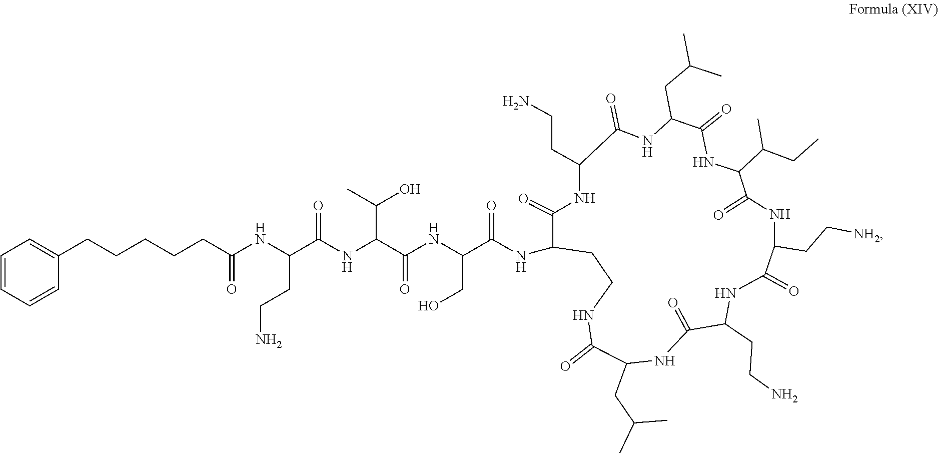 [SMILES: CCC(C)C1NC(=O)C(CC(C)C)NC(=O)C(CCN)NC(=O)C(NC(=O)C(CO)NC(=O)C(NC(=O)C(CCN)NC(=O)CCCCCc2ccccc2)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative thereof, 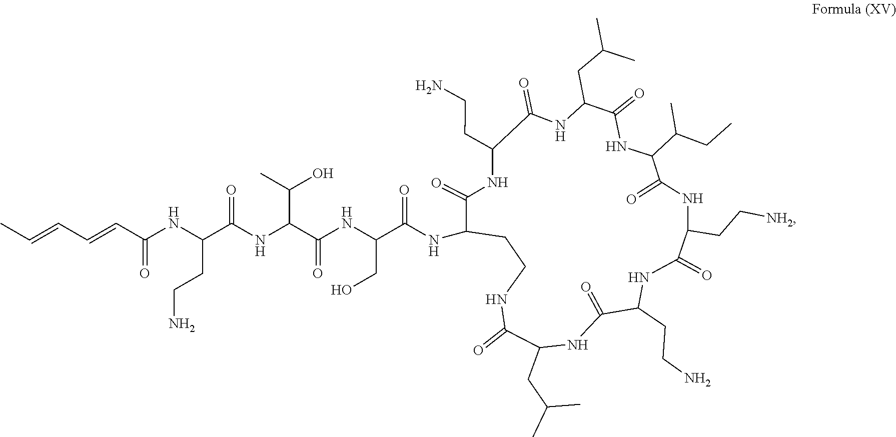 [SMILES: C/C=C/C=C/C(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]
or a racemate, an enantiomer, a diastereomer, a pharmaceutically acceptable salt, or a derivative 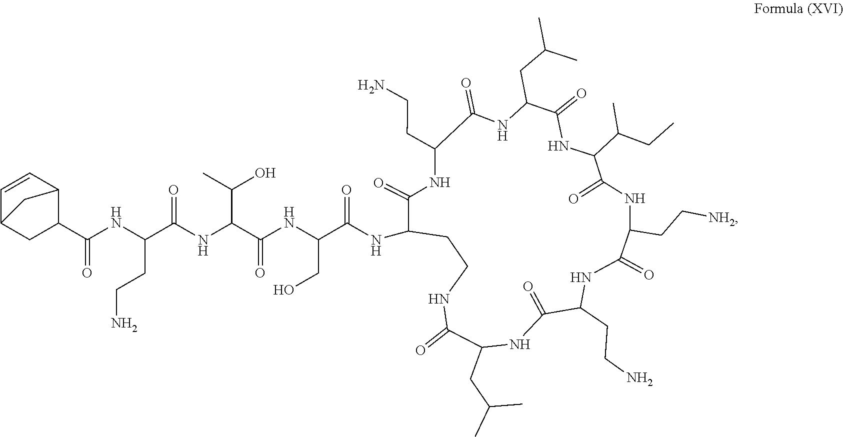 [SMILES: CCC(C)C1NC(=O)C(CC(C)C)NC(=O)C(CCN)NC(=O)C(NC(=O)C(CO)NC(=O)C(NC(=O)C(CCN)NC(=O)C2CC3C=CC2C3)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative thereof, 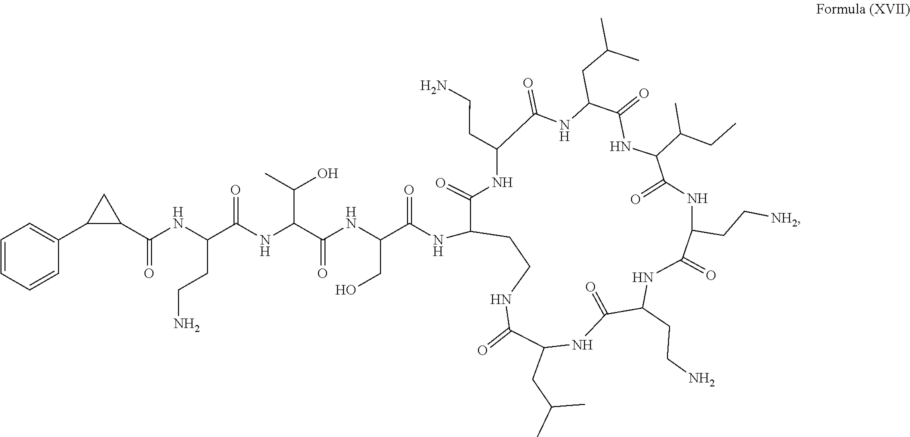 [SMILES: CCC(C)C1NC(=O)C(CC(C)C)NC(=O)C(CCN)NC(=O)C(NC(=O)C(CO)NC(=O)C(NC(=O)C(CCN)NC(=O)C2CC2c2ccccc2)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative 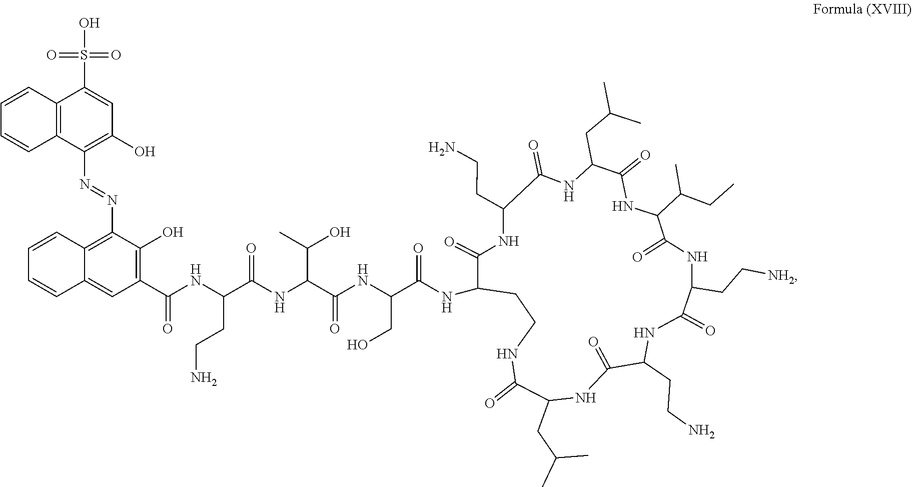 [SMILES: CCC(C)C1NC(=O)C(CC(C)C)NC(=O)C(CCN)NC(=O)C(NC(=O)C(CO)NC(=O)C(NC(=O)C(CCN)NC(=O)c2cc3ccccc3c(/N=N/c3c(O)cc(S(=O)(=O)O)c4ccccc34)c2O)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative thereof, 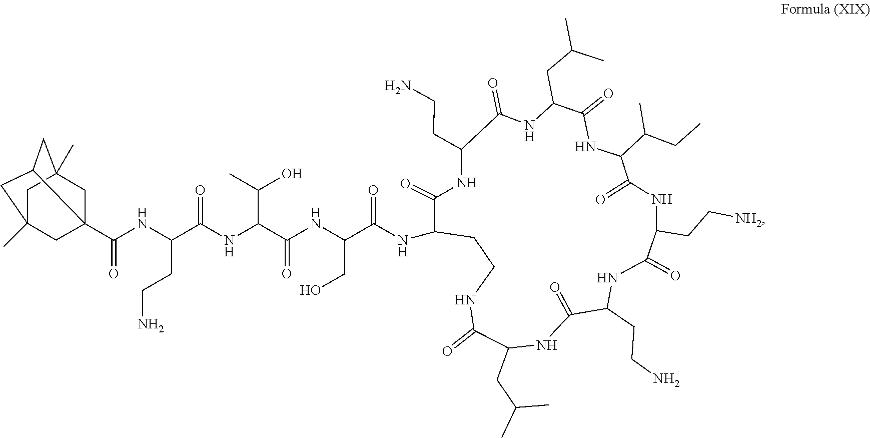 [SMILES: CCC(C)C1NC(=O)C(CC(C)C)NC(=O)C(CCN)NC(=O)C(NC(=O)C(CO)NC(=O)C(NC(=O)C(CCN)NC(=O)C23CC4(C)CC2CC(C)(C4)C3)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative thereof, and 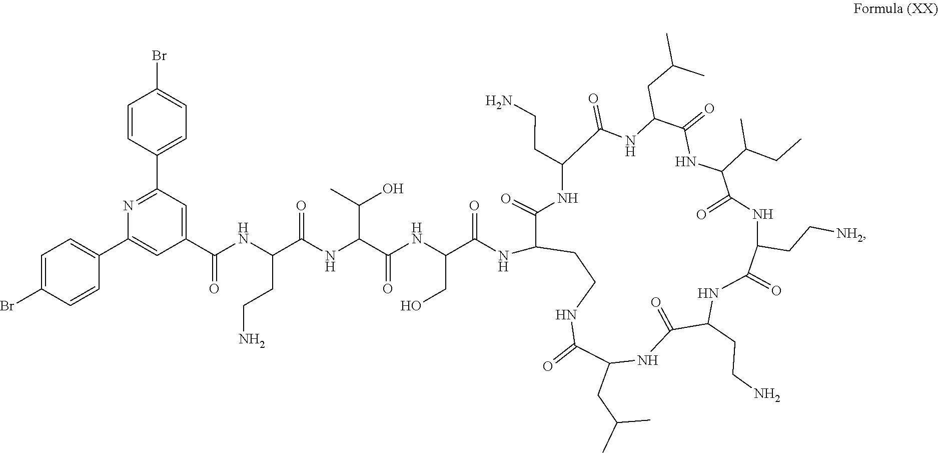 [SMILES: CCC(C)C1NC(=O)C(CC(C)C)NC(=O)C(CCN)NC(=O)C(NC(=O)C(CO)NC(=O)C(NC(=O)C(CCN)NC(=O)c2cc(-c3ccc(Br)cc3)nc(-c3ccc(Br)cc3)c2)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative thereof.

17. The isolated nucleic acid of claim 13, wherein the compound represented by Formula (I) is a compound represented by Formula (Ia) 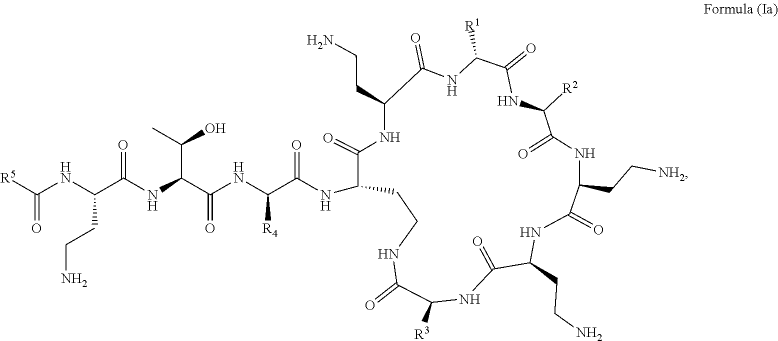
or a racemate, an enantiomer, a diastereomer, a pharmaceutically acceptable salt, or a derivative thereof,
wherein each R¹, R², R³, R⁴, R⁵, R⁶, R⁷, R⁸, R⁹, and R¹⁰ is independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, and any combinations thereof.

18. The isolated nucleic acid of claim 17, wherein each R¹, R², R³, R⁴, and R⁵ is independently selected from the group consisting of alkyl, alkenyl, aryl, aryl alkyl, aminoalkyl, hydroxyalkyl, and any combination thereof.

19. The isolated nucleic acid of claim 18, wherein each R¹, R², R³, R⁴, and R⁵ is independently selected from the group consisting of linear C₁-C₁₀ alkyl, branched C₁-C₁₀ alkyl, linear aryl-C₁-C₁₀ alkyl, branched aryl-C₁-C₁₀ alkyl, linear amino-C₁-C₁₀ alkyl, amino-branched C₁-C₁₀ alkyl, linear hydroxy-C₁-C₁₀ alkyl, hydroxy-branched C₁-C₁₀ alkyl, linear C₁-C₁₀ alkenyl, branched C₁-C₁₀ alkenyl, linear aryl-C₁-C₁₀ alkenyl, branched aryl-C₁-C₁₀ alkenyl, linear amino-C₁-C₁₀ alkenyl, amino-branched C₁-C₁₀ alkenyl, linear hydroxy-C₁-C₁₀ alkenyl, hydroxy-branched C₁-C₁₀ alkenyl, 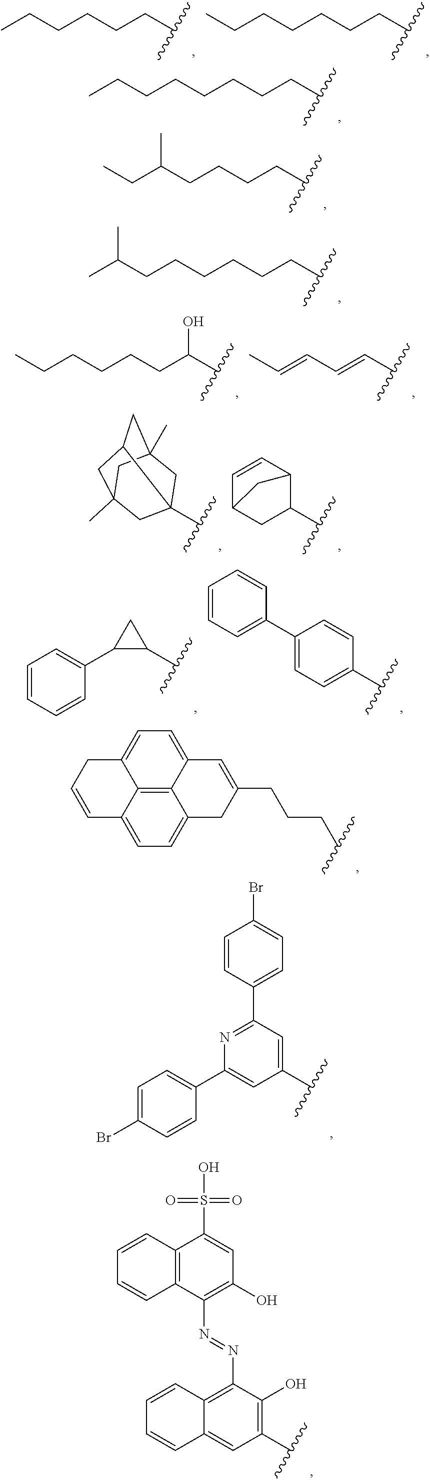
and any combination thereof.

20. The isolated nucleic acid of claim 17, wherein the compound represented by Formula (Ia) is a compound selected from the group consisting of: 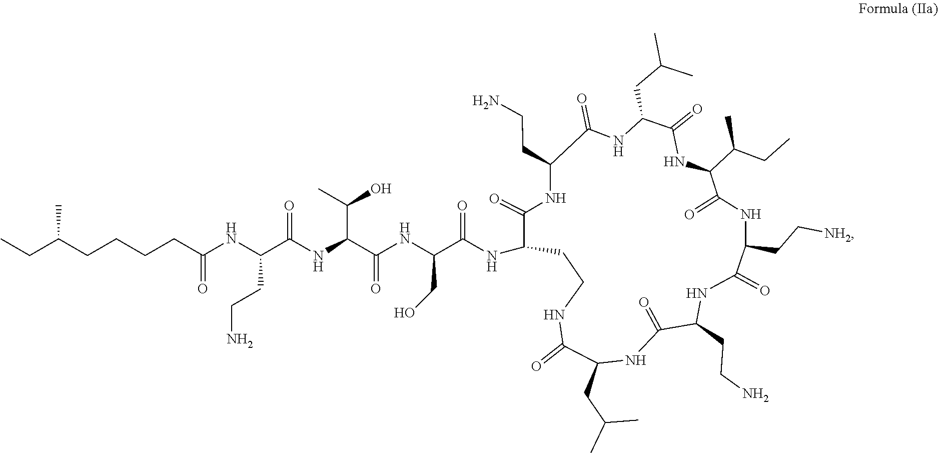 [SMILES: CC[C@H](C)CCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative thereof, 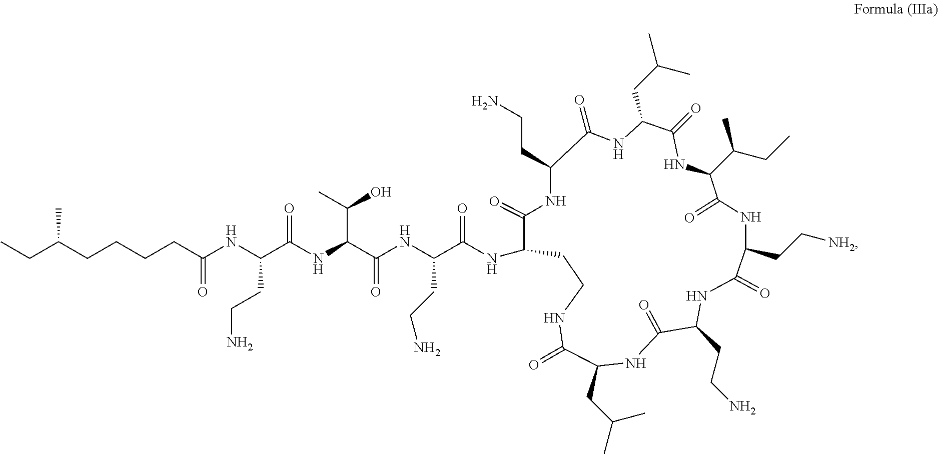 [SMILES: CC[C@H](C)CCCCC(=O)N[C@@H](CCN)C(=O)N[C@H](C(=O)N[C@@H](CCN)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative 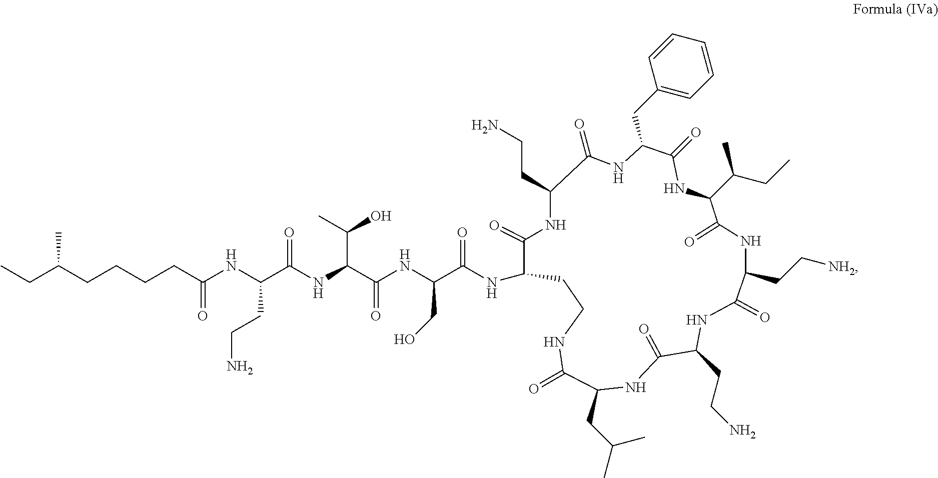 [SMILES: CC[C@H](C)CCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](Cc2ccccc2)NC(=O)[C@H](CCN)NC1=O)[C@@H](C)O]
or a racemate, an enantiomer, a diastereomer, a pharmaceutically acceptable salt, or a derivative thereof, 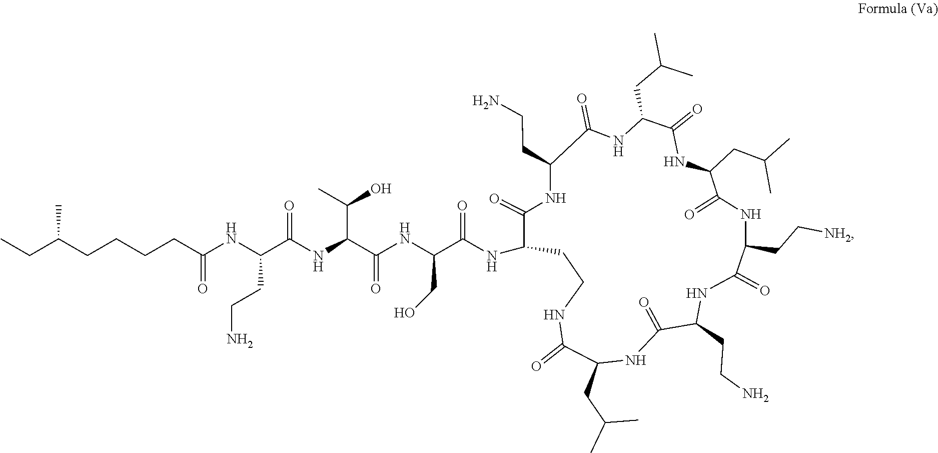 [SMILES: CC[C@H](C)CCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H](CC(C)C)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative 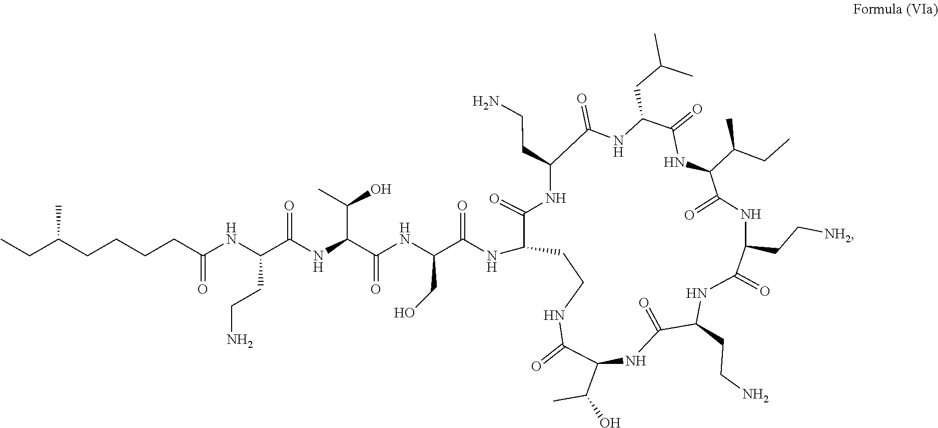 [SMILES: CC[C@H](C)CCCCC(=O)N[C@@H](CCN)C(=O)N[C@H](C(=O)N[C@H](CO)C(=O)N[C@H]1CCNC(=O)[C@H]([C@@H](C)O)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative thereof, 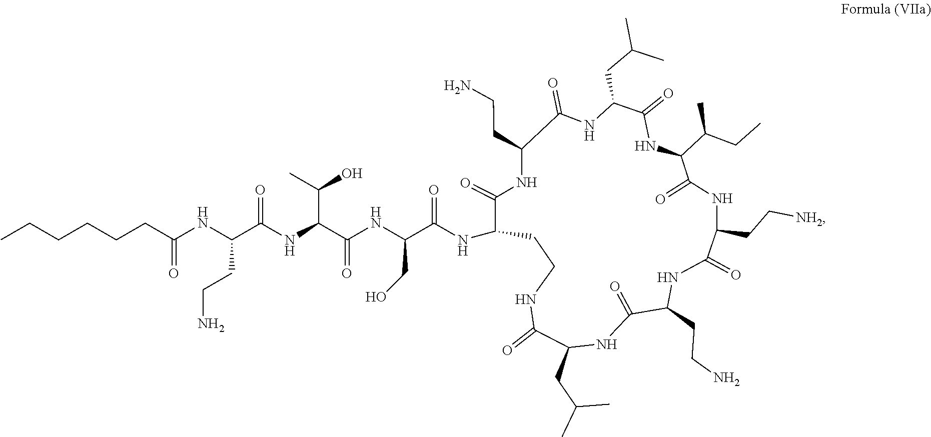 [SMILES: CCCCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative 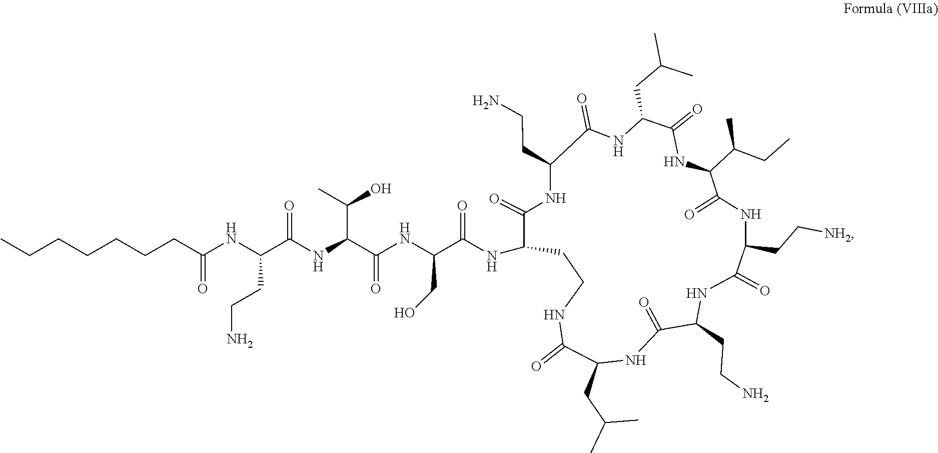 [SMILES: CCCCCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative 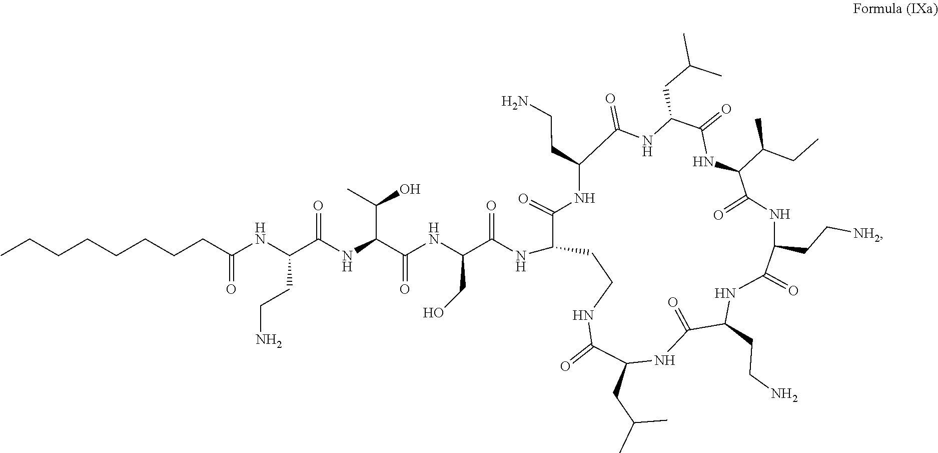 [SMILES: CCCCCCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative thereof, 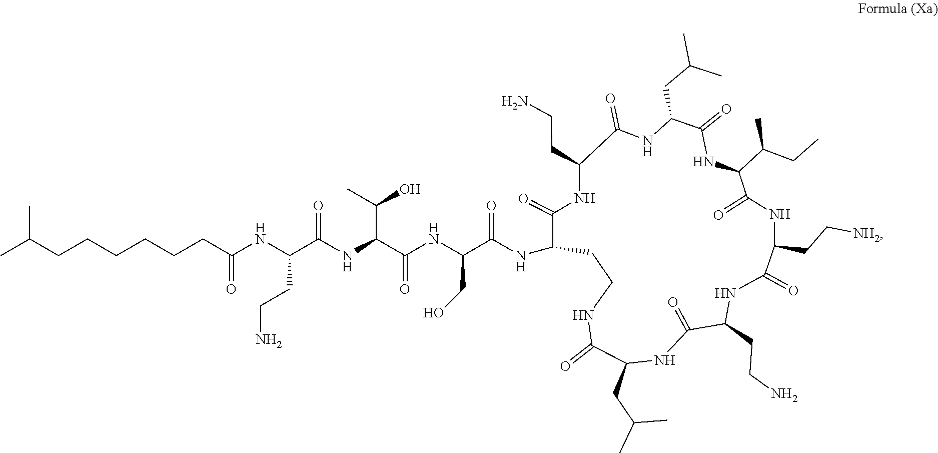 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)CCCCCCC(C)C)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative 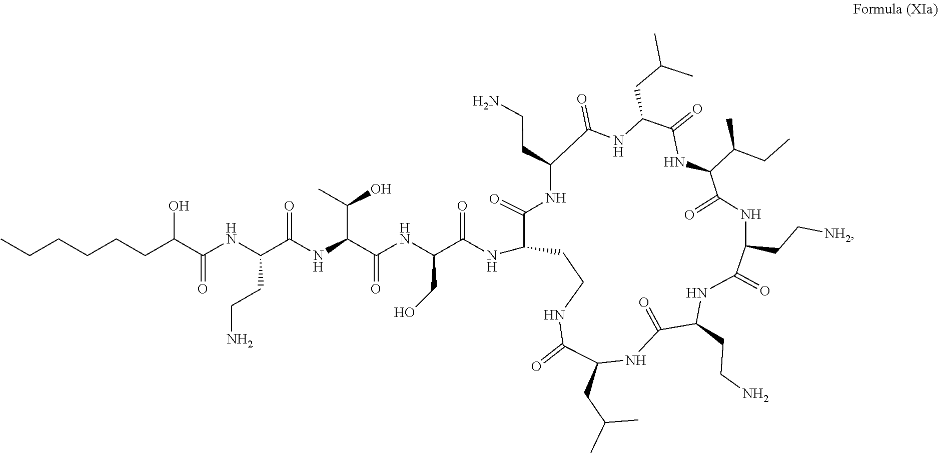 [SMILES: CCCCCCC(O)C(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative thereof, 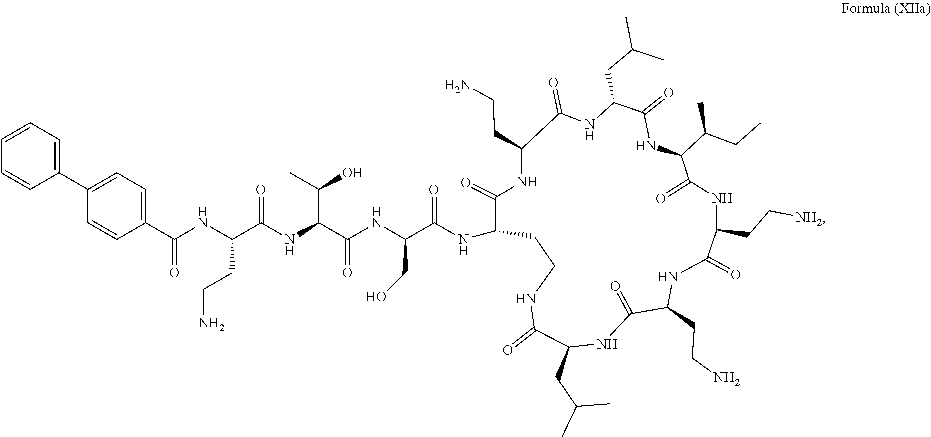 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)c2ccc(-c3ccccc3)cc2)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative 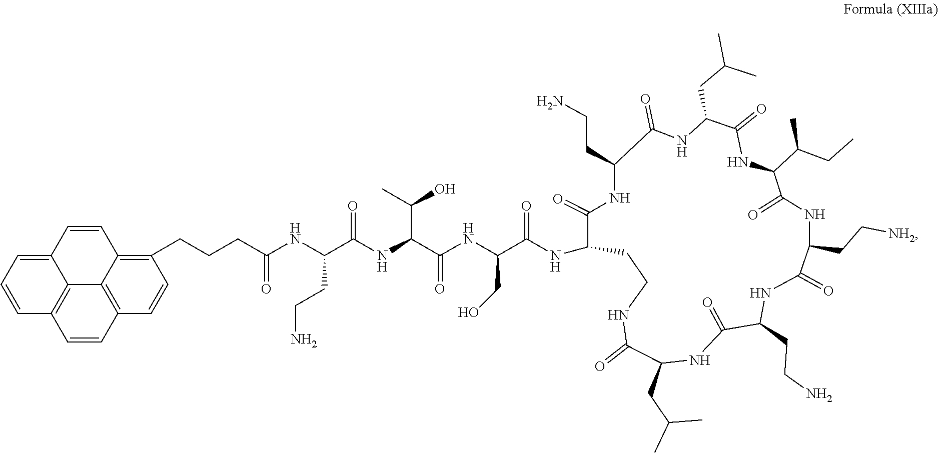 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)CCCc2ccc3ccc4cccc5ccc2c3c45)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative thereof, 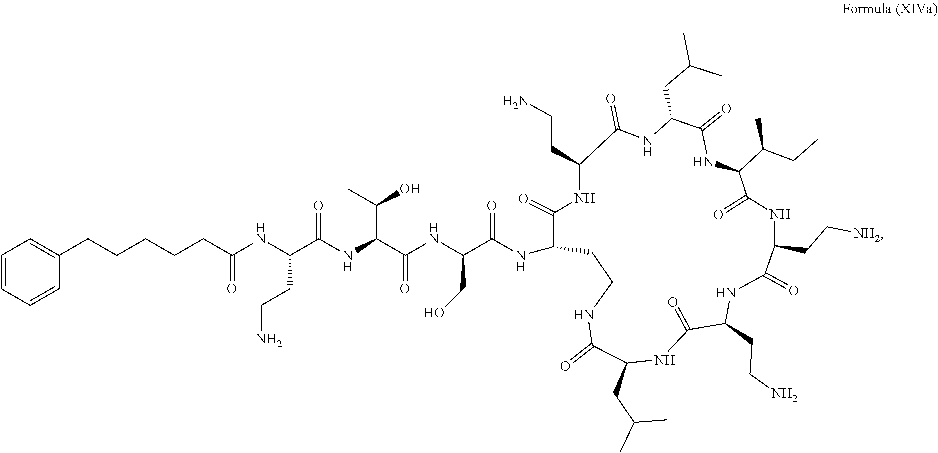 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)CCCCCc2ccccc2)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative 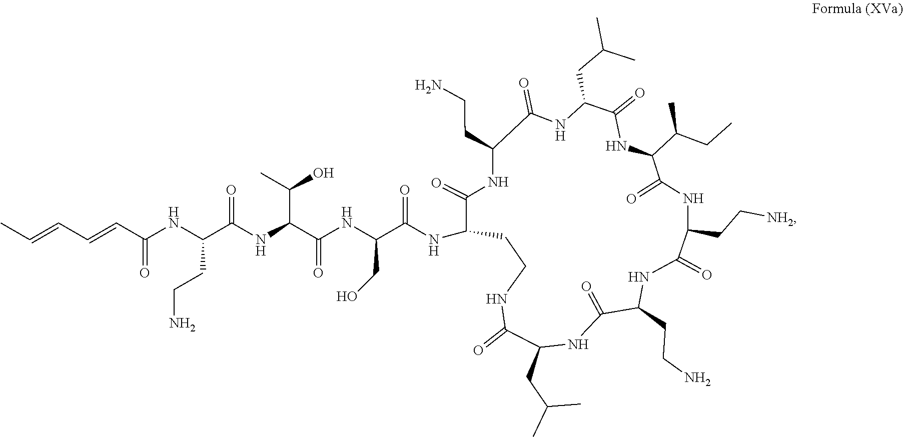 [SMILES: C/C=C/C=C/C(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative thereof, 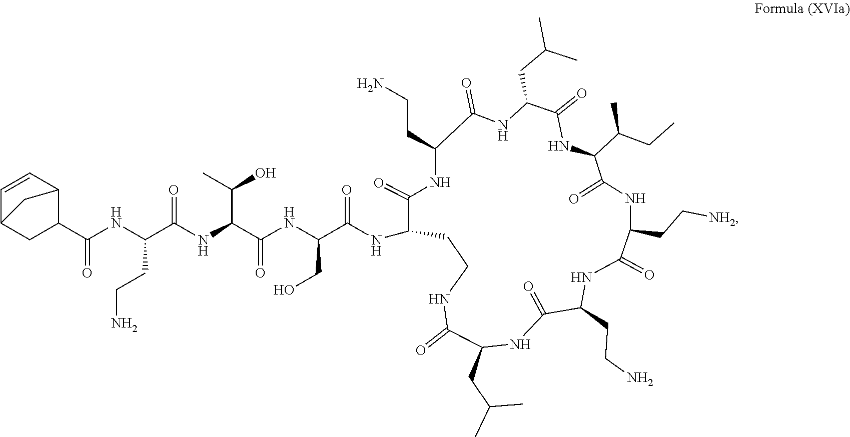 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)C2CC3C=CC2C3)[C@@H](C)O)CCNC(=O)[C@H](CC(C)C)NC(=O)[C@H](CCN)NC(=O)[C@H](CCN)NC1=O] 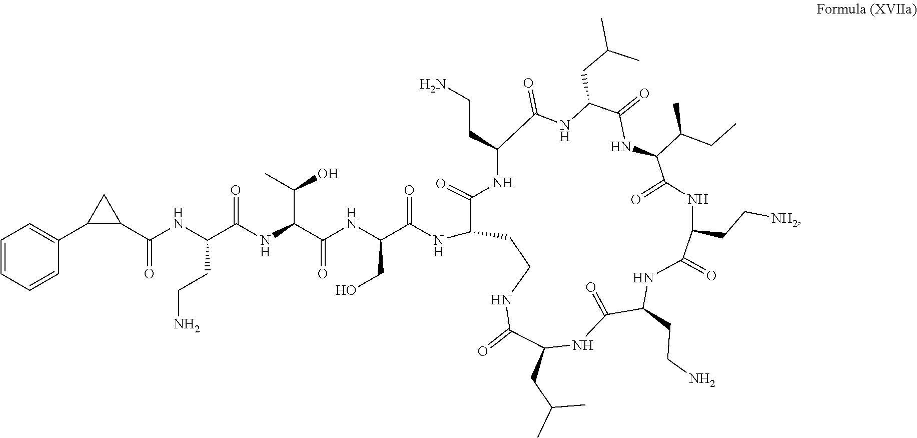 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)C2CC2c2ccccc2)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative thereof, 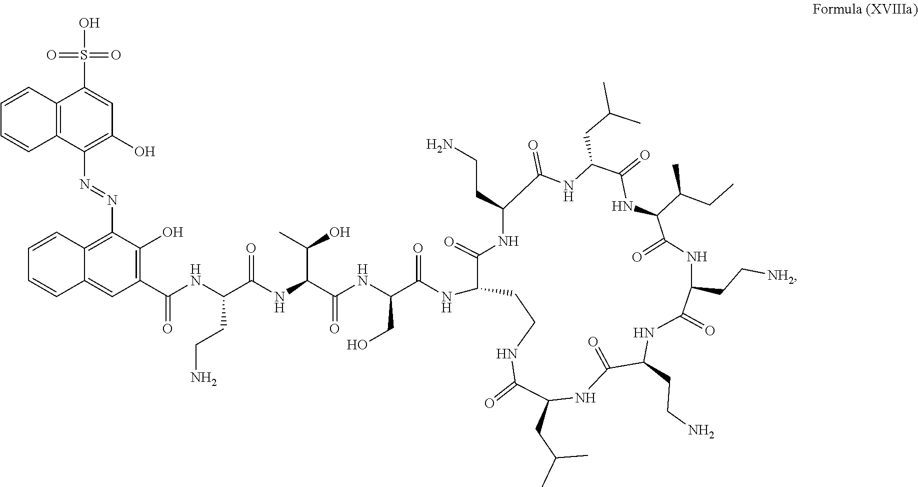 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)c2cc3ccccc3c(/N=N/c3c(O)cc(S(=O)(=O)O)c4ccccc34)c2O)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative 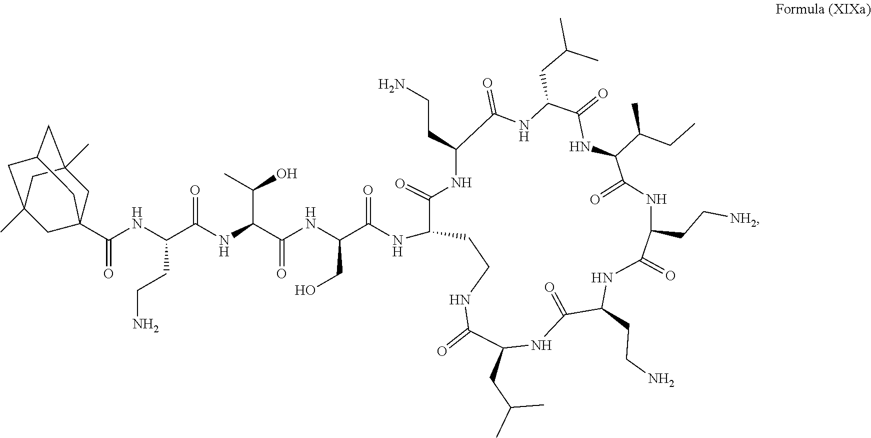 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)C23CC4CC(C)(CC(C)(C4)C2)C3)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative thereof, and 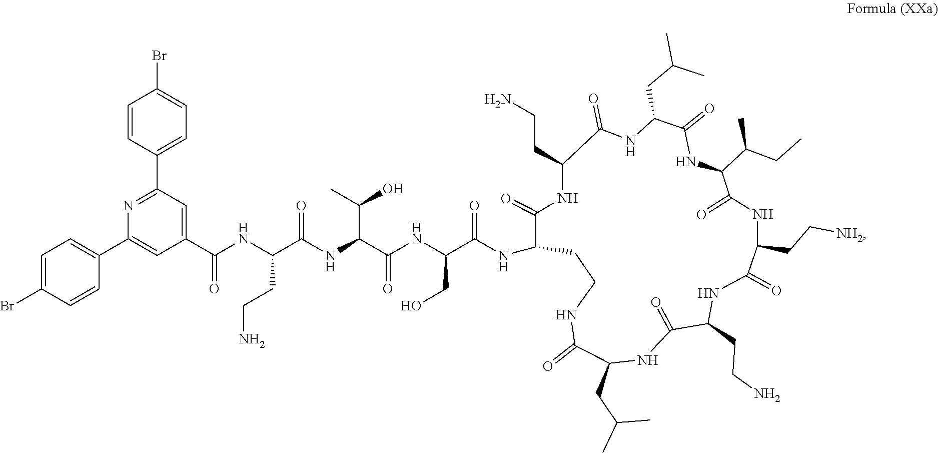 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)c2cc(-c3ccc(Br)cc3)nc(-c3ccc(Br)cc3)c2)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative thereof.

21. A genetically engineered cell, wherein the cell expresses a macolacin.

22. A method of treating or preventing a bacterial infection in a subject in need thereof, the method comprising administering a composition comprising a compound of claim 1 to the subject.

23. The method of claim 21, wherein the subject is exposed to or infected with a bacteria.

24. The method of claim 23, wherein the bacteria is a gram positive bacteria.

25. The method of claim 23, wherein the bacteria is a drug resistant bacteria.

26. The method of claim 22, wherein the method further comprises administering a second therapeutic.

27. The method of claim 26, wherein the second therapeutic is an antibiotic.

28. A method of inhibiting the growth of or killing a bacterial cell, the method comprising, contacting the bacterial cell with a composition comprising a compound of claim 1.

29. A method of biosynthesizing a macolacin, the method comprising providing a heterologous nucleic acid of the invention to a host, incubating the host in a growth medium, and isolating a macolacin from the host or the growth medium.

30. The method of claim 29, wherein the wherein the macolacin comprises a compound represented by Formula (I) 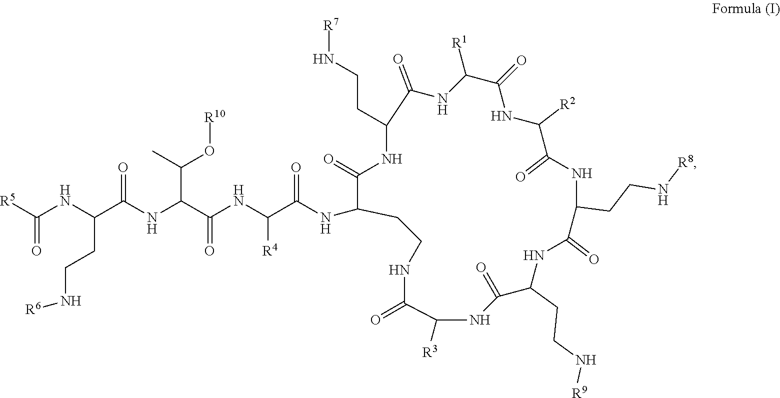
or a racemate, an enantiomer, a diastereomer, a pharmaceutically acceptable salt, or a derivative thereof,
wherein each R¹, R², R³, R⁴, R⁵, R⁶, R⁷, R⁸, R⁹, and R¹⁰ is independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, and any combinations thereof.

31. The method of claim 30, wherein each R¹, R², R³, R⁴, and R⁵ is independently selected from the group consisting of alkyl, alkenyl, aryl, aryl alkyl, aminoalkyl, hydroxyalkyl, and any combination thereof.

32. The method of claim 31, wherein each R¹, R², R³, R⁴, and R⁵ is independently selected from the group consisting of linear C₁-C₁₀ alkyl, branched C₁-C₁₀ alkyl, linear aryl-C₁-C₁₀ alkyl, branched aryl-C₁-C₁₀ alkyl, linear amino-C₁-C₁₀ alkyl, amino-branched C₁-C₁₀ alkyl, linear hydroxy-C₁-C₁₀ alkyl, hydroxy-branched C₁-C₁₀ alkyl, linear C₁-C₁₀ alkenyl, branched C₁-C₁₀ alkenyl, linear aryl-C₁-C₁₀ alkenyl, branched aryl-C₁-C₁₀ alkenyl, linear amino-C₁-C₁₀ alkenyl, amino-branched C₁-C₁₀ alkenyl, linear hydroxy-C₁-C₁₀ alkenyl, hydroxy-branched C₁-C₁₀ alkenyl, 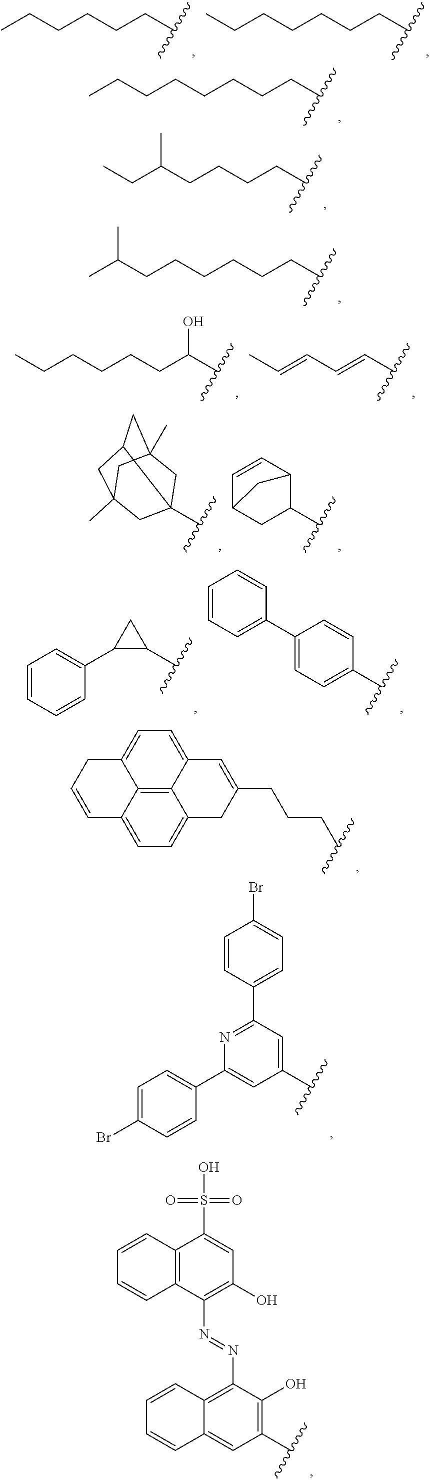
and any combination thereof.

33. The method of claim 30, wherein the compound represented by Formula (I) is a compound selected from the group consisting of: 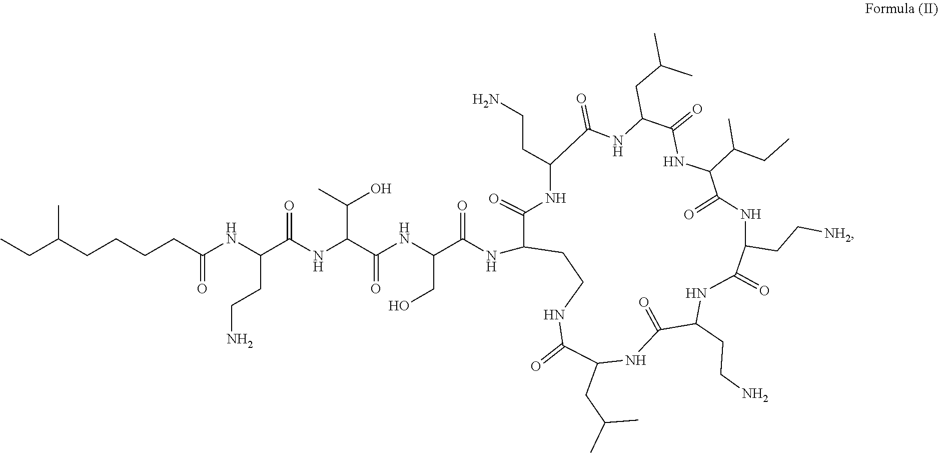 [SMILES: CCC(C)CCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]
or a racemate, an enantiomer, a diastereomer, a pharmaceutically acceptable salt, or a derivative thereof, 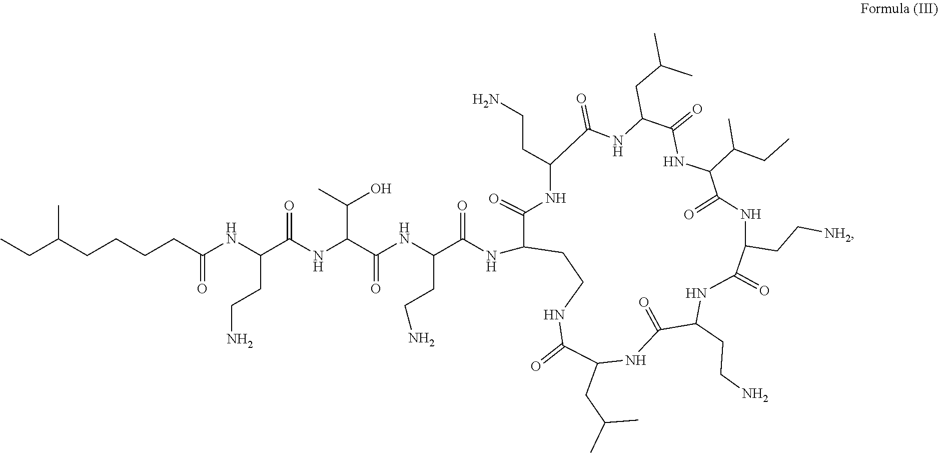 [SMILES: CCC(C)CCCCC(=O)NC(CCN)C(=O)NC(C(=O)NC(CCN)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]
or a racemate, an enantiomer, a diastereomer, a pharmaceutically acceptable salt, or a derivative 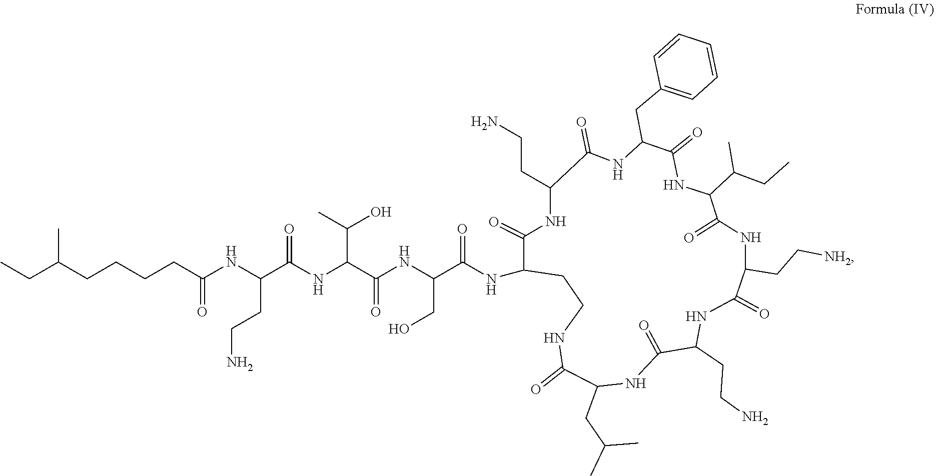 [SMILES: CCC(C)CCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(Cc2ccccc2)NC(=O)C(CCN)NC1=O)C(C)O]
or a racemate, an enantiomer, a diastereomer, a pharmaceutically acceptable salt, or a derivative thereof, 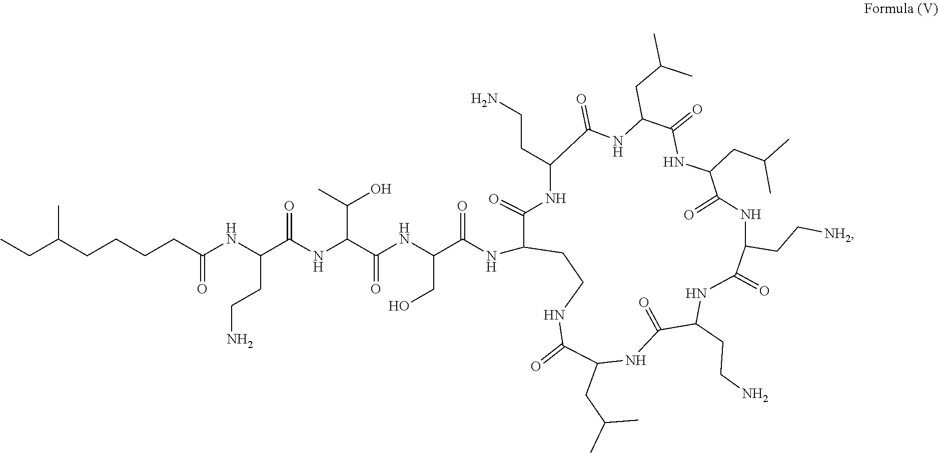 [SMILES: CCC(C)CCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(CC(C)C)NC(=O)C(CC(C)C)NC(=O)C(CCN)NC1=O)C(C)O]
or a racemate, an enantiomer, a diastereomer, a pharmaceutically acceptable salt, or a derivative 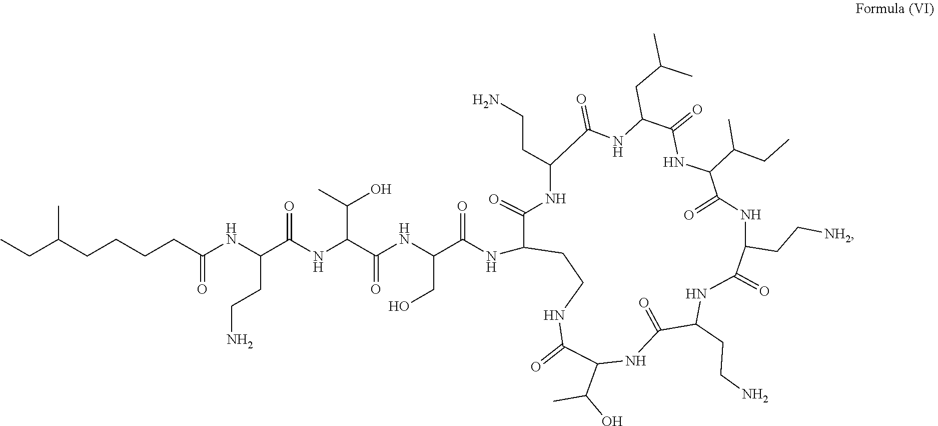 [SMILES: CCC(C)CCCCC(=O)NC(CCN)C(=O)NC(C(=O)NC(CO)C(=O)NC1CCNC(=O)C(C(C)O)NC(=O)C(CCN)NC(=O)C(CCN)NC(=O)C(C(C)CC)NC(=O)C(CC(C)C)NC(=O)C(CCN)NC1=O)C(C)O]
or a racemate, an enantiomer, a diastereomer, a pharmaceutically acceptable salt, or a derivative thereof, 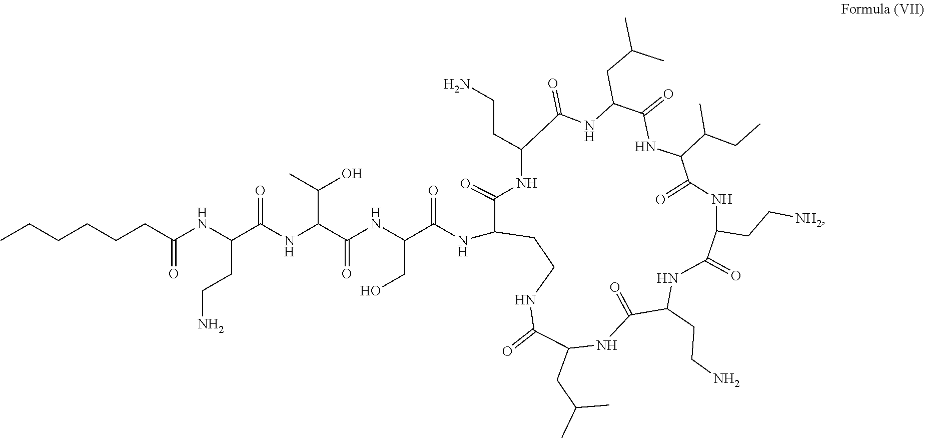 [SMILES: CCCCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]
or a racemate, an enantiomer, a diastereomer, a pharmaceutically acceptable salt, or a derivative 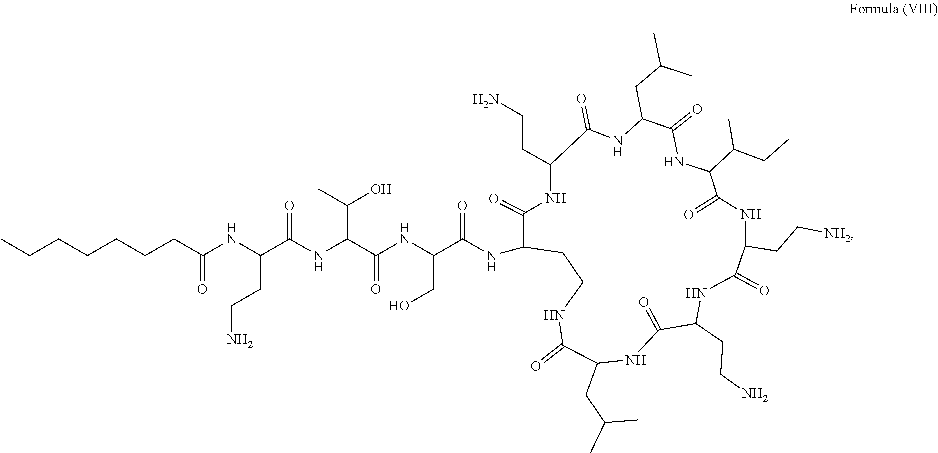 [SMILES: CCCCCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]
or a racemate, an enantiomer, a diastereomer, a pharmaceutically acceptable salt, or a derivative thereof, 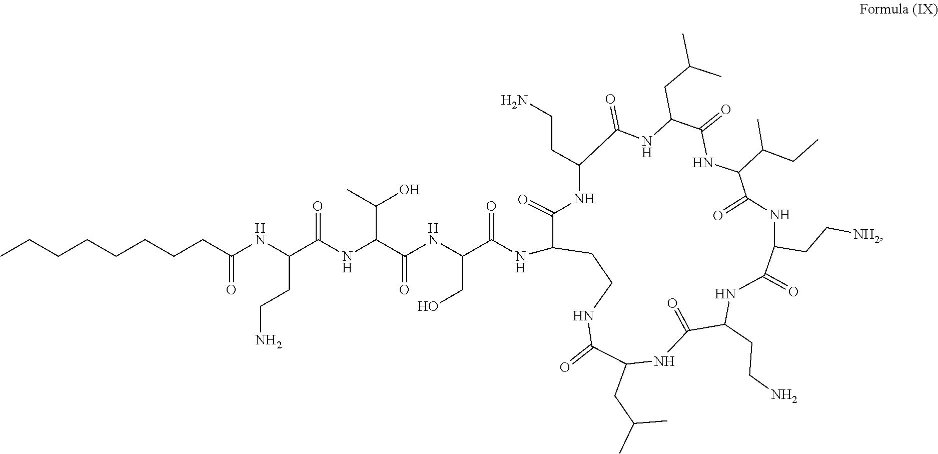 [SMILES: CCCCCCCCC(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]
or a racemate, an enantiomer, a diastereomer, a pharmaceutically acceptable salt, or a derivative thereof, 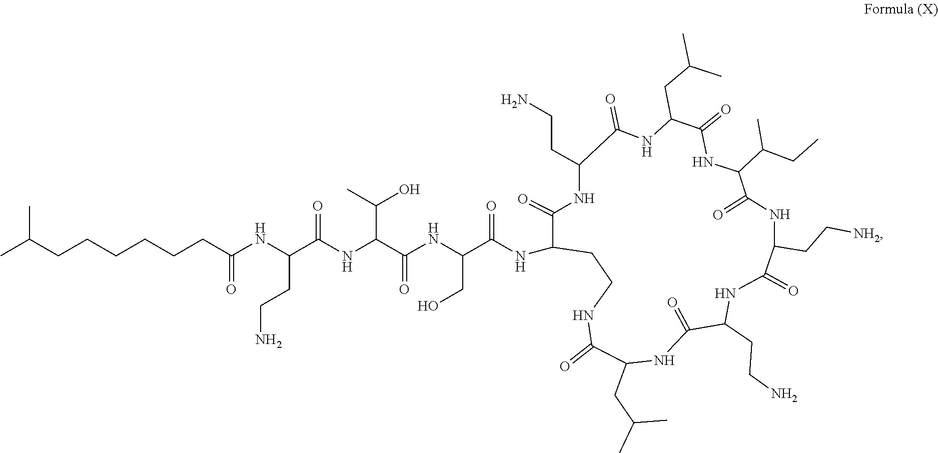 [SMILES: CCC(C)C1NC(=O)C(CC(C)C)NC(=O)C(CCN)NC(=O)C(NC(=O)C(CO)NC(=O)C(NC(=O)C(CCN)NC(=O)CCCCCCC(C)C)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative 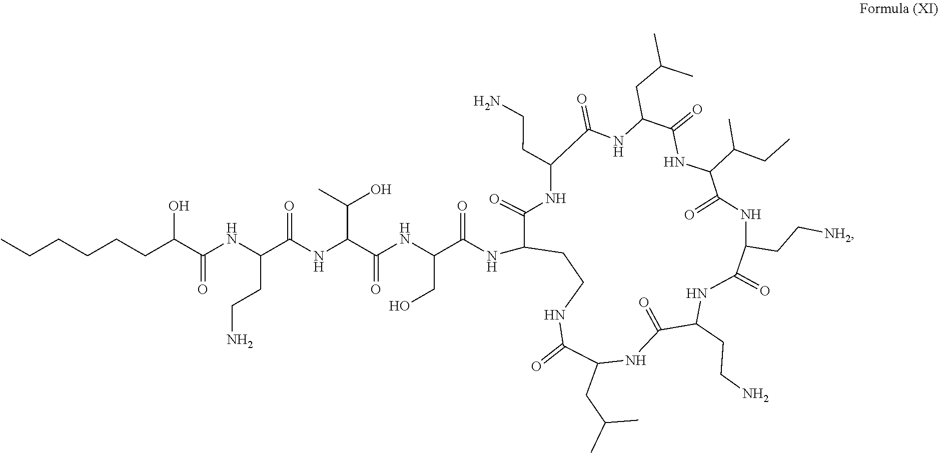 [SMILES: CCCCCCC(O)C(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]
or a racemate, an enantiomer, a diastereomer, a pharmaceutically acceptable salt, or a derivative thereof, 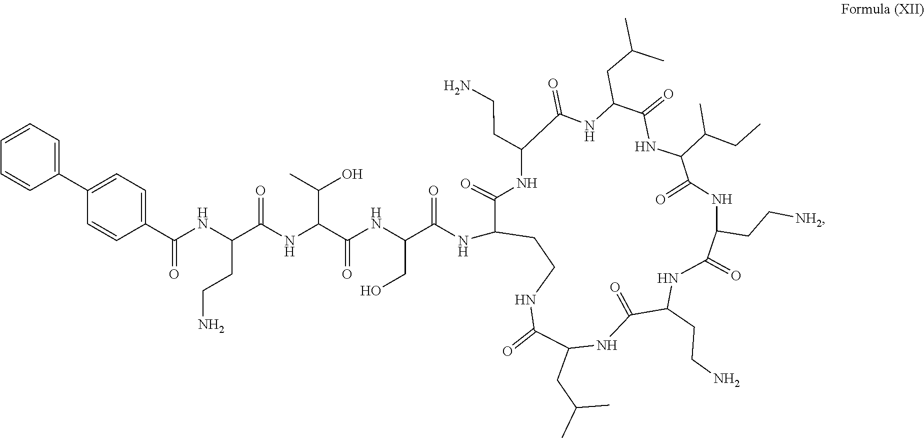 [SMILES: CCC(C)C1NC(=O)C(CC(C)C)NC(=O)C(CCN)NC(=O)C(NC(=O)C(CO)NC(=O)C(NC(=O)C(CCN)NC(=O)c2ccc(-c3ccccc3)cc2)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative 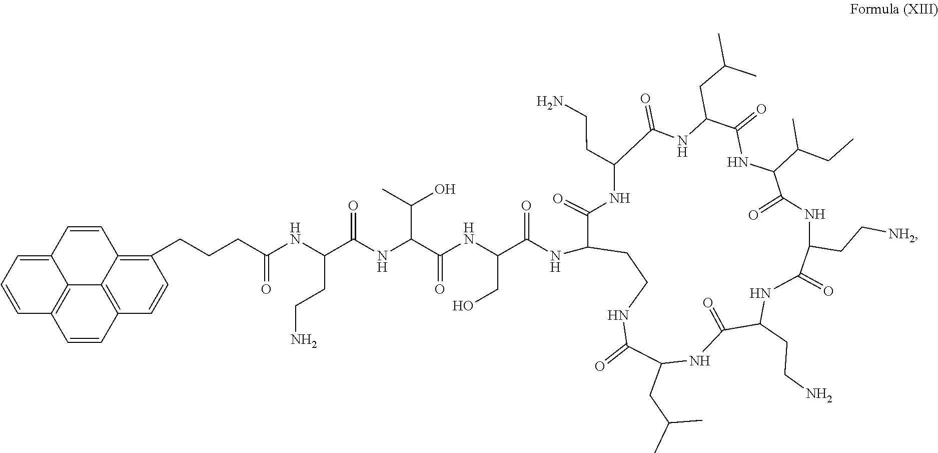 [SMILES: CCC(C)C1NC(=O)C(CC(C)C)NC(=O)C(CCN)NC(=O)C(NC(=O)C(CO)NC(=O)C(NC(=O)C(CCN)NC(=O)CCCc2ccc3ccc4cccc5ccc2c3c45)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative thereof, 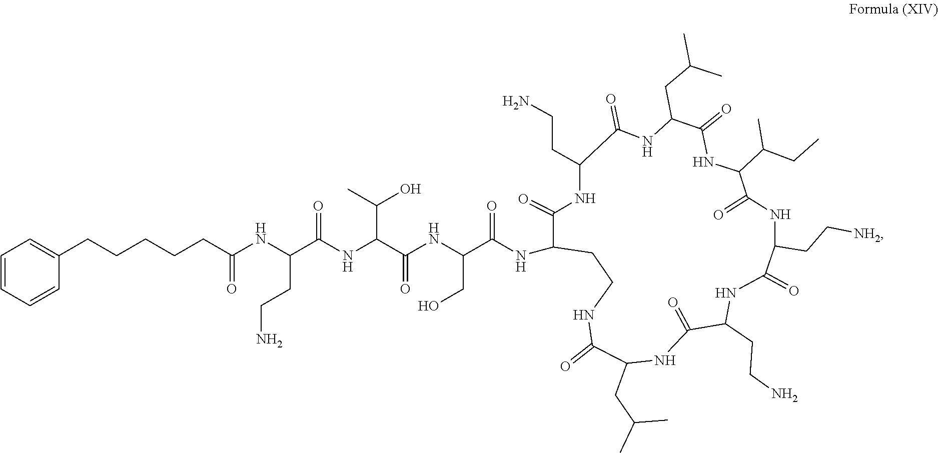 [SMILES: CCC(C)C1NC(=O)C(CC(C)C)NC(=O)C(CCN)NC(=O)C(NC(=O)C(CO)NC(=O)C(NC(=O)C(CCN)NC(=O)CCCCCc2ccccc2)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative thereof 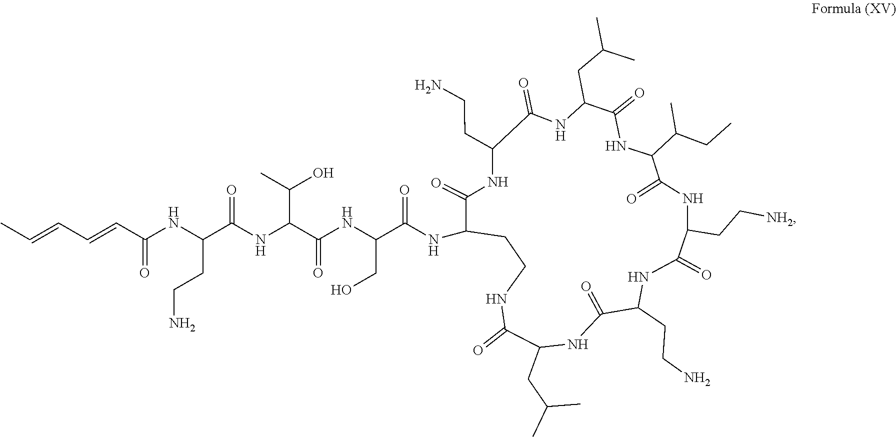 [SMILES: C/C=C/C=C/C(=O)NC(CCN)C(=O)NC(C(=O)NC(CO)C(=O)NC1CCNC(=O)C(CC(C)C)NC(=O)C(CCN)NC(=O)C(CCN)NC(=O)C(C(C)CC)NC(=O)C(CC(C)C)NC(=O)C(CCN)NC1=O)C(C)O]
or a racemate, an enantiomer, a diastereomer, a pharmaceutically acceptable salt, or a derivative thereof, 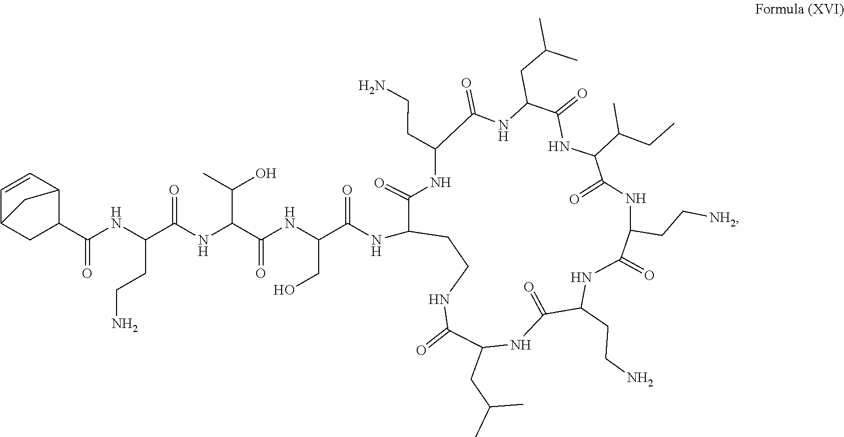 [SMILES: CCC(C)C1NC(=O)C(CC(C)C)NC(=O)C(CCN)NC(=O)C(NC(=O)C(CO)NC(=O)C(NC(=O)C(CCN)NC(=O)C2CC3C=CC2C3)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative 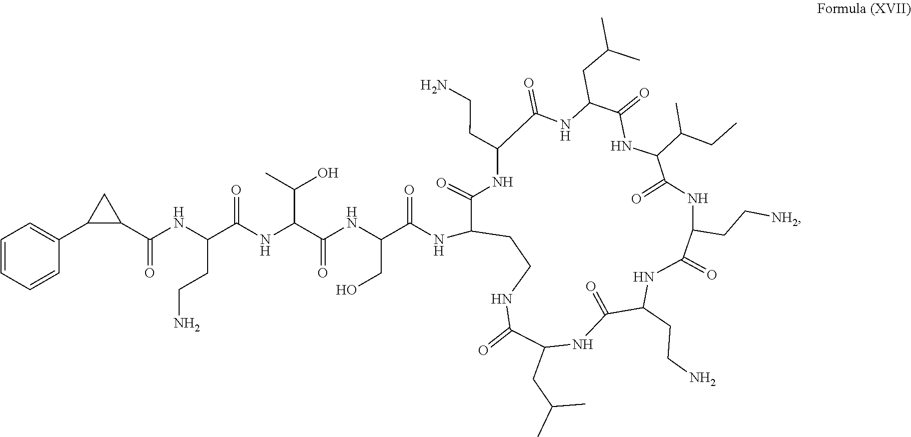 [SMILES: CCC(C)C1NC(=O)C(CC(C)C)NC(=O)C(CCN)NC(=O)C(NC(=O)C(CO)NC(=O)C(NC(=O)C(CCN)NC(=O)C2CC2c2ccccc2)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative thereof, 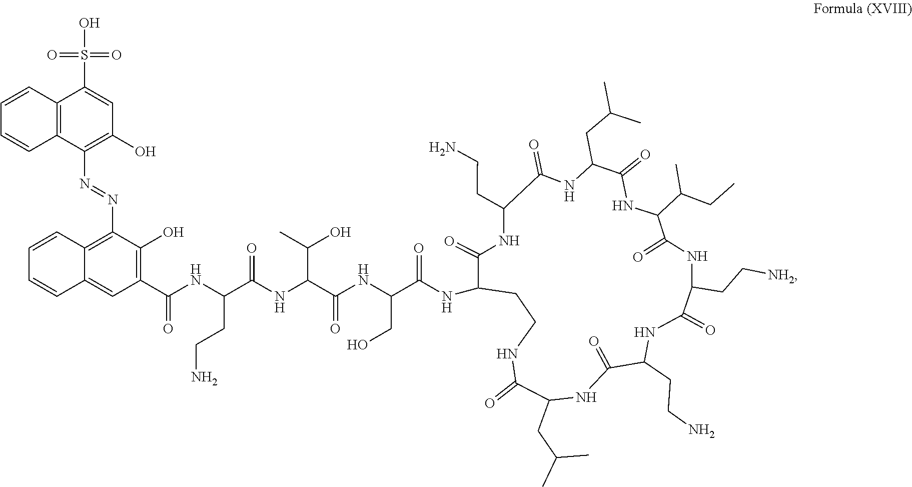 [SMILES: CCC(C)C1NC(=O)C(CC(C)C)NC(=O)C(CCN)NC(=O)C(NC(=O)C(CO)NC(=O)C(NC(=O)C(CCN)NC(=O)c2cc3ccccc3c(/N=N/c3c(O)cc(S(=O)(=O)O)c4ccccc34)c2O)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative 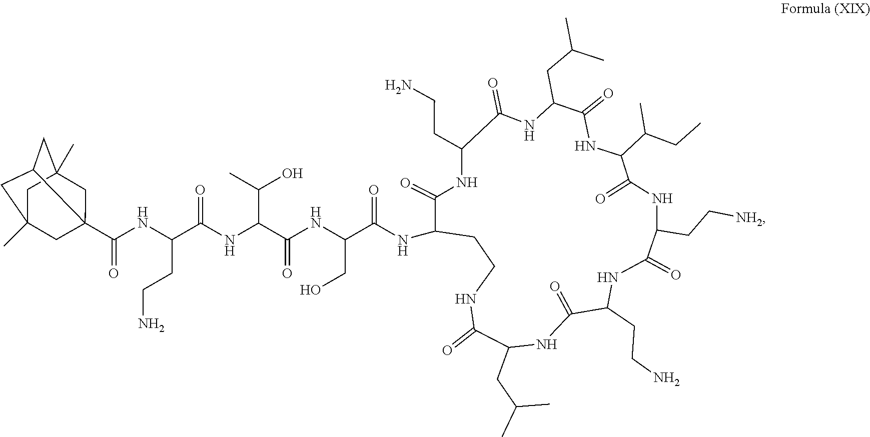 [SMILES: CCC(C)C1NC(=O)C(CC(C)C)NC(=O)C(CCN)NC(=O)C(NC(=O)C(CO)NC(=O)C(NC(=O)C(CCN)NC(=O)C23CC4(C)CC2CC(C)(C4)C3)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative thereof, and 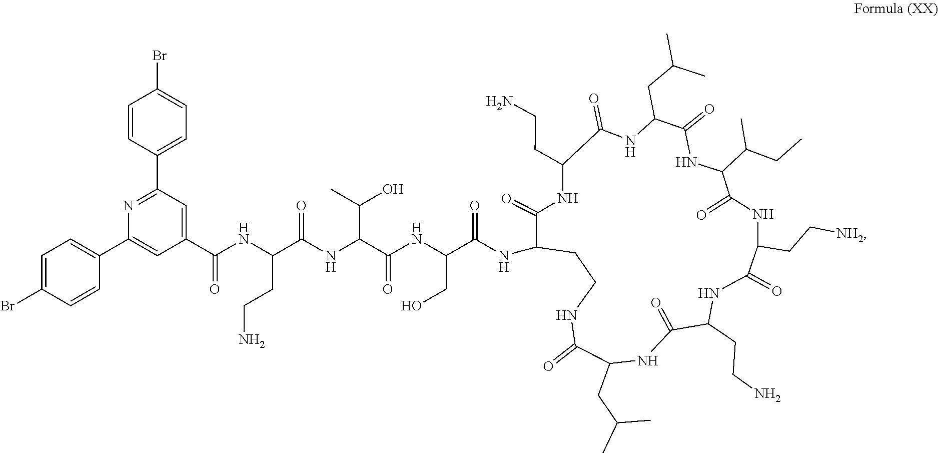 [SMILES: CCC(C)C1NC(=O)C(CC(C)C)NC(=O)C(CCN)NC(=O)C(NC(=O)C(CO)NC(=O)C(NC(=O)C(CCN)NC(=O)c2cc(-c3ccc(Br)cc3)nc(-c3ccc(Br)cc3)c2)C(C)O)CCNC(=O)C(CC(C)C)NC(=O)C(CCN)NC(=O)C(CCN)NC1=O]
or a racemate, an enantiomer, a diastereomer, a pharmaceutically acceptable salt, or a derivative thereof.

34. The method of claim 30, wherein the compound represented by Formula (I) is a compound represented by Formula (Ia) 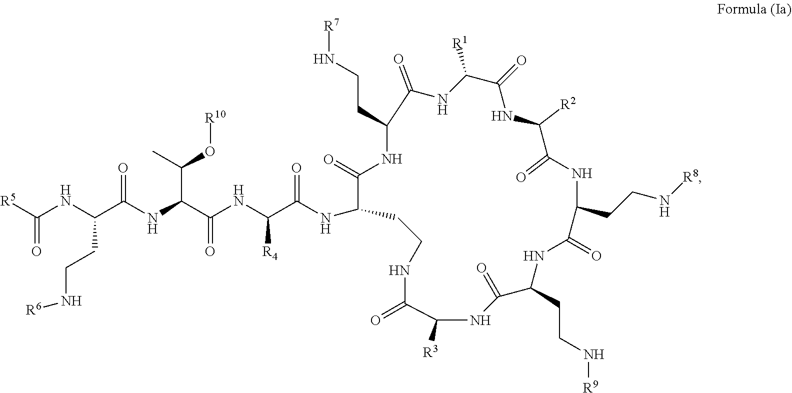
or a racemate, an enantiomer, a diastereomer, a pharmaceutically acceptable salt, or a derivative thereof,
wherein each R¹, R², R³, R⁴, R⁵, R⁶, R⁷, R⁸, R⁹, and R¹⁰ is independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, and any combinations thereof.

35. The method of claim 34, wherein each R¹, R², R³, R⁴, and R⁵ is independently selected from the group consisting of alkyl, alkenyl, aryl, aryl alkyl, aminoalkyl, hydroxyalkyl, and any combination thereof.

36. The method of claim 35, wherein each R¹, R², R³, R⁴, and R⁵ is independently selected from the group consisting of linear C₁-C₁₀ alkyl, branched C₁-C₁₀ alkyl, linear aryl-C₁-C₁₀ alkyl, branched aryl-C₁-C₁₀ alkyl, linear amino-C₁-C₁₀ alkyl, amino-branched C₁-C₁₀ alkyl, linear hydroxy-C₁-C₁₀ alkyl, hydroxy-branched C₁-C₁₀ alkyl, linear C₁-C₁₀ alkenyl, branched C₁-C₁₀ alkenyl, linear aryl-C₁-C₁₀ alkenyl, branched aryl-C₁-C₁₀ alkenyl, linear amino-C₁-C₁₀ alkenyl, amino-branched C₁-C₁₀ alkenyl, linear hydroxy-C₁-C₁₀ alkenyl, hydroxy-branched C₁-C₁₀ alkenyl, 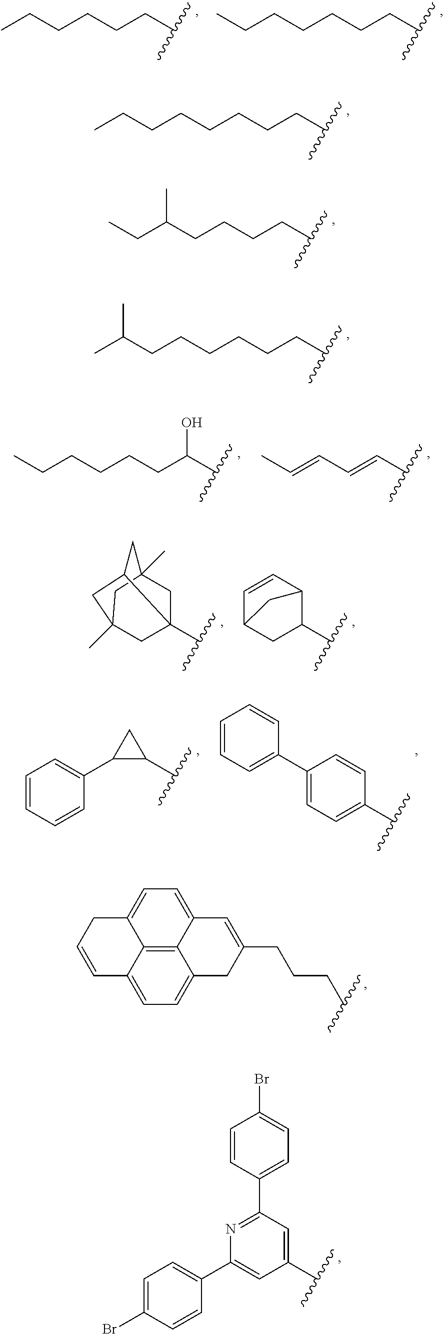 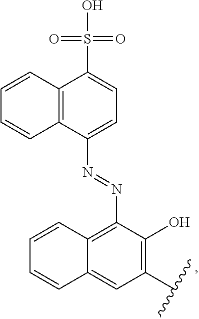 [SMILES: *c1cc2ccccc2c(/N=N/c2ccc(S(=O)(=O)O)c3ccccc23)c1O]
and any combination thereof.

37. The method of claim 34, wherein the compound represented by Formula (Ia) is a compound selected from the group consisting of: 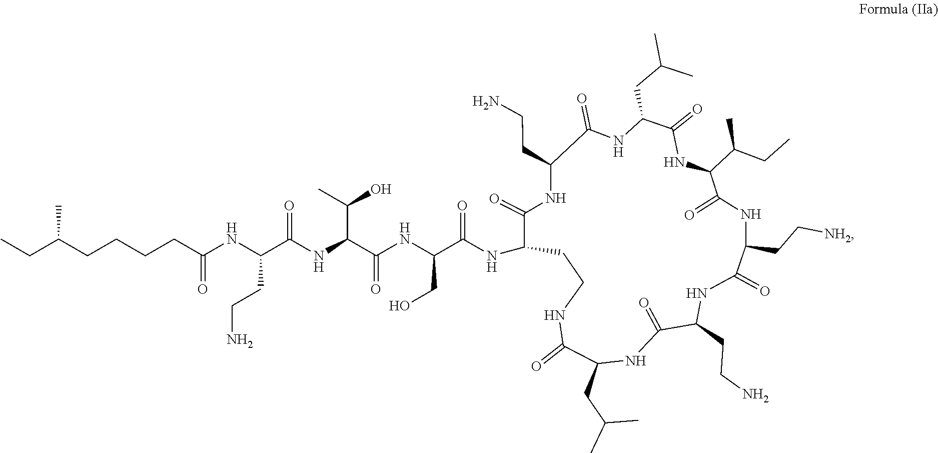 [SMILES: CC[C@H](C)CCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative 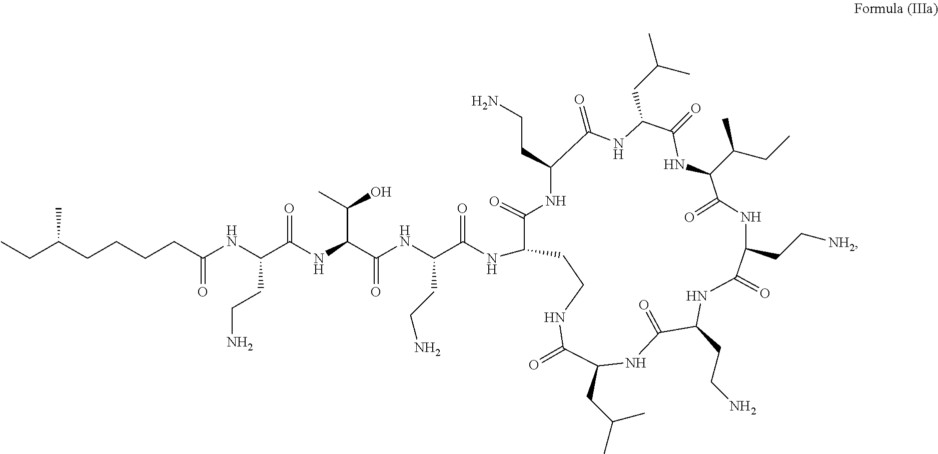 [SMILES: CC[C@H](C)CCCCC(=O)N[C@@H](CCN)C(=O)N[C@H](C(=O)N[C@@H](CCN)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative thereof, 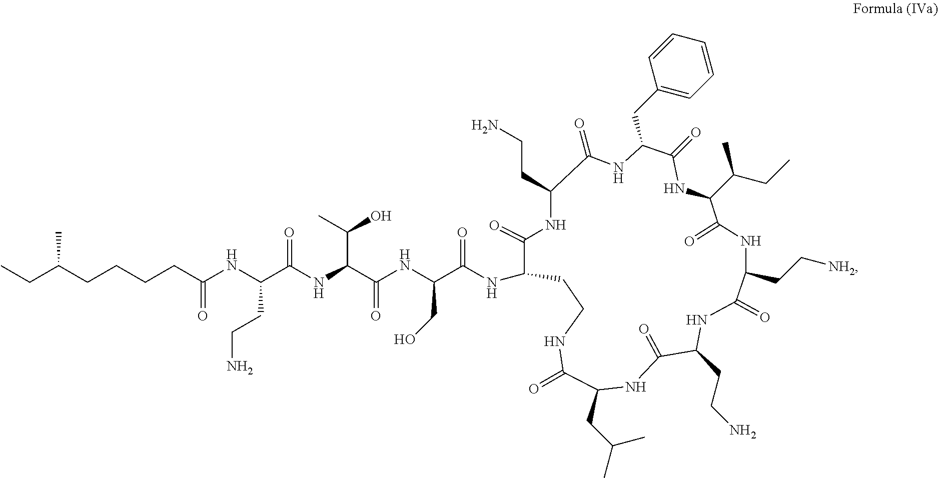 [SMILES: CC[C@H](C)CCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](Cc2ccccc2)NC(=O)[C@H](CCN)NC1=O)[C@@H](C)O]
or a racemate, an enantiomer, a diastereomer, a pharmaceutically acceptable salt, or a derivative 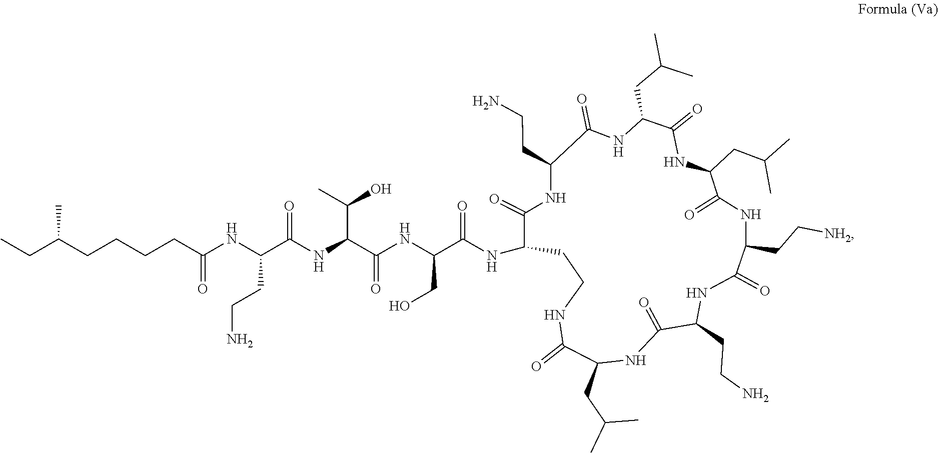 [SMILES: CC[C@H](C)CCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H](CC(C)C)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative thereof, 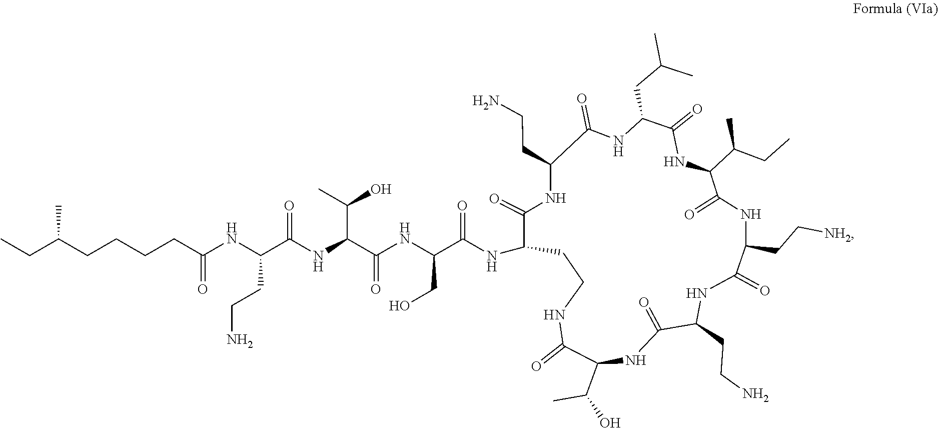 [SMILES: CC[C@H](C)CCCCC(=O)N[C@@H](CCN)C(=O)N[C@H](C(=O)N[C@H](CO)C(=O)N[C@H]1CCNC(=O)[C@H]([C@@H](C)O)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative 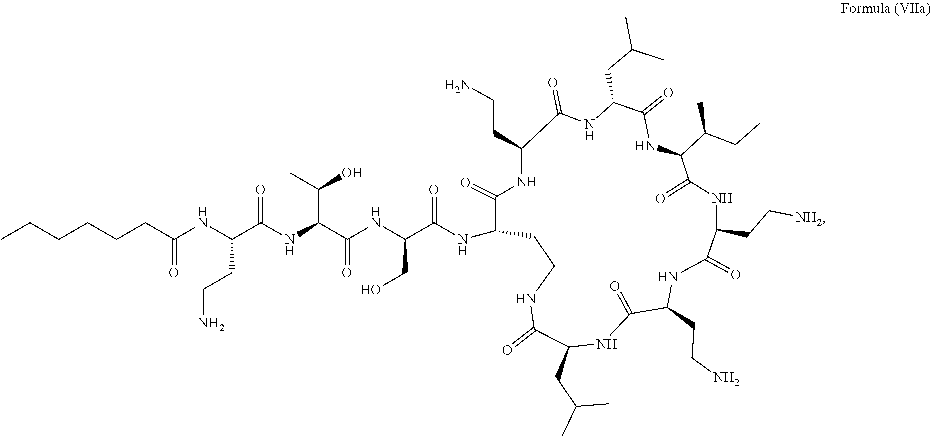 [SMILES: CCCCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative 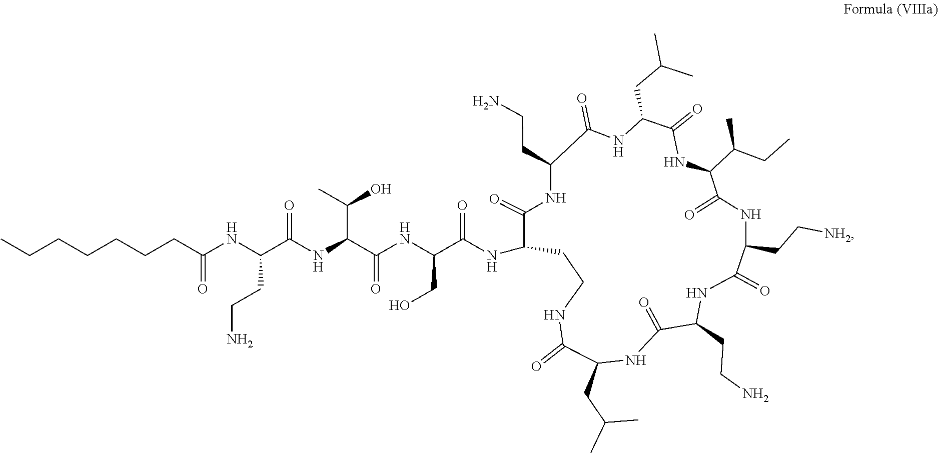 [SMILES: CCCCCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative thereof, 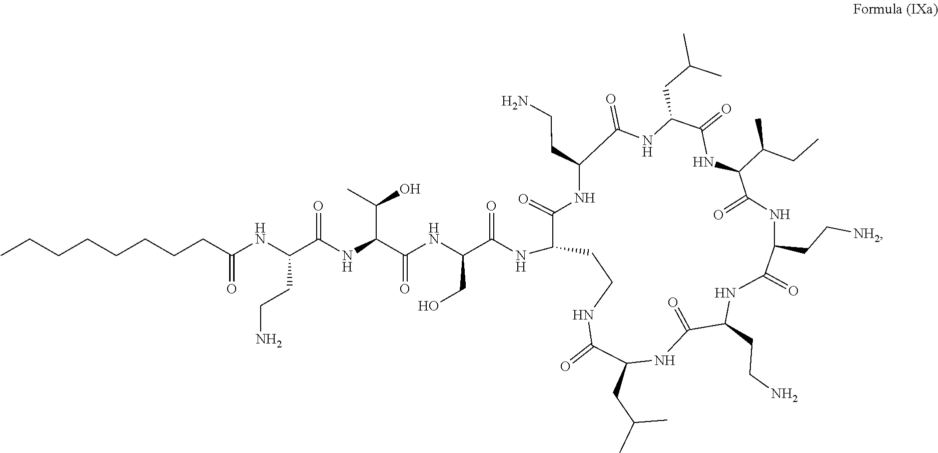 [SMILES: CCCCCCCCC(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative 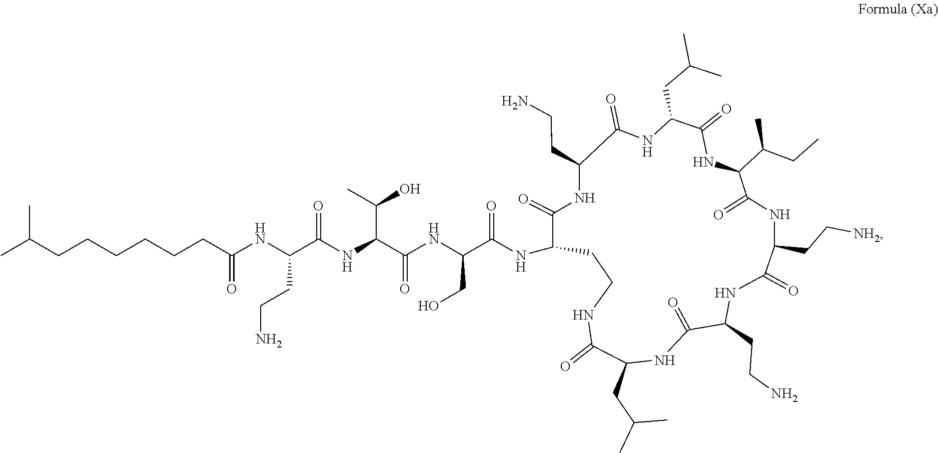 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)CCCCCCC(C)C)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative thereof, 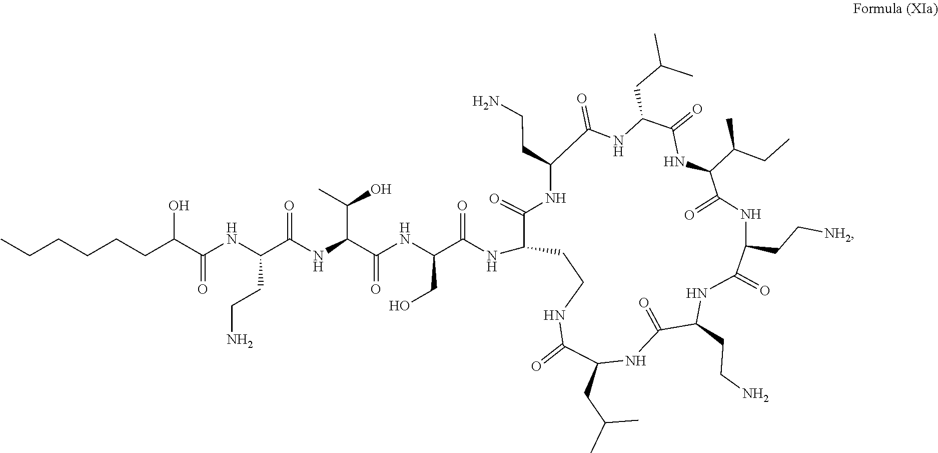 [SMILES: CCCCCCC(O)C(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative 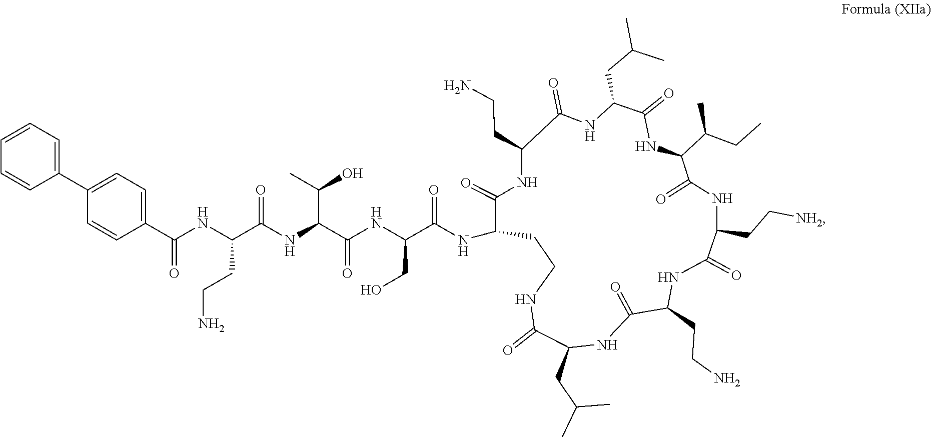 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)c2ccc(-c3ccccc3)cc2)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative thereof, 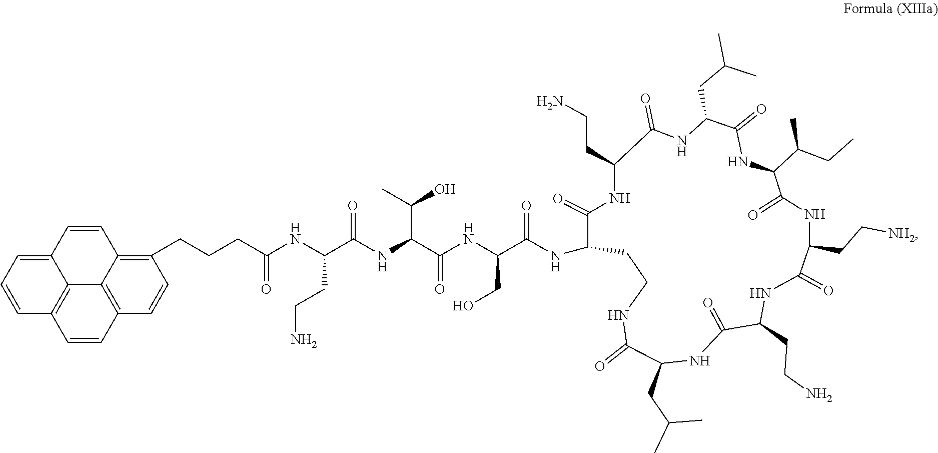 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)CCCc2ccc3ccc4cccc5ccc2c3c45)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative 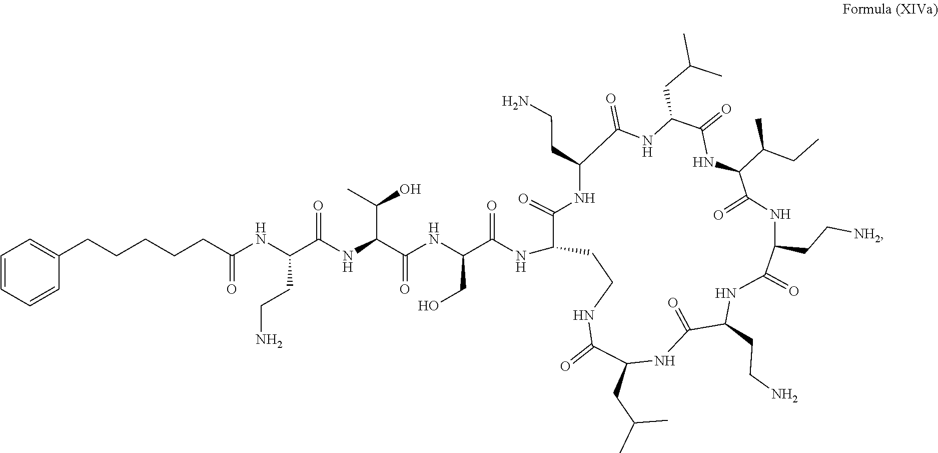 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)CCCCCc2ccccc2)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative thereof, 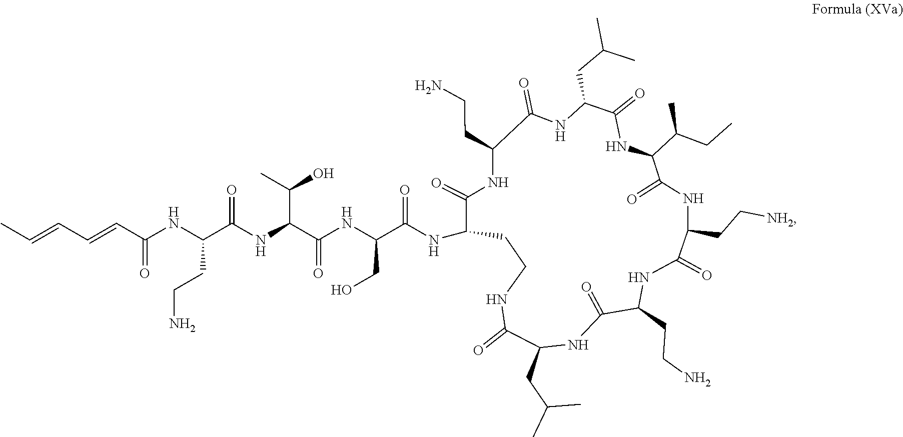 [SMILES: C/C=C/C=C/C(=O)N[C@@H](CCN)C(=O)N[C@H](C(=O)N[C@H](CO)C(=O)N[C@H]1CCNC(=O)[C@H](CC(C)C)NC(=O)[C@H](CCN)NC(=O)[C@H](CCN)NC(=O)[C@H]([C@@H](C)CC)NC(=O)[C@@H](CC(C)C)NC(=O)[C@H](CCN)NC1=O)[C@@H](C)O]
or a racemate, an enantiomer, a diastereomer, a pharmaceutically acceptable salt, or a derivative 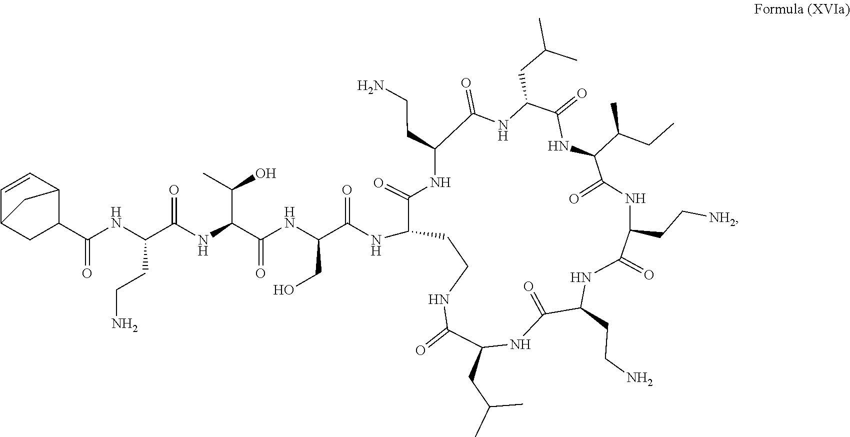 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)C2CC3C=CC2C3)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative thereof, 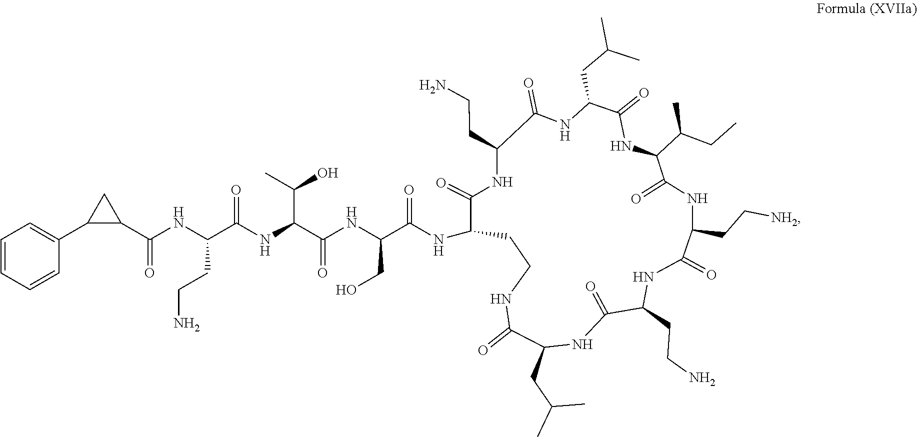 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)C2CC2c2ccccc2)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative 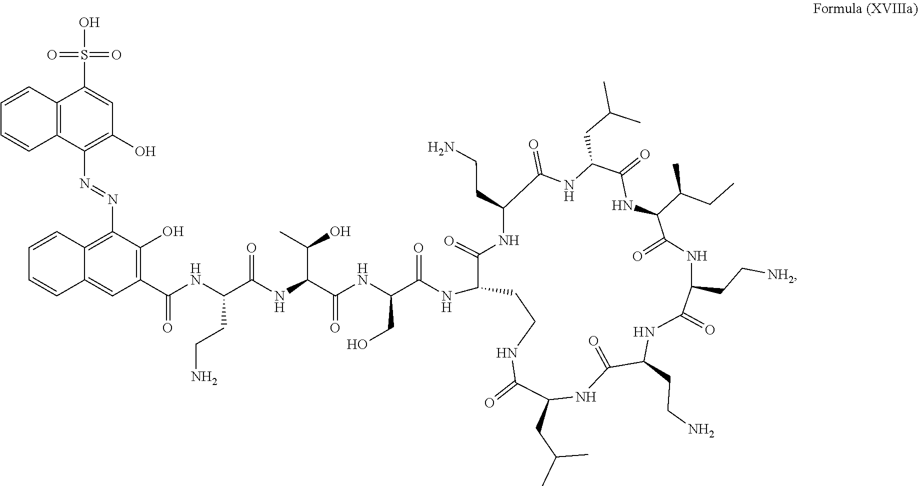 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)c2cc3ccccc3c(/N=N/c3c(O)cc(S(=O)(=O)O)c4ccccc34)c2O)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative thereof, 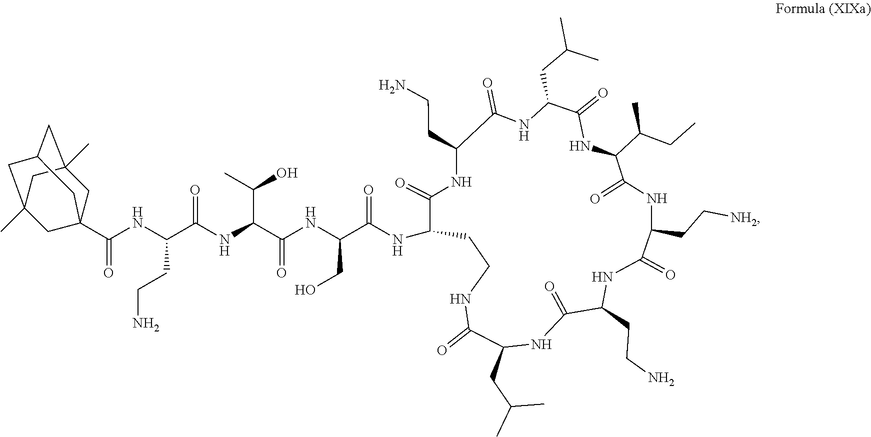 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)C23CC4CC(C)(CC(C)(C4)C2)C3)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative thereof, and 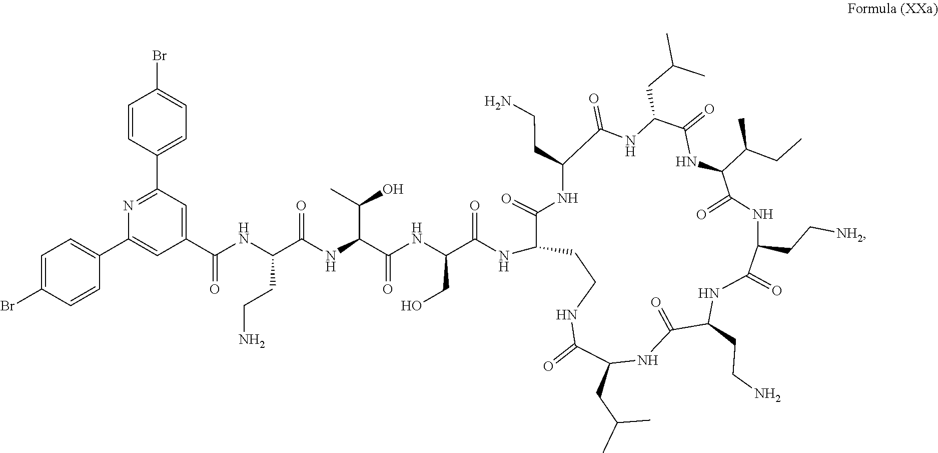 [SMILES: CC[C@H](C)[C@@H]1NC(=O)[C@@H](CC(C)C)NC(=O)[C@H](CCN)NC(=O)[C@@H](NC(=O)[C@@H](CO)NC(=O)[C@@H](NC(=O)[C@H](CCN)NC(=O)c2cc(-c3ccc(Br)cc3)nc(-c3ccc(Br)cc3)c2)[C@@H](C)O)CCNC(=O)[C@H](CC(C)C)NC(=O)[C@H](CCN)NC(=O)[C@H](CCN)NC1=O]
or a racemate, an enantiomer, a diastereomer, a pharmaceutically acceptable salt, or a derivative thereof.
